(19) United States
(12) Patent Application Publication
(10) Pub. No.: US 2022/0257789 A1
(43) Pub. Date: Aug. 18, 2022

(54) ANTI-TISSUE FACTOR ANTIBODY-DRUG CONJUGATES AND RELATED METHODS

(71) Applicants: Iconic Therapeutics, Inc., South San Francisco, CA (US); Zymeworks Inc., Vancouver (CA)

(72) Inventors: Jan-Willem Theunissen, South San Francisco, CA (US); Allen G. Cai, South San Francisco, CA (US); Thi-Sau Migone, South San Francisco, CA (US)

(21) Appl. No.: 17/624,541

(22) PCT Filed: Jul. 2, 2020
(86) PCT No.: PCT/US2020/040711

§ 371 Date: Jan. 3, 2022

Related U.S. Application Data
(60) Provisional application No. 62/870,644, filed on Jul. 3, 2019.

(51) Int. Cl. A61K 47/68; A61K 47/54

(52) U.S. Cl. CPC A61K 47/6849; A61K 47/545; A61K 47/6817

(57) ABSTRACT

Provided herein are antibodies that specifically bind to human tissue factor (TF), anti-TF antibody-drug conjugates (ADCs), and compositions comprising the antibodies or ADCs. Also provided herein are methods of making and using the antibodies or ADCs, such as therapeutic and diagnostic methods.

### CROSS REFERENCE TO RELATED APPLICATIONS

This application claims priority to and the benefit of U.S. Provisional Patent Application No. 62/870,644, filed on Jul. 3, 2019, the entire contents of which are incorporated by reference herein for all purposes.

### BACKGROUND

Blood coagulation involves a complex set of processes that result in blood clotting. Tissue factor (TF) plays an important role in these coagulation processes. TF is a cell surface receptor for the serine protease factor VIIa (FVIIa). The TF/FVIIa complex catalyzes conversion of the inactive protease factor X (FX) into the active protease factor Xa (FXa). FXa and its co-factor FVa form the prothrombinase complex, which generates thrombin from prothrombin. Thrombin converts soluble fibrinogen into insoluble strands of fibrin and catalyzes many other coagulation-related processes.

TF is over-expressed on multiple types of solid tumors. In cancer, TF/FVIIa signaling can support angiogenesis, tumor progression, and metastasis.

### SUMMARY

Provided herein are anti-TF antibody-drug conjugates, and related methods.

Provided herein is an antibody-drug conjugate comprising:
a. an antigen binding protein (Ab) which binds to the extracellular domain of human Tissue Factor (TF) (SEQ ID NO:810), wherein the Ab comprises a VH-CDR1, a VH-CDR2, a VH-CDR3, a VL-CDR1, a VL-CDR2, and a VL-CDR3, wherein
   i. the VH-CDR1 comprises SEQ ID NO: 872, the VH-CDR2 comprises SEQ ID NO: 873, the VH-CDR3 comprises SEQ ID NO: 874, the VL-CDR1 comprises SEQ ID NO: 875, the VL-CDR2 comprises SEQ ID NO: 876, and the VL-CDR3 comprises SEQ ID NO: 877,
   ii. the VH-CDR1, VH-CDR2, VH-CDR3, VL-CDR1, VL-CDR2, and VL-CDR3 are from the antibody designated 25A3,
   iii. the VH-CDR1, VH-CDR2, VH-CDR3, VL-CDR1, VL-CDR2, and VL-CDR3 are from the antibody designated 25A,
   iv. the VH-CDR1, VH-CDR2, VH-CDR3, VL-CDR1, VL-CDR2, and VL-CDR3 are from the antibody designated 25A5,
   v. the VH-CDR1, VH-CDR2, VH-CDR3, VL-CDR1, VL-CDR2, and VL-CDR3 are from the antibody designated 25A5-T, or
   vi. the VH-CDR1, VH-CDR2, VH-CDR3, VL-CDR1, VL-CDR2, and VL-CDR3 are from the antibody designated 25G1;

and
b. one or more linker-toxin moieties represented by Formula IV:

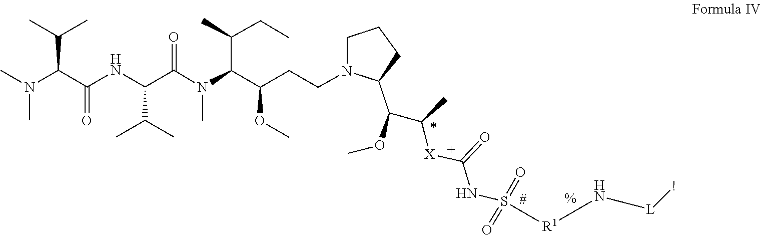

wherein:

X is *—C(O)NHCH(CH₂(R²))—⁺, wherein * and + represent the respective points of attachment as indicated in Formula IV, or X is absent;
L is a linker;
! represents the point of attachment of L to the Ab, where L is attached to the Ab through a covalent bond;
R¹ is selected from the group consisting of:

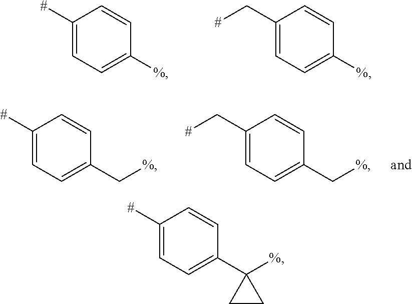

wherein # and % represent the respective points of attachment as indicated in Formula IV; and

R² is phenyl.

In some embodiments, R¹ is selected from the group consisting of:

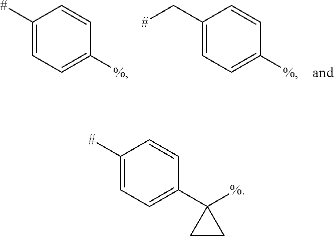

In some embodiments, X is absent.

In some embodiments, the linker-toxin moiety of Formula IV is represented by Formula V:

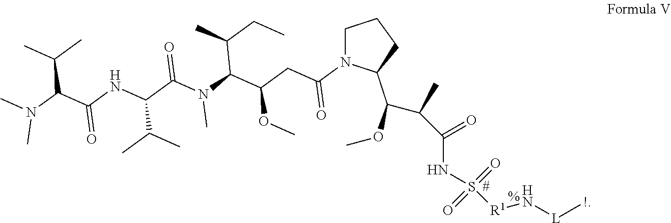

In some embodiments, R¹ is selected from the group consisting of:

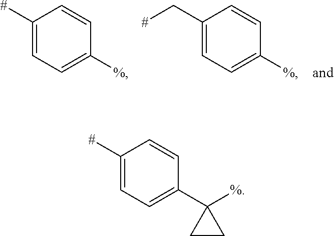

In some embodiments, R¹ is selected from the group consisting of:

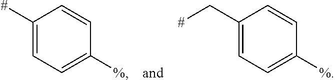

In some embodiments, R¹ is:

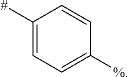

In some embodiments, L is a cleavable linker.

In some embodiments, L is a peptide-containing linker.

In some embodiments, L is a protease-cleavable linker.

In some embodiments, L is a linker selected from one of N-(β-maleimidopropyloxy)-N-hydroxy succinimide ester (BMPS), N-(ε-maleimidocaproyloxy) succinimide ester (EMCS), N-[γ-maleimidobutyryloxy]succinimide ester (GMBS), 1,6-hexane-bis-vinylsulfone (HBVS), succinimidyl 4-(N-maleimidomethyl)cyclohexane-1-carboxy-(6-amidocaproate) (LC-SMCC), m-maleimidobenzoyl-N-hydroxysuccinimide ester (MBS), 4-(4-N-Maleimidophenyl)butyric acid hydrazide (MPBH), succinimidyl 3-(bromoacetamido)propionate (SBAP), succinimidyl iodoacetate (SIA), succinimidyl (4-iodoacetyl)aminobenzoate (STAB), N-succinimidyl-3-(2-pyridyldithio) propionate (SPDP), N-succinimidyl-4-(2-pyridylthio)pentanoate (SPP), succinimidyl 4-(N-maleimidomethyl)cyclohexane-1-carboxylate (SMCC), succinimidyl 4-(p-maleimidophenyl)butyrate (SMPB), succinimidyl 6-[(β-maleimidopropionamido)hexanoate] (SMPH), iminothiolane (IT), sulfo-EMCS, sulfo-GMBS, sulfo-KMUS, sulfo-MBS, sulfo-SIAB, sulfo-SMCC, sulfo-SMPB, and succinimidyl-(4-vinylsulfone)benzoate (SVSB).

In some embodiments, L comprises a poly(ethylene)glycol chain of the formula:

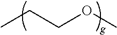

wherein g is an integer from 1-20.

In some embodiments, g is 3.

Provided herein is an antibody-drug conjugate of Formula VI:

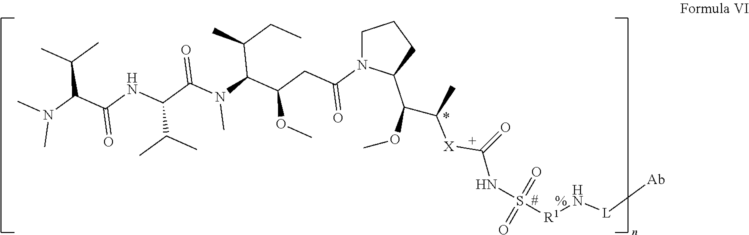

wherein:

Ab represents a tissue factor (TF) antibody;

n is an integer greater than or equal to 1;

X is *—C(O)NHCH(CH₂(R²))—⁺, wherein * and + represent the respective points of attachment as indicated in Formula VI, or X is absent;

L is a linker;

R¹ is selected from the group consisting of:

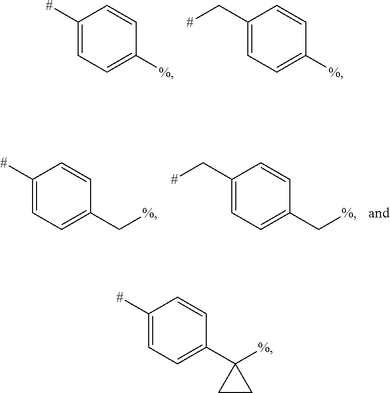

wherein # and % represent the respective points of attachment as indicated in Formula VI; and

R² is phenyl; and wherein
the Ab comprises a VH-CDR1, a VH-CDR2, a VH-CDR3, a VL-CDR1, a VL-CDR2, and a VL-CDR3, wherein
   i. the VH-CDR1 comprises SEQ ID NO: 872, the VH-CDR2 comprises SEQ ID NO: 873, the VH-CDR3 comprises SEQ ID NO: 874, the VL-CDR1 comprises SEQ ID NO: 875, the VL-CDR2 comprises SEQ ID NO: 876, and the VL-CDR3 comprises SEQ ID NO: 877,
   ii. the VH-CDR1, VH-CDR2, VH-CDR3, VL-CDR1, VL-CDR2, and VL-CDR3 are from the antibody designated 25A3,
   iii. the VH-CDR1, VH-CDR2, VH-CDR3, VL-CDR1, VL-CDR2, and VL-CDR3 are from the antibody designated 25A,
   iv. the VH-CDR1, VH-CDR2, VH-CDR3, VL-CDR1, VL-CDR2, and VL-CDR3 are from the antibody designated 25A5,
   v. the VH-CDR1, VH-CDR2, VH-CDR3, VL-CDR1, VL-CDR2, and VL-CDR3 are from the antibody designated 25A5-T, or
   vi. the VH-CDR1, VH-CDR2, VH-CDR3, VL-CDR1, VL-CDR2, and VL-CDR3 are from the antibody designated 25G1.

In some embodiments, R¹ is selected from the group consisting of:

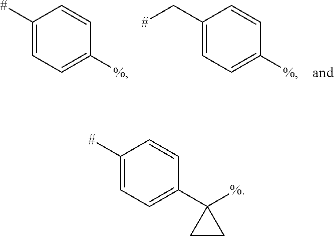

In some embodiments, X is absent.

In some embodiments, R¹ is selected from the group consisting of:

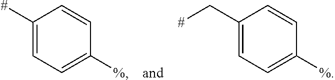

In some embodiments, R¹ is:

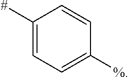

In some embodiments, L is a cleavable linker.

In some embodiments, L is a peptide-containing linker.

In some embodiments, L is a protease-cleavable linker.

In some embodiments, L is a linker selected from one of N-(β-maleimidopropyloxy)-N-hydroxy succinimide ester (BMPS), N-(ε-maleimidocaproyloxy) succinimide ester (EMCS), N-[γ-maleimidobutyryloxy]succinimide ester (GMBS), 1,6-hexane-bis-vinylsulfone (HBVS), succinimidyl 4-(N-maleimidomethyl)cyclohexane-1-carboxy-(6-amidocaproate) (LC-SMCC), m-maleimidobenzoyl-N-hydroxysuccinimide ester (MBS), 4-(4-N-Maleimidophenyl)butyric acid hydrazide (MPBH), succinimidyl 3-(bromoacetamido)propionate (SBAP), succinimidyl iodoacetate (SIA), succinimidyl (4-iodoacetyl)aminobenzoate (STAB), N-succinimidyl-3-(2-pyridyldithio) propionate (SPDP), N-succinimidyl-4-(2-pyridylthio)pentanoate (SPP), succinimidyl 4-(N-maleimidomethyl)cyclohexane-1-carboxylate (SMCC), succinimidyl 4-(p-maleimidophenyl)butyrate (SMPB), succinimidyl 6-[(β-maleimidopropionamido)hexanoate] (SMPH), iminothiolane (IT), sulfo-EMCS, sulfo-GMBS, sulfo-KMUS, sulfo-MBS, sulfo-SIAB, sulfo-SMCC, sulfo-SMPB, and succinimidyl-(4-vinylsulfone)benzoate (SVSB).

In some embodiments, L comprises a poly(ethylene)glycol chain of the formula:

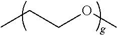

wherein g is an integer from 1-20.

In some embodiments, g is 3.

In some embodiments, L is represented by Formula VII:

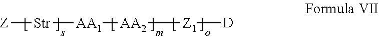

wherein:
Z represents a functional group that binds to a target group of the TF antibody;
   D represents the point of attachment to the amino group as indicated in Formula VI;
   Str is a stretcher;
   AA₁ and AA₂ are each independently an amino acid, wherein AA₁-[AA₂]ₘ forms a protease cleavage site;
   X₁ is a self-immolative group;
   s is an integer selected from 0 and 1;
   m is an integer selected from the group consisting of 1, 2, 3, and 4;
   o is an integer selected from 0, 1, and 2.

In some embodiments, n is an integer selected from the group consisting of 1, 2, 3, 4, and 5.

In some embodiments, [Str]ₛ is selected from the group consisting of alkylene, stretchers based on aliphatic acids, stretchers based on aliphatic diacids, stretchers based on aliphatic amines and stretchers based on aliphatic diamines.

In some embodiments, [Str]ₛ is selected from the group consisting of diglycolate-based stretchers, malonate-based stretchers, caproate-based stretchers and caproamide-based stretchers.

In some embodiments, [Str]ₛ is selected from the group consisting of glycine-based stretchers, polyethylene glycol-based stretchers, and monomethoxy polyethylene glycol-based stretchers.

In some embodiments, [Str]ₛ is:

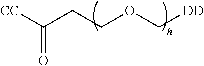

wherein
h is an integer from 1-20,
CC refers to the point of attachment to AA₁; and
DD refers to the point of attachment to Z.

In some embodiments, [Str]ₛ is selected from:

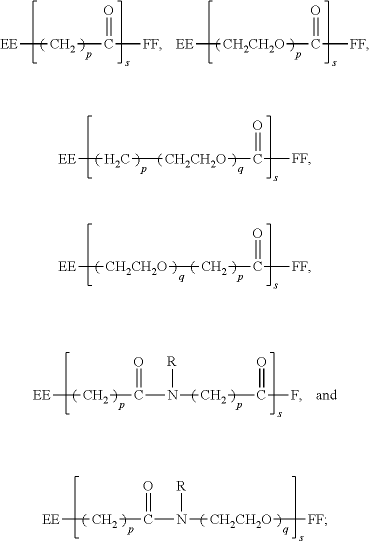

wherein:
EE and FF represent the points of attachment to Z and AA₁, respectively;
   R is selected from hydrogen and C₁-C₆ alkyl;
   each occurrence of p is independently an integer from 2 to 10; and
   each occurrence of q is independently an integer from 1 to 10.

In some embodiments, [Str]ₛ is selected from the group consisting of:

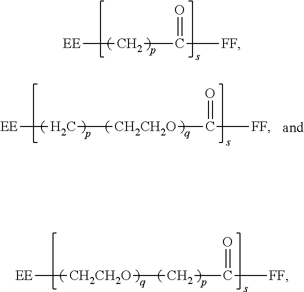

wherein:

EE and FF represent the points of attachment to Z and AA₁, respectively;

each occurrence of p is independently an integer from 2 to 10; and

each occurrence of q is independently an integer from 1 to 10.

In some embodiments, [Str]ₛ is selected from:

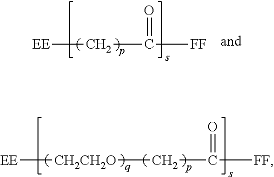

wherein:

EE and FF represent the points of attachment to Z and AA₁, respectively;

each occurrence of p is independently an integer from 2 to 6, and

q is an integer from 2 to 8.

In some embodiments, AA₁-[AA₂]ₘ is selected from Val-Lys, Ala-Lys, Phe-Lys, Val-Cit, Phe-Cit, Leu-Cit, Ile-Cit, Trp-Cit, Phe-Arg, Ala-Phe, Val-Ala, Met-Lys, Asn-Lys, Ile-Pro, Ile-Val, Asp-Val, His-Val, Met-(D)Lys, Asn-(D)Lys, Val-(D)Asp, NorVal-(D)Asp, Ala-(D)Asp, Me₃Lys-Pro, PhenylGly-(D)Lys, Met-(D)Lys, Asn-(D)Lys, Pro-(D)Lys, Met-(D)Lys, Met-Cit-Val, Gly-Cit-Val, (D)Phe-Phe-Lys, (D)Ala-Phe-Lys, Gly-Phe-Leu-Gly, and Ala-Leu-Ala-Leu.

In some embodiments, m is selected from 1, 2 and 3.

In some embodiments, m is 1.

In some embodiments, AA₁-[AA₂]ₘ is a dipeptide selected from Val-Lys, Ala-Lys, Phe-Lys, Val-Cit, Phe-Cit, Leu-Cit, Ile-Cit and Trp-Cit.

In some embodiments, each X₁ is independently selected from p-aminobenzyloxycarbonyl (PABC), p-aminobenzyl ether (PABE) and methylated ethylene diamine (MED).

In some embodiments, s is 1 and h is 3.

In some embodiments, s is 1.

In some embodiments, o is 0.

Provided herein is an antibody drug-conjugate comprising a linker-toxin moiety of the Formula VIII:

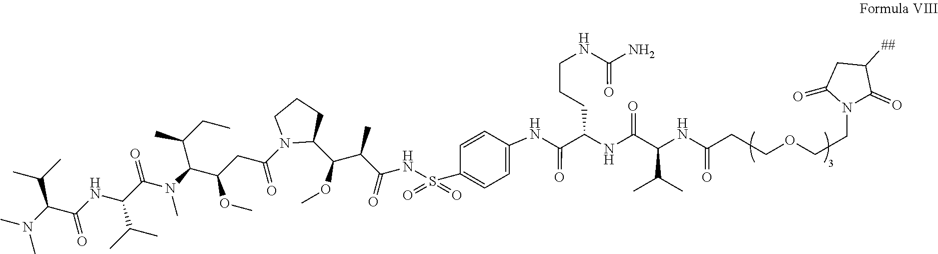

wherein ## represents the point of attachment of the linker-toxin moiety to the TF antibody and the linker-toxin moiety is attached to the TF antibody through a covalent bond.

Provided herein is an antibody-drug conjugate of Formula IX:

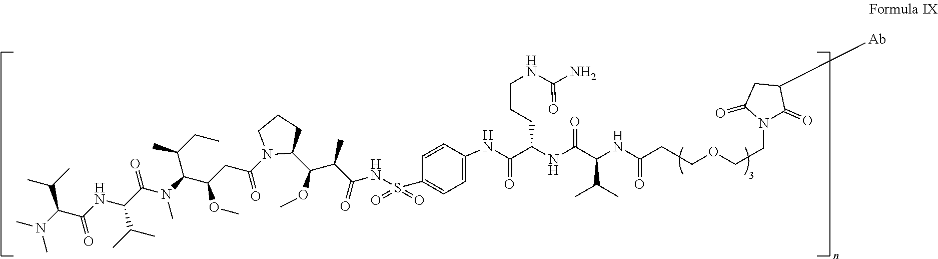

wherein:
Ab is a tissue factor (TF) antibody, wherein the Ab comprises a VH-CDR1, a VH-CDR2, a VH-CDR3, a VL-CDR1, a VL-CDR2, and a VL-CDR3, wherein
   i. the VH-CDR1 comprises SEQ ID NO: 872, the VH-CDR2 comprises SEQ ID NO: 873, the VH-CDR3 comprises SEQ ID NO: 874, the VL-CDR1 comprises SEQ ID NO: 875, the VL-CDR2 comprises SEQ ID NO: 876, and the VL-CDR3 comprises SEQ ID NO: 877,
   ii. the VH-CDR1, VH-CDR2, VH-CDR3, VL-CDR1, VL-CDR2, and VL-CDR3 are from the antibody designated 25A3,
   iii. the VH-CDR1, VH-CDR2, VH-CDR3, VL-CDR1, VL-CDR2, and VL-CDR3 are from the antibody designated 25A,
   iv. the VH-CDR1, VH-CDR2, VH-CDR3, VL-CDR1, VL-CDR2, and VL-CDR3 are from the antibody designated 25A5,
   v. the VH-CDR1, VH-CDR2, VH-CDR3, VL-CDR1, VL-CDR2, and VL-CDR3 are from the antibody designated 25A5-T, or
   vi. the VH-CDR1, VH-CDR2, VH-CDR3, VL-CDR1, VL-CDR2, and VL-CDR3 are from the antibody designated 25G1,

n is an integer greater than or equal to 1, and

the succinimidyl group is attached to the Ab through a covalent bond.

In some embodiments, n is selected from the group consisting of 1, 2, 3, 4, and 5.

In some embodiments, n is selected from the group consisting of 2, 3, and 4.

Provided herein is an antibody-drug conjugate comprising a linker as represented by Formula X:

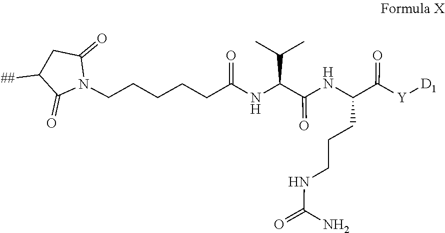

wherein:
## is the point of attachment to the antibody and the succinimidyl group is attached to the antibody through a covalent bond;
Y is one or more additional linker components, or is absent; and
D₁ is the point of attachment to a cytotoxic agent, and wherein
the Ab comprises a VH-CDR1, a VH-CDR2, a VH-CDR3, a VL-CDR1, a VL-CDR2, and a VL-CDR3, wherein
   i. the VH-CDR1 comprises SEQ ID NO: 872, the VH-CDR2 comprises SEQ ID NO: 873, the VH-CDR3 comprises SEQ ID NO: 874, the VL-CDR1 comprises SEQ ID NO: 875, the VL-CDR2 comprises SEQ ID NO: 876, and the VL-CDR3 comprises SEQ ID NO: 877,
   ii. the VH-CDR1, VH-CDR2, VH-CDR3, VL-CDR1, VL-CDR2, and VL-CDR3 are from the antibody designated 25A3,
   iii. the VH-CDR1, VH-CDR2, VH-CDR3, VL-CDR1, VL-CDR2, and VL-CDR3 are from the antibody designated 25A,
   iv. the VH-CDR1, VH-CDR2, VH-CDR3, VL-CDR1, VL-CDR2, and VL-CDR3 are from the antibody designated 25A5,
   v. the VH-CDR1, VH-CDR2, VH-CDR3, VL-CDR1, VL-CDR2, and VL-CDR3 are from the antibody designated 25A5-T, or
   vi. the VH-CDR1, VH-CDR2, VH-CDR3, VL-CDR1, VL-CDR2, and VL-CDR3 are from the antibody designated 25G1.

Provided herein is an antibody-drug conjugate comprising a linker as represented by Formula XI:

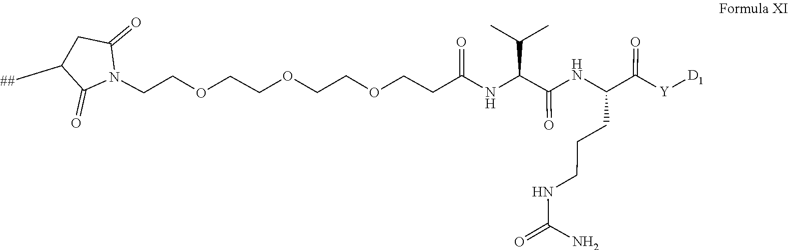

wherein:
## is the point of attachment to the antibody and the succinimidyl group is attached to the antibody through a covalent bond;
Y is one or more additional linker components, or is absent; and
D₁ is the point of attachment to a cytotoxic agent, and wherein
   the Ab comprises a VH-CDR1, a VH-CDR2, a VH-CDR3, a VL-CDR1, a VL-CDR2, and a VL-CDR3, wherein
      i. the VH-CDR1 comprises SEQ ID NO: 872, the VH-CDR2 comprises SEQ ID NO: 873, the VH-CDR3 comprises SEQ ID NO: 874, the VL-CDR1 comprises SEQ ID NO: 875, the VL-CDR2 comprises SEQ ID NO: 876, and the VL-CDR3 comprises SEQ ID NO: 877,
   ii. the VH-CDR1, VH-CDR2, VH-CDR3, VL-CDR1, VL-CDR2, and VL-CDR3 are from the antibody designated 25A3,
   iii. the VH-CDR1, VH-CDR2, VH-CDR3, VL-CDR1, VL-CDR2, and VL-CDR3 are from the antibody designated 25A,
   iv. the VH-CDR1, VH-CDR2, VH-CDR3, VL-CDR1, VL-CDR2, and VL-CDR3 are from the antibody designated 25A5,
   v. the VH-CDR1, VH-CDR2, VH-CDR3, VL-CDR1, VL-CDR2, and VL-CDR3 are from the antibody designated 25A5-T, or
   vi. the VH-CDR1, VH-CDR2, VH-CDR3, VL-CDR1, VL-CDR2, and VL-CDR3 are from the antibody designated 25G1.

In some embodiments, the cytotoxic agent is selected from the group consisting of a diagnostic agent, a metal chelator, an enzyme, a fluorescent compound, a bioluminescent compound, or a chemiluminescent compound.

In some embodiments, the cytotoxic agent is a cytotoxic payload having an improved safety profile.

In some embodiments, the Ab comprises:
a. a VH sequence that is SEQ ID NO: 868 and a VL sequence that is SEQ ID NO: 869,
b. a VH that is SEQ ID NO: 151 and a VL sequence that is SEQ ID NO: 152,
c. a VH sequence that is SEQ ID NO: 113 and a VL sequence that is SEQ ID NO: 114,
d. a VH sequence that is SEQ ID NO: 189 and a VL sequence that is SEQ ID NO: 190,
e. a VH sequence that is SEQ ID NO: 836 and a VL sequence that is SEQ ID NO: 837, or
f. a VH sequence that is SEQ ID NO: 265 and a VL sequence that is SEQ ID NO: 266.

In some embodiments, the Ab comprises:

a. a heavy chain sequence that is

QVQLVQSGAEVKKPGASVKVSCKASGYTFDx[V/A]YGISWVRQAPGQ
GLEWMGWIAPYx[N/S]GNTNYAQKLQGRVTMTTDTSTSTAYMELRSL
RSDDTAVYYCARDAGTYSPFGYGMDVWGQGTTVTVSSASTKGPSVF
PLAPSSKSTSGGTAALGCLVKDYFPEPVTVSWNSGALTSGVHTFPAVL
QSSGLYSLSSVVTVPSSSLGTQTYICNVNHKPSNTKVDKRVEPKSCDK
THTCPPCPAPELLGGPSVFLFPPKPKDTLMISRTPEVTCVVVDVSHEDP
EVKFNWYVDGVEVHNAKTKPREEQYNSTYRVVSVLTVLHQDWLNG
KEYKCKVSNKALPAPIEKTISKAKGQPREPQVYTLPPSREEMTKNQVS
LTCLVKGFYPSDIAVEWESNGQPENNYKTTPPVLDSDGSFFLYSKLTV
DKSRWQQGNVFSCSVMHEALHNHYTQKSLSLSPG

and a light chain sequence that is

DIQMTQSPSTLSASVGDRVTITCx[R/Q]ASx[Q/E]SIx[S/N]x[S/N]
WLAWYQQKPGKAPKLLIYKAx[S/Y]x[S/N]LEx[S/Y]GVPSRFSGSGS
GTEFTLTISSLQPDDFATYYCQx[Q/L]FQx[S/K]LPPFTFGGGTKVEIK
RTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSF
NRGEC,

b. a heavy chain sequence that is

QVQLVQSGAEVKKPGASVKVSCKASGYTFDVYGISWVRQAPGQGLE
WMGWIAPYSGNTNYAQKLQGRVTMTTDTSTSTAYMELRSLRSDDTA
VYYCARDAGTYSPFGYGMDVWGQGTTVTVSSASTKGPSVFPLAPSS
KSTSGGTAALGCLVKDYFPEPVTVSWNSGALTSGVHTFPAVLQSSG
LYSLSSVVTVPSSSLGTQTYICNVNHKPSNTKVDKRVEPKSCDKTH
TCPPCPAPELLGGPSVFLFPPKPKDTLMISRTPEVTCVVVDVSHED
PEVKFNWYVDGVEVHNAKTKPREEQYNSTYRVVSVLTVLHQDWLNG
KEYKCKVSNKALPAPIEKTISKAKGQPREPQVYTLPPSREEMTKNQ
VSLTCLVKGFYPSDIAVEWESNGQPENNYKTTPPVLDSDGSFFLYS
KLTVDKSRWQQGNVFSCSVMHEALHNHYTQKSLSLSPG

and a light chain sequence that is

DIQMTQSPSTLSASVGDRVTITCQASQSINNWLAWYQQKPGKAPKLLIYKA
YNLESGVPSRFSGSGSGTEFTLTISSLQPDDFATYYCQLFQSLPPFTFGGG
TKVEIKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLSS
PVTKSFNRGEC,

c. a heavy chain sequence that is

QVQLVQSGAEVKKPGASVKVSCKASGYTFDVYGISWVRQAPGQGLEWMGWI
APYSGNTNYAQKLQGRVTMTTDTSTSTAYMELRSLRSDDTAVYYCARDAGT
YSPFGYGMDVWGQGTTVTVSSASTKGPSVFPLAPSSKSTSGGTAALGCLVK
DYFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPKD
TLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDG
SFFLYSKLTVDKSRWQQGNVFSCSVMHEALHNHYTQKSLSLSPG

and a light chain sequence that is

DIQMTQSPSTLSASVGDRVTITCRASQSISSWLAWYQQKPGKAPKLLIYKA
SSLESGVPSRFSGSGSGTEFTLTISSLQPDDFATYYCQQFQSLPPFTFGGG
TKVEIKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLSS
PVTKSFNRGEC,

d. a heavy chain sequence that is

QVQLVQSGAEVKKPGASVKVSCKASGYTFDAYGISWVRQAPGQGLEWMGWI
APYSGNTNYAQKLQGRVTMTTDTSTSTAYMELRSLRSDDTAVYYCARDAGT
YSPFGYGMDVWGQGTTVTVSSASTKGPSVFPLAPSSKSTSGGTAALGCLVK
DYFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPKD
TLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDG
SFFLYSKLTVDKSRWQQGNVFSCSVMHEALHNHYTQKSLSLSPG

and a light chain sequence that is

DIQMTQSPSTLSASVGDRVTITCRASESISNWLAWYQQKPGKAPKLLIYKA
YSLEYGVPSRFSGSGSGTEFTLTISSLQPDDFATYYCQQFQKLPPFTFGGG
TKVEIKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLSS
PVTKSFNRGEC,

e. a heavy chain sequence that is

QVQLVQSGAEVKKPGASVKVSCKASGYTFRSYGISWVRQAPGQGLEWMGWV
APYSGNTNYAQKLQGRVTMTTDTSTSTAYMELRSLRSDDTAVYYCARDAGT
YSPYGYGMDVWGQGTTVTVSSASTKGPSVFPLAPSSKSTSGGTAALGCLVK
DYFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPKD
TLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDG
SFFLYSKLTVDKSRWQQGNVFSCSVMHEALHNHYTQKSLSLSPG,

and a light chain sequence that is

DIQMTQSPSTLSASVGDRVTITCRASHSIDSWLAWYQQKPGKAPKLLIYKA
SYLESGVPSRFSGSGSGTEFTLTISSLQPDDFATYYCQLFQSLPPFTFGGG
TKVEIKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLSS
PVTKSFNRGEC,

or

f. a heavy chain sequence that is

QVQLVQSGAEVKKPGASVKVSCKASGYTFDVYGISWVRQAPGQGLEWMGWI
APYSGNTNYAQKLQGRVTMTTDTSTSTAYMELRSLRSDDTAVYYCARDAGT
YSPFGYGMDVWGQGTTVTVSSASTKGPSVFPLAPSSKSTSGGTAALGCLVK
DYFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPKD
TLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDG
SFFLYSKLTVDKSRWQQGNVFSCSVMHEALHNHYTQKSLSLSPG

and a light chain sequence that is

DIQMTQSPSTLSASVGDRVTITCRASESISNWLAWYQQKPGKAPKLLIYKA
YSLEYGVPSRFSGSGSGTEFTLTISSLQPDDFATYYCQQFQKLPPFTFGGG
TKVEIKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLSS
PVTKSFNRGEC.

Provided herein is an antibody-drug conjugate of Formula IX:

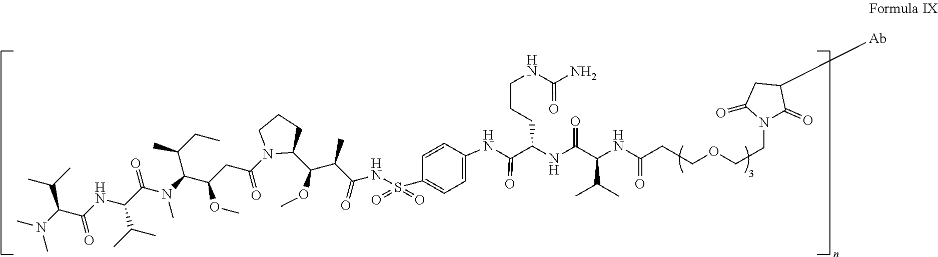

wherein:
Ab is a tissue factor (TF) antibody, wherein the Ab comprises a VH-CDR1, a VH-CDR2, a VH-CDR3, a VL-CDR1, a VL-CDR2, and a VL-CDR3 from the antibody designated 25A3, and

n is an integer greater than or equal to 1.

In some embodiments, n is selected from the group consisting of 1, 2, 3, 4, and 5.

In some embodiments, n is selected from the group consisting of 2, 3, and 4.

In some embodiments, the Ab comprises a VH sequence that is SEQ ID NO: 151 and a VL sequence that is SEQ ID NO: 152.

In some embodiments, the Ab comprises a full heavy chain sequence that is

QVQLVQSGAEVKKPGASVKVSCKASGYTFDVYGISWVRQAPGQGLEWMGW
IAPYSGNTNYAQKLQGRVTMTTDTSTSTAYMELRSLRSDDTAVYYCARDA
GTYSPFGYGMDVWGQGTTVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
P

G and a light chain sequence that is

DIQMTQSPSTLSASVGDRVTITCQASQSINNWLAWYQQKPGKAPKLLIYK
AYNLESGVPSRFSGSGSGTEFTLTISSLQPDDFATYYCQLFQSLPPFTFG
GGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC.

Provided herein is an antibody-drug conjugate of Formula IX:

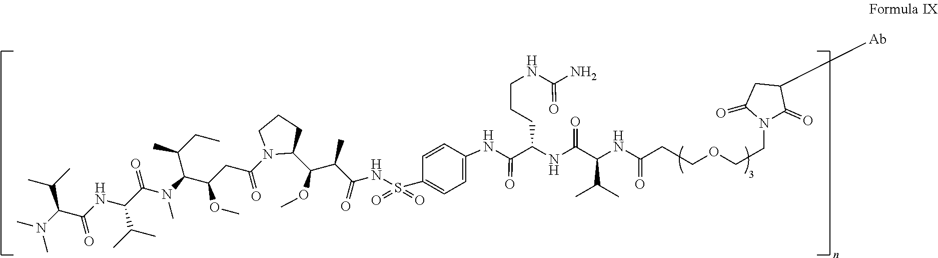

wherein:
Ab is a tissue factor (TF) antibody, wherein the Ab comprises a heavy chain sequence that is

QVQLVQSGAEVKKPGASVKVSCKASGYTFDVYGISWVRQAPGQGLEWMGW
IAPYSGNTNYAQKLQGRVTMTTDTSTSTAYMELRSLRSDDTAVYYCARDA
GTYSPFGYGMDVWGQGTTVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PG

and a light chain sequence that is

DIQMTQSPSTLSASVGDRVTITCQASQSINNWLAWYQQKPGKAPKLLIYKA
YNLESGVPSRFSGSGSGTEFTLTISSLQPDDFATYYCQLFQSLPPFTFGGG
TKVEIKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLSS
PVTKSFNRGEC,

and

n is an integer greater than or equal to 1.

In some embodiments, n is selected from the group consisting of 1, 2, 3, 4, and 5.

In some embodiments, n is selected from the group consisting of 2, 3, and 4.

Provided herein is an antibody-drug conjugate comprising an antibody (Ab) and one or more linker-toxins of the following structure:

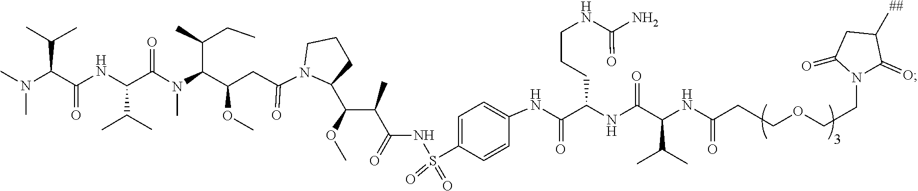

wherein:

Ab is a tissue factor (TF) antibody, wherein the Ab comprises a VH-CDR1, a VH-CDR2, a VH-CDR3, a VL-CDR1, a VL-CDR2, and a VL-CDR3 from the antibody designated 25A3;

the one or more linker-toxins are attached to the Ab through a covalent bond; and

## represents a point of attachment of the linker-toxin to the Ab.

Provided herein is an antibody-drug conjugate composition comprising the antibody-drug conjugate disclosed herein, wherein the composition comprises a multiplicity of drug-antibody ratio (DAR) species, wherein the average DAR of the composition is 2-4.

Provided herein is an antibody-drug conjugate comprising an antibody (Ab) and one or more linker-toxins of the following structure:

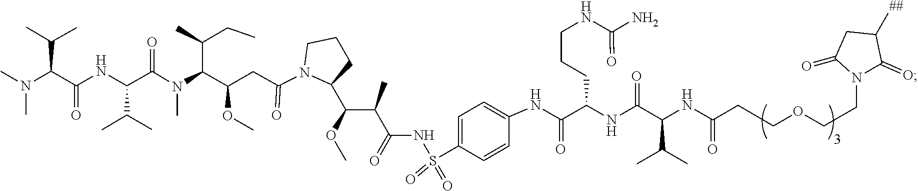

wherein:
Ab is a tissue factor (TF) antibody, wherein the Ab comprises a heavy chain sequence that is

QVQLVQSGAEVKKPGASVKVSCKASGYTFDVYGISWVRQAPGQGLEWMGWI
APYSGNTNYAQKLQGRVTMTTDTSTSTAYMELRSLRSDDTAVYYCARDAGT
YSPFGYGMDVWGQGTTVTVSSASTKGPSVFPLAPSSKSTSGGTAALGCLVK
DYFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPKD
TLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDG
SFFLYSKLTVDKSRWQQGNVFSCSVMHEALHNHYTQKSLSLSPG

and a light chain sequence that is

DIQMTQSPSTLSASVGDRVTITCQASQSINNWLAWYQQKPGKAPKLLIYKA
YNLESGVPSRFSGSGSGTEFTLTISSLQPDDFATYYCQLFQSLPPFTFGGG
TKVEIKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLSS
PVTKSFNRGEC,

and
the one or more linker-toxins are attached to the Ab through a covalent bond; and
## represents a point of attachment of the linker-toxin to the Ab.

Provided herein is an antibody-drug conjugate composition comprising the antibody-drug conjugate disclosed herein, wherein the composition comprises a multiplicity of drug-antibody ratio (DAR) species, wherein the average DAR of the composition is 2-4.

In some embodiments, the Ab is multispecific.

In some embodiments, the Ab is a Fab, Fab′, F(ab′)2, Fv, scFv, (scFv)2, single chain antibody molecule, dual variable domain antibody, single variable domain antibody, linear antibody, or V domain antibody.

In some embodiments, the antibody comprises a scaffold, optionally wherein the scaffold is Fc, optionally human Fc.

In some embodiments, the antibody comprises a heavy chain constant region of a class selected from IgG, IgA, IgD, IgE, and IgM.

In some embodiments, the antibody comprises a heavy chain constant region of the class IgG, wherein the heavy chain constant region is from a subclass selected from IgG1, IgG2, IgG3, and IgG4.

In some embodiments, the antibody comprises a heavy chain constant region of IgG1.

In some embodiments, the Fc comprises one or more modifications, wherein the one or more modifications result in increased half-life, increased antibody-dependent cellular cytotoxicity (ADCC), increased antibody-dependent cellular phagocytosis (ADCP), increased complement-dependent cytotoxicity (CDC), or decreased effector function, compared with the Fc without the one or more modifications.

Provided herein is a pharmaceutical composition comprising the antibody-drug conjugate as disclosed herein and a pharmaceutically acceptable carrier.

Provided herein is a method of treating or preventing a disease or condition in a subject in need thereof, comprising administering to the subject an effective amount of the antibody-drug conjugate disclosed herein or the pharmaceutical composition as disclosed herein.

In some embodiments, the disease or condition is cancer.

In some embodiments, the cancer is selected from the group consisting of: head and neck cancer, ovarian cancer, gastric cancer, esophageal cancer, cervical cancer, prostate cancer, pancreatic cancer, estrogen receptors negative (ER−) breast cancer, progesterone receptors negative (PR−) breast cancer, HER2 negative (HER2−) triple negative breast cancer, glioblastoma, lung cancer, bladder cancer, melanoma, and kidney cancer.

In some embodiments, the disease or condition involves neovascularization.

In some embodiments, the disease or condition involving neovascularization is cancer.

In some embodiments, the disease or condition involves vascular inflammation.

In some embodiments, the method further comprises administering one or more additional therapeutic agents to the subject.

In some embodiments, the composition further comprises the one or more additional therapeutic agents.

In some embodiments, the additional therapeutic agent is formulated in a different pharmaceutical composition.

In some embodiments, the additional therapeutic agent is administered prior to administering the composition.

In some embodiments, the additional therapeutic agent is administered after administering the composition.

In some embodiments, the additional therapeutic agent is administered contemporaneously with the composition.

In some embodiments, the subject is a human subject.

Provided herein is a process for preparing an antibody-drug conjugate, the process comprising:

(A) reacting a nucleophilic or an electrophilic group on an antigen binding protein (Ab) which binds to the extracellular domain of human Tissue Factor (TF) (SEQ ID NO:810) with a bifunctional linker to form an Ab-linker intermediate, and reacting the Ab-linker intermediate with the —NH₂ group of general Formula I

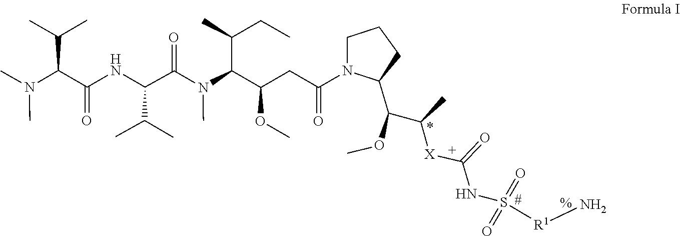

wherein:
X is *—C(O)NHCH(CH₂(R²))—⁺, wherein * and + represent the respective points of attachment as indicated in Formula I, or X is absent;
R¹ is selected from the group consisting of:

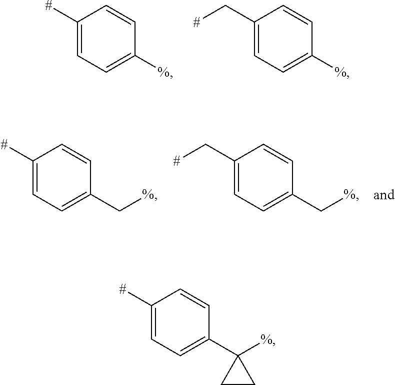

wherein # and % represent the respective points of attachment as indicated in Formula I; and R² is phenyl,

to provide the antibody drug conjugate; or

(B) reacting the —NH₂ group on the auristatin derivative of general Formula I with a bifunctional linker to form a linker-toxin intermediate, and reacting the linker-toxin intermediate with a nucleophilic or an electrophilic group on an antigen binding protein (Ab) which binds to the extracellular domain of human Tissue Factor (TF) (SEQ ID NO: 810) to provide the antibody-drug conjugate, wherein, in (A) or (B),
(a) the Ab comprises a VH-CDR1, a VH-CDR2, a VH-CDR3, a VL-CDR1, a VL-CDR2, and a VL-CDR3, wherein
   i. the VH-CDR1 comprises SEQ ID NO: 872, the VH-CDR2 comprises SEQ ID NO: 873, the VH-CDR3 comprises SEQ ID NO: 874, the VL-CDR1 comprises SEQ ID NO: 875, the VL-CDR2 comprises SEQ ID NO: 876, and the VL-CDR3 comprises SEQ ID NO: 877,
   ii. the VH-CDR1, VH-CDR2, VH-CDR3, VL-CDR1, VL-CDR2, and VL-CDR3 are from the antibody designated 25A3,
   iii. the VH-CDR1, VH-CDR2, VH-CDR3, VL-CDR1, VL-CDR2, and VL-CDR3 are from the antibody designated 25A,
   iv. the VH-CDR1, VH-CDR2, VH-CDR3, VL-CDR1, VL-CDR2, and VL-CDR3 are from the antibody designated 25A5,
   v. the VH-CDR1, VH-CDR2, VH-CDR3, VL-CDR1, VL-CDR2, and VL-CDR3 are from the antibody designated 25A5-T, or
   vi. the VH-CDR1, VH-CDR2, VH-CDR3, VL-CDR1, VL-CDR2, and VL-CDR3 are from the antibody designated 25G1; and
(b) the antibody-drug conjugate comprises one or more moieties represented by Formula IV:

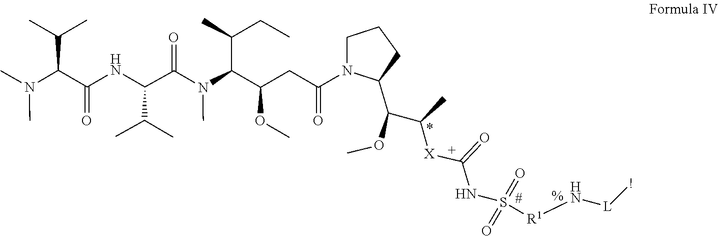

wherein:

X is *—C(O)NHCH(CH₂(R²))—⁺, wherein * and + represent the respective points of attachment as indicated in Formula IV, or X is absent;

L is a linker;

! represents the point of attachment of L to the Ab, where L is attached to the Ab through a covalent bond;

R¹ is selected from the group consisting of:

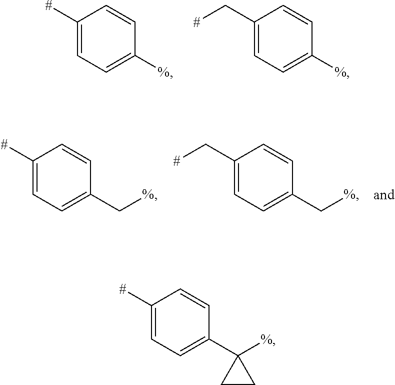

wherein # and % represent the respective points of attachment as indicated in Formula VI; and

R² is phenyl.

Provided herein is a process for preparing an antibody-drug conjugate, the process comprising:

(A) reacting a nucleophilic or an electrophilic group on an antigen binding protein (Ab) which binds to the extracellular domain of human Tissue Factor (TF) (SEQ ID NO:810) with a first linker component of a bifunctional linker that comprises two or more linker components followed by sequential addition of the remaining linker component(s) to form an Ab-linker intermediate, and reacting the Ab-linker intermediate with the —NH₂ group of a compound of general Formula I:

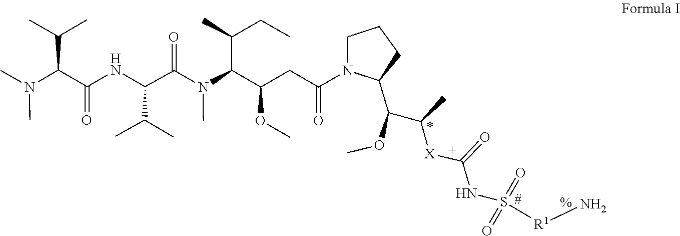

wherein:
X is *—C(O)NHCH(CH₂(R²))—⁺, wherein * and + represent the respective points of attachment as indicated in Formula I, or X is absent;
R¹ is selected from the group consisting of:

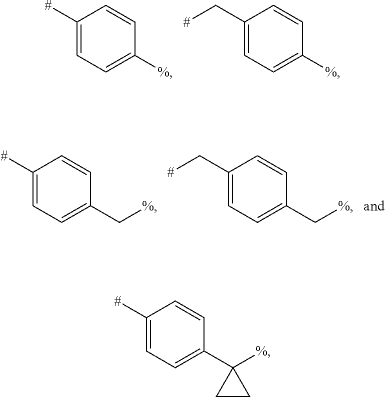

wherein # and % represent the respective points of attachment as indicated in Formula I; and R² is phenyl,

to provide the antibody drug conjugate; or

(B) reacting the —NH₂ group on the compound of general Formula I with a first linker component of a bifunctional linker that comprises two or more linker components followed by sequential addition of the remaining linker component(s) to form a linker-toxin intermediate, and reacting the linker-toxin intermediate with a nucleophilic or an electrophilic group on an antigen binding protein (Ab) which binds to the extracellular domain of human Tissue Factor (TF) (SEQ ID NO: 810) to provide the antibody-drug conjugate, wherein, in (A) or (B),
(a) the Ab comprises a VH-CDR1, a VH-CDR2, a VH-CDR3, a VL-CDR1, a VL-CDR2, and a VL-CDR3, wherein
   i. the VH-CDR1 comprises SEQ ID NO: 872, the VH-CDR2 comprises SEQ ID NO: 873, the VH-CDR3 comprises SEQ ID NO: 874, the VL-CDR1 comprises SEQ ID NO: 875, the VL-CDR2 comprises SEQ ID NO: 876, and the VL-CDR3 comprises SEQ ID NO: 877,
   ii. the VH-CDR1, VH-CDR2, VH-CDR3, VL-CDR1, VL-CDR2, and VL-CDR3 are from the antibody designated 25A3,
   iii. the VH-CDR1, VH-CDR2, VH-CDR3, VL-CDR1, VL-CDR2, and VL-CDR3 are from the antibody designated 25A,
   iv. the VH-CDR1, VH-CDR2, VH-CDR3, VL-CDR1, VL-CDR2, and VL-CDR3 are from the antibody designated 25A5,
   v. the VH-CDR1, VH-CDR2, VH-CDR3, VL-CDR1, VL-CDR2, and VL-CDR3 are from the antibody designated 25A5-T, or
   vi. the VH-CDR1, VH-CDR2, VH-CDR3, VL-CDR1, VL-CDR2, and VL-CDR3 are from the antibody designated 25G1; and
(b) the antibody-drug conjugate comprises one or more moieties represented by Formula IV:

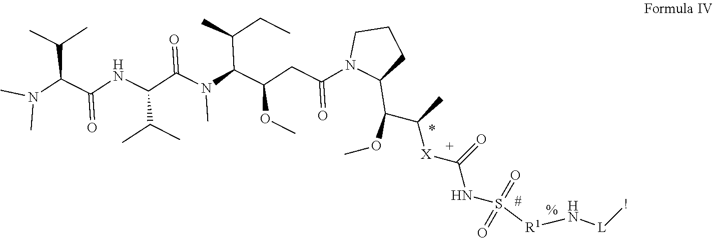

wherein:

X is *—C(O)NHCH(CH₂(R²))—⁺, wherein * and + represent the respective points of attachment as indicated in Formula IV, or X is absent;

L is a linker;

! represents the point of attachment of L to the Ab, where L is attached to the Ab through a covalent bond;

R¹ is selected from the group consisting of:

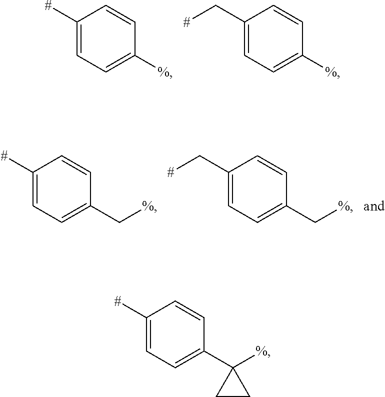

wherein # and % represent the respective points of attachment as indicated in Formula VI; and

R² is phenyl.

In some embodiments, the nucleophilic or electrophilic group on the Ab is a thiol or an amine.

In some embodiments, the process further comprises treating the Ab with a reducing agent to reduce one or more disulfide linkages in the Ab to provide the nucleophilic thiol group.

In some embodiments, L is represented by:

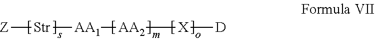

wherein:

Z represents a functional group that binds to a target group of the Ab;

D represents the point of attachment to the amino group as indicated in Formula X;
Str is a stretcher;
AA₁ and AA₂ are each independently an amino acid, wherein AA₁-[AA₂]ₘ forms a protease cleavage site;
   X is a self-immolative group;
   s is an integer selected from 0 and 1;
   m is an integer selected from the group consisting of 1, 2, 3, and 4; and
   is an integer selected from 0, 1, and 2.

Provided herein is a kit comprising the antibody-drug conjugate as disclosed herein or the pharmaceutical composition as disclosed herein, and instructions for use.

### BRIEF DESCRIPTION OF THE DRAWINGS

These and other features, aspects, and advantages of the present invention will become better understood with regard to the following description, and accompanying drawings, where:

FIG. 1 shows the structure of the linker-toxin referred to as Linker-Toxin A (also referred to herein as LT-A).

FIG. 2A shows the structure of a linker-toxin moiety of linker-toxin A (LT-A) as attached to an antibody, where ## represents a point of attachment to a tissue factor (TF) antibody. FIG. 2B shows a depiction of an antibody drug conjugate comprising a linker-toxin moiety of Linker-Toxin A (LT-A) and a TF antibody.

FIG. 3A shows the cell viability as indicated by CTG luminescence and the calculated IC₅₀ in TF-positive A431 cells after a 4 h incubation with isotype control or 25A3-LT-A, followed by washout and 68 h of culture. FIG. 3B shows the cell viability as indicated by CTG luminescence and the calculated IC₅₀ in TF-positive A431 cells after a 3-day incubation with either isotype control or 25A3-LT-A.

FIG. 4A shows results from a MDA-MB231 triple negative breast carcinoma cell line xenograft study in immune compromised mice. Animals were treated with the anti-TF antibody drug conjugate 25A3-LT-A, isotype control LT-A, or vehicle control intraperitoneally (i.p.) once weekly for 2 weeks, and body weight and tumor size was assessed biweekly. FIG. 4B shows results from a HPAF-II pancreatic carcinoma cell line xenograft study in immune compromised mice. Animals were treated with the anti-TF antibody drug conjugate 25A3-LT-A, isotype control LT-A, or vehicle control intraperitoneally (i.p.) once weekly for 2 weeks, and body weight and tumor size was assessed biweekly.

FIGS. 5A-5D show the results from a single administration, dose ranging study. Immune compromised mice with TF-positive HPAF-II pancreatic carcinoma cell xenografts were treated i.p. once when the average tumor size was 200 mm³ (arrow) with the indicated doses of anti-TF antibody drug conjugate 25A3-LT-A or vehicle control. FIG. 5A shows the mean tumor volume measurements±standard error of the mean (SEM) for each of the experimental groups. FIG. 5B shows the tumor volume measurements for individual mice treated with 5 mg/kg of 25A3-LT-A. FIG. 5C shows the tumor volume measurements for individual mice treated with 7.5 mg/kg of 25A3-LT-A. FIG. 5D shows the tumor volume measurements for individual mice treated with 10 mg/kg of 25A3-LT-A.

FIG. 6 shows the mean concentration-time profiles from a HPAF-II pancreatic carcinoma cell line xenograft study in immune compromised mice. Animals were treated i.p. once with either 2.5 mg/kg or 10 mg/kg of the anti-TF antibody-drug conjugate 25A3-LT-A, and the concentration of 25A3-LT-A was measured using a PK assay that detects the intact molecule.

FIGS. 7A-7D show the results from a late intervention study in which TF-positive HPAF-II pancreatic carcinoma cell xenografts were treated i.p. once when the average tumor size was 500 mm³ with 7.5 mg/kg or 10 mg/kg of anti-TF antibody drug conjugate 25A3-LT-A or vehicle control (PBS). FIG. 7A shows mean tumor volume measurements±SEM for each of the experimental groups. FIG. 7B shows the tumor volume measurements for individual mice treated with the vehicle control (PBS). FIG. 7C shows the tumor volume measurements for individual mice treated with 7.5 mg/kg of 25A3-LT-A. FIG. 7D shows the tumor volume measurements for individual mice treated with 10 mg/kg of 25A3-LT-A.

FIGS. 8A-8E show the results of immune compromised mice, having patient derived xenografts, that were treated i.p. with 10 mg/kg of 25A3-LT-A or the vehicle control (PBS) once when average tumor size was 200 mm³. Tumor size assessments were performed bi-weekly. The plots show mean tumor volume±SEM. FIG. 8A shows the tumor volume measurements for CTG-0353 mice. Prior to treatment, these mice had been implanted with gastric tumor fragments. FIG. 8B shows the tumor volume measurements for CTG-0707 mice. Prior to treatment, these mice had been implanted with gastric tumor fragments. FIG. 8C shows the tumor volume measurements for CTG-0786 mice. Prior to treatment, these mice had been implanted with head & neck cancer tumor fragments. FIG. 8D shows the tumor volume measurements for CTG-1076 mice. Prior to treatment, these mice had been implanted with bladder tumor fragments. FIG. 8E shows the tumor volume measurements for CTG-1130 mice. Prior to treatment, these mice had been implanted with head & neck cancer tumor fragments.

FIGS. 9A-9E show the immunostains from patient derived xenograft tumor samples collected from immune compromised mice. Biopsy specimens were sectioned and stained for TF expression. FIG. 9A shows a representative immunostain for CTG-0353 mice that had been implanted with gastric tumor fragments. FIG. 9B shows a representative immunostain for CTG-0707 mice that had been implanted with gastric tumor fragments. FIG. 9C shows a representative immunostain for CTG-0786 mice that had been implanted with head & neck cancer tumor fragments. FIG. 9D shows a representative immunostain for CTG-1076 mice that had been implanted with bladder tumor fragments. FIG. 9E shows a representative immunostain for CTG-1130 mice that had been implanted with head & neck cancer tumor fragments.

FIGS. 10A-10E show results of patient derived xenograft tumor samples collected from immune compromised mice. The plots show mean tumor volume±SEM. FIG. 10A shows the tumor volume measurements for HN2574 mice. Prior to treatment, these mice had been implanted with head & neck cancer tumor fragments. FIG. 10B shows the tumor volume measurements for ES0147 mice. Prior to treatment, these mice had been implanted with esophageal tumor fragments. FIG. 10C shows the tumor volume measurements for ES0214 mice. Prior to treatment, these mice had been implanted with esophageal tumor fragments. FIG. 10D shows the tumor volume measurements for PA1332 mice. Prior to treatment, these mice had been implanted with pancreatic tumor fragments. FIG. 10E shows the tumor volume measurements for PA6262 mice. Prior to treatment, these mice had been implanted with pancreatic tumor fragments.

FIGS. 11A-11E show the immunostains from patient derived xenograft tumor samples collected from immune compromised mice. Biopsy specimens were sectioned and stained for TF expression. FIG. 11A shows a representative immunostain for HN2574 mice that had been implanted with head & neck cancer tumor fragments. FIG. 11B shows a representative immunostain for ES0147 mice that had been implanted with esophageal tumor fragments. FIG. 11C shows a representative immunostain for ES0214 mice that had been implanted with esophageal tumor fragments. FIG. 11D shows a representative immunostain for PA1332 mice that had been implanted with pancreatic tumor fragments. FIG. 11E shows a representative immunostain for PA6262 mice that had been implanted with pancreatic tumor fragments.

FIG. 12 shows TF immunostains and H-scores for three ovarian or cervical cancer tumor patient-derived xenografts, as indicated.

FIG. 13A shows results from a TF-positive gastric patient derived xenograft study in immune compromised mice. Animals were treated with 25A3-LT-A or isotype control-LT-A i.p. once, and body weight and tumor size was assessed biweekly. FIG. 13B shows results from a TF-positive lung patient derived xenograft study in immune compromised mice. Animals were treated with 25A3-LT-A or isotype control-LT-A i.p. once, and body weight and tumor size was assessed biweekly.

FIG. 14A shows the mean aspartate aminotransferase (AST) levels in cynomolgus (“cyno”) monkeys treated with the indicated doses of 25A3-LT-A or 25A3-MMAE on days 1, 22, and 36 of the study. FIG. 14B shows the mean alanine aminotransferase (ALT) levels in cyno monkeys treated with the indicated doses of 25A3-LT-A or 25A3-MMAE on days 1, 22, and 36 of the study.

FIG. 15A shows the mean neutrophil counts for cyno monkeys treated with the indicated doses of 25A3-MMAE on days 1, 22, and 36 of the study. FIG. 15B shows the mean neutrophil counts for cyno monkeys treated with the indicated doses of 25A3-LT-A on days 1, 22, and 36 of the study. Historical average comes from baseline values collected from monkey colonies at Charles River (n of monkeys >500).

FIGS. 16A-16C show the neutrophil counts for individual cyno monkeys treated with the indicated doses of 25A3-MMAE in the indicated treatment groups. Historical average comes from baseline values collected from monkey colonies at Charles River (n of monkeys >500). FIG. 16A shows the neutrophil count for monkeys treated with 1.5 mg/kg of 25A3-MMAE. FIG. 16B shows the neutrophil count for monkeys treated with 3 mg/kg of 25A3-MMAE. FIG. 16C shows the neutrophil count for monkeys treated with 6 mg/kg of 25A3-MMAE.

FIGS. 17A-17D show the neutrophil counts for individual cyno monkeys treated with the indicated doses of 25A3-LT-A in the indicated treatment groups. Historical average comes from baseline values collected from monkey colonies at Charles River (n of monkeys >500). FIG. 17A shows the neutrophil count for monkeys treated with 3 mg/kg of 25A3-LT-A. FIG. 17B shows the neutrophil count for monkeys treated with 6 mg/kg of 25A3-LT-A. FIG. 17C shows the neutrophil count for monkeys treated with 12 mg/kg of 25A3-LT-A. FIG. 17D shows the neutrophil count for monkeys treated with 18 mg/kg of 25A3-LT-A.

FIG. 18 shows the monocyte count for cyno monkeys treated with the indicated doses of 25A3-LT-A or 25A3-MMAE on days 1, 22, and 36 of the study.

### DETAILED DESCRIPTION

### 1. Definitions

Unless otherwise defined, all terms of art, notations and other scientific terminology used herein are intended to have the meanings commonly understood by those of skill in the art. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, N.Y. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise. The terms “include,” “such as,” and the like are intended to convey inclusion without limitation, unless otherwise specifically indicated.

As used herein, the term “comprising” also specifically includes embodiments “consisting of” and “consisting essentially of” the recited elements, unless specifically indicated otherwise.

The term “about” indicates and encompasses an indicated value and a range above and below that value. In certain embodiments, the term “about” indicates the designated value±10%, ±5%, or ±1%. In certain embodiments, where applicable, the term “about” indicates the designated value(s)±one standard deviation of that value(s).

The terms “Tissue Factor,” “TF,” “platelet tissue factor,” “factor III,” “thromboplastin,” and “CD142” are used interchangeably herein to refer to TF, or any variants (e.g., splice variants and allelic variants), isoforms, and species homologs of TF that are naturally expressed by cells, or that are expressed by cells transfected with a TF gene. In some aspects, the TF protein is a TF protein naturally expressed by a primate (e.g., a monkey or a human), a rodent (e.g., a mouse or a rat), a dog, a camel, a cat, a cow, a goat, a horse, a pig or a sheep. In some aspects, the TF protein is human TF (hTF; SEQ ID NO:809). In some aspects, the TF protein is cynomolgus TF (cTF; SEQ ID NO:813). In some aspects, the TF protein is mouse TF (mTF; SEQ ID NO:817). In some aspects, the TF protein is pig TF (pTF; SEQ ID NO:824). TF is a cell surface receptor for the serine protease factor VIIa. It is often times constitutively expressed by certain cells surrounding blood vessels and in some disease settings.

The term “antibody-drug conjugate” or “ADC” refers to a conjugate comprising an antibody conjugated to one or more cytotoxic agents, optionally through one or more linkers. The term “anti-TF antibody-drug conjugate” or “anti-TF ADC” refers to a conjugate comprising an anti-TF antibody conjugated to one or more cytotoxic agents, optionally through one or more linkers.

As used herein, the terms “TF antibody,” “anti-TF antibody” are synonymous.

The term “cytotoxic agent,” as used herein, refers to a substance that inhibits or prevents a cellular function and/or causes cell death or destruction. The cytotoxic agent can be an anti-angiogenic agent, a pro-apoptotic agent, an anti-mitotic agent, an anti-kinase agent, an alkylating agent, a hormone, a hormone agonist, a hormone antagonist, a chemokine, a drug, a prodrug, a toxin, an enzyme, an antimetabolite, an antibiotic, an alkaloid, or a radioactive isotope. Exemplary cytotoxic agents include calicheamycin, camptothecin, carboplatin, irinotecan, SN-38, carboplatin, camptothecan, cyclophosphamide, cytarabine, dacarbazine, docetaxel, dactinomycin, daunorubicin, doxorubicin, doxorubicin, etoposide, idarubicin, topotecan, vinca alkaloid, maytansinoid, maytansinoid analog, pyrrolobenzodiazepine, taxoid, duocarmycin, dolastatin, auristatin, and derivatives thereof.

A “linker” refers to a molecule that connects one composition to another, e.g., an antibody to an agent. Linkers described herein can conjugate an antibody to a cytotoxic agent. Exemplary linkers include a labile linker, an acid labile linker, a photolabile linker, a charged linker, a disulfide-containing linker, a peptidase-sensitive linker, a □-glucuronide-linker, a dimethyl linker, a thio-ether linker, and a hydrophilic linker. A linker can be cleavable or non-cleavable.

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, Pa. Briefly, each heavy chain typically comprises a heavy chain variable region (V_{H}) and a heavy chain constant region (C_{H}). The heavy chain constant region typically comprises three domains, abbreviated C_{H1}, C_{H2}, and C_{H3}. Each light chain typically comprises a light chain variable region (V_{L}) and a light chain constant region. The light chain constant region typically comprises one domain, abbreviated C_{L}.

The term “antibody” is used herein in its broadest sense and includes certain types of immunoglobulin molecules comprising one or more antigen-binding domains that specifically bind to an antigen or epitope. An antibody specifically includes intact antibodies (e.g., intact immunoglobulins), antibody fragments, and multi-specific antibodies.

The term “alternative scaffold” refers to a molecule in which one or more regions may be diversified to produce one or more antigen-binding domains that specifically bind to an antigen or epitope. In some embodiments, the antigen-binding domain binds the antigen or epitope with specificity and affinity similar to that of an antibody. Exemplary alternative scaffolds include those derived from fibronectin (e.g., Adnectins™), the β-sandwich (e.g., iMab), lipocalin (e.g., Anticalins®), EETI-II/AGRP, BPTI/LACI-D1/ITI-D2 (e.g., Kunitz domains), thioredoxin peptide aptamers, protein A (e.g., Affibody®), ankyrin repeats (e.g., DARPins), gamma-B-crystallin/ubiquitin (e.g., Affilins), CTLD3 (e.g., Tetranectins), Fynomers, and (LDLR-A module) (e.g., Avimers). Additional information on alternative scaffolds is provided in Binz et al., Nat. Biotechnol., 2005 23:1257-1268; Skerra, Current Opin. in Biotech., 2007 18:295-304; and Silacci et al., J. Biol. Chem., 2014, 289:14392-14398; each of which is incorporated by reference in its entirety.

The term “antigen-binding domain” means the portion of an antibody that is capable of specifically binding to an antigen or epitope. One example of an antigen-binding domain is an antigen-binding domain formed by a V_{H}-V_{L} dimer of an antibody. Another example of an antigen-binding domain is an antigen-binding domain formed by diversification of certain loops from the tenth fibronectin type III domain of an Adnectin. Antigen-binding domains can be found in various contexts including antibodies and chimeric antigen receptors (CARs), for example CARs derived from antibodies or antibody fragments such as scFvs.

The terms “full length antibody,” “intact antibody,” and “whole antibody” are used herein interchangeably to refer to an antibody having a structure substantially similar to a naturally occurring antibody structure and having heavy chains that comprise an Fc region. For example, when used to refer to an IgG molecule, a “full length antibody” is an antibody that comprises two heavy chains and two light chains.

The term “Fc region” means the C-terminal region of an immunoglobulin heavy chain that, in naturally occurring antibodies, interacts with Fc receptors and certain proteins of the complement system. The structures of the Fc regions of various immunoglobulins, and the glycosylation sites contained therein, are known in the art. See Schroeder and Cavacini, J. Allergy Clin. Immunol., 2010, 125:S41-52, incorporated by reference in its entirety. The Fc region may be a naturally occurring Fc region, or an Fc region modified as described in the art or elsewhere in this disclosure.

The V_{H} and V_{L} regions may be further subdivided into regions of hypervariability (“hypervariable regions (HVRs);” also called “complementarity determining regions” (CDRs)) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1-FR2-CDR2-FR3-CDR3-FR4. The CDRs are involved in antigen binding, and influence antigen specificity and binding affinity of the antibody. See Kabat et al., Sequences of Proteins of Immunological Interest 5th ed. (1991) Public Health Service, National Institutes of Health, Bethesda, Md., incorporated by reference in its entirety.

A “Complementary Determining Region (CDR)” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. CDRs are variable region sequences interspersed within the framework region sequences. CDRs are well recognized in the art and have been defined by, for example, Kabat as the regions of most hypervariability within the antibody variable (V) domains. See Kabat et al., J Biol Chem, 1977, 252:6609-6616 and Kabat, Adv Protein Chem, 1978, 32:1-75, each of which is incorporated by reference in its entirety. CDRs have also been defined structurally by Chothia as those residues that are not part of the conserved β-sheet framework, and thus are able to adapt different conformations. See Chothia and Lesk, J Mol Biol, 1987, 196:901-917, incorporated by reference in its entirety. Both the Kabat and Chothia nomenclatures are well known in the art. AbM, Contact and IMGT also define CDRs. CDR positions within a canonical antibody variable domain have been determined by comparison of numerous structures. See Morea et al., Methods, 2000, 20:267-279 and Al-Lazikani et al., J Mol Biol, 1997, 273:927-48, each of which is incorporated by reference in its entirety. Because the number of residues within a hypervariable region varies in different antibodies, additional residues relative to the canonical positions are conventionally numbered with a, b, c and so forth next to the residue number in the canonical variable domain numbering scheme (Al-Lazikani et al., supra). Such terminology is well known to those skilled in the art.

A number of hypervariable region delineations are in use and are included herein. The Kabat CDRs are based on sequence variability and are the most commonly used. See Kabat et al. (1992) Sequences of Proteins of Immunological Interest, DIANE Publishing: 2719, incorporated by reference in its entirety. Chothia refers instead to the location of the structural loops (Chothia and Lesk, supra). The AbM hypervariable regions represent a compromise between the Kabat CDRs and Chothia structural loops, and are used by Oxford Molecular's AbM antibody modeling software. The Contact hypervariable regions are based on an analysis of the available complex crystal structures. The residues from each of these hypervariable regions are noted in Table 1.

More recently, a universal numbering system ImMunoGeneTics (IMGT) Information System™ has been developed and widely adopted. See Lefranc et al., Dev Comp Immunol, 2003, 27:55-77, incorporated by reference in its entirety. IMGT is an integrated information system specializing in immunoglobulins (IG), T cell receptors (TR) and major histocompatibility complex (MHC) of human and other vertebrates. The IMGT CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. Correspondence between the Kabat, Chothia and IMGT numbering is also well known in the art (Lefranc et al., supra). An Exemplary system, shown herein, combines Kabat and Chothia CDR definitions.

TABLE 1

| | Exemplary | | | | | |
|---|---|---|---|---|---|---|
| | (Kabat + | | | | | |
| | Chothia) | Kabat | Chothia | AbM | Contact | IMGT |
| VH CDR1 | 26-35 | 31-35 | 26-32 | 26-35 | 30-35 | 27-38 |
| VH CDR2 | 50-65 | 50-65 | 52a-55 | 50-58 | 47-58 | 56-65 |
| VH CDR3 | 95-102 | 95-102 | 96-101 | 95-102 | 93-101 | 105-117 |
| VL CDR1 | 24-34 | 24-34 | 26-32 | 24-34 | 30-36 | 27-38 |
| VL CDR2 | 50-56 | 50-56 | 50-52 | 50-56 | 46-55 | 56-65 |
| VL CDR3 | 89-97 | 89-97 | 91-96 | 89-97 | 89-96 | 105-117 |

The light chain from any vertebrate species can be assigned to one of two types, called kappa (κ) and lambda (λ), based on the sequence of its constant domain.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The term “constant region” or “constant domain” refers to a carboxy terminal portion of the light and heavy chain which is not directly involved in binding of the antibody to antigen but exhibits various effector function, such as interaction with the Fc receptor. The terms refer to the portion of an immunoglobulin molecule having a more conserved amino acid sequence relative to the other portion of the immunoglobulin, the variable domain, which contains the antigen-binding site. The constant domain contains the C_{H1}, C_{H2} and C_{H3} domains of the heavy chain and the C_{L} domain of the light chain.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

An “antibody fragment” comprises a portion of an intact antibody, such as the antigen-binding or variable region of an intact antibody. Antibody fragments include, for example, Fv fragments, Fab fragments, F(ab′)₂ fragments, Fab′ fragments, scFv (sFv) fragments, and scFv-Fc fragments.

“Fv” fragments comprise a non-covalently-linked dimer of one heavy chain variable domain and one light chain variable domain.

“Fab” fragments comprise, in addition to the heavy and light chain variable domains, the constant domain of the light chain and the first constant domain (C_{H1}) of the heavy chain. Fab fragments may be generated, for example, by recombinant methods or by papain digestion of a full-length antibody.

“F(ab′)₂” fragments contain two Fab′ fragments joined, near the hinge region, by disulfide bonds. F(ab′)₂ fragments may be generated, for example, by recombinant methods or by pepsin digestion of an intact antibody. The F(ab′) fragments can be dissociated, for example, by treatment with ß-mercaptoethanol.

“Single-chain Fv” or “sFv” or “scFv” antibody fragments comprise a V_{H} domain and a V_{L} domain in a single polypeptide chain. The V_{H} and V_{L} are generally linked by a peptide linker. See Plückthun A. (1994). Any suitable linker may be used. In some embodiments, the linker is a (GGGGS)ₙ (SEQ ID NO:823). In some embodiments, n=1, 2, 3, 4, 5, or 6. See Antibodies from Escherichia coli. In Rosenberg M. & Moore G. P. (Eds.), The Pharmacology of Monoclonal Antibodies vol. 113 (pp. 269-315). Springer-Verlag, New York, incorporated by reference in its entirety.

“scFv-Fc” fragments comprise an scFv attached to an Fc domain. For example, an Fc domain may be attached to the C-terminal of the scFv. The Fc domain may follow the V_{H} or V_{L}, depending on the orientation of the variable domains in the scFv (i.e., V_{H}-V_{L} or V_{L}-V_{H}). Any suitable Fc domain known in the art or described herein may be used.

The term “single domain antibody” refers to a molecule in which one variable domain of an antibody specifically binds to an antigen without the presence of the other variable domain. Single domain antibodies, and fragments thereof, are described in Arabi Ghahroudi et al., FEBS Letters, 1998, 414:521-526 and Muyldermans et al., Trends in Biochem. Sci., 2001, 26:230-245, each of which is incorporated by reference in its entirety. Single domain antibodies are also known as sdAbs or nanobodies.

A “multispecific antibody” is an antibody that comprises two or more different antigen-binding domains that collectively specifically bind two or more different epitopes. The two or more different epitopes may be epitopes on the same antigen (e.g., a single TF molecule expressed by a cell) or on different antigens (e.g., a TF molecule and a non-TF molecule). In some aspects, a multi-specific antibody binds two different epitopes (i.e., a “bispecific antibody”). In some aspects, a multi-specific antibody binds three different epitopes (i.e., a “trispecific antibody”). In some aspects, a multi-specific antibody binds four different epitopes (i.e., a “quadspecific antibody”). In some aspects, a multi-specific antibody binds five different epitopes (i.e., a “quintspecific antibody”). In some aspects, a multi-specific antibody binds 6, 7, 8, or more different epitopes. Each binding specificity may be present in any suitable valency. Examples of multispecific antibodies are provided elsewhere in this disclosure.

A “monospecific antibody” is an antibody that comprises one or more binding sites that specifically bind to a single epitope. An example of a monospecific antibody is a naturally occurring IgG molecule which, while divalent (i.e., having two antigen-binding domains), recognizes the same epitope at each of the two antigen-binding domains. The binding specificity may be present in any suitable valency.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “chimeric antibody” refers to an antibody in which a portion of the heavy and/or light chain is derived from a particular source or species, while the remainder of the heavy and/or light chain is derived from a different source or species.

“Humanized” forms of non-human antibodies are chimeric antibodies that contain minimal sequence derived from the non-human antibody. A humanized antibody is generally a human antibody (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

An “isolated antibody” or “isolated nucleic acid” is an antibody or nucleic acid that has been separated and/or recovered from a component of its natural environment. Components of the natural environment may include enzymes, hormones, and other proteinaceous or nonproteinaceous materials. In some embodiments, an isolated antibody is purified to a degree sufficient to obtain at least 15 residues of N-terminal or internal amino acid sequence, for example by use of a spinning cup sequenator. In some embodiments, an isolated antibody is purified to homogeneity by gel electrophoresis (e.g., SDS-PAGE) under reducing or nonreducing conditions, with detection by Coomassie blue or silver stain. In some embodiments, an isolated antibody may include an antibody in situ within recombinant cells, since at least one component of the antibody's natural environment is not present. In some aspects, an isolated antibody or isolated nucleic acid is prepared by at least one purification step. In some embodiments, an isolated antibody or isolated nucleic acid is purified to at least 80%, 85%, 90%, 95%, or 99% by weight. In some embodiments, an isolated antibody or isolated nucleic acid is purified to at least 80%, 85%, 90%, 95%, or 99% by volume. In some embodiments, an isolated antibody or isolated nucleic acid is provided as a solution comprising at least 85%, 90%, 95%, 98%, 99% to 100% antibody or nucleic acid by weight. In some embodiments, an isolated antibody or isolated nucleic acid is provided as a solution comprising at least 85%, 90%, 95%, 98%, 99% to 100% antibody or nucleic acid by volume.

“Affinity” refers to the strength of the sum total of non-covalent interactions between a single binding site of a molecule (e.g., an antibody) and its binding partner (e.g., an antigen or epitope). Unless indicated otherwise, as used herein, “affinity” refers to intrinsic binding affinity, which reflects a 1:1 interaction between members of a binding pair (e.g., antibody and antigen or epitope). The affinity of a molecule X for its partner Y can be represented by the dissociation equilibrium constant (K_{D}). The kinetic components that contribute to the dissociation equilibrium constant are described in more detail below. Affinity can be measured by common methods known in the art, including those described herein, such as surface plasmon resonance (SPR) technology (e.g., BIACORE®) or biolayer interferometry (e.g., FORTEBIO®).

With regard to the binding of an antibody to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule and comparing it to binding to a non-target molecule. Specific binding can also be determined by competition with a control molecule that mimics the epitope recognized on the target molecule. In that case, specific binding is indicated if the binding of the antibody to the target molecule is competitively inhibited by the control molecule. In some aspects, the affinity of a TF antibody for a non-target molecule is less than about 50% of the affinity for TF. In some aspects, the affinity of a TF antibody for a non-target molecule is less than about 40% of the affinity for TF. In some aspects, the affinity of a TF antibody for a non-target molecule is less than about 30% of the affinity for TF. In some aspects, the affinity of a TF antibody for a non-target molecule is less than about 20% of the affinity for TF. In some aspects, the affinity of a TF antibody for a non-target molecule is less than about 10% of the affinity for TF. In some aspects, the affinity of a TF antibody for a non-target molecule is less than about 1% of the affinity for TF. In some aspects, the affinity of a TF antibody for a non-target molecule is less than about 0.1% of the affinity for TF.

The term “k_{d}” (sec⁻¹), as used herein, refers to the dissociation rate constant of a particular antibody-antigen interaction. This value is also referred to as the k_{off} value.

The term “kₐ” (M⁻¹×sec⁻¹), as used herein, refers to the association rate constant of a particular antibody-antigen interaction. This value is also referred to as the kₒₙ value.

The term “K_{D}” (M), as used herein, refers to the dissociation equilibrium constant of a particular antibody-antigen interaction. K_{D}=k_{d}/kₐ. In some embodiments, the affinity of an antibody is described in terms of the K_{D} for an interaction between such antibody and its antigen. For clarity, as known in the art, a smaller K_{D} value indicates a higher affinity interaction, while a larger K_{D} value indicates a lower affinity interaction.

The term “K_{A}” (M⁻¹), as used herein, refers to the association equilibrium constant of a particular antibody-antigen interaction. K_{A}=kₐ/k_{d}.

An “affinity matured” antibody is an antibody with one or more alterations (e.g., in one or more CDRs or FRs) relative to a parent antibody (i.e., an antibody from which the altered antibody is derived or designed) that result in an improvement in the affinity of the antibody for its antigen, compared to the parent antibody which does not possess the alteration(s). In some embodiments, an affinity matured antibody has nanomolar or picomolar affinity for the target antigen. Affinity matured antibodies may be produced using a variety of methods known in the art. For example, Marks et al. (Bio/Technology, 1992, 10:779-783, incorporated by reference in its entirety) describes affinity maturation by V_{H} and V_{L} domain shuffling. Random mutagenesis of CDR and/or framework residues is described by, for example, Barbas et al., Proc. Nat. Acad. Sci. U.S.A., 1994, 91:3809-3813; Schier et al., Gene, 1995, 169:147-155; Yelton et al., J. Immunol., 1995, 155:1994-2004; Jackson et al., J. Immunol., 1995, 154:3310-33199; and Hawkins et al, J. Mol. Biol., 1992, 226:889-896; each of which is incorporated by reference in its entirety.

“Fc effector functions” refer to those biological activities mediated by the Fc region of an antibody, which activities may vary depending on the antibody isotype. Examples of antibody effector functions include C1q binding to activate complement dependent cytotoxicity (CDC), Fc receptor binding to activate antibody-dependent cellular cytotoxicity (ADCC), and antibody dependent cellular phagocytosis (ADCP).

When used herein in the context of two or more antibodies, the term “competes with” or “cross-competes with” indicates that the two or more antibodies compete for binding to an antigen (e.g., TF). In one exemplary assay, TF is coated on a surface and contacted with a first TF antibody, after which a second TF antibody is added. In another exemplary assay, first a TF antibody is coated on a surface and contacted with TF, and then a second TF antibody is added. If the presence of the first TF antibody reduces binding of the second TF antibody, in either assay, then the antibodies compete with each other. The term “competes with” also includes combinations of antibodies where one antibody reduces binding of another antibody, but where no competition is observed when the antibodies are added in the reverse order. However, in some embodiments, the first and second antibodies inhibit binding of each other, regardless of the order in which they are added. In some embodiments, one antibody reduces binding of another antibody to its antigen by at least 25%, at least 50%, at least 60%, at least 70%, at least 80%, at least 85%, at least 90%, or at least 95%. A skilled artisan can select the concentrations of the antibodies used in the competition assays based on the affinities of the antibodies for TF and the valency of the antibodies. The assays described in this definition are illustrative, and a skilled artisan can utilize any suitable assay to determine if antibodies compete with each other. Suitable assays are described, for example, in Cox et al., “Immunoassay Methods,” in Assay Guidance Manual [Internet], Updated Dec. 24, 2014 (www.ncbi.nlm.nih.gov/books/NBK92434/; accessed Sep. 29, 2015); Silman et al., Cytometry, 2001, 44:30-37; and Finco et al., J. Pharm. Biomed. Anal., 2011, 54:351-358; each of which is incorporated by reference in its entirety. As provided in Example 8 of PCT/US2019/12427, filed on Jan. 4, 2019, antibodies of group 25 and antibodies of group 43 compete with each other for binding to human TF, while antibodies from groups 1, 29, 39, and 54 do not compete for binding to human TF with antibodies of groups 25 and 43.

As used herein, an antibody that binds specifically to a human antigen is considered to bind the same antigen of mouse origin when a K_{D} value can be measured on a ForteBio Octet with the mouse antigen. An antibody that binds specifically to a human antigen is considered to be “cross-reactive” with the same antigen of mouse origin when the K_{D} value for the mouse antigen is no greater than 20 times the corresponding K_{D} value for the respective human antigen. For example, the antibody M1593 described in U.S. Pat. Nos. 8,722,044, 8,951,525, and 8,999,333, each of which is herein incorporated by reference for all purposes, the humanized 5G9 antibody described in Ngo et al., 2007, Int J Cancer, 120(6):1261-1267, incorporated by reference in its entirety, and chimeric ALT-836 antibody described in Hong et al, 2012, J Nucl Med, 53(11):1748-1754, incorporated by reference in its entirety, do not bind to mouse TF. As provided in Examples 1 and 2 of PCT/US2019/12427, filed on Jan. 4, 2019, TF antibodies from groups 25 and 43 bind to mouse TF, e.g., the TF antibodies 25G, 25G1, 25G9, and 43D8 are cross-reactive with mouse TF.

As used herein, an antibody that binds specifically to a human antigen is considered to be “cross-reactive” with the same antigen of cynomolgus monkey origin when the K_{D} value for the cynomolgus monkey antigen is no greater than 15 times the corresponding K_{D} value for the respective human antigen. As provided in Example 1 of PCT/US2019/12427, filed on Jan. 4, 2019, all tested antibodies from groups 1, 25, 29, 39, 43, and 54 are cross-reactive with cynomolgus monkey TF.

The term “epitope” means a portion of an antigen that is specifically bound by an antibody. Epitopes frequently include surface-accessible amino acid residues and/or sugar side chains and may have specific three dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding, and other amino acid residues, which are not directly involved in the binding. The epitope to which an antibody binds can be determined using known techniques for epitope determination such as, for example, testing for antibody binding to TF variants with different point-mutations, or to chimeric TF variants.

Percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

A “conservative substitution” or a “conservative amino acid substitution,” refers to the substitution of an amino acid with a chemically or functionally similar amino acid. Conservative substitution tables providing similar amino acids are well known in the art. By way of example, the groups of amino acids provided in Tables 2-4 are, in some embodiments, considered conservative substitutions for one another.

TABLE 2
Selected groups of amino acids that are considered
conservative substitutions for one another, in certain embodiments.

| | |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

TABLE 3
Additional selected groups of amino acids that are considered
conservative substitutions for one another, in certain embodiments.

| | |
|---|---|
| Group 1 | A, S, and T |
| Group 2 | D and E |
| Group 3 | N and Q |
| Group 4 | R and K |
| Group 5 | I, L, and M |
| Group 6 | F, Y, and W |

TABLE 4
Further selected groups of amino acids that are considered
conservative substitutions for one another, in certain embodiments.

| | |
|---|---|
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, for example, in Creighton, Proteins: Structures and Molecular Properties 2nd ed. (1993) W. H. Freeman & Co., New York, N.Y. An antibody generated by making one or more conservative substitutions of amino acid residues in a parent antibody is referred to as a “conservatively modified variant.”

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which an exogenous nucleic acid has been introduced, and the progeny of such cells. Host cells include “transformants” (or “transformed cells”) and “transfectants” (or “transfected cells”), which each include the primary transformed or transfected cell and progeny derived therefrom. Such progeny may not be completely identical in nucleic acid content to a parent cell, and may contain mutations.

The term “treating” (and variations thereof such as “treat” or “treatment”) refers to clinical intervention in an attempt to alter the natural course of a disease or condition in a subject in need thereof. Treatment can be performed both for prophylaxis and during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “therapeutically effective amount” or “effective amount” refers to an amount of an antibody or pharmaceutical composition provided herein that, when administered to a subject, is effective to treat a disease or disorder.

As used herein, the term “subject” means a mammalian subject. Exemplary subjects include humans, monkeys, dogs, cats, mice, rats, cows, horses, camels, goats, rabbits, pigs and sheep. In certain embodiments, the subject is a human. In some embodiments the subject has a disease or condition that can be treated with an antibody provided herein. In some aspects, the disease or condition is a cancer. In some aspects, the disease or condition involves neovascularization or vascular inflammation. In certain aspects, the disease or condition involving neovascularization is cancer.

The term “package insert” is used to refer to instructions customarily included in commercial packages of therapeutic or diagnostic products (e.g., kits) that contain information about the indications, usage, dosage, administration, combination therapy, contraindications and/or warnings concerning the use of such therapeutic or diagnostic products.

A “chemotherapeutic agent” refers to a chemical compound useful in the treatment of cancer. Chemotherapeutic agents include “anti-hormonal agents” or “endocrine therapeutics” which act to regulate, reduce, block, or inhibit the effects of hormones that can promote the growth of cancer.

The term “cytostatic agent” refers to a compound or composition which arrests growth of a cell either in vitro or in vivo. In some embodiments, a cytostatic agent is an agent that reduces the percentage of cells in S phase. In some embodiments, a cytostatic agent reduces the percentage of cells in S phase by at least about 20%, at least about 40%, at least about 60%, or at least about 80%.

The term “pharmaceutical composition” refers to a preparation which is in such form as to permit the biological activity of an active ingredient contained therein to be effective in treating a subject, and which contains no additional components which are unacceptably toxic to the subject in the amounts provided in the pharmaceutical composition.

The terms “modulate” and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

The term “agonize” refers to the activation of receptor signaling to induce a biological response associated with activation of the receptor. An “agonist” is an entity that binds to and agonizes a receptor.

The term “antagonize” refers to the inhibition of receptor signaling to inhibit a biological response associated with activation of the receptor. An “antagonist” is an entity that binds to and antagonizes a receptor.

“Alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me (—CH₃), Et (—CH₂CH₃), iPr (—CH(CH₃)₂), nPr (—CH₂CH₂CH₃), n-Bu (—CH₂CH₂CH₂CH₃), or i-Bu (—CH₂CH(CH₃)₂).

“Alkylene” refers to an alkyl group wherein two hydrogens are removed to provide a divalent radical, and which may be substituted or unsubstituted. Unsubstituted alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted alkylene groups, e.g., substituted with one or more alkyl (methyl) groups, include but are not limited to, substituted methylene (—CH(CH₃)—, (—C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), and the like.

“Halo” or “halogen” refers to fluoro (F), chloro (Cl), bromo (Br), and iodo (I). In certain embodiments, the halo group is either fluoro or chloro.

As used herein, the term “self-immolative group” refers to a moiety or residue that provides stable bond formation between two groups of a compound or conjugate, but which becomes labile upon activation (e.g., nucleophilic attack) leading to rapid cleavage of the moiety or residue and separation of the two groups. The chemistry of self-immolative groups is described, for example, in Alouane, A. et al., “Self-immolative spacers: kinetic aspects, structure-property relationships, and applications”, Angew. Chem. Int. Ed., 2015, 54, 7492-7509 and Kolakowski, R. V. et al., “The methylene alkoxy carbamate self-immolative unit: Utilization of the targeted delivery of alcohol-containing payloads with antibody-drug conjugates”, Angew. Chem. Int. Ed., 2016, 55, 7948-7951.

### 2. TF Antibodies

2.1. TF Binding

Provided herein are isolated antibodies that specifically bind to TF. In some aspects, the TF is hTF (SEQ ID NO:809). In some aspects, the TF is cTF (SEQ ID NO:813). In some aspects, the TF is mTF (SEQ ID NO:817). In some aspects, the TF is rabbit TF (SEQ ID NO:832). In some aspects, the TF is pTF (SEQ ID NO:824). In some embodiments, the antibodies provided herein specifically bind to hTF (SEQ ID NO:809), cTF (SEQ ID NO:813), mTF (SEQ ID NO:817), rabbit TF (SEQ ID NO:832), and pTF (SEQ ID NO:824). In some embodiments, the antibodies provided herein specifically bind to hTF (SEQ ID NO:809), cTF (SEQ ID NO:813), mTF (SEQ ID NO:817), and pTF (SEQ ID NO:824). In some embodiments, the antibodies provided herein specifically bind to hTF (SEQ ID NO:809), cTF (SEQ ID NO:813), and mTF (SEQ ID NO:817). In some embodiments, the antibodies provided herein specifically bind to hTF (SEQ ID NO:809) and cTF (SEQ ID NO:813). In some embodiments, the antibodies provided herein do not bind mTF (SEQ ID NO:817). In some embodiments, the antibodies provided herein do not bind pTF (SEQ ID NO:824). In some embodiments, the antibodies provided herein do not bind rabbit TF (SEQ ID NO:832).

In various embodiments, the antibodies provided herein specifically bind to the extracellular domain of human TF (SEQ ID NO:810).

In some embodiments, the binding between an antibody provided herein and a variant TF extracellular domain comprising a mutation at amino acid residue 149 of the sequence shown in SEQ ID NO:810 is less than 50% of the binding between the antibody provided herein and the extracellular domain of TF of the sequence shown in SEQ ID NO:810, as determined by the median fluorescence intensity value of the antibody relative to an isotype control in a live cell staining assay. In some embodiments, the mutation at amino acid residue 149 of the sequence shown in SEQ ID NO:810 is K149N.

In some embodiments, the binding between an antibody provided herein and a variant TF extracellular domain comprising a mutation at amino acid residue 68 of the sequence shown in SEQ ID NO:810 is greater than 50% of the binding between the antibody provided herein and the extracellular domain of TF of the sequence shown in SEQ ID NO:810, as determined by the median fluorescence intensity value of the antibody relative to an isotype control in a live cell staining assay. In some embodiments, the mutation at amino acid residue 68 of the sequence shown in SEQ ID NO:810 is K68N.

In some embodiments, the binding between an antibody provided herein and a variant TF extracellular domain comprising mutations at amino acid residues 171 and 197 of the sequence shown in SEQ ID NO:810 is less than 50% of the binding between the antibody provided herein and the extracellular domain of TF of the sequence shown in SEQ ID NO:810, as determined by the median fluorescence intensity value of the antibody relative to an isotype control in a live cell staining assay. In some embodiments, the mutations at amino acid residues 171 and 197 of the sequence shown in SEQ ID NO:810 are N171H and T197K.

In some embodiments, the binding between an antibody provided herein and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between an antibody provided herein and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between an antibody provided herein and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between an antibody provided herein and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between an antibody provided herein and a human TF extracellular domain with amino acid residues 159-219 of the sequence shown in SEQ ID NO:810 replaced by rat TF extracellular domain amino acid residues 164-224 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between an antibody provided herein and a human TF extracellular domain with amino acid residues 159-189 of the sequence shown in SEQ ID NO:810 replaced by rat TF extracellular domain amino acid residues 164-194 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between an antibody provided herein and a human TF extracellular domain with amino acid residues 159-174 of the sequence shown in SEQ ID NO:810 replaced by rat TF extracellular domain amino acid residues 164-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between an antibody provided herein and a human TF extracellular domain with amino acid residues 167-174 of the sequence shown in SEQ ID NO:810 replaced by rat TF extracellular domain amino acid residues 172-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between an antibody provided herein and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the antibody provided herein and the extracellular domain of TF of the sequence shown in SEQ ID NO:810, as determined by the median fluorescence intensity value of the antibody relative to an isotype control in a live cell staining assay.

In some embodiments, the binding between an antibody provided herein and a variant TF extracellular domain comprising a mutation at amino acid residue 149 of the sequence shown in SEQ ID NO:810 is less than 50% of the binding between the antibody provided herein and the extracellular domain of TF of the sequence shown in SEQ ID NO:810; the binding between an antibody provided herein and a variant TF extracellular domain comprising a mutation at amino acid residue 68 of the sequence shown in SEQ ID NO:810 is greater than 50% of the binding between the antibody provided herein and the extracellular domain of TF of the sequence shown in SEQ ID NO:810; the binding between an antibody provided herein and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the antibody and the extracellular domain of TF of the sequence shown in SEQ ID NO:810; the binding between an antibody provided herein and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the antibody and the extracellular domain of TF of the sequence shown in SEQ ID NO:810; the binding between an antibody provided herein and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the antibody and the extracellular domain of TF of the sequence shown in SEQ ID NO:810; the binding between an antibody provided herein and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the antibody and the extracellular domain of TF of the sequence shown in SEQ ID NO:810; and the binding between an antibody provided herein and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the antibody provided herein and the extracellular domain of TF of the sequence shown in SEQ ID NO:810, as determined by the median fluorescence intensity value of the antibody relative to an isotype control in a live cell staining assay. In some embodiments, the mutation at amino acid residue 149 of the sequence shown in SEQ ID NO:810 is K149N; and the mutation at amino acid residue 68 of the sequence shown in SEQ ID NO:810 is K68N.

In some embodiments, the binding between an antibody provided herein and a variant TF extracellular domain comprising a mutation at amino acid residue 149 of the sequence shown in SEQ ID NO:810 is less than 50% of the binding between the antibody provided herein and the extracellular domain of TF of the sequence shown in SEQ ID NO:810; the binding between an antibody provided herein and a variant TF extracellular domain comprising a mutation at amino acid residue 68 of the sequence shown in SEQ ID NO:810 is greater than 50% of the binding between the antibody provided herein and the extracellular domain of TF of the sequence shown in SEQ ID NO:810; the binding between an antibody provided herein and a variant TF extracellular domain comprising mutations at amino acid residues 171 and 197 of the sequence shown in SEQ ID NO:810 is less than 50% of the binding between the antibody provided herein and the extracellular domain of TF of the sequence shown in SEQ ID NO:810; the binding between an antibody provided herein and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the antibody and the extracellular domain of TF of the sequence shown in SEQ ID NO:810; the binding between an antibody provided herein and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the antibody and the extracellular domain of TF of the sequence shown in SEQ ID NO:810; the binding between an antibody provided herein and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the antibody and the extracellular domain of TF of the sequence shown in SEQ ID NO:810; the binding between an antibody provided herein and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the antibody and the extracellular domain of TF of the sequence shown in SEQ ID NO:810; the binding between an antibody provided herein and a human TF extracellular domain with amino acid residues 159-219 of the sequence shown in SEQ ID NO:810 replaced by rat TF extracellular domain amino acid residues 164-224 of the sequence shown in SEQ ID NO:838 is less than 50% of the binding between the antibody and the extracellular domain of TF of the sequence shown in SEQ ID NO:810; the binding between an antibody provided herein and a human TF extracellular domain with amino acid residues 159-189 of the sequence shown in SEQ ID NO:810 replaced by rat TF extracellular domain amino acid residues 164-194 of the sequence shown in SEQ ID NO:838 is less than 50% of the binding between the antibody and the extracellular domain of TF of the sequence shown in SEQ ID NO:810; the binding between an antibody provided herein and a human TF extracellular domain with amino acid residues 159-174 of the sequence shown in SEQ ID NO:810 replaced by rat TF extracellular domain amino acid residues 164-179 of the sequence shown in SEQ ID NO:838 is less than 50% of the binding between the antibody and the extracellular domain of TF of the sequence shown in SEQ ID NO:810; the binding between an antibody provided herein and a human TF extracellular domain with amino acid residues 167-174 of the sequence shown in SEQ ID NO:810 replaced by rat TF extracellular domain amino acid residues 172-179 of the sequence shown in SEQ ID NO:838 is less than 50% of the binding between the antibody and the extracellular domain of TF of the sequence shown in SEQ ID NO:810; and the binding between an antibody provided herein and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the antibody provided herein and the extracellular domain of TF of the sequence shown in SEQ ID NO:810, as determined by the median fluorescence intensity value of the antibody relative to an isotype control in a live cell staining assay. In some embodiments, the mutation at amino acid residue 149 of the sequence shown in SEQ ID NO:810 is K149N; the mutation at amino acid residue 68 of the sequence shown in SEQ ID NO:810 is K68N; and the mutations at amino acid residues 171 and 197 of the sequence shown in SEQ ID NO:810 are N171H and T197K.

In some embodiments, the antibodies provided herein are inert in inhibiting human thrombin generation as determined by thrombin generation assay (TGA) compared to a reference antibody M1593, wherein the reference antibody M1593 comprises a V_{H} sequence of SEQ ID NO:821 and a V_{L} sequence of SEQ ID NO:822.

In some embodiments, the antibodies provided herein do not inhibit human thrombin generation as determined by thrombin generation assay (TGA). In certain embodiments, the antibodies provided herein allow human thrombin generation as determined by thrombin generation assay (TGA).

In some embodiments, the antibodies provided herein bind human TF at a human TF binding site that is distinct from a human TF binding site bound by human FX. In certain embodiments, the antibodies provided herein do not interfere with the ability of TF:FVIIa to convert FX into FXa.

In some embodiments, the antibodies provided herein bind human TF at a human TF binding site that is distinct from a human TF binding site bound by human FVIIa. In certain embodiments, the antibodies provided herein do not compete for binding to human TF with human FVIIa.

In some embodiments, the antibodies provided herein bind to the extracellular domain of human TF, bind human TF at a human TF binding site that is distinct from a human TF binding site bound by human FVIIa, bind human TF at a human TF binding site that is distinct from a human TF binding site bound by human FX, and allow human thrombin generation as determined by thrombin generation assay (TGA).

In some embodiments, the antibodies provided herein bind to the extracellular domain of human TF, do not inhibit human thrombin generation as determined by thrombin generation assay (TGA), do not interfere with the ability of TF:FVIIa to convert FX into FXa, and do not compete for binding to human TF with human FVIIa.

In some embodiments, the antibodies provided herein bind to the extracellular domain of human TF at a human TF binding site that is distinct from a human TF binding site bound by human FVIIa, do not inhibit human thrombin generation as determined by thrombin generation assay (TGA), allow human thrombin generation as determined by thrombin generation assay (TGA), bind to human TF at a human TF binding site that is distinct from a human TF binding site bound by human FX, do not interfere with the ability of TF:FVIIa to convert FX into FXa, and do not compete for binding to human TF with human FVIIa.

In some embodiments, the antibodies provided herein inhibit FVIIa-dependent TF signaling.

In some embodiments, the antibodies provided herein bind to the extracellular domain of human TF at a human TF binding site that is distinct from a human TF binding site bound by human FVIIa, do not inhibit human thrombin generation as determined by thrombin generation assay (TGA), allow human thrombin generation as determined by thrombin generation assay (TGA), bind to human TF at a human TF binding site that is distinct from a human TF binding site bound by human FX, do not interfere with the ability of TF:FVIIa to convert FX into FXa, do not compete for binding to human TF with human FVIIa, and bind to cynomolgus and mouse TF.

In some embodiments, the antibodies provided herein bind to the extracellular domain of human TF at a human TF binding site that is distinct from a human TF binding site bound by human FVIIa, do not inhibit human thrombin generation as determined by thrombin generation assay (TGA), allow human thrombin generation as determined by thrombin generation assay (TGA), bind to human TF at a human TF binding site that is distinct from a human TF binding site bound by human FX, do not interfere with the ability of TF:FVIIa to convert FX into FXa, do not compete for binding to human TF with human FVIIa, bind to cynomolgus, mouse, and pig TF.

In some embodiments, the antibodies provided herein bind to the extracellular domain of human TF, inhibit FVIIa-dependent TF signaling, and bind to cynomolgus TF.

2.2. Sequences of TF Antibodies

2.2.1. Heavy Chain

In some embodiments, an antibody provided herein comprises a heavy chain sequence. Illustrative heavy chain sequences are provided in Table 22. The heavy chain sequence may be a heavy chain sequence from the antibody clone identified as 25A. The heavy chain sequence may be a heavy chain sequence from the antibody clone identified as 25A3. The heavy chain sequence may be a heavy chain sequence from the antibody clone identified as 25A5. The heavy chain sequence may be a heavy chain sequence from the antibody clone identified as 25A5T. The heavy chain sequence may be a heavy chain sequence from the antibody clone identified as 25G. The heavy chain sequence may be a heavy chain sequence from the antibody clone identified as 25G1. The heavy chain sequence may be a heavy chain sequence from the antibody clone identified as 25G9.

2.2.2. Light Chain

In some embodiments, an antibody provided herein comprises a light chain sequence. Illustrative light chain sequences are provided in Table 22. The light chain sequence may be a light chain sequence from the antibody clone identified as 25A. The light chain sequence may be a light chain sequence from the antibody clone identified as 25A3. The light chain sequence may be a light chain sequence from the antibody clone identified as 25A5. The light chain sequence may be a light chain sequence from the antibody clone identified as 25A5T. The light chain sequence may be a light chain sequence from the antibody clone identified as 25G. The light chain sequence may be a light chain sequence from the antibody clone identified as 25G1. The light chain sequence may be a light chain sequence from the antibody clone identified as 25G9.

2.2.3. V_{H} Domains

In some embodiments, an antibody provided herein comprises a V_{H} sequence selected from SEQ ID NOs: 113, 151, 189, 836, 227, 265, 303, 763, 868 and 870. In some embodiments, an antibody provided herein comprises a V_{H} sequence of SEQ ID NO:113. In some embodiments, an antibody provided herein comprises a V_{H} sequence of SEQ ID NO:151. In some embodiments, an antibody provided herein comprises a V_{H} sequence of SEQ ID NO:189. In some embodiments, an antibody provided herein comprises a V_{H} sequence of SEQ ID NO:836. In some embodiments, an antibody provided herein comprises a V_{H} sequence of SEQ ID NO:227. In some embodiments, an antibody provided herein comprises a V_{H} sequence of SEQ ID NO:265. In some embodiments, an antibody provided herein comprises a V_{H} sequence of SEQ ID NO:303. In some embodiments, an antibody provided herein comprises a V_{H} sequence of SEQ ID NO: 763. In some embodiments, an antibody provided herein comprises a V_{H} sequence of SEQ ID NO: 868.

In some embodiments, an antibody provided herein comprises a V_{H} sequence having at least about 50%, 60%, 70%, 80%, 90%, 95%, or 99% identity to an illustrative V_{H} sequence selected from SEQ ID NOs: 113, 151, 189, 836, 227, 265, 303, 763, 868, and 870. In some embodiments, an antibody provided herein comprises a V_{H} sequence selected from SEQ ID NOs: 113, 151, 189, 836, 227, 265, 303, 763, 868, and 870, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

2.2.4. V_{L} Domains

In some embodiments, an antibody provided herein comprises a V_{L} sequence selected from SEQ ID NOs: 114, 152, 190, 837, 228, 266, 304, 764, 869, and 871. In some embodiments, an antibody provided herein comprises a V_{L} sequence of SEQ ID NO:114. In some embodiments, an antibody provided herein comprises a V_{L} sequence of SEQ ID NO:152. In some embodiments, an antibody provided herein comprises a V_{L} sequence of SEQ ID NO:190. In some embodiments, an antibody provided herein comprises a V_{L} sequence of SEQ ID NO:837. In some embodiments, an antibody provided herein comprises a V_{L} sequence of SEQ ID NO:228. In some embodiments, an antibody provided herein comprises a V_{L} sequence of SEQ ID NO:266. In some embodiments, an antibody provided herein comprises a V_{L} sequence of SEQ ID NO:304. In some embodiments, an antibody provided herein comprises a V_{L} sequence of SEQ ID NO: 764. In some embodiments, an antibody provided herein comprises a V_{L} sequence of SEQ ID NO: 869. In some embodiments, an antibody provided herein comprises a V_{L} sequence of SEQ ID NO: 871.

In some embodiments, an antibody provided herein comprises a V_{L} sequence having at least about 50%, 60%, 70%, 80%, 90%, 95%, or 99% identity to an illustrative VL sequence selected from SEQ ID NOs: 114, 152, 190, 837, 228, 266, 304, 764, 869, and 871. In some embodiments, an antibody provided herein comprises a V_{L} sequence selected from SEQ ID NOs: 114, 152, 190, 837, 228, 266, 304, 764, 869, and 871, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

2.2.5. V_{H}-V_{L} Combinations

In some embodiments, an antibody provided herein comprises a V_{H} sequence selected from SEQ ID NOs: 113, 151, 189, 836, 227, 265, 303, 763, 868, and 870 and a V_{L} sequence selected from SEQ ID NOs: 114, 152, 190, 837, 228, 266, 304, 764, 869, and 871.

In some embodiments, an antibody provided herein comprises a VH sequence of SEQ ID NO:113 and a VL sequence of SEQ ID NO:114. In some embodiments, an antibody provided herein comprises a VH sequence of SEQ ID NO:151 and a VL sequence of SEQ ID NO:152. In some embodiments, an antibody provided herein comprises a VH sequence of SEQ ID NO:189 and a VL sequence of SEQ ID NO:190. In some embodiments, an antibody provided herein comprises a VH sequence of SEQ ID NO:836 and a VL sequence of SEQ ID NO:837. In some embodiments, an antibody provided herein comprises a VH sequence of SEQ ID NO:227 and a VL sequence of SEQ ID NO:228. In some embodiments, an antibody provided herein comprises a VH sequence of SEQ ID NO:265 and a VL sequence of SEQ ID NO:266. In some embodiments, an antibody provided herein comprises a VH sequence of SEQ ID NO:303 and a VL sequence of SEQ ID NO:304. In some embodiments, an antibody provided herein comprises a VH sequence of SEQ ID NO:763 and a VL sequence of SEQ ID NO:764. In some embodiments, an antibody provided herein comprises a VH sequence of SEQ ID NO:868 and a VL sequence of SEQ ID NO:869. In some embodiments, an antibody provided herein comprises a VH sequence of SEQ ID NO:870 and a VL sequence of SEQ ID NO:871.

In some embodiments, an antibody provided herein comprises a V_{H} sequence having at least about 50%, 60%, 70%, 80%, 90%, 95%, or 99% identity to an illustrative V_{H} sequence selected from SEQ ID NOs: 113, 151, 189, 836, 227, 265, 303, 763, 868, and 870, and a V_{L} sequence having at least about 50%, 60%, 70%, 80%, 90%, 95%, or 99% identity to an illustrative VL sequence selected from SEQ ID NOs: 114, 152, 190, 837, 228, 266, 304, 764, 869, and 871. In some embodiments, an antibody provided herein comprises a V_{H} sequence selected from SEQ ID NOs: 113, 151, 189, 836, 227, 265, 303, 763, 868, and 870, with up to 1, 2, 3, 4, 5, 6, 7, 8, 9, 10, 11, 12, 13, 14, 15, 16, 17, 18, 19, 20, 21, 22, 23, 24, or 25 amino acid substitutions, and a V_{L} sequence selected from SEQ ID NOs: 114, 152, 190, 837, 228, 266, 304, 764, 869, and 871, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

2.2.6. CDRs

In some embodiments, an antibody provided herein comprises one to three CDRs of a V_{H} domain selected from SEQ ID NOs: 113, 151, 189, 836, 227, 265, 303, 763, 868, and 870. In some embodiments, an antibody provided herein comprises two to three CDRs of a V_{H} domain selected from SEQ ID NOs: 113, 151, 189, 836, 227, 265, 303, 763, 868, and 870. In some embodiments, an antibody provided herein comprises three CDRs of a V_{H} domain selected from SEQ ID NOs: 113, 151, 189, 836, 227, 265, 303, 763, 868, and 870. In some aspects, the CDRs are Exemplary CDRs. In some aspects, the CDRs are Kabat CDRs. In some aspects, the CDRs are Chothia CDRs. In some aspects, the CDRs are AbM CDRs. In some aspects, the CDRs are Contact CDRs. In some aspects, the CDRs are IMGT CDRs.

In some embodiments, the CDRs are CDRs having at least about 50%, 75%, 80%, 85%, 90%, or 95% identity with a CDR-H1, CDR-H2, or CDR-H3 of SEQ ID NOs: 113, 151, 189, 836, 227, 265, 303, 763, 868, and 870. In some embodiments, the CDR-H1 is a CDR-H1 of a V_{H} domain selected from SEQ ID NOs: 113, 151, 189, 836, 227, 265, 303, 763, 868, and 870, with up to 1, 2, 3, 4, or 5 amino acid substitutions. In some embodiments, the CDR-H2 is a CDR-H2 of a V_{H} domain selected from SEQ ID NOs: 113, 151, 189, 836, 227, 265, 303, 763, 868, and 870, with up to 1, 2, 3, 4, 5, 6, 7, or 8 amino acid substitutions. In some embodiments, the CDR-H3 is a CDR-H3 of a V_{H} domain selected from SEQ ID NOs: 113, 151, 189, 836, 227, 265, 303, 763, 868, and 870, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises one to three CDRs of a V_{L} domain selected from SEQ ID NOs: 114, 152, 190, 837, 228, 266, 304, 764, 869, and 871. In some embodiments, an antibody provided herein comprises two to three CDRs of a V_{L} domain selected from SEQ ID NOs: 114, 152, 190, 837, 228, 266, 304, 764, 869, and 871. In some embodiments, an antibody provided herein comprises three CDRs of a V_{L} domain selected from SEQ ID NOs: 114, 152, 190, 837, 228, 266, 304, 764, 869, and 871. In some aspects, the CDRs are Exemplary CDRs. In some aspects, the CDRs are Kabat CDRs. In some aspects, the CDRs are Chothia CDRs. In some aspects, the CDRs are AbM CDRs. In some aspects, the CDRs are Contact CDRs. In some aspects, the CDRs are IMGT CDRs.

In some embodiments, the CDRs are CDRs having at least about 50%, 75%, 80%, 85%, 90%, or 95% identity with a CDR-L1, CDR-L2, or CDR-L3 of SEQ ID NOs: 114, 152, 190, 837, 228, 266, 304, 764, 869, and 871. In some embodiments, the CDR-L1 is a CDR-L1 of a V_{L} domain selected from SEQ ID NOs: 114, 152, 190, 837, 228, 266, 304, 764, 869, and 871, with up to 1, 2, 3, 4, or 5 amino acid substitutions. In some embodiments, the CDR-L2 is a CDR-L2 of a V_{L} domain selected from SEQ ID NOs: 114, 152, 190, 837, 228, 266, 304, 764, 869, and 871, with up to 1, 2, 3, 4, 5, 6, 7, or 8 amino acid substitutions. In some embodiments, the CDR-L3 is a CDR-L3 of a V_{L} domain selected from SEQ ID NOs: 114, 152, 190, 837, 228, 266, 304, 764, 869, and 871, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises one to three CDRs of a V_{H} domain selected from SEQ ID NOs: 113, 151, 189, 836, 227, 265, 303, 763, 868, and 870 and one to three CDRs of a V_{L} domain selected from SEQ ID NOs: 114, 152, 190, 837, 228, 266, 304, 764, 869, and 871. In some embodiments, an antibody provided herein comprises two to three CDRs of a V_{H} domain selected from SEQ ID NOs: 113, 151, 189, 836, 227, 265, 303, 763, 868, and 870 and two to three CDRs of a V_{L} domain selected from SEQ ID NOs: 114, 152, 190, 837, 228, 266, 304, 764, 869, and 871. In some embodiments, an antibody provided herein comprises three CDRs of a V_{H} domain selected from SEQ ID NOs: 113, 151, 189, 836, 227, 265, 303, 763, 868, and 870 and three CDRs of a V_{L} domain selected from SEQ ID NOs: 114, 152, 190, 837, 228, 266, 304, 764, 869, and 871. In some aspects, the CDRs are Exemplary CDRs. In some aspects, the CDRs are Kabat CDRs. In some aspects, the CDRs are Chothia CDRs. In some aspects, the CDRs are AbM CDRs. In some aspects, the CDRs are Contact CDRs. In some aspects, the CDRs are IMGT CDRs.

In some embodiments, the CDRs are CDRs having at least about 50%, 75%, 80%, 85%, 90%, or 95% identity with a CDR-H1, CDR-H2, or CDR-H3 of SEQ ID NOs: 113, 151, 189, 836, 227, 265, 303, 763, 868, and 870 and at least about 50%, 75%, 80%, 85%, 90%, or 95% identity with a CDR-L1, CDR-L2, or CDR-L3 of SEQ ID NOs: 114, 152, 190, 837, 228, 266, 304, 764, 869, and 871. In some embodiments, the CDR-H1 is a CDR-H1 of a V_{H} domain selected from SEQ ID NOs: 113, 151, 189, 836, 227, 265, 303, 763, 868, and 870, with up to 1, 2, 3, 4, or 5 amino acid substitutions; the CDR-H2 is a CDR-H2 of a V_{H} domain selected from SEQ ID NOs: 113, 151, 189, 836, 227, 265, 303, 763, 868, and 870, with up to 1, 2, 3, 4, 5, 6, 7, or 8 amino acid substitutions; the CDR-H3 is a CDR-H3 of a V_{H} domain selected from SEQ ID NOs: 113, 151, 189, 836, 227, 265, 303, 763, 868, and 870, with up to 1, 2, 3, 4, 5, 6, 7, or 8 amino acid substitutions; the CDR-L1 is a CDR-L1 of a V_{L} domain selected from SEQ ID NOs: 114, 152, 190, 837, 228, 266, 304, 764, 869, and 871, with up to 1, 2, 3, 4, 5, or 6 amino acid substitutions; the CDR-L2 is a CDR-L2 of a V_{L} domain selected from SEQ ID NOs: 114, 152, 190, 837, 228, 266, 304, 764, 869, and 871, with up to 1, 2, 3, or 4 amino acid substitutions; and the CDR-L3 is a CDR-L3 of a VL domain selected from SEQ ID NOs: 114, 152, 190, 837, 228, 266, 304, 764, 869, and 871, with up to 1, 2, 3, 4, or 5 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

25A CDRs

In some embodiments, the antibody comprises a heavy chain CDR sequence from antibody clone 25A. Antibody 25A CDR sequences as determined by the Exemplary, Kabat, Chothia, AbM, Contact, and IMGT numbering systems are shown in Table 7. In some embodiments, the antibody comprises a CDR-H3 sequence that is 50%, 75%, 80%, 85%, 90%, 95%, or 100% identical to the CDR-H3 sequence from antibody clone 25A. In some embodiments, the antibody comprises a CDR-H2 sequence that is 50%, 75%, 80%, 85%, 90%, 95%, or 100% identical to the CDR-H2 sequence from antibody clone 25A. In some embodiments, the antibody comprises a CDR-H1 sequence that is 50%, 75%, 80%, 85%, 90%, 95%, or 100% identical to the CDR-H1 sequence from antibody clone 25A. In some embodiments, the antibody comprises two heavy chain CDRs that are 50%, 75%, 80%, 85%, 90%, 95%, or 100% identical to the corresponding two heavy chain CDRs from antibody clone 25A. In some embodiments, the antibody comprises three heavy chain CDRs that are 50%, 75%, 80%, 85%, 90%, 95%, or 100% identical to the three heavy chain CDRs from antibody clone 25A.

In some embodiments, the antibody comprises a light chain CDR from antibody clone 25A. In some embodiments, the antibody comprises a CDR-L3 sequence that is 50%, 75%, 80%, 85%, 90%, 95%, or 100% identical to the CDR-L3 sequence from antibody clone 25A. In some embodiments, the antibody comprises a CDR-L2 sequence that is 50%, 75%, 80%, 85%, 90%, 95%, or 100% identical to the CDR-L2 sequence from antibody clone 25A. In some embodiments, the antibody comprises a CDR-L1 sequence that is 50%, 75%, 80%, 85%, 90%, 95%, or 100% identical to the CDR-L1 sequence from antibody clone 25A. In some embodiments, the antibody comprises two light chain CDRs that are 50%, 75%, 80%, 85%, 90%, 95%, or 100% identical to the corresponding two light chain CDRs from antibody clone 25A. In some embodiments, the antibody comprises three light chain CDRs that are 50%, 75%, 80%, 85%, 90%, 95%, or 100% identical to the three light chain CDRs from antibody clone 25A.

In some embodiments, the antibody comprises a CHR—H3 sequence that is 50%, 75%, 80%, 85%, 90%, 95%, or 100% identical to the CDR-H3 sequence from antibody clone 25A and CDR-L3 sequence that is 50%, 75%, 80%, 85%, 90%, 95%, or 100% identical to the CDR-L3 sequence from antibody clone 25A. In some embodiments, the antibody comprises six CDR sequences that are 50%, 75%, 80%, 85%, 90%, 95%, or 100% identical to the corresponding six CDRs from antibody clone 25A.

25A3 CDRs

In some embodiments, the antibody comprises a heavy chain CDR sequence from antibody clone 25A3. Antibody 25A3 CDR sequences as determined by the Exemplary, Kabat, Chothia, AbM, Contact, and IMGT numbering systems are shown in Table 8. In some embodiments, the antibody comprises a CDR-H3 sequence that is 50%, 75%, 80%, 85%, 90%, 95%, or 100% identical to the CDR-H3 sequence from antibody clone 25A3. In some embodiments, the antibody comprises a CDR-H2 sequence that is 50%, 75%, 80%, 85%, 90%, 95%, or 100% identical to the CDR-H2 sequence from antibody clone 25A3. In some embodiments, the antibody comprises a CDR-H1 sequence that is 50%, 75%, 80%, 85%, 90%, 95%, or 100% identical to the CDR-H1 sequence from antibody clone 25A3. In some embodiments, the antibody comprises two heavy chain CDRs that are 50%, 75%, 80%, 85%, 90%, 95%, or 100% identical to the corresponding two heavy chain CDRs from antibody clone 25A3. In some embodiments, the antibody comprises three heavy chain CDRs that are 50%, 75%, 80%, 85%, 90%, 95%, or 100% identical to the three heavy chain CDRs from antibody clone 25A3.

In some embodiments, the antibody comprises a light chain CDR from antibody clone 25A3. In some embodiments, the antibody comprises a CDR-L3 sequence that is 50%, 75%, 80%, 85%, 90%, 95%, or 100% identical to the CDR-L3 sequence from antibody clone 25A3. In some embodiments, the antibody comprises a CDR-L2 sequence that is 50%, 75%, 80%, 85%, 90%, 95%, or 100% identical to the CDR-L2 sequence from antibody clone 25A3. In some embodiments, the antibody comprises a CDR-L1 sequence that is 50%, 75%, 80%, 85%, 90%, 95%, or 100% identical to the CDR-L1 sequence from antibody clone 25A3. In some embodiments, the antibody comprises two light chain CDRs that are 50%, 75%, 80%, 85%, 90%, 95%, or 100% identical to the corresponding two light chain CDRs from antibody clone 25A3. In some embodiments, the antibody comprises three light chain CDRs that are 50%, 75%, 80%, 85%, 90%, 95%, or 100% identical to the three light chain CDRs from antibody clone 25A3.

In some embodiments, the antibody comprises a CHR—H3 sequence that is 50%, 75%, 80%, 85%, 90%, 95%, or 100% identical to the CDR-H3 sequence from antibody clone 25A3 and CDR-L3 sequence that is 50%, 75%, 80%, 85%, 90%, 95%, or 100% identical to the CDR-L3 sequence from antibody clone 25A3. In some embodiments, the antibody comprises six CDR sequences that are 50%, 75%, 80%, 85%, 90%, 95%, or 100% identical to the corresponding six CDRs from antibody clone 25A3.

25A5 CDRs

In some embodiments, the antibody comprises a heavy chain CDR sequence from antibody clone 25A5. Antibody 25A5 CDR sequences as determined by the Exemplary, Kabat, Chothia, AbM, Contact, and IMGT numbering systems are shown in Table 9. In some embodiments, the antibody comprises a CDR-H3 sequence that is 50%, 75%, 80%, 85%, 90%, 95%, or 100% identical to the CDR-H3 sequence from antibody clone 25A5. In some embodiments, the antibody comprises a CDR-H2 sequence that is 50%, 75%, 80%, 85%, 90%, 95%, or 100% identical to the CDR-H2 sequence from antibody clone 25A5. In some embodiments, the antibody comprises a CDR-H1 sequence that is 50%, 75%, 80%, 85%, 90%, 95%, or 100% identical to the CDR-H1 sequence from antibody clone 25A5. In some embodiments, the antibody comprises two heavy chain CDRs that are 50%, 75%, 80%, 85%, 90%, 95%, or 100% identical to the corresponding two heavy chain CDRs from antibody clone 25A5. In some embodiments, the antibody comprises three heavy chain CDRs that are 50%, 75%, 80%, 85%, 90%, 95%, or 100% identical to the three heavy chain CDRs from antibody clone 25A5.

In some embodiments, the antibody comprises a light chain CDR from antibody clone 25A5. In some embodiments, the antibody comprises a CDR-L3 sequence that is 50%, 75%, 80%, 85%, 90%, 95%, or 100% identical to the CDR-L3 sequence from antibody clone 25A5. In some embodiments, the antibody comprises a CDR-L2 sequence that is 50%, 75%, 80%, 85%, 90%, 95%, or 100% identical to the CDR-L2 sequence from antibody clone 25A5. In some embodiments, the antibody comprises a CDR-L1 sequence that is 50%, 75%, 80%, 85%, 90%, 95%, or 100% identical to the CDR-L1 sequence from antibody clone 25A5. In some embodiments, the antibody comprises two light chain CDRs that are 50%, 75%, 80%, 85%, 90%, 95%, or 100% identical to the corresponding two light chain CDRs from antibody clone 25A5. In some embodiments, the antibody comprises three light chain CDRs that are 50%, 75%, 80%, 85%, 90%, 95%, or 100% identical to the three light chain CDRs from antibody clone 25A5.

In some embodiments, the antibody comprises a CHR—H3 sequence that is 50%, 75%, 80%, 85%, 90%, 95%, or 100% identical to the CDR-H3 sequence from antibody clone 25A5 and CDR-L3 sequence that is 50%, 75%, 80%, 85%, 90%, 95%, or 100% identical to the CDR-L3 sequence from antibody clone 25A5. In some embodiments, the antibody comprises six CDR sequences that are 50%, 75%, 80%, 85%, 90%, 95%, or 100% identical to the corresponding six CDRs from antibody clone 25A5.

25A5-T CDRs

In some embodiments, the antibody comprises a heavy chain CDR sequence from antibody clone 25A5-T. Antibody 25A5-T CDR sequences as determined by the Exemplary, Kabat, Chothia, AbM, Contact, and IMGT numbering systems are shown in Table 10. In some embodiments, the antibody comprises a CDR-H3 sequence that is 50%, 75%, 80%, 85%, 90%, 95%, or 100% identical to the CDR-H3 sequence from antibody clone 25A5-T. In some embodiments, the antibody comprises a CDR-H2 sequence that is 50%, 75%, 80%, 85%, 90%, 95%, or 100% identical to the CDR-H2 sequence from antibody clone 25A5-T. In some embodiments, the antibody comprises a CDR-H1 sequence that is 50%, 75%, 80%, 85%, 90%, 95%, or 100% identical to the CDR-H1 sequence from antibody clone 25A5-T. In some embodiments, the antibody comprises two heavy chain CDRs that are 50%, 75%, 80%, 85%, 90%, 95%, or 100% identical to the corresponding two heavy chain CDRs from antibody clone 25A5-T. In some embodiments, the antibody comprises three heavy chain CDRs that are 50%, 75%, 80%, 85%, 90%, 95%, or 100% identical to the three heavy chain CDRs from antibody clone 25A5-T.

In some embodiments, the antibody comprises a light chain CDR from antibody clone 25A5-T. In some embodiments, the antibody comprises a CDR-L3 sequence that is 50%, 75%, 80%, 85%, 90%, 95%, or 100% identical to the CDR-L3 sequence from antibody clone 25A5-T. In some embodiments, the antibody comprises a CDR-L2 sequence that is 50%, 75%, 80%, 85%, 90%, 95%, or 100% identical to the CDR-L2 sequence from antibody clone 25A5-T. In some embodiments, the antibody comprises a CDR-L1 sequence that is 50%, 75%, 80%, 85%, 90%, 95%, or 100% identical to the CDR-L1 sequence from antibody clone 25A5-T. In some embodiments, the antibody comprises two light chain CDRs that are 50%, 75%, 80%, 85%, 90%, 95%, or 100% identical to the corresponding two light chain CDRs from antibody clone 25A5-T. In some embodiments, the antibody comprises three light chain CDRs that are 50%, 75%, 80%, 85%, 90%, 95%, or 100% identical to the three light chain CDRs from antibody clone 25A5-T.

In some embodiments, the antibody comprises a CHR—H3 sequence that is 50%, 75%, 80%, 85%, 90%, 95%, or 100% identical to the CDR-H3 sequence from antibody clone 25A5-T and CDR-L3 sequence that is 50%, 75%, 80%, 85%, 90%, 95%, or 100% identical to the CDR-L3 sequence from antibody clone 25A5-T. In some embodiments, the antibody comprises six CDR sequences that are 50%, 75%, 80%, 85%, 90%, 95%, or 100% identical to the corresponding six CDRs from antibody clone 25A5-T.

25G CDRs

In some embodiments, the antibody comprises a heavy chain CDR sequence from antibody clone 25G. Antibody 25G CDR sequences as determined by the Exemplary, Kabat, Chothia, AbM, Contact, and IMGT numbering systems are shown in Table 11. In some embodiments, the antibody comprises a CDR-H3 sequence that is 50%, 75%, 80%, 85%, 90%, 95%, or 100% identical to the CDR-H3 sequence from antibody clone 25G. In some embodiments, the antibody comprises a CDR-H2 sequence that is 50%, 75%, 80%, 85%, 90%, 95%, or 100% identical to the CDR-H2 sequence from antibody clone 25G. In some embodiments, the antibody comprises a CDR-H1 sequence that is 50%, 75%, 80%, 85%, 90%, 95%, or 100% identical to the CDR-H1 sequence from antibody clone 25G. In some embodiments, the antibody comprises two heavy chain CDRs that are 50%, 75%, 80%, 85%, 90%, 95%, or 100% identical to the corresponding two heavy chain CDRs from antibody clone 25G. In some embodiments, the antibody comprises three heavy chain CDRs that are 50%, 75%, 80%, 85%, 90%, 95%, or 100% identical to the three heavy chain CDRs from antibody clone 25G.

In some embodiments, the antibody comprises a light chain CDR from antibody clone 25G. In some embodiments, the antibody comprises a CDR-L3 sequence that is 50%, 75%, 80%, 85%, 90%, 95%, or 100% identical to the CDR-L3 sequence from antibody clone 25G. In some embodiments, the antibody comprises a CDR-L2 sequence that is 50%, 75%, 80%, 85%, 90%, 95%, or 100% identical to the CDR-L2 sequence from antibody clone 25G. In some embodiments, the antibody comprises a CDR-L1 sequence that is 50%, 75%, 80%, 85%, 90%, 95%, or 100% identical to the CDR-L1 sequence from antibody clone 25G. In some embodiments, the antibody comprises two light chain CDRs that are 50%, 75%, 80%, 85%, 90%, 95%, or 100% identical to the corresponding two light chain CDRs from antibody clone 25G. In some embodiments, the antibody comprises three light chain CDRs that are 50%, 75%, 80%, 85%, 90%, 95%, or 100% identical to the three light chain CDRs from antibody clone 25G.

In some embodiments, the antibody comprises a CHR—H3 sequence that is 50%, 75%, 80%, 85%, 90%, 95%, or 100% identical to the CDR-H3 sequence from antibody clone 25G and CDR-L3 sequence that is 50%, 75%, 80%, 85%, 90%, 95%, or 100% identical to the CDR-L3 sequence from antibody clone 25G. In some embodiments, the antibody comprises six CDR sequences that are 50%, 75%, 80%, 85%, 90%, 95%, or 100% identical to the corresponding six CDRs from antibody clone 25G.

25G1 CDRs

In some embodiments, the antibody comprises a heavy chain CDR sequence from antibody clone 25G1. Antibody 25G1 CDR sequences as determined by the Exemplary, Kabat, Chothia, AbM, Contact, and IMGT numbering systems are shown in Table 12. In some embodiments, the antibody comprises a CDR-H3 sequence that is 50%, 75%, 80%, 85%, 90%, 95%, or 100% identical to the CDR-H3 sequence from antibody clone 25G1. In some embodiments, the antibody comprises a CDR-H2 sequence that is 50%, 75%, 80%, 85%, 90%, 95%, or 100% identical to the CDR-H2 sequence from antibody clone 25G1. In some embodiments, the antibody comprises a CDR-H1 sequence that is 50%, 75%, 80%, 85%, 90%, 95%, or 100% identical to the CDR-H1 sequence from antibody clone 25G1. In some embodiments, the antibody comprises two heavy chain CDRs that are 50%, 75%, 80%, 85%, 90%, 95%, or 100% identical to the corresponding two heavy chain CDRs from antibody clone 25G1. In some embodiments, the antibody comprises three heavy chain CDRs that are 50%, 75%, 80%, 85%, 90%, 95%, or 100% identical to the three heavy chain CDRs from antibody clone 25G1.

In some embodiments, the antibody comprises a light chain CDR from antibody clone 25G1. In some embodiments, the antibody comprises a CDR-L3 sequence that is 50%, 75%, 80%, 85%, 90%, 95%, or 100% identical to the CDR-L3 sequence from antibody clone 25G1. In some embodiments, the antibody comprises a CDR-L2 sequence that is 50%, 75%, 80%, 85%, 90%, 95%, or 100% identical to the CDR-L2 sequence from antibody clone 25G1. In some embodiments, the antibody comprises a CDR-L1 sequence that is 50%, 75%, 80%, 85%, 90%, 95%, or 100% identical to the CDR-L1 sequence from antibody clone 25G1. In some embodiments, the antibody comprises two light chain CDRs that are 50%, 75%, 80%, 85%, 90%, 95%, or 100% identical to the corresponding two light chain CDRs from antibody clone 25G1. In some embodiments, the antibody comprises three light chain CDRs that are 50%, 75%, 80%, 85%, 90%, 95%, or 100% identical to the three light chain CDRs from antibody clone 25G1.

In some embodiments, the antibody comprises a CHR—H3 sequence that is 50%, 75%, 80%, 85%, 90%, 95%, or 100% identical to the CDR-H3 sequence from antibody clone 25G1 and CDR-L3 sequence that is 50%, 75%, 80%, 85%, 90%, 95%, or 100% identical to the CDR-L3 sequence from antibody clone 25G1. In some embodiments, the antibody comprises six CDR sequences that are 50%, 75%, 80%, 85%, 90%, 95%, or 100% identical to the corresponding six CDRs from antibody clone 25G1.

25G9 CDRs

In some embodiments, the antibody comprises a heavy chain CDR sequence from antibody clone 25G9. Antibody 25G9 CDR sequences as determined by the Exemplary, Kabat, Chothia, AbM, Contact, and IMGT numbering systems are shown in Table 13. In some embodiments, the antibody comprises a CDR-H3 sequence that is 50%, 75%, 80%, 85%, 90%, 95%, or 100% identical to the CDR-H3 sequence from antibody clone 25G9. In some embodiments, the antibody comprises a CDR-H2 sequence that is 50%, 75%, 80%, 85%, 90%, 95%, or 100% identical to the CDR-H2 sequence from antibody clone 25G9. In some embodiments, the antibody comprises a CDR-H1 sequence that is 50%, 75%, 80%, 85%, 90%, 95%, or 100% identical to the CDR-H1 sequence from antibody clone 25G9. In some embodiments, the antibody comprises two heavy chain CDRs that are 50%, 75%, 80%, 85%, 90%, 95%, or 100% identical to the corresponding two heavy chain CDRs from antibody clone 25G9. In some embodiments, the antibody comprises three heavy chain CDRs that are 50%, 75%, 80%, 85%, 90%, 95%, or 100% identical to the three heavy chain CDRs from antibody clone 25G9.

In some embodiments, the antibody comprises a light chain CDR from antibody clone 25G9. In some embodiments, the antibody comprises a CDR-L3 sequence that is 50%, 75%, 80%, 85%, 90%, 95%, or 100% identical to the CDR-L3 sequence from antibody clone 25G9. In some embodiments, the antibody comprises a CDR-L2 sequence that is 50%, 75%, 80%, 85%, 90%, 95%, or 100% identical to the CDR-L2 sequence from antibody clone 25G9. In some embodiments, the antibody comprises a CDR-L1 sequence that is 50%, 75%, 80%, 85%, 90%, 95%, or 100% identical to the CDR-L1 sequence from antibody clone 25G9. In some embodiments, the antibody comprises two light chain CDRs that are 50%, 75%, 80%, 85%, 90%, 95%, or 100% identical to the corresponding two light chain CDRs from antibody clone 25G9. In some embodiments, the antibody comprises three light chain CDRs that are 50%, 75%, 80%, 85%, 90%, 95%, or 100% identical to the three light chain CDRs from antibody clone 25G9.

In some embodiments, the antibody comprises a CHR—H3 sequence that is 50%, 75%, 80%, 85%, 90%, 95%, or 100% identical to the CDR-H3 sequence from antibody clone 25G9 and CDR-L3 sequence that is 50%, 75%, 80%, 85%, 90%, 95%, or 100% identical to the CDR-L3 sequence from antibody clone 25G9. In some embodiments, the antibody comprises six CDR sequences that are 50%, 75%, 80%, 85%, 90%, 95%, or 100% identical to the corresponding six CDRs from antibody clone 25G9.

25 Consensus CDRs

In some embodiments, the antibody comprises a heavy chain consensus CDR sequence from the antibody group identified as Group 25. Antibody Group 25 consensus CDR sequences as determined by Kabat and Chothia numbering systems are shown in Table 14. In some embodiments, the antibody comprises a CDR-H3 sequence that is 50%, 75%, 80%, 85%, 90%, 95%, or 100% identical to the CDR-H3 consensus sequence from the antibody group identified as Group 25. In some embodiments, the antibody comprises a CDR-H2 sequence that is 50%, 75%, 80%, 85%, 90%, 95%, or 100% identical to the consensus CDR-H2 sequence from the antibody group identified as Group 25. In some embodiments, the antibody comprises a CDR-H1 sequence that is 50%, 75%, 80%, 85%, 90%, 95%, or 100% identical to the consensus CDR-H1 sequence from the antibody group identified as Group 25. In some embodiments, the antibody comprises two heavy chain CDRs that are 50%, 75%, 80%, 85%, 90%, 95%, or 100% identical to the corresponding two consensus heavy chain CDRs from the antibody group identified as Group 25. In some embodiments, the antibody comprises three heavy chain CDRs that are 50%, 75%, 80%, 85%, 90%, 95%, or 100% identical to the three consensus heavy chain CDRs from the antibody group identified as Group 25.

In some embodiments, the antibody comprises a light chain consensus CDR from the antibody group identified as Group 25. In some embodiments, the antibody comprises a CDR-L3 sequence that is 50%, 75%, 80%, 85%, 90%, 95%, or 100% identical to the consensus CDR-L3 sequence from the antibody group identified as Group 25. In some embodiments, the antibody comprises a CDR-L2 sequence that is 50%, 75%, 80%, 85%, 90%, 95%, or 100% identical to the consensus CDR-L2 sequence from the antibody group identified as Group 25. In some embodiments, the antibody comprises a CDR-L1 sequence that is 50%, 75%, 80%, 85%, 90%, 95%, or 100% identical to the consensus CDR-L1 sequence from the antibody group identified as Group 25. In some embodiments, the antibody comprises two light chain CDRs that are 50%, 75%, 80%, 85%, 90%, 95%, or 100% identical to the corresponding two consensus light chain CDRs from the antibody group identified as Group 25. In some embodiments, the antibody comprises three light chain CDRs that are 50%, 75%, 80%, 85%, 90%, 95%, or 100% identical to the corresponding three consensus light chain CDRs from the antibody group identified as Group 25.

In some embodiments, the antibody comprises a CHR—H3 sequence that is 50%, 75%, 80%, 85%, 90%, 95%, or 100% identical to the consensus CDR-H3 sequence from the antibody group identified as Group 25 and CDR-L3 sequence that is 50%, 75%, 80%, 85%, 90%, 95%, or 100% identical to the consensus CDR-L3 sequence from the antibody group identified as Group 25. In some embodiments, the antibody comprises six CDR sequences that are 50%, 75%, 80%, 85%, 90%, 95%, or 100% identical to the corresponding six consensus CDRs from the antibody group identified as Group 25.

25A Consensus CDRs

In some embodiments, the antibody comprises a heavy chain consensus CDR sequence from antibody group lineage 25A. Consensus CDR sequences for antibody group lineage 25A as determined by the Kabat and Chothia numbering systems are shown in Table 21. In some embodiments, the antibody comprises a CDR-H3 sequence that is 50%, 75%, 80%, 85%, 90%, 95%, or 100% identical to the consensus CDR-H3 sequence from antibody group lineage 25A. In some embodiments, the antibody comprises a CDR-H2 sequence that is 50%, 75%, 80%, 85%, 90%, 95%, or 100% identical to the consensus CDR-H2 sequence from antibody group lineage 25A. In some embodiments, the antibody comprises a CDR-H1 sequence that is 50%, 75%, 80%, 85%, 90%, 95%, or 100% identical to the consensus CDR-H1 sequence from antibody group lineage 25A. In some embodiments, the antibody comprises two heavy chain CDRs that are 50%, 75%, 80%, 85%, 90%, 95%, or 100% identical to the corresponding consensus two heavy chain CDRs from antibody group lineage 25A. In some embodiments, the antibody comprises three heavy chain CDRs that are 50%, 75%, 80%, 85%, 90%, 95%, or 100% identical to the three consensus heavy chain CDRs from antibody group lineage 25A.

In some embodiments, the antibody comprises a light chain CDR from antibody group lineage 25A. In some embodiments, the antibody comprises a CDR-L3 sequence that is 50%, 75%, 80%, 85%, 90%, 95%, or 100% identical to the consensus CDR-L3 sequence from antibody group lineage 25A. In some embodiments, the antibody comprises a CDR-L2 sequence that is 50%, 75%, 80%, 85%, 90%, 95%, or 100% identical to the consensus CDR-L2 sequence from antibody group lineage 25A. In some embodiments, the antibody comprises a CDR-L1 sequence that is 50%, 75%, 80%, 85%, 90%, 95%, or 100% identical to the consensus CDR-L1 sequence from antibody group lineage 25A. In some embodiments, the antibody comprises two light chain CDRs that are 50%, 75%, 80%, 85%, 90%, 95%, or 100% identical to the corresponding consensus two light chain CDRs from antibody group lineage 25A. In some embodiments, the antibody comprises three light chain CDRs that are 50%, 75%, 80%, 85%, 90%, 95%, or 100% identical to the three light chain consensus CDRs from antibody group lineage 25A.

In some embodiments, the antibody comprises a CHR—H3 sequence that is 50%, 75%, 80%, 85%, 90%, 95%, or 100% identical to the consensus CDR-H3 sequence from antibody group lineage 25A and CDR-L3 sequence that is 50%, 75%, 80%, 85%, 90%, 95%, or 100% identical to the consensus CDR-L3 sequence from antibody group lineage 25A. In some embodiments, the antibody comprises six CDR sequences that are 50%, 75%, 80%, 85%, 90%, 95%, or 100% identical to the corresponding six consensus CDRs from antibody group lineage 25A.

25G Consensus CDRs

In some embodiments, the antibody comprises a heavy chain consensus CDR sequence from antibody group lineage 25G. Consensus CDR sequences for antibody group lineage 25G as determined by the Kabat and Chothia numbering systems are shown in Table 21. In some embodiments, the antibody comprises a CDR-H3 sequence that is 50%, 75%, 80%, 85%, 90%, 95%, or 100% identical to the consensus CDR-H3 sequence from antibody group lineage 25G. In some embodiments, the antibody comprises a CDR-H2 sequence that is 50%, 75%, 80%, 85%, 90%, 95%, or 100% identical to the consensus CDR-H2 sequence from antibody group lineage 25G. In some embodiments, the antibody comprises a CDR-H1 sequence that is 50%, 75%, 80%, 85%, 90%, 95%, or 100% identical to the consensus CDR-H1 sequence from antibody group lineage 25G. In some embodiments, the antibody comprises two heavy chain CDRs that are 50%, 75%, 80%, 85%, 90%, 95%, or 100% identical to the corresponding consensus two heavy chain CDRs from antibody group lineage 25G. In some embodiments, the antibody comprises three heavy chain CDRs that are 50%, 75%, 80%, 85%, 90%, 95%, or 100% identical to the three consensus heavy chain CDRs from antibody group lineage 25G.

In some embodiments, the antibody comprises a light chain CDR from antibody group lineage 25G. In some embodiments, the antibody comprises a CDR-L3 sequence that is 50%, 75%, 80%, 85%, 90%, 95%, or 100% identical to the consensus CDR-L3 sequence from antibody group lineage 25G. In some embodiments, the antibody comprises a CDR-L2 sequence that is 50%, 75%, 80%, 85%, 90%, 95%, or 100% identical to the consensus CDR-L2 sequence from antibody group lineage 25G. In some embodiments, the antibody comprises a CDR-L1 sequence that is 50%, 75%, 80%, 85%, 90%, 95%, or 100% identical to the consensus CDR-L1 sequence from antibody group lineage 25G. In some embodiments, the antibody comprises two light chain CDRs that are 50%, 75%, 80%, 85%, 90%, 95%, or 100% identical to the corresponding consensus two light chain CDRs from antibody group lineage 25G. In some embodiments, the antibody comprises three light chain CDRs that are 50%, 75%, 80%, 85%, 90%, 95%, or 100% identical to the three light chain consensus CDRs from antibody group lineage 25G.

In some embodiments, the antibody comprises a CHR—H3 sequence that is 50%, 75%, 80%, 85%, 90%, 95%, or 100% identical to the consensus CDR-H3 sequence from antibody group lineage 25G and CDR-L3 sequence that is 50%, 75%, 80%, 85%, 90%, 95%, or 100% identical to the consensus CDR-L3 sequence from antibody group lineage 25G. In some embodiments, the antibody comprises six CDR sequences that are 50%, 75%, 80%, 85%, 90%, 95%, or 100% identical to the corresponding six consensus CDRs from antibody group lineage 25G.

Variant CDRs

In some embodiments of any of the antibodies provided herein, the antibody CDRs may comprise up to 1, 2, 3, 4, 5, 6, 7, or 8 amino acid substitutions to any of the CDR sequences described herein. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

2.2.7. Functional Properties of Antibody Variants

As described above, and elsewhere in this disclosure, provided herein are antibody variants defined based on percent identity to an illustrative antibody sequence provided herein, or substitution of amino acid residues in comparison to an illustrative antibody sequence provided herein.

In some embodiments, a variant of an antibody provided herein has specificity for hTF. In some embodiments, a variant of an antibody provided herein has specificity for cTF. In some embodiments, a variant of an antibody provided herein has specificity for mTF. In some embodiments, a variant of an antibody provided herein has specificity for hTF and cTF. In some embodiments, a variant of an antibody provided herein has specificity for hTF and mTF. In some embodiments, a variant of an antibody provided herein has specificity for cTF and mTF. In some embodiments, a variant of an antibody provided herein has specificity for hTF, cTF and mTF.

In some embodiments, a variant of an antibody that is derived from an illustrative antibody sequence provided herein retains affinity, as measured by K_{D}, for hTF that is within about 1.5-fold, about 2-fold, about 3-fold, about 4-fold, about 5-fold, about 6-fold, about 7-fold, about 8-fold, about 9-fold or about 10-fold the affinity of such illustrative antibody. In some embodiments, a variant of an antibody that is derived from an illustrative antibody sequence provided herein retains affinity, as measured by K_{D}, for cTF that is within about 1.5-fold, about 2-fold, about 3-fold, about 4-fold, about 5-fold, about 6-fold, about 7-fold, about 8-fold, about 9-fold or about 10-fold the affinity of such illustrative antibody. In some embodiments, a variant of an antibody that is derived from an illustrative antibody sequence provided herein retains affinity, as measured by K_{D}, for mTF that is within about 1.5-fold, about 2-fold, about 3-fold, about 4-fold, about 5-fold, about 6-fold, about 7-fold, about 8-fold, about 9-fold or about 10-fold the affinity of such illustrative antibody. In some embodiments, a variant of an antibody that is derived from an illustrative antibody sequence provided herein retains affinity, as measured by K_{D}, for both hTF and cTF that is within about 1.5-fold, about 2-fold, about 3-fold, about 4-fold, about 5-fold, about 6-fold, about 7-fold, about 8-fold, about 9-fold or about 10-fold the affinity of such illustrative antibody. In some embodiments, a variant of an antibody that is derived from an illustrative antibody sequence provided herein retains affinity, as measured by K_{D}, for both hTF and mTF that is within about 1.5-fold, about 2-fold, about 3-fold, about 4-fold, about 5-fold, about 6-fold, about 7-fold, about 8-fold, about 9-fold or about 10-fold the affinity of such illustrative antibody. In some embodiments, a variant of an antibody that is derived from an illustrative antibody sequence provided herein retains affinity, as measured by K_{D}, for both cTF and mTF that is within about 1.5-fold, about 2-fold, about 3-fold, about 4-fold, about 5-fold, about 6-fold, about 7-fold, about 8-fold, about 9-fold or about 10-fold the affinity of such illustrative antibody. In some embodiments, a variant of an antibody that is derived from an illustrative antibody sequence provided herein retains affinity, as measured by K_{D}, for all three of hTF, cTF and mTF that is within about 1.5-fold, about 2-fold, about 3-fold, about 4-fold, about 5-fold, about 6-fold, about 7-fold, about 8-fold, about 9-fold or about 10-fold the affinity of such illustrative antibody.

In some embodiments, a variant of an antibody provided herein retains the ability to inhibit TF signaling, as measured by one or more assays or biological effects described herein. In some embodiments, a variant of an antibody provided herein retains the normal function of TF in the blood coagulation processes.

In some embodiments, a variant of an antibody provided herein competes for binding to TF with an antibody selected from 25A, 25A3, 25A5, 25A5-T, 25G, 25G1, and 25G9, each as provided in Table 5 of this disclosure.

In some embodiments, a variant of an antibody provided herein allows human thrombin generation as determined by thrombin generation assay (TGA). In some embodiments, a variant of an antibody provided herein does not inhibit human thrombin generation as determined by thrombin generation assay (TGA).

In some embodiments, a variant of an antibody provided herein binds human TF at a human TF binding site that is distinct from a human TF binding site bound by human FX. In some embodiments, a variant of an antibody provided herein does not interfere with the ability of TF:FVIIa to convert FX into FXa.

In some embodiments, a variant of an antibody provided herein binds human TF at a human TF binding site that is distinct from a human TF binding site bound by human FVIIa. In some embodiments, a variant of an antibody provided herein does not compete for binding to human TF with human FVIIa.

In some embodiments, a variant of an antibody provided herein inhibits FVIIa-dependent TF signaling.

In some embodiments, a variant of an antibody provided herein binds mouse TF (SEQ ID NO:817). In some embodiments, a variant of an antibody provided herein binds mouse TF with an affinity lower (as indicated by higher K_{D}) than the affinity of the antibody for hTF. In some embodiments, a variant of an antibody provided herein does not bind mTF.

In some embodiments, a variant of an antibody provided herein binds pig TF (SEQ ID NO:824). In some embodiments, a variant of an antibody provided herein binds pig TF with an affinity lower (as indicated by higher K_{D}) than the affinity of the antibody for hTF. In some embodiments, a variant of an antibody provided herein does not bind pTF.

In some embodiments, a variant of an antibody provided herein binds the same epitope of TF as such antibody.

2.2.8. Other Functional Properties of Antibodies

In some embodiments, an antibody provided herein has one or more of the characteristics listed in the following (a)-(dd): (a) binds human TF at a human TF binding site that is distinct from a human TF binding site bound by human FVIIa; (b) does not inhibit human thrombin generation as determined by thrombin generation assay (TGA); (c) does not reduce the thrombin peak on a thrombin generation curve (Peak IIa) compared to an isotype control; (d) does not increase the time from the assay start to the thrombin peak on a thrombin generation curve (ttPeak) compared to an isotype control; (e) does not decrease the endogenous thrombin potential (ETP) as determined by the area under a thrombin generation curve compared to an isotype control; (f) allows human thrombin generation as determined by thrombin generation assay (TGA); (g) maintains the thrombin peak on a thrombin generation curve (Peak IIa) compared to an isotype control; (h) maintains the time from the assay start to the thrombin peak on a thrombin generation curve (ttPeak) compared to an isotype control; (i) preserves the endogenous thrombin potential (ETP) as determined by the area under a thrombin generation curve compared to an isotype control; (j) binds human TF at a human TF binding site that is distinct from a human TF binding site bound by human FX; (k) does not interfere with the ability of TF:FVIIa to convert FX into FXa; (l) does not compete for binding to human TF with human FVIIa; (m) inhibits FVIIa-dependent TF signaling; (n) binds to cynomolgus TF; (o) binds to mouse TF; (p) binds to rabbit TF; (q) binds to pig TF; (s) the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; (t) the binding between the antibody and a variant TF extracellular domain comprising a mutation at amino acid residue 68 of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay; (u) the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; (v) the binding between the antibody and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (w) the binding between the antibody and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (x) the binding between the antibody and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (y) the binding between the antibody and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (z) the binding between the antibody and a human TF extracellular domain with amino acid residues 159-219 of the sequence shown in SEQ ID NO:810 replaced by rat TF extracellular domain amino acid residues 164-224 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (aa) the binding between the antibody and a human TF extracellular domain with amino acid residues 159-189 of the sequence shown in SEQ ID NO:810 replaced by rat TF extracellular domain amino acid residues 164-194 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (bb) the binding between the antibody and a human TF extracellular domain with amino acid residues 159-174 of the sequence shown in SEQ ID NO:810 replaced by rat TF extracellular domain amino acid residues 164-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (cc) the binding between the antibody and a human TF extracellular domain with amino acid residues 167-174 of the sequence shown in SEQ ID NO:810 replaced by rat TF extracellular domain amino acid residues 172-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; and (dd) the binding between the antibody and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay. In some embodiments, an antibody provided herein has two or more of the characteristics listed in the foregoing (a)-(dd). In some embodiments, an antibody provided herein has three or more of the characteristics listed in the foregoing (a)-(dd). In some embodiments, an antibody provided herein has four or more of the characteristics listed in the foregoing (a)-(dd). In some embodiments, an antibody provided herein has five or more of the characteristics listed in the foregoing (a)-(dd). In some embodiments, an antibody provided herein has six or more of the characteristics listed in the foregoing (a)-(dd). In some embodiments, an antibody provided herein has seven or more of the characteristics listed in the foregoing (a)-(dd). In some embodiments, an antibody provided herein has eight or more of the characteristics listed in the foregoing (a)-(dd). In some embodiments, an antibody provided herein has nine or more of the characteristics listed in the foregoing (a)-(dd). In some embodiments, an antibody provided herein has ten or more of the characteristics listed in the foregoing (a)-(dd). In some embodiments, an antibody provided herein has eleven or more of the characteristics listed in the foregoing (a)-(dd). In some embodiments, an antibody provided herein has twelve or more of the characteristics listed in the foregoing (a)-(dd). In some embodiments, an antibody provided herein has thirteen or more of the characteristics listed in the foregoing (a)-(dd). In some embodiments, an antibody provided herein has fourteen or more of the characteristics listed in the foregoing (a)-(dd). In some embodiments, an antibody provided herein has fifteen or more of the characteristics listed in the foregoing (a)-(dd). In some embodiments, an antibody provided herein has sixteen or more of the characteristics listed in the foregoing (a)-(dd). In some embodiments, an antibody provided herein has seventeen or more of the characteristics listed in the foregoing (a)-(dd). In some embodiments, an antibody provided herein has eighteen or more of the characteristics listed in the foregoing (a)-(dd). In some embodiments, an antibody provided herein has nineteen or more of the characteristics listed in the foregoing (a)-(dd). In some embodiments, an antibody provided herein has twenty or more of the characteristics listed in the foregoing (a)-(dd). In some embodiments, an antibody provided herein has twenty-one or more of the characteristics listed in the foregoing (a)-(dd). In some embodiments, an antibody provided herein has twenty-two or more of the characteristics listed in the foregoing (a)-(dd). In some embodiments, an antibody provided herein has twenty-three of the characteristics listed in the foregoing (a)-(dd). In some embodiments, an antibody provided herein has twenty-four of the characteristics listed in the foregoing (a)-(dd). In some embodiments, an antibody provided herein has twenty-five of the characteristics listed in the foregoing (a)-(dd). In some embodiments, an antibody provided herein has twenty-six of the characteristics listed in the foregoing (a)-(dd). In some embodiments, an antibody provided herein has twenty-seven of the characteristics listed in the foregoing (a)-(dd). In some embodiments, an antibody provided herein has twenty-eight of the characteristics listed in the foregoing (a)-(dd). In some embodiments, an antibody provided herein has twenty-nine of the characteristics listed in the foregoing (a)-(dd). In some embodiments, an antibody provided herein has all thirty of the characteristics listed in the foregoing (a)-(dd).

In some embodiments, an antibody provided herein has one or more of the characteristics listed in the following (a)-(dd): (a) binds human TF at a human TF binding site that is distinct from a human TF binding site bound by human FVIIa; (b) does not inhibit human thrombin generation as determined by thrombin generation assay (TGA); (c) does not reduce the thrombin peak on a thrombin generation curve (Peak IIa) compared to an isotype control; (d) does not increase the time from the assay start to the thrombin peak on a thrombin generation curve (ttPeak) compared to an isotype control; (e) does not decrease the endogenous thrombin potential (ETP) as determined by the area under a thrombin generation curve compared to an isotype control; (f) allows human thrombin generation as determined by thrombin generation assay (TGA); (g) maintains the thrombin peak on a thrombin generation curve (Peak IIa) compared to an isotype control; (h) maintains the time from the assay start to the thrombin peak on a thrombin generation curve (ttPeak) compared to an isotype control; (i) preserves the endogenous thrombin potential (ETP) as determined by the area under a thrombin generation curve compared to an isotype control; (j) binds human TF at a human TF binding site that is distinct from a human TF binding site bound by human FX; (k) does not interfere with the ability of TF:FVIIa to convert FX into FXa; (l) does not compete for binding to human TF with human FVIIa; (m) inhibits FVIIa-dependent TF signaling; (n) binds to cynomolgus TF; (o) binds to mouse TF; (p) binds to rabbit TF; (q) binds to pig TF; (s) the binding between the antibody and a variant TF extracellular domain comprising a mutation K149N of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; (t) the binding between the antibody and a variant TF extracellular domain comprising a mutation K68N of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay; (u) the binding between the antibody and a variant TF extracellular domain comprising mutations N171H and T197K of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; (v) the binding between the antibody and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (w) the binding between the antibody and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (x) the binding between the antibody and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (y) the binding between the antibody and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (z) the binding between the antibody and a human TF extracellular domain with amino acid residues 159-219 of the sequence shown in SEQ ID NO:810 replaced by rat TF extracellular domain amino acid residues 164-224 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (aa) the binding between the antibody and a human TF extracellular domain with amino acid residues 159-189 of the sequence shown in SEQ ID NO:810 replaced by rat TF extracellular domain amino acid residues 164-194 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (bb) the binding between the antibody and a human TF extracellular domain with amino acid residues 159-174 of the sequence shown in SEQ ID NO:810 replaced by rat TF extracellular domain amino acid residues 164-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (cc) the binding between the antibody and a human TF extracellular domain with amino acid residues 167-174 of the sequence shown in SEQ ID NO:810 replaced by rat TF extracellular domain amino acid residues 172-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; and (dd) the binding between the antibody and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay. In some embodiments, an antibody provided herein has two or more of the characteristics listed in the foregoing (a)-(dd). In some embodiments, an antibody provided herein has three or more of the characteristics listed in the foregoing (a)-(dd). In some embodiments, an antibody provided herein has four or more of the characteristics listed in the foregoing (a)-(dd). In some embodiments, an antibody provided herein has five or more of the characteristics listed in the foregoing (a)-(dd). In some embodiments, an antibody provided herein has six or more of the characteristics listed in the foregoing (a)-(dd). In some embodiments, an antibody provided herein has seven or more of the characteristics listed in the foregoing (a)-(dd). In some embodiments, an antibody provided herein has eight or more of the characteristics listed in the foregoing (a)-(dd). In some embodiments, an antibody provided herein has nine or more of the characteristics listed in the foregoing (a)-(dd). In some embodiments, an antibody provided herein has ten or more of the characteristics listed in the foregoing (a)-(dd). In some embodiments, an antibody provided herein has eleven or more of the characteristics listed in the foregoing (a)-(dd). In some embodiments, an antibody provided herein has twelve or more of the characteristics listed in the foregoing (a)-(dd). In some embodiments, an antibody provided herein has thirteen or more of the characteristics listed in the foregoing (a)-(dd). In some embodiments, an antibody provided herein has fourteen or more of the characteristics listed in the foregoing (a)-(dd). In some embodiments, an antibody provided herein has fifteen or more of the characteristics listed in the foregoing (a)-(dd). In some embodiments, an antibody provided herein has sixteen or more of the characteristics listed in the foregoing (a)-(dd). In some embodiments, an antibody provided herein has seventeen or more of the characteristics listed in the foregoing (a)-(dd). In some embodiments, an antibody provided herein has eighteen or more of the characteristics listed in the foregoing (a)-(dd). In some embodiments, an antibody provided herein has nineteen or more of the characteristics listed in the foregoing (a)-(dd). In some embodiments, an antibody provided herein has twenty or more of the characteristics listed in the foregoing (a)-(dd). In some embodiments, an antibody provided herein has twenty-one or more of the characteristics listed in the foregoing (a)-(dd). In some embodiments, an antibody provided herein has twenty-two or more of the characteristics listed in the foregoing (a)-(dd). In some embodiments, an antibody provided herein has twenty-three of the characteristics listed in the foregoing (a)-(dd). In some embodiments, an antibody provided herein has twenty-four of the characteristics listed in the foregoing (a)-(dd). In some embodiments, an antibody provided herein has twenty-five of the characteristics listed in the foregoing (a)-(dd). In some embodiments, an antibody provided herein has twenty-six of the characteristics listed in the foregoing (a)-(dd). In some embodiments, an antibody provided herein has twenty-seven of the characteristics listed in the foregoing (a)-(dd). In some embodiments, an antibody provided herein has twenty-eight of the characteristics listed in the foregoing (a)-(dd). In some embodiments, an antibody provided herein has twenty-nine of the characteristics listed in the foregoing (a)-(dd). In some embodiments, an antibody provided herein has all thirty of the characteristics listed in the foregoing (a)-(dd).

In some embodiments, an antibody provided herein exhibits a combination of characteristics comprising two or more of characteristics listed in the following (a)-(dd): (a) binds human TF at a human TF binding site that is distinct from a human TF binding site bound by human FVIIa; (b) does not inhibit human thrombin generation as determined by thrombin generation assay (TGA); (c) does not reduce the thrombin peak on a thrombin generation curve (Peak IIa) compared to an isotype control; (d) does not increase the time from the assay start to the thrombin peak on a thrombin generation curve (ttPeak) compared to an isotype control; (e) does not decrease the endogenous thrombin potential (ETP) as determined by the area under a thrombin generation curve compared to an isotype control; (f) allows human thrombin generation as determined by thrombin generation assay (TGA); (g) maintains the thrombin peak on a thrombin generation curve (Peak IIa) compared to an isotype control; (h) maintains the time from the assay start to the thrombin peak on a thrombin generation curve (ttPeak) compared to an isotype control; (i) preserves the endogenous thrombin potential (ETP) as determined by the area under a thrombin generation curve compared to an isotype control; (j) binds human TF at a human TF binding site that is distinct from a human TF binding site bound by human FX; (k) does not interfere with the ability of TF:FVIIa to convert FX into FXa; (l) does not compete for binding to human TF with human FVIIa; (m) inhibits FVIIa-dependent TF signaling; (n) binds to cynomolgus TF; (o) binds to mouse TF; (p) binds to rabbit TF; (q) binds to pig TF; (s) the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; (t) the binding between the antibody and a variant TF extracellular domain comprising a mutation at amino acid residue 68 of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay; (u) the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; (v) the binding between the antibody and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (w) the binding between the antibody and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (x) the binding between the antibody and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (y) the binding between the antibody and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (z) the binding between the antibody and a human TF extracellular domain with amino acid residues 159-219 of the sequence shown in SEQ ID NO:810 replaced by rat TF extracellular domain amino acid residues 164-224 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (aa) the binding between the antibody and a human TF extracellular domain with amino acid residues 159-189 of the sequence shown in SEQ ID NO:810 replaced by rat TF extracellular domain amino acid residues 164-194 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (bb) the binding between the antibody and a human TF extracellular domain with amino acid residues 159-174 of the sequence shown in SEQ ID NO:810 replaced by rat TF extracellular domain amino acid residues 164-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (cc) the binding between the antibody and a human TF extracellular domain with amino acid residues 167-174 of the sequence shown in SEQ ID NO:810 replaced by rat TF extracellular domain amino acid residues 172-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; and (dd) the binding between the antibody and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of characteristics comprising two or more of characteristics listed in the following (a)-(dd): (a) binds human TF at a human TF binding site that is distinct from a human TF binding site bound by human FVIIa; (b) does not inhibit human thrombin generation as determined by thrombin generation assay (TGA); (c) does not reduce the thrombin peak on a thrombin generation curve (Peak IIa) compared to an isotype control; (d) does not increase the time from the assay start to the thrombin peak on a thrombin generation curve (ttPeak) compared to an isotype control; (e) does not decrease the endogenous thrombin potential (ETP) as determined by the area under a thrombin generation curve compared to an isotype control; (f) allows human thrombin generation as determined by thrombin generation assay (TGA); (g) maintains the thrombin peak on a thrombin generation curve (Peak IIa) compared to an isotype control; (h) maintains the time from the assay start to the thrombin peak on a thrombin generation curve (ttPeak) compared to an isotype control; (i) preserves the endogenous thrombin potential (ETP) as determined by the area under a thrombin generation curve compared to an isotype control; (j) binds human TF at a human TF binding site that is distinct from a human TF binding site bound by human FX; (k) does not interfere with the ability of TF:FVIIa to convert FX into FXa; (1) does not compete for binding to human TF with human FVIIa; (m) inhibits FVIIa-dependent TF signaling; (n) binds to cynomolgus TF; (o) binds to mouse TF; (p) binds to rabbit TF; (q) binds to pig TF; (s) the binding between the antibody and a variant TF extracellular domain comprising a mutation K149N of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; (t) the binding between the antibody and a variant TF extracellular domain comprising a mutation K68N of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay; (u) the binding between the antibody and a variant TF extracellular domain comprising mutations N171H and T197K of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; (v) the binding between the antibody and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (w) the binding between the antibody and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (x) the binding between the antibody and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (y) the binding between the antibody and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (z) the binding between the antibody and a human TF extracellular domain with amino acid residues 159-219 of the sequence shown in SEQ ID NO:810 replaced by rat TF extracellular domain amino acid residues 164-224 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (aa) the binding between the antibody and a human TF extracellular domain with amino acid residues 159-189 of the sequence shown in SEQ ID NO:810 replaced by rat TF extracellular domain amino acid residues 164-194 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (bb) the binding between the antibody and a human TF extracellular domain with amino acid residues 159-174 of the sequence shown in SEQ ID NO:810 replaced by rat TF extracellular domain amino acid residues 164-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (cc) the binding between the antibody and a human TF extracellular domain with amino acid residues 167-174 of the sequence shown in SEQ ID NO:810 replaced by rat TF extracellular domain amino acid residues 172-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; and (dd) the binding between the antibody and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; does not inhibit human thrombin generation as determined by thrombin generation assay (TGA); and the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; does not inhibit human thrombin generation as determined by thrombin generation assay (TGA); and the binding between the antibody and a variant TF extracellular domain comprising mutations N171H and T197K of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; allows human thrombin generation as determined by thrombin generation assay (TGA); and the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; allows human thrombin generation as determined by thrombin generation assay (TGA); and the binding between the antibody and a variant TF extracellular domain comprising mutations N171H and T197K of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; does not inhibit human thrombin generation as determined by thrombin generation assay (TGA); the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; does not inhibit human thrombin generation as determined by thrombin generation assay (TGA); the binding between the antibody and a variant TF extracellular domain comprising a mutation K149N of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and the binding between the antibody and a variant TF extracellular domain comprising mutations N171H and T197K of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; allows human thrombin generation as determined by thrombin generation assay (TGA); the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; allows human thrombin generation as determined by thrombin generation assay (TGA); the binding between the antibody and a variant TF extracellular domain comprising a mutation K149N of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and the binding between the antibody and a variant TF extracellular domain comprising mutations N171H and T197K of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; does not inhibit human thrombin generation as determined by thrombin generation assay (TGA); binds to cynomolgus TF; the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; does not inhibit human thrombin generation as determined by thrombin generation assay (TGA); binds to cynomolgus TF; the binding between the antibody and a variant TF extracellular domain comprising a mutation K149N of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and the binding between the antibody and a variant TF extracellular domain comprising mutations N171H and T197K of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; allows human thrombin generation as determined by thrombin generation assay (TGA); binds to cynomolgus TF; the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; allows human thrombin generation as determined by thrombin generation assay (TGA); binds to cynomolgus TF; the binding between the antibody and a variant TF extracellular domain comprising a mutation K149N of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and the binding between the antibody and a variant TF extracellular domain comprising mutations N171H and T197K of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

2.3. Affinity and Other Properties of TF Antibodies

2.3.1. Affinity of TF Antibodies

In some embodiments, the affinity of an antibody provided herein for TF as indicated by K_{D}, is less than about 10⁻⁵ M, less than about 10⁻⁶ M, less than about 10⁻⁷ M, less than about 10⁻⁸ M, less than about 10⁻⁹ M, less than about 10⁻¹⁰ M, less than about 10⁻¹¹ M, or less than about 10⁻¹² M. In some embodiments, the affinity of the antibody is between about 10⁻⁷ M and 10⁻¹² M. In some embodiments, the affinity of the antibody is between about 10⁻⁷ M and 10⁻¹¹ M. In some embodiments, the affinity of the antibody is between about 10⁻⁷ M and 10⁻¹⁰ M. In some embodiments, the affinity of the antibody is between about 10⁻⁷ M and 10⁻⁹ M. In some embodiments, the affinity of the antibody is between about 10⁻⁷ M and 10⁻⁸ M. In some embodiments, the affinity of the antibody is between about 10⁻⁸ M and 10⁻¹² M. In some embodiments, the affinity of the antibody is between about 10⁻⁸ M and 10⁻¹¹ M. In some embodiments, the affinity of the antibody is between about 10⁻⁹ M and 10⁻¹¹ M. In some embodiments, the affinity of the antibody is between about 10⁻¹⁰ M and 10⁻¹¹ M.

In some embodiments, the K_{D} value of an antibody provided herein for cTF is no more than 15× of the K_{D} value of the antibody for hTF. In some embodiments, the K_{D} value of an antibody provided herein for cTF is no more than 10× of the K_{D} value of the antibody for hTF. In some embodiments, the K_{D} value of an antibody provided herein for cTF is no more than 8× of the K_{D} value of the antibody for hTF. In some embodiments, the K_{D} value of an antibody provided herein for cTF is no more than 5× of the K_{D} value of the antibody for hTF. In some embodiments, the K_{D} value of an antibody provided herein for cTF is no more than 3× of the K_{D} value of the antibody for hTF. In some embodiments, the K_{D} value of an antibody provided herein for cTF is no more than 2× of the K_{D} value of the antibody for hTF.

In some embodiments, the K_{D} value of an antibody provided herein for mTF is no more than 20× of the K_{D} value of the antibody for hTF. In some embodiments, the K_{D} value of an antibody provided herein for mTF is no more than 15× of the K_{D} value of the antibody for hTF. In some embodiments, the K_{D} value of an antibody provided herein for mTF is no more than 10× of the K_{D} value of the antibody for hTF. In some embodiments, the K_{D} value of an antibody provided herein for mTF is no more than 5× of the K_{D} value of the antibody for hTF. In some embodiments, the K_{D} value of an antibody provided herein for mTF is no more than 2× of the K_{D} value of the antibody for hTF.

In some embodiments, the affinity of an antibody provided herein for hTF as indicated by K_{D} measured by Biacore, as set forth in Table 5 of PCT/US2019/12427, filed on Jan. 4, 2019 is selected from about 0.31 nM, about 6.20 nM, about 0.36 nM, about 0.08 nM, about 23.0 nM, about 0.94 nM, about 13.3 nM, about 0.47 nM, about 0.09 nM, about 1.75 nM, about 0.07 nM, about 0.14 nM, about 2.09 nM, about 0.06 nM, about 0.15 nM, about 1.46 nM, about 1.60 nM, and about 0.42 nM. In some embodiments, such affinity as indicated by K_{D} ranges from about 23.0 nM to about 0.06 nM. In some embodiments, such is about 23.0 nM or less.

In some embodiments, the affinity of an antibody provided herein for hTF as indicated by K_{D} measured by ForteBio, as set forth in Table 5 of PCT/US2019/12427, filed on Jan. 4, 2019 is selected from about 1.28 nM, about 2.20 nM, about 8.45 nM, about 1.67 nM, about 0.64 nM, about 21.9 nM, about 3.97 nM, about 35.8 nM, about 3.30 nM, about 2.32 nM, about 0.83 nM, about 2.40 nM, about 0.96 nM, about 0.86 nM, about 3.84 nM, about 1.02 nM, about 1.61 nM, about 2.52 nM, about 2.28 nM, and about 1.59 nM. In some embodiments, such affinity as indicated by K_{D} ranges from about 35.8 nM to about 0.64 nM. In some embodiments, such K_{D} is about 35.8 nM or less.

In some embodiments, the affinity of an antibody provided herein for cTF as indicated by K_{D} measured by Biacore, as set forth in Table 5 of PCT/US2019/12427, filed on Jan. 4, 2019 is selected from about 0.26 nM, about 5.42 nM, about 0.21 nM, about 0.04 nM, about 18.0 nM, about 0.78 nM, about 16.4 nM, about 5.06 nM, about 0.08 nM, about 5.64 nM, about 0.12 nM, about 0.24 nM, about 5.66 nM, about 0.39 nM, about 5.69 nM, about 6.42 nM, and about 1.83 nM. In some embodiments, such affinity as indicated by K_{D} ranges from about 18.0 nM to about 0.04 nM. In some embodiments, such K_{D} is about 18.0 nM or less.

In some embodiments, the affinity of an antibody provided herein for cTF as indicated by K_{D} measured by ForteBio, as set forth in Table 5 of PCT/US2019/12427, filed on Jan. 4, 2019 is selected from about 1.43 nM, about 2.70 nM, about 7.65 nM, about 1.36 nM, about 0.76 nM, about 17.5 nM, about 4.99 nM, about 42.9 nM, about 12.0 nM, about 15.0 nM, about 0.57 nM, about 3.40 nM, about 1.05 nM, about 0.94 nM, about 4.12 nM, about 1.11 nM, about 1.96 nM, about 4.07 nM, about 2.71 nM, and about 4.16 nM. In some embodiments, such affinity as indicated by K_{D} ranges from about 42.9 nM to about 0.57 nM. In some embodiments, such K_{D} is about 42.9 nM or less.

In some embodiments, the affinity of an antibody provided herein for mTF as indicated by K_{D} measured by Biacore, as set forth in Table 5 of PCT/US2019/12427, filed on Jan. 4, 2019 is selected from about 5.4 nM, about 2.9 nM, about 21 nM, and about 2.4 nM. In some embodiments, such affinity as indicated by K_{D} ranges from about 21 nM to about 2.4 nM. In some embodiments, such K_{D} is about 21 nM or less.

In some embodiments, the affinity of an antibody provided herein for mTF as indicated by K_{D} measured by ForteBio, as set forth in Table 5 of PCT/US2019/12427, filed on Jan. 4, 2019 is selected from about 263 nM, about 131 nM, about 188 nM, about 114 nM, about 34.2 nM, about 9.16 nM, about 161 nM, about 72.1 nM, about 360 nM, about 281 nM, about 41.4 nM, about 6.12 nM, about 121 nM, and about 140 nM. In some embodiments, such affinity as indicated by K_{D} ranges from about 360 nM to about 6.12 nM. In some embodiments, such K_{D} is about 360 nM or less.

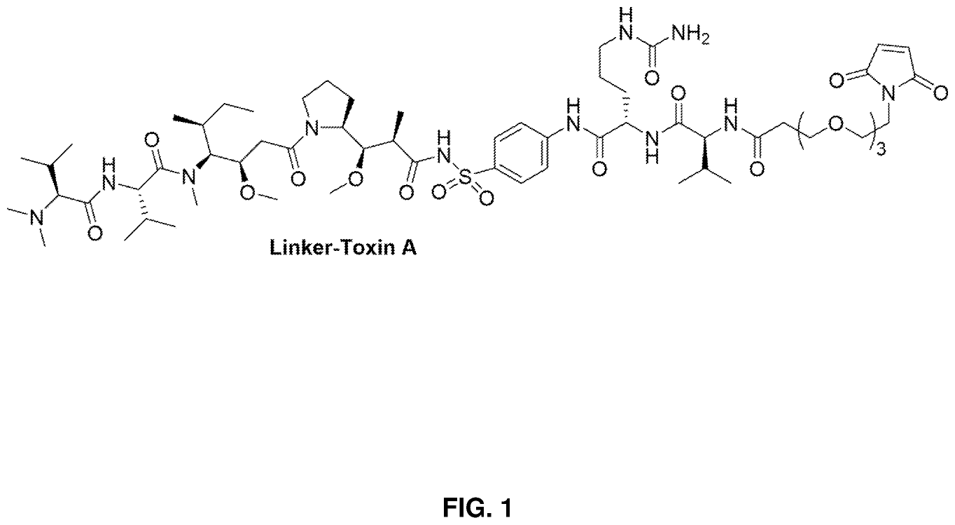

In some embodiments, the affinity of an antibody provided herein for hTF as indicated by EC₅₀ measured with human TF-positive HCT-116 cells, as set forth in FIGS. 1A and 1B of PCT/US2019/12427, filed on Jan. 4, 2019 is selected from about 50 pM, about 58 pM, about 169 pM, about 77 pM, about 88 pM, about 134 pM, about 85 pM, about 237 pM, about 152 pM, about 39 pM, about 559 pM, about 280 pM, about 255 pM, about 147 pM, about 94 pM, about 117 pM, about 687 pM, about 532 pM, and about 239 pM. In some embodiments, such affinity ranges from about 687 pM to about 39 pM. In some embodiments, such EC₅₀ is about 687 pM or less.

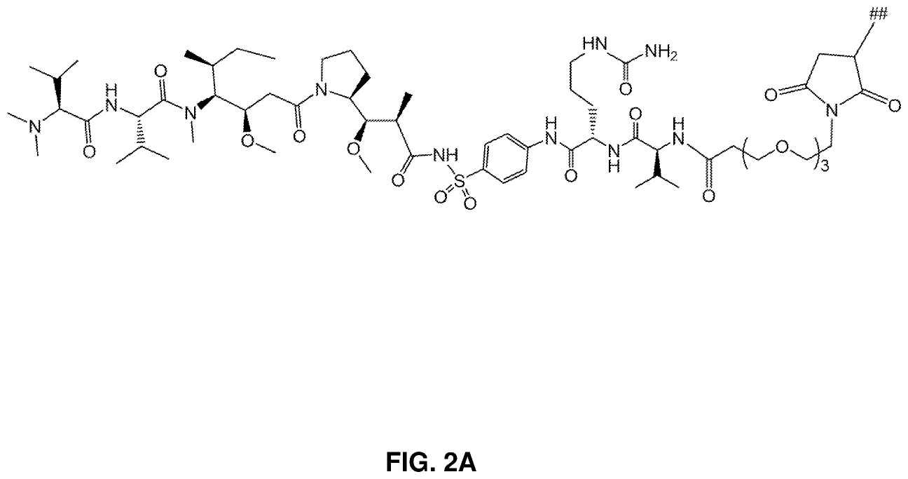

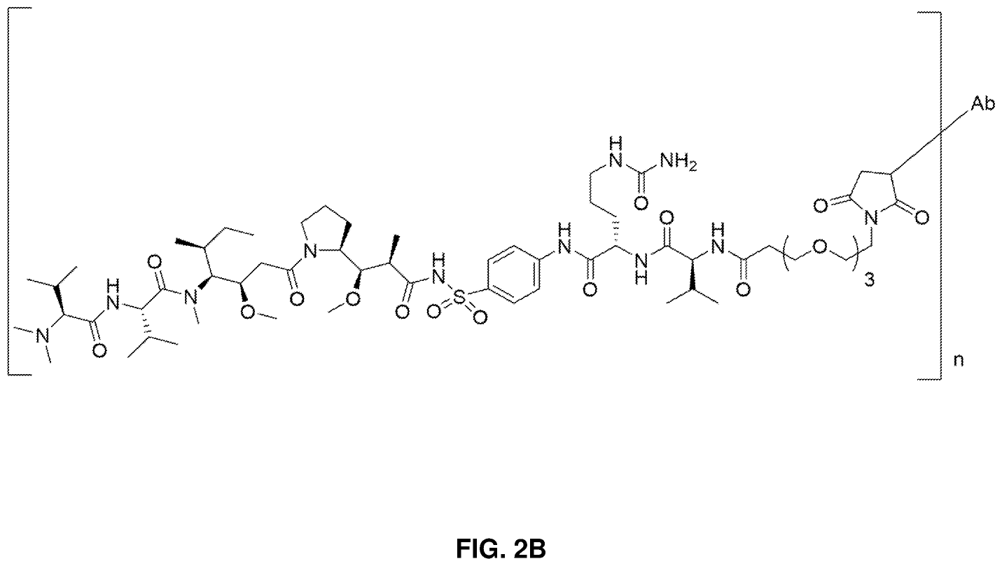

In some embodiments, the affinity of an antibody provided herein for mTF as indicated by EC₅₀ measured with mouse TF-positive CHO cells, as set forth in FIGS. 2A and 2B of PCT/US2019/12427, filed on Jan. 4, 2019 is selected from about 455 nM, about 87 nM, about 11 nM, about 3.9 nM, about 3.0 nM, about 3.4 nM, about 255 nM, about 2.9 nM, about 3.6 nM, and about 4.0 nM. In some embodiments, such affinity ranges from about 455 nM to about 2.9 nM. In some embodiments, such EC₅₀ is about 455 pM or less.

In some embodiments, the K_{D} value of an antibody provided herein for pTF is no more than 20× of the K_{D} value of the antibody for hTF. In some embodiments, the K_{D} value of an antibody provided herein for pTF is no more than 15× of the K_{D} value of the antibody for hTF. In some embodiments, the K_{D} value of an antibody provided herein for pTF is no more than 10× of the K_{D} value of the antibody for hTF. In some embodiments, the K_{D} value of an antibody provided herein for pTF is no more than 5× of the K_{D} value of the antibody for hTF. In some embodiments, the K_{D} value of an antibody provided herein for pTF is no more than 2× of the K_{D} value of the antibody for hTF.

In some embodiments, the affinity of an antibody provided herein for pTF as indicated by K_{D} measured by Biacore, as set forth in Table 40 of PCT/US2019/12427, filed on Jan. 4, 2019 is 3.31 nM or 12.9 nM.

2.3.2. Thrombin Generation in the Presence of TF Antibodies

In some embodiments, the TF antibodies provided herein do not inhibit human thrombin generation as determined by thrombin generation assay (TGA). In certain embodiments, the TF antibodies provided herein allow human thrombin generation as determined by thrombin generation assay (TGA).

In some embodiments, the percent peak thrombin generation (% Peak IIa) is at least 40% in the presence of no less than 100 nM TF antibody compared to the control conditions without the antibody, as determined by thrombin generation assay (TGA). In some embodiments, the % Peak IIa is at least 50% in the presence of no less than 100 nM TF antibody compared to the control conditions without the antibody, as determined by thrombin generation assay (TGA). In some embodiments, the % Peak IIa is at least 60% in the presence of no less than 100 nM TF antibody compared to the control conditions without the antibody, as determined by thrombin generation assay (TGA). In some embodiments, the % Peak IIa is at least 70% in the presence of no less than 100 nM TF antibody compared to the control conditions without the antibody, as determined by thrombin generation assay (TGA). In some embodiments, the % Peak IIa is at least 80% in the presence of no less than 100 nM TF antibody compared to the control conditions without the antibody, as determined by thrombin generation assay (TGA). In some embodiments, the % Peak IIa is at least 90% in the presence of no less than 100 nM TF antibody compared to the control conditions without the antibody, as determined by thrombin generation assay (TGA). In some embodiments, the % Peak IIa is at least 95% in the presence of no less than 100 nM TF antibody compared to the control conditions without the antibody, as determined by thrombin generation assay (TGA). In some embodiments, the % Peak IIa is at least 99% in the presence of no less than 100 nM TF antibody compared to the control conditions without the antibody, as determined by thrombin generation assay (TGA).

In some embodiments, the % Peak IIa is at least 40% in the presence of no less than 50 nM TF antibody compared to the control conditions without the antibody, as determined by thrombin generation assay (TGA). In some embodiments, the % Peak IIa is at least 50% in the presence of no less than 50 nM TF antibody compared to the control conditions without the antibody, as determined by thrombin generation assay (TGA). In some embodiments, the % Peak IIa is at least 60% in the presence of no less than 50 nM TF antibody compared to the control conditions without the antibody, as determined by thrombin generation assay (TGA). In some embodiments, the % Peak IIa is at least 70% in the presence of no less than 50 nM TF antibody compared to the control conditions without the antibody, as determined by thrombin generation assay (TGA). In some embodiments, the % Peak IIa is at least 80% in the presence of no less than 50 nM TF antibody compared to the control conditions without the antibody, as determined by thrombin generation assay (TGA). In some embodiments, the % Peak IIa is at least 90% in the presence of no less than 50 nM TF antibody compared to the control conditions without the antibody, as determined by thrombin generation assay (TGA). In some embodiments, the % Peak IIa is at least 95% in the presence of no less than 50 nM TF antibody compared to the control conditions without the antibody, as determined by thrombin generation assay (TGA). In some embodiments, the % Peak IIa is at least 99% in the presence of no less than 50 nM TF antibody compared to the control conditions without the antibody, as determined by thrombin generation assay (TGA).

In some embodiments, the % Peak IIa is at least 60% in the presence of no less than 10 nM TF antibody compared to the control conditions without the antibody, as determined by thrombin generation assay (TGA). In some embodiments, the % Peak IIa is at least 70% in the presence of no less than 10 nM TF antibody compared to the control conditions without the antibody, as determined by thrombin generation assay (TGA). In some embodiments, the % Peak IIa is at least 80% in the presence of no less than 10 nM TF antibody compared to the control conditions without the antibody, as determined by thrombin generation assay (TGA). In some embodiments, the % Peak IIa is at least 90% in the presence of no less than 10 nM TF antibody compared to the control conditions without the antibody, as determined by thrombin generation assay (TGA). In some embodiments, the % Peak IIa is at least 95% in the presence of no less than 10 nM TF antibody compared to the control conditions without the antibody, as determined by thrombin generation assay (TGA). In some embodiments, the % Peak IIa is at least 99% in the presence of no less than 10 nM TF antibody compared to the control conditions without the antibody, as determined by thrombin generation assay (TGA).

In some embodiments, the % Peak IIa in the presence of 100 nM TF antibody, as set forth in Table 6 and Table 37 of PCT/US2019/12427, filed on Jan. 4, 2019 is selected from about 99%, about 100%, about 103%, about 64%, about 52%, about 87%, about 96%, about 98%, and about 53% compared to the control conditions without the antibody, as determined by thrombin generation assay (TGA) without antibody pre-incubation. In some embodiments, such % Peak IIa ranges from about 52% to about 103%. In some embodiments, such % Peak IIa is about 52% or more.

In some embodiments, the % Peak IIa in the presence of 50 nM TF antibody, as set forth in Table 6 and Table 37 of PCT/US2019/12427, filed on Jan. 4, 2019 is selected from about 99%, about 100%, about 103%, about 67%, about 58%, about 89%, about 96%, about 98%, about 68%, about 62%, and about 88% compared to the control conditions without the antibody, as determined by thrombin generation assay (TGA) without antibody pre-incubation. In some embodiments, such % Peak IIa ranges from about 58% to about 103%. In some embodiments, such % Peak IIa is about 58% or more.

In some embodiments, the % Peak IIa in the presence of 10 nM TF antibody, as set forth in Table 6 and Table 37 of PCT/US2019/12427, filed on Jan. 4, 2019 is selected from about 100%, about 99%, about 103%, about 87%, about 83%, about 95%, about 98%, about 86%, and about 96% compared to the control conditions without the antibody, as determined by thrombin generation assay (TGA) without antibody pre-incubation. In some embodiments, such % Peak IIa ranges from about 83% to about 103%. In some embodiments, such % Peak IIa is about 83% or more.

In some embodiments, the % Peak IIa in the presence of 100 nM TF antibody, as set forth in Table 7 and Table 38 of PCT/US2019/12427, filed on Jan. 4, 2019 is selected from about 108%, about 105%, about 111%, about 58%, about 47%, about 91%, about 103%, about 109%, about 107%, and about 45% compared to the control conditions without the antibody, as determined by thrombin generation assay (TGA) with 10 min antibody pre-incubation. In some embodiments, such % Peak IIa ranges from about 45% to about 111%. In some embodiments, such % Peak IIa is about 45% or more.

In some embodiments, the % Peak IIa in the presence of 50 nM TF antibody, as set forth in Table 7 and Table 38 of PCT/US2019/12427, filed on Jan. 4, 2019 is selected from about 107%, about 104%, about 114%, about 62%, about 49%, about 87%, about 105%, about 109%, about 55%, and about 92% compared to the control conditions without the antibody, as determined by thrombin generation assay (TGA) with 10 min antibody pre-incubation. In some embodiments, such % Peak IIa ranges from about 49% to about 114%. In some embodiments, such % Peak IIa is about 49% or more.

In some embodiments, the % Peak IIa in the presence of 10 nM TF antibody, as set forth in Table 7 and Table 38 of PCT/US2019/12427, filed on Jan. 4, 2019 is selected from about 105%, about 114%, about 76%, about 68%, about 94%, about 108%, about 104%, about 74%, and about 93% compared to the control conditions without the antibody, as determined by thrombin generation assay (TGA) with 10 min antibody pre-incubation. In some embodiments, such % Peak IIa ranges from about 68% to about 114%. In some embodiments, such % Peak IIa is about 68% or more.

In some embodiments, the percent endogenous thrombin potential (% ETP) is at least 80% in the presence of no less than 100 nM TF antibody compared to the control conditions without the antibody, as determined by thrombin generation assay (TGA). In some embodiments, the % ETP is at least 90% in the presence of no less than 100 nM TF antibody compared to the control conditions without the antibody, as determined by thrombin generation assay (TGA). In some embodiments, the % ETP is at least 95% in the presence of no less than 100 nM TF antibody compared to the control conditions without the antibody, as determined by thrombin generation assay (TGA). In some embodiments, the % ETP is at least 99% in the presence of no less than 100 nM TF antibody compared to the control conditions without the antibody, as determined by thrombin generation assay (TGA).

In some embodiments, the % ETP is at least 80% in the presence of no less than 50 nM TF antibody compared to the control conditions without the antibody, as determined by thrombin generation assay (TGA). In some embodiments, the % ETP is at least 90% in the presence of no less than 50 nM TF antibody compared to the control conditions without the antibody, as determined by thrombin generation assay (TGA). In some embodiments, the % ETP is at least 95% in the presence of no less than 50 nM TF antibody compared to the control conditions without the antibody, as determined by thrombin generation assay (TGA). In some embodiments, the % ETP is at least 99% in the presence of no less than 50 nM TF antibody compared to the control conditions without the antibody, as determined by thrombin generation assay (TGA).

In some embodiments, the % ETP is at least 80% in the presence of no less than 10 nM TF antibody compared to the control conditions without the antibody, as determined by thrombin generation assay (TGA). In some embodiments, the % ETP is at least 90% in the presence of no less than 10 nM TF antibody compared to the control conditions without the antibody, as determined by thrombin generation assay (TGA). In some embodiments, the % ETP is at least 95% in the presence of no less than 10 nM TF antibody compared to the control conditions without the antibody, as determined by thrombin generation assay (TGA). In some embodiments, the % ETP is at least 99% in the presence of no less than 10 nM TF antibody compared to the control conditions without the antibody, as determined by thrombin generation assay (TGA).

In some embodiments, the % ETP in the presence of 100 nM TF antibody, as set forth in Table 6 and Table 37 of PCT/US2019/12427, filed on Jan. 4, 2019 is selected from about 108%, about 103%, about 109%, about 100%, about 96%, about 102%, about 105%, and about 92% compared to the control conditions without the antibody, as determined by thrombin generation assay (TGA) without antibody pre-incubation. In some embodiments, such % ETP ranges from about 92% to about 109%. In some embodiments, such % ETP is about 92% or more.

In some embodiments, the % ETP in the presence of 50 nM TF antibody, as set forth in Table 6 and Table 37 of PCT/US2019/12427, filed on Jan. 4, 2019 is selected from about 108%, about 103%, about 111%, about 101%, about 97%, about 104%, about 106%, about 93%, about 96%, and about 105% compared to the control conditions without the antibody, as determined by thrombin generation assay (TGA) without antibody pre-incubation. In some embodiments, such % ETP ranges from about 93% to about 111%. In some embodiments, such % ETP is about 93% or more.

In some embodiments, the % ETP in the presence of 10 nM TF antibody, as set forth in Table 6 and Table 37 of PCT/US2019/12427, filed on Jan. 4, 2019 is selected from about 106%, about 109%, about 105%, about 104%, about 107%, about 99%, about 101%, and about 102% compared to the control conditions without the antibody, as determined by thrombin generation assay (TGA) without antibody pre-incubation. In some embodiments, such % ETP ranges from about 99% to about 109%. In some embodiments, such % ETP is about 99% or more.

In some embodiments, the % ETP in the presence of 100 nM TF antibody, as set forth in Table 7 and Table 38 of PCT/US2019/12427, filed on Jan. 4, 2019 is selected from about 110%, about 104%, about 106%, about 98%, about 95%, about 108%, about 107%, about 96%, about 92%, and about 103% compared to the control conditions without the antibody, as determined by thrombin generation assay (TGA) with 10 min antibody pre-incubation. In some embodiments, such % ETP ranges from about 92% to about 110%. In some embodiments, such % ETP is about 92% or more.

In some embodiments, the % ETP in the presence of 50 nM TF antibody, as set forth in Table 7 and Table 38 of PCT/US2019/12427, filed on Jan. 4, 2019 is selected from about 110%, about 106%, about 108%, about 103%, about 96%, about 109%, about 102%, about 104%, about 94%, and about 98% compared to the control conditions without the antibody, as determined by thrombin generation assay (TGA) with 10 min antibody pre-incubation. In some embodiments, such % ETP ranges from about 94% to about 110%. In some embodiments, such % ETP is about 94% or more.

In some embodiments, the % ETP in the presence of 10 nM TF antibody, as set forth in Table 7 and Table 38 of PCT/US2019/12427, filed on Jan. 4, 2019 is selected from about 107%, about 106%, about 110%, about 103%, about 100%, about 105%, about 102%, and about 101% compared to the control conditions without the antibody, as determined by thrombin generation assay (TGA) with 10 min antibody pre-incubation. In some embodiments, such % ETP ranges from about 100% to about 110%. In some embodiments, such % ETP is about 100% or more.

2.3.3. FXa Conversion in the Presence of TF Antibodies

In some embodiments, the antibodies provided herein bind human TF at a human TF binding site that is distinct from a human TF binding site bound by human FX. In certain embodiments, the antibodies provided herein do not interfere with the ability of TF:FVIIa to convert FX into FXa.

In some embodiments, the percentage of FXa conversion (% FXa) is at least 75% in the presence of no less than 100 nM TF antibody compared to the control conditions without the antibody. In some embodiments, the % FXa is at least 80% in the presence of no less than 100 nM TF antibody compared to the control conditions without the antibody. In some embodiments, the % FXa is at least 85% in the presence of no less than 100 nM TF antibody compared to the control conditions without the antibody. In some embodiments, the % FXa is at least 90% in the presence of no less than 100 nM TF antibody compared to the control conditions without the antibody. In some embodiments, the % FXa is at least 95% in the presence of no less than 100 nM TF antibody compared to the control conditions without the antibody.

In some embodiments, the % FXa is at least 75% in the presence of no less than 50 nM TF antibody compared to the control conditions without the antibody. In some embodiments, the % FXa is at least 80% in the presence of no less than 50 nM TF antibody compared to the control conditions without the antibody. In some embodiments, the % FXa is at least 85% in the presence of no less than 50 nM TF antibody compared to the control conditions without the antibody. In some embodiments, the % FXa is at least 90% in the presence of no less than 50 nM TF antibody compared to the control conditions without the antibody. In some embodiments, the % FXa is at least 95% in the presence of no less than 50 nM TF antibody compared to the control conditions without the antibody.

In some embodiments, the % FXa is at least 75% in the presence of no less than 25 nM TF antibody compared to the control conditions without the antibody. In some embodiments, the % FXa is at least 80% in the presence of no less than 25 nM TF antibody compared to the control conditions without the antibody. In some embodiments, the % FXa is at least 85% in the presence of no less than 25 nM TF antibody compared to the control conditions without the antibody. In some embodiments, the % FXa is at least 90% in the presence of no less than 25 nM TF antibody compared to the control conditions without the antibody. In some embodiments, the % FXa is at least 95% in the presence of no less than 25 nM TF antibody compared to the control conditions without the antibody.

In some embodiments, the % FXa is at least 75% in the presence of no less than 12.5 nM TF antibody compared to the control conditions without the antibody. In some embodiments, the % FXa is at least 80% in the presence of no less than 12.5 nM TF antibody compared to the control conditions without the antibody. In some embodiments, the % FXa is at least 85% in the presence of no less than 12.5 nM TF antibody compared to the control conditions without the antibody. In some embodiments, % FXa is at least 90% in the presence of no less than 12.5 nM TF antibody compared to the control conditions without the antibody. In some embodiments, the % FXa is at least 95% in the presence of no less than 12.5 nM TF antibody compared to the control conditions without the antibody.

In some embodiments, the % FXa in the presence of 100 nM TF antibody, as set forth in Table 8 of PCT/US2019/12427, filed on Jan. 4, 2019 is selected from about 89%, about 96%, about 116%, about 108%, about 117%, about 105%, about 112%, about 106%, about 103%, about 111%, about 98%, and about 101% compared to the control conditions without the antibody. In some embodiments, such % FXa ranges from about 89% to about 117%. In some embodiments, such % FXa is about 89% or more.

In some embodiments, the % FXa in the presence of 50 nM TF antibody, as set forth in Table 8 of PCT/US2019/12427, filed on Jan. 4, 2019 is selected from about 94%, about 93%, about 78%, about 102%, about 99%, about 104%, about 105%, about 108%, about 107%, about 97%, and about 106% compared to the control conditions without the antibody. In some embodiments, such % FXa ranges from about 78% to about 108%. In some embodiments, such % FXa is about 78% or more.

In some embodiments, the % FXa in the presence of 25 nM TF antibody, as set forth in Table 8 of PCT/US2019/12427, filed on Jan. 4, 2019 is selected from about 81%, about 89%, about 85%, about 109%, about 96%, about 97%, about 108%, about 104%, about 103%, about 112%, and about 89% compared to the control conditions without the antibody. In some embodiments, such % FXa ranges from about 81% to about 112%. In some embodiments, such % FXa is about 81% or more.

In some embodiments, the % FXa in the presence of 12.5 nM TF antibody, as set forth in Table 8 of PCT/US2019/12427, filed on Jan. 4, 2019 is selected from about 87%, about 89%, about 82%, about 99%, about 101%, about 98%, about 113%, about 106%, about 115%, about 110%, about 120%, about 85%, and about 108% compared to the control conditions without the antibody. In some embodiments, such % FXa ranges from about 82% to about 120%. In some embodiments, such % FXa is about 82% or more.

2.3.4. FVIIa Binding in the Presence of TF Antibodies

In some embodiments, the antibodies provided herein bind human TF at a human TF binding site that is distinct from a human TF binding site bound by human FVIIa. In certain embodiments, the antibodies provided herein do not compete for binding to human TF with human FVIIa.

In some embodiments, the percentage of FVIIa binding (% FVIIa) is at least 75% in the presence of no less than 250 nM TF antibody compared to the control conditions without the antibody. In some embodiments, the % FVIIa is at least 80% in the presence of no less than 250 nM TF antibody compared to the control conditions without the antibody. In some embodiments, the % FVIIa is at least 85% in the presence of no less than 250 nM TF antibody compared to the control conditions without the antibody. In some embodiments, the % FVIIa is at least 90% in the presence of no less than 250 nM TF antibody compared to the control conditions without the antibody. In some embodiments, the % FVIIa is at least 95% in the presence of no less than 250 nM TF antibody compared to the control conditions without the antibody.

In some embodiments, the % FVIIa is at least 75% in the presence of no less than 83 nM TF antibody compared to the control conditions without the antibody. In some embodiments, the % FVIIa is at least 80% in the presence of no less than 83 nM TF antibody compared to the control conditions without the antibody. In some embodiments, the % FVIIa is at least 85% in the presence of no less than 83 nM TF antibody compared to the control conditions without the antibody. In some embodiments, the % FVIIa is at least 90% in the presence of no less than 83 nM TF antibody compared to the control conditions without the antibody. In some embodiments, the % FVIIa is at least 95% in the presence of no less than 83 nM TF antibody compared to the control conditions without the antibody.

In some embodiments, the % FVIIa is at least 75% in the presence of no less than 28 nM TF antibody compared to the control conditions without the antibody. In some embodiments, the % FVIIa is at least 80% in the presence of no less than 28 nM TF antibody compared to the control conditions without the antibody. In some embodiments, the % FVIIa is at least 85% in the presence of no less than 28 nM TF antibody compared to the control conditions without the antibody. In some embodiments, the % FVIIa is at least 90% in the presence of no less than 28 nM TF antibody compared to the control conditions without the antibody. In some embodiments, the % FVIIa is at least 95% in the presence of no less than 28 nM TF antibody compared to the control conditions without the antibody.

In some embodiments, the % FVIIa is at least 75% in the presence of no less than 9.25 nM TF antibody compared to the control conditions without the antibody. In some embodiments, the % FVIIa is at least 80% in the presence of no less than 9.25 nM TF antibody compared to the control conditions without the antibody. In some embodiments, the % FVIIa is at least 85% in the presence of no less than 9.25 nM TF antibody compared to the control conditions without the antibody. In some embodiments, the % FVIIa is at least 90% in the presence of no less than 9.25 nM TF antibody compared to the control conditions without the antibody. In some embodiments, the % FVIIa is at least 95% in the presence of no less than 9.25 nM TF antibody compared to the control conditions without the antibody.

In some embodiments, the % FVIIa in the presence of 250 nM TF antibody, as set forth in Table 9 of PCT/US2019/12427, filed on Jan. 4, 2019 is selected from about 98%, about 87%, about 80%, about 92%, about 95%, about 89%, about 91%, about 97%, about 94%, about 101%, and about 96% compared to the control conditions without the antibody. In some embodiments, such % FVIIa ranges from about 80% to about 101%. In some embodiments, such % FVIIa is about 80% or more.

In some embodiments, the % FVIIa in the presence of 83 nM TF antibody, as set forth in Table 9 of PCT/US2019/12427, filed on Jan. 4, 2019 is selected from about 97%, about 88%, about 77%, about 93%, about 94%, about 91%, about 98%, about 100%, and about 92% compared to the control conditions without the antibody. In some embodiments, such % FVIIa ranges from about 77% to about 100%. In some embodiments, such % FVIIa is about 77% or more.

In some embodiments, the % FVIIa in the presence of 28 nM TF antibody, as set forth in Table 9 of PCT/US2019/12427, filed on Jan. 4, 2019 is selected from about 101%, about 87%, about 79%, about 96%, about 93%, about 95%, about 98%, about 100%, about 102%, about 99%, about 92%, and about 91% compared to the control conditions without the antibody. In some embodiments, such % FVIIa ranges from about 79% to about 102%. In some embodiments, such % FVIIa is about 79% or more.

In some embodiments, the % FVIIa in the presence of 9.25 nM TF antibody, as set forth in Table 9 of PCT/US2019/12427, filed on Jan. 4, 2019 is selected from about 100%, about 90%, about 76%, about 97%, about 93%, about 99%, about 98%, about 102%, about 101%, and about 95% compared to the control conditions without the antibody. In some embodiments, such % FVIIa ranges from about 76% to about 102%. In some embodiments, such % FVIIa is about 76% or more.

2.3.5. FVIIa-dependent TF Signaling in the Presence of TF Antibodies

In some embodiments, the antibodies provided herein inhibit FVIIa-dependent TF signaling. In some embodiments, the inhibition of FVIIa-dependent TF signaling is measured by the reduction of IL8. In some embodiments, the inhibition of FVIIa-dependent TF signaling is measured by the reduction of GM-CSF.

In some embodiments, the Interleukin 8 concentration (IL8 conc) is reduced by at least 70% in the presence of no less than 100 nM TF antibody compared to the control conditions without the antibody. In some embodiments, the IL8 conc is reduced by at least 80% in the presence of no less than 100 nM TF antibody compared to the control conditions without the antibody. In some embodiments, the IL8 conc is reduced by at least 90% in the presence of no less than 100 nM TF antibody compared to the control conditions without the antibody.

In some embodiments, the IL8 conc is reduced by at least 70% in the presence of no less than 40 nM TF antibody compared to the control conditions without the antibody. In some embodiments, the IL8 conc is reduced by at least 80% in the presence of no less than 40 nM TF antibody compared to the control conditions without the antibody. In some embodiments, the IL8 conc is reduced by at least 90% in the presence of no less than 40 nM TF antibody compared to the control conditions without the antibody.

In some embodiments, the IL8 conc is reduced by at least 60% in the presence of no less than 16 nM TF antibody compared to the control conditions without the antibody. In some embodiments, the IL8 conc is reduced by at least 70% in the presence of no less than 16 nM TF antibody compared to the control conditions without the antibody. In some embodiments, the IL8 conc is reduced by at least 80% in the presence of no less than 16 nM TF antibody compared to the control conditions without the antibody. In some embodiments, the IL8 conc is reduced by at least 90% in the presence of no less than 16 nM TF antibody compared to the control conditions without the antibody.

In some embodiments, the IL8 conc is reduced by at least 50% in the presence of no less than 6.4 nM TF antibody compared to the control conditions without the antibody. In some embodiments, the IL8 conc is reduced by at least 60% in the presence of no less than 6.4 nM TF antibody compared to the control conditions without the antibody. In some embodiments, the IL8 conc is reduced by at least 70% in the presence of no less than 6.4 nM TF antibody compared to the control conditions without the antibody. In some embodiments, the IL8 conc is reduced by at least 80% in the presence of no less than 6.4 nM TF antibody compared to the control conditions without the antibody. In some embodiments, the IL8 conc is reduced by at least 90% in the presence of no less than 6.4 nM TF antibody compared to the control conditions without the antibody.

In some embodiments, the Granulocyte-Macrophage Colony-Stimulating Factor concentration (GM-CSF conc) is reduced by at least 70% in the presence of no less than 100 nM TF antibody compared to the control conditions without the antibody. In some embodiments, the GM-CSF conc is reduced by at least 80% in the presence of no less than 100 nM TF antibody compared to the control conditions without the antibody. In some embodiments, the GM-CSF conc is reduced by at least 90% in the presence of no less than 100 nM TF antibody compared to the control conditions without the antibody.

In some embodiments, the GM-CSF conc is reduced by at least 70% in the presence of no less than 40 nM TF antibody compared to the control conditions without the antibody. In some embodiments, the GM-CSF conc is reduced by at least 80% in the presence of no less than 40 nM TF antibody compared to the control conditions without the antibody. In some embodiments, the GM-CSF conc is reduced by at least 90% in the presence of no less than 40 nM TF antibody compared to the control conditions without the antibody.

In some embodiments, the GM-CSF conc is reduced by at least 60% in the presence of no less than 16 nM TF antibody compared to the control conditions without the antibody. In some embodiments, the GM-CSF conc is reduced by at least 70% in the presence of no less than 16 nM TF antibody compared to the control conditions without the antibody. In some embodiments, the GM-CSF conc is reduced by at least 80% in the presence of no less than 16 nM TF antibody compared to the control conditions without the antibody. In some embodiments, the GM-CSF conc is reduced by at least 90% in the presence of no less than 16 nM TF antibody compared to the control conditions without the antibody.

In some embodiments, the GM-CSF conc is reduced by at least 50% in the presence of no less than 6.4 nM TF antibody compared to the control conditions without the antibody. In some embodiments, the GM-CSF conc is reduced by at least 60% in the presence of no less than 6.4 nM TF antibody compared to the control conditions without the antibody. In some embodiments, the GM-CSF conc is reduced by at least 70% in the presence of no less than 6.4 nM TF antibody compared to the control conditions without the antibody. In some embodiments, the GM-CSF conc is reduced by at least 80% in the presence of no less than 6.4 nM TF antibody compared to the control conditions without the antibody. In some embodiments, the GM-CSF conc is reduced by at least 90% in the presence of no less than 6.4 nM TF antibody compared to the control conditions without the antibody.

In some embodiments, the percentage of Interleukin 8 (% IL8) in the presence of 100 nM TF antibody, as set forth in Table 10 of PCT/US2019/12427, filed on Jan. 4, 2019 is selected from about 2%, about 9%, about 8%, about 6%, about 13%, about 1%, about 3%, about 4%, and about 5% compared to the control conditions without the antibody. In some embodiments, such % IL8 ranges from about 1% to about 13%. In some embodiments, such % IL8 is about 13% or less.

In some embodiments, the % IL8 in the presence of 40 nM TF antibody, as set forth in Table 10 of PCT/US2019/12427, filed on Jan. 4, 2019 is selected from about 2%, about 8%, about 7%, about 10%, about 14%, about 4%, about 5%, and about 6% compared to the control conditions without the antibody. In some embodiments, such % IL8 ranges from about 2% to about 14%. In some embodiments, such % IL8 is about 14% or less.

In some embodiments, the % IL8 in the presence of 16 nM TF antibody, as set forth in Table 10 of PCT/US2019/12427, filed on Jan. 4, 2019 is selected from about 2%, about 3%, about 10%, about 8%, about 7%, about 16%, about 9%, about 15%, about 5%, and about 6% compared to the control conditions without the antibody. In some embodiments, such % IL8 ranges from about 2% to about 16%. In some embodiments, such % IL8 is about 16% or less.

In some embodiments, the % IL8 in the presence of 6.4 nM TF antibody, as set forth in Table 10 of PCT/US2019/12427, filed on Jan. 4, 2019 is selected from about 3%, about 4%, about 11%, about 9%, about 14%, about 22%, about 12%, about 6%, about 5%, about 15%, about 21%, and about 8% compared to the control conditions without the antibody. In some embodiments, such % IL8 ranges from about 3% to about 22%. In some embodiments, such % IL8 is about 22% or less.

In some embodiments, the percentage of Granulocyte-Macrophage Colony-Stimulating Factor (% GM-CSF) in the presence of 100 nM TF antibody, as set forth in Table 11 of PCT/US2019/12427, filed on Jan. 4, 2019 is selected from about 6%, about 7%, about 22%, about 20%, about 12%, about 19%, about 17%, about 25%, about 5%, about 14%, about 11%, and about 10% compared to the control conditions without the antibody. In some embodiments, such % GM-CSF ranges from about 5% to about 25%. In some embodiments, such % GM-CSF is about 25% or less.

In some embodiments, the % GM-CSF in the presence of 40 nM TF antibody, as set forth in Table 11 of PCT/US2019/12427, filed on Jan. 4, 2019 is selected from about 6%, about 7%, about 19%, about 15%, about 18%, about 16%, about 26%, about 5%, about 13%, about 11%, and about 10% compared to the control conditions without the antibody. In some embodiments, such % GM-CSF ranges from about 5% to about 26%. In some embodiments, such % GM-CSF is about 26% or less.

In some embodiments, the % GM-CSF in the presence of 16 nM TF antibody, as set forth in Table 11 of PCT/US2019/12427, filed on Jan. 4, 2019 is selected from about 6%, about 7%, about 22%, about 19%, about 14%, about 32%, about 17%, about 26%, about 5%, about 12%, about 13%, about 9%, about 11%, and about 15% compared to the control conditions without the antibody. In some embodiments, such % GM-CSF ranges from about 5% to about 32%. In some embodiments, such % GM-CSF is about 32% or less.

In some embodiments, the % GM-CSF in the presence of 6.4 nM TF antibody, as set forth in Table 11 of PCT/US2019/12427, filed on Jan. 4, 2019 is selected from about 8%, about 9%, about 24%, about 20%, about 18%, about 39%, about 34%, about 15%, about 21%, about 16%, about 17%, and about 10% compared to the control conditions without the antibody. In some embodiments, such % GM-CSF ranges from about 8% to about 39%. In some embodiments, such % GM-CSF is about 39% or less.

2.4. Germlines

The antibodies provided herein may comprise any suitable V_{H} and V_{L} germline sequences.

In some embodiments, the V_{H} region of an antibody provided herein is from the VH3 germline. In some embodiments, the V_{H} region of an antibody provided herein is from the VH1 germline. In some embodiments, the V_{H} region of an antibody provided herein is from the VH4 germline.

In some embodiments, the V_{H} region of an antibody provided herein is from the VH3-23 germline. In some embodiments, the V_{H} region of an antibody provided herein is from the VH1-18 germline. In some embodiments, the V_{H} region of an antibody provided herein is from the VH3-30 germline. In some embodiments, the V_{H} region of an antibody provided herein is from the VH1-69 germline. In some embodiments, the V_{H} region of an antibody provided herein is from the VH4-31 germline. In some embodiments, the V_{H} region of an antibody provided herein is from the VH4-34 germline. In some embodiments, the V_{H} region of an antibody provided herein is from the VH1-46 germline.

In some embodiments, the V_{L} region of an antibody provided herein is from the VK1 germline. In some embodiments, the V_{L} region of an antibody provided herein is from the VK4 germline. In some embodiments, the V_{L} region of an antibody provided herein is from the VK3 germline

In some embodiments, the V_{L} region of an antibody provided herein is from the VK1-05 germline. In some embodiments, the V_{L} region of an antibody provided herein is from the VK4-01 germline. In some embodiments, the V_{L} region of an antibody provided herein is from the VK3-15 germline. In some embodiments, the V_{L} region of an antibody provided herein is from the VK3-20 germline. In some embodiments, the V_{L} region of an antibody provided herein is from the VK1-33 germline.

2.5. Monospecific and Multispecific TF Antibodies

In some embodiments, the antibodies provided herein are monospecific antibodies.

In some embodiments, the antibodies provided herein are multispecific antibodies.

In some embodiments, a multispecific antibody provided herein binds more than one antigen. In some embodiments, a multispecific antibody binds two antigens. In some embodiments, a multispecific antibody binds three antigens. In some embodiments, a multispecific antibody binds four antigens. In some embodiments, a multispecific antibody binds five antigens.

In some embodiments, a multispecific antibody provided herein binds more than one epitope on a TF antigen. In some embodiments, a multispecific antibody binds two epitopes on a TF antigen. In some embodiments, a multispecific antibody binds three epitopes on a TF antigen.

Many multispecific antibody constructs are known in the art, and the antibodies provided herein may be provided in the form of any suitable multispecific suitable construct.

In some embodiments, the multispecific antibody comprises an immunoglobulin comprising at least two different heavy chain variable regions each paired with a common light chain variable region (i.e., a “common light chain antibody”). The common light chain variable region forms a distinct antigen-binding domain with each of the two different heavy chain variable regions. See Merchant et al., Nature Biotechnol., 1998, 16:677-681, incorporated by reference in its entirety.

In some embodiments, the multispecific antibody comprises an immunoglobulin comprising an antibody or fragment thereof attached to one or more of the N- or C-termini of the heavy or light chains of such immunoglobulin. See Coloma and Morrison, Nature Biotechnol., 1997, 15:159-163, incorporated by reference in its entirety. In some aspects, such antibody comprises a tetravalent bispecific antibody.

In some embodiments, the multispecific antibody comprises a hybrid immunoglobulin comprising at least two different heavy chain variable regions and at least two different light chain variable regions. See Milstein and Cuello, Nature, 1983, 305:537-540; and Staerz and Bevan, Proc. Natl. Acad. Sci. USA, 1986, 83:1453-1457; each of which is incorporated by reference in its entirety.

In some embodiments, the multispecific antibody comprises immunoglobulin chains with alterations to reduce the formation of side products that do not have multispecificity. In some aspects, the antibodies comprise one or more “knobs-into-holes” modifications as described in U.S. Pat. No. 5,731,168, incorporated by reference in its entirety.

In some embodiments, the multispecific antibody comprises immunoglobulin chains with one or more electrostatic modifications to promote the assembly of Fc hetero-multimers. See WO 2009/089004, incorporated by reference in its entirety.

In some embodiments, the multispecific antibody comprises a bispecific single chain molecule. See Traunecker et al., EMBO J., 1991, 10:3655-3659; and Gruber et al., J. Immunol., 1994, 152:5368-5374; each of which is incorporated by reference in its entirety.

In some embodiments, the multispecific antibody comprises a heavy chain variable domain and a light chain variable domain connected by a polypeptide linker, where the length of the linker is selected to promote assembly of multispecific antibodies with the desired multispecificity. For example, monospecific scFvs generally form when a heavy chain variable domain and light chain variable domain are connected by a polypeptide linker of more than 12 amino acid residues. See U.S. Pat. Nos. 4,946,778 and 5,132,405, each of which is incorporated by reference in its entirety. In some embodiments, reduction of the polypeptide linker length to less than 12 amino acid residues prevents pairing of heavy and light chain variable domains on the same polypeptide chain, thereby allowing pairing of heavy and light chain variable domains from one chain with the complementary domains on another chain. The resulting antibodies therefore have multispecificity, with the specificity of each binding site contributed by more than one polypeptide chain. Polypeptide chains comprising heavy and light chain variable domains that are joined by linkers between 3 and 12 amino acid residues form predominantly dimers (termed diabodies). With linkers between 0 and 2 amino acid residues, trimers (termed triabodies) and tetramers (termed tetrabodies) are favored. However, the exact type of oligomerization appears to depend on the amino acid residue composition and the order of the variable domain in each polypeptide chain (e.g., V_{H}-linker-V_{L} vs. V_{L}-linker-V_{H}), in addition to the linker length. A skilled person can select the appropriate linker length based on the desired multispecificity.

In some embodiments, the multispecific antibody comprises a diabody. See Hollinger et al., Proc. Natl. Acad. Sci. USA, 1993, 90:6444-6448, incorporated by reference in its entirety. In some embodiments, the multispecific antibody comprises a triabody. See Todorovska et al., J. Immunol. Methods, 2001, 248:47-66, incorporated by reference in its entirety. In some embodiments, the multispecific antibody comprises a tetrabody. See id., incorporated by reference in its entirety.

In some embodiments, the multispecific antibody comprises a trispecific F(ab′)3 derivative. See Tutt et al. J. Immunol., 1991, 147:60-69, incorporated by reference in its entirety.

In some embodiments, the multispecific antibody comprises a cross-linked antibody. See U.S. Pat. No. 4,676,980; Brennan et al., Science, 1985, 229:81-83; Staerz, et al. Nature, 1985, 314:628-631; and EP 0453082; each of which is incorporated by reference in its entirety.

In some embodiments, the multispecific antibody comprises antigen-binding domains assembled by leucine zippers. See Kostelny et al., J. Immunol., 1992, 148:1547-1553, incorporated by reference in its entirety.

In some embodiments, the multispecific antibody comprises complementary protein domains. In some aspects, the complementary protein domains comprise an anchoring domain (AD) and a dimerization and docking domain (DDD). In some embodiments, the AD and DDD bind to each other and thereby enable assembly of multispecific antibody structures via the “dock and lock” (DNL) approach. Antibodies of many specificities may be assembled, including bispecific antibodies, trispecific antibodies, tetraspecific antibodies, quintspecific antibodies, and hexaspecific antibodies. Multispecific antibodies comprising complementary protein domains are described, for example, in U.S. Pat. Nos. 7,521,056; 7,550,143; 7,534,866; and 7,527,787; each of which is incorporated by reference in its entirety.

In some embodiments, the multispecific antibody comprises a dual action Fab (DAF) antibody as described in U.S. Pat. Pub. No. 2008/0069820, incorporated by reference in its entirety.

In some embodiments, the multispecific antibody comprises an antibody formed by reduction of two parental molecules followed by mixing of the two parental molecules and reoxidation to assembly a hybrid structure. See Carlring et al., PLoS One, 2011, 6:e22533, incorporated by reference in its entirety.

In some embodiments, the multispecific antibody comprises a DVD-Ig™. A DVD-Ig™ is a dual variable domain immunoglobulin that can bind to two or more antigens. DVD-Igs™ are described in U.S. Pat. No. 7,612,181, incorporated by reference in its entirety.

In some embodiments, the multispecific antibody comprises a DART™. DARTs™ are described in Moore et al., Blood, 2011, 117:454-451, incorporated by reference in its entirety.

In some embodiments, the multispecific antibody comprises a DuoBody®. DuoBodies® are described in Labrijn et al., Proc. Natl. Acad. Sci. USA, 2013, 110:5145-5150; Gramer et al., mAbs, 2013, 5:962-972; and Labrijn et al., Nature Protocols, 2014, 9:2450-2463; each of which is incorporated by reference in its entirety.

In some embodiments, the multispecific antibody comprises an antibody fragment attached to another antibody or fragment. The attachment can be covalent or non-covalent. When the attachment is covalent, it may be in the form of a fusion protein or via a chemical linker. Illustrative examples of multispecific antibodies comprising antibody fragments attached to other antibodies include tetravalent bispecific antibodies, where an scFv is fused to the C-terminus of the C_{H3} from an IgG. See Coloma and Morrison, Nature Biotechnol., 1997, 15:159-163. Other examples include antibodies in which a Fab molecule is attached to the constant region of an immunoglobulin. See Miler et al., J. Immunol., 2003, 170:4854-4861, incorporated by reference in its entirety. Any suitable fragment may be used, including any of the fragments described herein or known in the art.

In some embodiments, the multispecific antibody comprises a CovX-Body. CovX-Bodies are described, for example, in Doppalapudi et al., Proc. Natl. Acad. Sci. USA, 2010, 107:22611-22616, incorporated by reference in its entirety.

In some embodiments, the multispecific antibody comprises an Fcab antibody, where one or more antigen-binding domains are introduced into an Fc region. Fcab antibodies are described in Wozniak-Knopp et al., Protein Eng. Des. Sel., 2010, 23:289-297, incorporated by reference in its entirety.

In some embodiments, the multispecific antibody comprises a TandAb® antibody. TandAb® antibodies are described in Kipriyanov et al., J. Mol. Biol., 1999, 293:41-56 and Zhukovsky et al., Blood, 2013, 122:5116, each of which is incorporated by reference in its entirety.

In some embodiments, the multispecific antibody comprises a tandem Fab. Tandem Fabs are described in WO 2015/103072, incorporated by reference in its entirety.

In some embodiments, the multispecific antibody comprises a Zybody™ Zybodies™ are described in LaFleur et al., mAbs, 2013, 5:208-218, incorporated by reference in its entirety.

2.6. Glycosylation Variants

In certain embodiments, an antibody provided herein may be altered to increase, decrease or eliminate the extent to which it is glycosylated. Glycosylation of polypeptides is typically either “N-linked” or “O-linked.”

“N-linked” glycosylation refers to the attachment of a carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site.

“O-linked” glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition or deletion of N-linked glycosylation sites to or from an antibody provided herein may be accomplished by altering the amino acid sequence such that one or more of the above-described tripeptide sequences is created or removed. Addition or deletion of O-linked glycosylation sites may be accomplished by addition, deletion, or substitution of one or more serine or threonine residues in or to (as the case may be) the sequence of an antibody.

In some embodiments, an antibody provided herein comprises a glycosylation motif that is different from a naturally occurring antibody. Any suitable naturally occurring glycosylation motif can be modified in the antibodies provided herein. The structural and glycosylation properties of immunoglobulins, for example, are known in the art and summarized, for example, in Schroeder and Cavacini, J. Allergy Clin. Immunol., 2010, 125:S41-52, incorporated by reference in its entirety.

In some embodiments, an antibody provided herein comprises an IgG1 Fc region with modification to the oligosaccharide attached to asparagine 297 (Asn 297). Naturally occurring IgG1 antibodies produced by mammalian cells typically comprise a branched, biantennary oligosaccharide that is generally attached by an N-linkage to Asn 297 of the C_{H2} domain of the Fc region. See Wright et al., TIBTECH, 1997, 15:26-32, incorporated by reference in its entirety. The oligosaccharide attached to Asn 297 may include various carbohydrates such as mannose, N-acetyl glucosamine (GlcNAc), galactose, and sialic acid, as well as a fucose attached to a GlcNAc in the “stem” of the biantennary oligosaccharide structure.

In some embodiments, the oligosaccharide attached to Asn 297 is modified to create antibodies having altered ADCC. In some embodiments, the oligosaccharide is altered to improve ADCC. In some embodiments, the oligosaccharide is altered to reduce ADCC.

In some aspects, an antibody provided herein comprises an IgG1 domain with reduced fucose content at position Asn 297 compared to a naturally occurring IgG1 domain. Such Fc domains are known to have improved ADCC. See Shields et al., J. Biol. Chem., 2002, 277:26733-26740, incorporated by reference in its entirety. In some aspects, such antibodies do not comprise any fucose at position Asn 297. The amount of fucose may be determined using any suitable method, for example as described in WO 2008/077546, incorporated by reference in its entirety.

In some embodiments, an antibody provided herein comprises a bisected oligosaccharide, such as a biantennary oligosaccharide attached to the Fc region of the antibody that is bisected by GlcNAc. Such antibody variants may have reduced fucosylation and/or improved ADCC function. Examples of such antibody variants are described, for example, in WO 2003/011878; U.S. Pat. No. 6,602,684; and U.S. Pat. Pub. No. 2005/0123546; each of which is incorporated by reference in its entirety.

Other illustrative glycosylation variants which may be incorporated into the antibodies provided herein are described, for example, in U.S. Pat. Pub. Nos. 2003/0157108, 2004/0093621, 2003/0157108, 2003/0115614, 2002/0164328, 2004/0093621, 2004/0132140, 2004/0110704, 2004/0110282, 2004/0109865; International Pat. Pub. Nos. 2000/61739, 2001/29246, 2003/085119, 2003/084570, 2005/035586, 2005/035778; 2005/053742, 2002/031140; Okazaki et al., J. Mol. Biol., 2004, 336:1239-1249; and Yamane-Ohnuki et al., Biotech. Bioeng., 2004, 87: 614-622; each of which is incorporated by reference in its entirety.

In some embodiments, an antibody provided herein comprises an Fc region with at least one galactose residue in the oligosaccharide attached to the Fc region. Such antibody variants may have improved CDC function. Examples of such antibody variants are described, for example, in WO 1997/30087; WO 1998/58964; and WO 1999/22764; each of which is incorporated by reference in its entirety.

Examples of cell lines capable of producing defucosylated antibodies include Lec13 CHO cells, which are deficient in protein fucosylation (see Ripka et al., Arch. Biochem. Biophys., 1986, 249:533-545; U.S. Pat. Pub. No. 2003/0157108; WO 2004/056312; each of which is incorporated by reference in its entirety), and knockout cell lines, such as alpha-1,6-fucosyltransferase gene or FUT8 knockout CHO cells (see Yamane-Ohnuki et al., Biotech. Bioeng., 2004, 87: 614-622; Kanda et al., Biotechnol. Bioeng., 2006, 94:680-688; and WO 2003/085107; each of which is incorporated by reference in its entirety).

In some embodiments, an antibody provided herein is an aglycosylated antibody. An aglycosylated antibody can be produced using any method known in the art or described herein. In some aspects, an aglycosylated antibody is produced by modifying the antibody to remove all glycosylation sites. In some aspects, the glycosylation sites are removed only from the Fc region of the antibody. In some aspects, an aglycosylated antibody is produced by expressing the antibody in an organism that is not capable of glycosylation, such as E. coli, or by expressing the antibody in a cell-free reaction mixture.

In some embodiments, an antibody provided herein has a constant region with reduced effector function compared to a native IgG1 antibody. In some embodiments, the affinity of a constant region of an Fc region of an antibody provided herein for Fc receptor is less than the affinity of a native IgG1 constant region for such Fc receptor.

2.7. Constant Regions, Fc Region and Amino Acid Sequence Variants

In some embodiments, an antibody provided herein comprises one or more constant regions.

In some embodiments, the antibody comprises a human Ig constant domain. In some embodiments, the antibody comprises a constant region from a human IgA, IgG, IgE, IgD, or IgM antibody. In some embodiments, the antibody comprises a constant region from human IgG. The human IgG can be human IgG1, human IgG2, human IgG3, or human IgG4.

In some embodiments, the antibody comprises a human IgG1 CH1 domain. In some embodiments, the human IgG1 CH1 domain sequence is as follows:

ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKK
V.

In some embodiments, the human IgG1 CH1 domain is from a particular allotype. Human IgG1 allotypes suitable for any of the antibodies herein are described in http://www.imgt.org/IMGTrepertoire/Proteins/allotypes/human/IGH/IGHC/G1m_allotypes.html, which is hereby incorporated by reference in its entirety. In particular embodiments, the allotype is G1m3, also referred to herein as IGHG1*03. The G1m3, also known as IGHG1*03 allotype, is described in http://www.imgt.org/IMGTrepertoire/Proteins/allotypes/human/IGH/IGHC/Glm_allotypes.html, which is hereby incorporated by reference in its entirety.

In some embodiments, the human IgG1 CH1 region of allotype IGHG1*03 comprises the CH1 domain sequence

ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRV.

In some embodiments, an antibody provided herein comprises an Fc region. The Fc region can be from a human IgA, IgG, IgE, IgD, or IgM antibody.

In some embodiments, the antibody comprises a human IgG Fc region. The human IgG Fc region can be a human IgG1 Fc region, human IgG2 Fc region, human IgG3 Fc region, human IgG4 Fc region.

In particular embodiments, the antibody comprises a human IgG1 Fc region. The human IgG1 Fc region may comprise a hinge sequence. In some embodiments, the hinge sequence is EPKSCDKTHTCP.

The human IgG1 Fc region may comprise a human IgG1 CH2 domain sequence. In some embodiments, the human I2G1 CH2 domain sequence is as follows:

PCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAK.

The human IgG1 Fc region may comprise a human IgG1 CH3 domain sequence. The human IgG1 CH3 domain sequence is as follows:

GQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKS
LSLSPG.

In some embodiments, the human IgG1 CH3 domain sequence further comprises a C-terminal lysine (K).

In some embodiments, the human IgG1 Fc region comprises the following sequence:

PCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSV
MHEALHNHYTQKSLSLSPG.

In some embodiments, the human IgG1 Fc region sequence further comprises a C-terminal lysine (K).

In some embodiments, the human IgG1 Fc region comprises the following sequence:

EPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG.

In some embodiments, the human IgG1 Fc region sequence further comprises a C-terminal lysine (K).

In some embodiments, the human IgG1 Fc region is of a particular allotype. Human IgG1 allotypes suitable for any of the antibodies herein are described in http://www.imgt.org/IMGTrepertoire/Proteins/allotypes/human/IGH/IGHC/G1m_allotypes.html, which is hereby incorporated by reference in its entirety. In particular embodiments, the allotype is G1m3, also referred to herein as IGHG1*03. The G1m3, also known as IGHG1*03 allotype, is described in http://www.imgt.org/IMGTrepertoire/Proteins/allotypes/human/IGH/IGHC/G1m_allotypes.html, which is hereby incorporated by reference in its entirety.

In some embodiments, the human IgG1 allotype IGHG1*03 Fc region comprises the following CH2 sequence:

PCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAK.

In some embodiments, the human IgG1 allotype IGHG1*03 Fc region comprises the following CH3 sequence:

GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPG.

In some embodiments, the CH3 region of the human IgG1 allotype IGHG1*03 Fc region further comprises a C-terminal lysine (K).

In some embodiments, the human IgG1 allotype IGHG1*03 Fc region comprises the following Fc sequence:

PCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSV
MHEALHNHYTQKSLSLSPG.

In some embodiments, the human IgG1 allotype IGHG1*03 Fc region sequence further comprises a C-terminal lysine (K).

In some embodiments, the human IgG1 allotype IGHG1*03 Fc region comprises the following Fc sequence:

EPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPG.

In some embodiments, the human IgG1 allotype IGHG1*03 Fc region sequence further comprises a C-terminal lysine (K).

In certain embodiments, an antibody provided herein comprises an Fc region with one or more amino acid substitutions, insertions, or deletions in comparison to a naturally occurring Fc region. In some aspects, such substitutions, insertions, or deletions yield antibodies with altered stability, glycosylation, or other characteristics. In some aspects, such substitutions, insertions, or deletions yield aglycosylated antibodies.

In some aspects, the Fc region of an antibody provided herein is modified to yield an antibody with altered affinity for an Fc receptor, or an antibody that is more immunologically inert. In some embodiments, the antibody variants provided herein possess some, but not all, effector functions. Such antibodies may be useful, for example, when the half-life of the antibody is important in vivo, but when certain effector functions (e.g., complement activation and ADCC) are unnecessary or deleterious.

In some embodiments, the Fc region of an antibody provided herein is a human IgG4 Fc region comprising one or more of the hinge stabilizing mutations S228P and L235E. See Aalberse et al., Immunology, 2002, 105:9-19, incorporated by reference in its entirety. In some embodiments, the IgG4 Fc region comprises one or more of the following mutations: E233P, F234V, and L235A. See Armour et al., Mol. Immunol., 2003, 40:585-593, incorporated by reference in its entirety. In some embodiments, the IgG4 Fc region comprises a deletion at position G236.

In some embodiments, the Fc region of an antibody provided herein is a human IgG1 Fc region comprising one or more mutations to reduce Fc receptor binding. In some aspects, the one or more mutations are in residues selected from S228 (e.g., S228A), L234 (e.g., L234A), L235 (e.g., L235A), D265 (e.g., D265A), and N297 (e.g., N297A). In some aspects, the antibody comprises a PVA236 mutation. PVA236 means that the amino acid sequence ELLG, from amino acid position 233 to 236 of IgG1 or EFLG of IgG4, is replaced by PVA. See U.S. Pat. No. 9,150,641, incorporated by reference in its entirety.

In some embodiments, the Fc region of an antibody provided herein is modified as described in Armour et al., Eur. J. Immunol., 1999, 29:2613-2624; WO 1999/058572; and/or U.K. Pat. App. No. 98099518; each of which is incorporated by reference in its entirety.

In some embodiments, the Fc region of an antibody provided herein is a human IgG2 Fc region comprising one or more of mutations A330S and P331S.

In some embodiments, the Fc region of an antibody provided herein has an amino acid substitution at one or more positions selected from 238, 265, 269, 270, 297, 327 and 329. See U.S. Pat. No. 6,737,056, incorporated by reference in its entirety. Such Fc mutants include Fc mutants with substitutions at two or more of amino acid positions 265, 269, 270, 297 and 327, including the so-called “DANA” Fc mutant with substitution of residues 265 and 297 with alanine. See U.S. Pat. No. 7,332,581, incorporated by reference in its entirety. In some embodiments, the antibody comprises an alanine at amino acid position 265. In some embodiments, the antibody comprises an alanine at amino acid position 297.

In certain embodiments, an antibody provided herein comprises an Fc region with one or more amino acid substitutions which improve ADCC, such as a substitution at one or more of positions 298, 333, and 334 of the Fc region. In some embodiments, an antibody provided herein comprises an Fc region with one or more amino acid substitutions at positions 239, 332, and 330, as described in Lazar et al., Proc. Natl. Acad. Sci. USA, 2006, 103:4005-4010, incorporated by reference in its entirety.

In some embodiments, an antibody provided herein comprises one or more alterations that improves or diminishes C1q binding and/or CDC. See U.S. Pat. No. 6,194,551; WO 99/51642; and Idusogie et al., J. Immunol., 2000, 164:4178-4184; each of which is incorporated by reference in its entirety.

In some embodiments, an antibody provided herein comprises one or more alterations to increase half-life. Antibodies with increased half-lives and improved binding to the neonatal Fc receptor (FcRn) are described, for example, in Hinton et al., J. Immunol., 2006, 176:346-356; and U.S. Pat. Pub. No. 2005/0014934; each of which is incorporated by reference in its entirety. Such Fc variants include those with substitutions at one or more of Fc region residues: 238, 250, 256, 265, 272, 286, 303, 305, 307, 311, 312, 314, 317, 340, 356, 360, 362, 376, 378, 380, 382, 413, 424, 428, and 434 of an IgG.

In some embodiments, an antibody provided herein comprises one or more Fc region variants as described in U.S. Pat. Nos. 7,371,826, 5,648,260, and 5,624,821; Duncan and Winter, Nature, 1988, 322:738-740; and WO 94/29351; each of which is incorporated by reference in its entirety.

2.8. Pyroglutamate

As is known in the art, both glutamate (E) and glutamine (Q) at the N-termini of recombinant proteins can cyclize spontaneously to form pyroglutamate (pE) in vitro and in vivo. See Liu et al., J. Biol. Chem., 2011, 286:11211-11217, incorporated by reference in its entirety.

In some embodiments, provided herein are antibodies comprising a polypeptide sequence having a pE residue at the N-terminal position. In some embodiments, provided herein are antibodies comprising a polypeptide sequence in which the N-terminal residue has been converted from Q to pE. In some embodiments, provided herein are antibodies comprising a polypeptide sequence in which the N-terminal residue has been converted from E to pE.

2.9. Cysteine Engineered Antibody Variants

In certain embodiments, provided herein are cysteine engineered antibodies, also known as “thioMAbs,” in which one or more residues of the antibody are substituted with cysteine residues. In particular embodiments, the substituted residues occur at solvent accessible sites of the antibody. By substituting such residues with cysteine, reactive thiol groups are introduced at solvent accessible sites of the antibody and may be used to conjugate the antibody to other moieties, such as drug moieties or linker-drug moieties, for example, to create an immunoconjugate.

In certain embodiments, any one or more of the following residues may be substituted with cysteine: V205 of the light chain; A118 of the heavy chain Fc region; and S400 of the heavy chain Fc region. Cysteine engineered antibodies may be generated as described, for example, in U.S. Pat. No. 7,521,541, which is incorporated by reference in its entirety.

### 3. Anti-TF Antibody-Drug Conjugates

Provided herein are antibody-drug conjugates (ADCs) comprising an antibody that binds specifically to TF and a cytotoxic agent. In some embodiments, the cytotoxic agent is linked directly to the anti-TF antibody. In some embodiments, the cytotoxic agent is linked indirectly to the anti-TF antibody.

In some embodiments, the ADCs further comprise a linker. In some embodiments, the linker links the anti-TF antibody to the cytotoxic agent.

The number of cytotoxic agents conjugated to an antibody in an ADC is defined as the drug-antibody ratio or DAR. As is known in the art, the majority of conjugation methods yield an ADC composition that includes various DAR species, with the reported DAR being the average of the individual DAR species. Thus, when the ADCs described herein are defined as having a specific DAR, it is to be understood that the number provided represents the average of the individual DAR species in the ADC composition. In some embodiments, the ADCs provided herein have a drug-antibody ratio (DAR) of 1. In some embodiments, the ADCs provided herein have a DAR of 2. In some embodiments, the ADCs provided herein have a DAR of 3. In some embodiments, the ADCs provided herein have a DAR of 4. In some embodiments, the ADCs provided herein have a DAR of 5. In some embodiments, the ADCs provided herein have a DAR of 1-2, 1-3, 1-4, 1-5, 2-3, 2-4, 2-5, 3-4, 3-5, 4-5, 1, 2, 3, 4, or 5. In some embodiments, the ADCs provided herein have a DAR greater than 5. In some embodiments, the DAR is measured by UV/vis spectroscopy, hydrophobic interaction chromatography (HIC), and/or reverse phase liquid chromatography separation with time-of-flight detection and mass characterization (RP-UPLC/Mass spectrometry). In some embodiments, distribution of drug-linked forms (for example, the fraction of DAR0, DAR1, DAR2, etc. species) may also be analyzed by various techniques known in the art, including MS (with or without an accompanying chromatographic separation step), hydrophobic interaction chromatography, reverse-phase HPLC or iso-electric focusing gel electrophoresis (IEF) (see, for example, Sun et al., Bioconj Chem., 28:1371-81 (2017); Wakankar et al., mAbs, 3:161-172 (2011)).

### 4. Methods for Making TF Antibodies

4.1. TF Antigen Preparation

The TF antigen used for isolation of the antibodies provided herein may be intact TF or a fragment of TF. The TF antigen may be, for example, in the form of an isolated protein or a protein expressed on the surface of a cell.

In some embodiments, the TF antigen is a non-naturally occurring variant of TF, such as a TF protein having an amino acid sequence or post-translational modification that does not occur in nature.

In some embodiments, the TF antigen is truncated by removal of, for example, intracellular or membrane-spanning sequences, or signal sequences. In some embodiments, the TF antigen is fused at its C-terminus to a human IgG1 Fc domain or a polyhistidine tag.

4.2. Methods of Making Monoclonal Antibodies

Monoclonal antibodies may be obtained, for example, using the hybridoma method first described by Kohler et al., Nature, 1975, 256:495-497 (incorporated by reference in its entirety), and/or by recombinant DNA methods (see e.g., U.S. Pat. No. 4,816,567, incorporated by reference in its entirety). Monoclonal antibodies may also be obtained, for example, using phage-display libraries (see e.g., U.S. Pat. No. 8,258,082, which is incorporated by reference in its entirety) or, alternatively, using yeast-based libraries (see e.g., U.S. Pat. No. 8,691,730, which is incorporated by reference in its entirety).

In the hybridoma method, a mouse or other appropriate host animal is immunized to elicit lymphocytes that produce or are capable of producing antibodies that will specifically bind to the protein used for immunization. Alternatively, lymphocytes may be immunized in vitro. Lymphocytes are then fused with myeloma cells using a suitable fusing agent, such as polyethylene glycol, to form a hybridoma cell. See Goding J. W., Monoclonal Antibodies: Principles and Practice 3^{rd} ed. (1986) Academic Press, San Diego, Calif., incorporated by reference in its entirety.

The hybridoma cells are seeded and grown in a suitable culture medium that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Useful myeloma cells are those that fuse efficiently, support stable high-level production of antibody by the selected antibody-producing cells, and are sensitive media conditions, such as the presence or absence of HAT medium. Among these, preferred myeloma cell lines are murine myeloma lines, such as those derived from MOP-21 and MC-11 mouse tumors (available from the Salk Institute Cell Distribution Center, San Diego, Calif.), and SP-2 or X63-Ag8-653 cells (available from the American Type Culture Collection, Rockville, Md.). Human myeloma and mouse-human heteromyeloma cell lines also have been described for the production of human monoclonal antibodies. See e.g., Kozbor, J. Immunol., 1984, 133:3001, incorporated by reference in its entirety.

After the identification of hybridoma cells that produce antibodies of the desired specificity, affinity, and/or biological activity, selected clones may be subcloned by limiting dilution procedures and grown by standard methods. See Goding, supra. Suitable culture media for this purpose include, for example, D-MEM or RPMI-1640 medium. In addition, the hybridoma cells may be grown in vivo as ascites tumors in an animal.

DNA encoding the monoclonal antibodies may be readily isolated and sequenced using conventional procedures (e.g., by using oligonucleotide probes that are capable of binding specifically to genes encoding the heavy and light chains of the monoclonal antibodies). Thus, the hybridoma cells can serve as a useful source of DNA encoding antibodies with the desired properties. Once isolated, the DNA may be placed into expression vectors, which are then transfected into host cells such as bacteria (e.g., E. coli), yeast (e.g., Saccharomyces or Pichia sp.), COS cells, Chinese hamster ovary (CHO) cells, or myeloma cells that do not otherwise produce antibody, to produce the monoclonal antibodies.

4.3. Methods of Making Chimeric Antibodies

Illustrative methods of making chimeric antibodies are described, for example, in U.S. Pat. No. 4,816,567; and Morrison et al., Proc. Natl. Acad. Sci. USA, 1984, 81:6851-6855; each of which is incorporated by reference in its entirety. In some embodiments, a chimeric antibody is made by using recombinant techniques to combine a non-human variable region (e.g., a variable region derived from a mouse, rat, hamster, rabbit, or non-human primate, such as a monkey) with a human constant region.

4.4. Methods of Making Humanized Antibodies

Humanized antibodies may be generated by replacing most, or all, of the structural portions of a non-human monoclonal antibody with corresponding human antibody sequences. Consequently, a hybrid molecule is generated in which only the antigen-specific variable, or CDR, is composed of non-human sequence. Methods to obtain humanized antibodies include those described in, for example, Winter and Milstein, Nature, 1991, 349:293-299; Rader et al., Proc. Nat. Acad. Sci. U.S.A., 1998, 95:8910-8915; Steinberger et al., J. Biol. Chem., 2000, 275:36073-36078; Queen et al., Proc. Natl. Acad. Sci. U.S.A., 1989, 86:10029-10033; and U.S. Pat. Nos. 5,585,089, 5,693,761, 5,693,762, and 6,180,370; each of which is incorporated by reference in its entirety.

4.5. Methods of Making Human Antibodies

Human antibodies can be generated by a variety of techniques known in the art, for example by using transgenic animals (e.g., humanized mice). See, e.g., Jakobovits et al., Proc. Natl. Acad. Sci. U.S.A., 1993, 90:2551; Jakobovits et al., Nature, 1993, 362:255-258; Bruggermann et al., Year in Immuno., 1993, 7:33; and U.S. Pat. Nos. 5,591,669, 5,589,369 and 5,545,807; each of which is incorporated by reference in its entirety. Human antibodies can also be derived from phage-display libraries (see e.g., Hoogenboom et al., J. Mol. Biol., 1991, 227:381-388; Marks et al., J. Mol. Biol., 1991, 222:581-597; and U.S. Pat. Nos. 5,565,332 and 5,573,905; each of which is incorporated by reference in its entirety). Human antibodies may also be generated by in vitro activated B cells (see e.g., U.S. Pat. Nos. 5,567,610 and 5,229,275, each of which is incorporated by reference in its entirety). Human antibodies may also be derived from yeast-based libraries (see e.g., U.S. Pat. No. 8,691,730, incorporated by reference in its entirety).

4.6. Methods of Making Antibody Fragments

The antibody fragments provided herein may be made by any suitable method, including the illustrative methods described herein or those known in the art. Suitable methods include recombinant techniques and proteolytic digestion of whole antibodies. Illustrative methods of making antibody fragments are described, for example, in Hudson et al., Nat. Med., 2003, 9:129-134, incorporated by reference in its entirety. Methods of making scFv antibodies are described, for example, in Plückthun, in The Pharmacology of Monoclonal Antibodies, vol. 113, Rosenburg and Moore eds., Springer-Verlag, New York, pp. 269-315 (1994); WO 93/16185; and U.S. Pat. Nos. 5,571,894 and 5,587,458; each of which is incorporated by reference in its entirety.

4.7. Methods of Making Alternative Scaffolds

The alternative scaffolds provided herein may be made by any suitable method, including the illustrative methods described herein or those known in the art. For example, methods of preparing Adnectins™ are described in Emanuel et al., mAbs, 2011, 3:38-48, incorporated by reference in its entirety. Methods of preparing iMabs are described in U.S. Pat. Pub. No. 2003/0215914, incorporated by reference in its entirety. Methods of preparing Anticalins® are described in Vogt and Skerra, Chem. Biochem., 2004, 5:191-199, incorporated by reference in its entirety. Methods of preparing Kunitz domains are described in Wagner et al., Biochem. & Biophys. Res. Comm., 1992, 186:118-1145, incorporated by reference in its entirety. Methods of preparing thioredoxin peptide aptamers are provided in Geyer and Brent, Meth. Enzymol., 2000, 328:171-208, incorporated by reference in its entirety. Methods of preparing Affibodies are provided in Fernandez, Curr. Opinion in Biotech., 2004, 15:364-373, incorporated by reference in its entirety. Methods of preparing DARPins are provided in Zahnd et al., J. Mol. Biol., 2007, 369:1015-1028, incorporated by reference in its entirety. Methods of preparing Affilins are provided in Ebersbach et al., J. Mol. Biol., 2007, 372:172-185, incorporated by reference in its entirety. Methods of preparing Tetranectins are provided in Graversen et al., J. Biol. Chem., 2000, 275:37390-37396, incorporated by reference in its entirety. Methods of preparing Avimers are provided in Silverman et al., Nature Biotech., 2005, 23:1556-1561, incorporated by reference in its entirety. Methods of preparing Fynomers are provided in Silacci et al., J. Biol. Chem., 2014, 289:14392-14398, incorporated by reference in its entirety.

Further information on alternative scaffolds is provided in Binz et al., Nat. Biotechnol., 2005 23:1257-1268; and Skerra, Current Opin. in Biotech., 2007 18:295-304, each of which is incorporated by reference in its entirety.

4.8. Methods of Making Multispecific Antibodies

The multispecific antibodies provided herein may be made by any suitable method, including the illustrative methods described herein or those known in the art. Methods of making common light chain antibodies are described in Merchant et al., Nature Biotechnol., 1998, 16:677-681, incorporated by reference in its entirety. Methods of making tetravalent bispecific antibodies are described in Coloma and Morrison, Nature Biotechnol., 1997, 15:159-163, incorporated by reference in its entirety. Methods of making hybrid immunoglobulins are described in Milstein and Cuello, Nature, 1983, 305:537-540; and Staerz and Bevan, Proc. Natl. Acad. Sci. USA, 1986, 83:1453-1457; each of which is incorporated by reference in its entirety. Methods of making immunoglobulins with knobs-into-holes modification are described in U.S. Pat. No. 5,731,168, incorporated by reference in its entirety. Methods of making immunoglobulins with electrostatic modifications are provided in WO 2009/089004, incorporated by reference in its entirety. Methods of making bispecific single chain antibodies are described in Traunecker et al., EMBO J., 1991, 10:3655-3659; and Gruber et al., J. Immunol., 1994, 152:5368-5374; each of which is incorporated by reference in its entirety. Methods of making single-chain antibodies, whose linker length may be varied, are described in U.S. Pat. Nos. 4,946,778 and 5,132,405, each of which is incorporated by reference in its entirety. Methods of making diabodies are described in Hollinger et al., Proc. Natl. Acad. Sci. USA, 1993, 90:6444-6448, incorporated by reference in its entirety. Methods of making triabodies and tetrabodies are described in Todorovska et al., J. Immunol. Methods, 2001, 248:47-66, incorporated by reference in its entirety. Methods of making trispecific F(ab′)3 derivatives are described in Tutt et al. J. Immunol., 1991, 147:60-69, incorporated by reference in its entirety. Methods of making cross-linked antibodies are described in U.S. Pat. No. 4,676,980; Brennan et al., Science, 1985, 229:81-83; Staerz, et al. Nature, 1985, 314:628-631; and EP 0453082; each of which is incorporated by reference in its entirety. Methods of making antigen-binding domains assembled by leucine zippers are described in Kostelny et al., J. Immunol., 1992, 148:1547-1553, incorporated by reference in its entirety. Methods of making antibodies via the DNL approach are described in U.S. Pat. Nos. 7,521,056; 7,550,143; 7,534,866; and 7,527,787; each of which is incorporated by reference in its entirety. Methods of making hybrids of antibody and non-antibody molecules are described in WO 93/08829, incorporated by reference in its entirety, for examples of such antibodies. Methods of making DAF antibodies are described in U.S. Pat. Pub. No. 2008/0069820, incorporated by reference in its entirety. Methods of making antibodies via reduction and oxidation are described in Carlring et al., PLoS One, 2011, 6:e22533, incorporated by reference in its entirety. Methods of making DVD-Igs™ are described in U.S. Pat. No. 7,612,181, incorporated by reference in its entirety. Methods of making DARTs™ are described in Moore et al., Blood, 2011, 117:454-451, incorporated by reference in its entirety. Methods of making DuoBodies® are described in Labrijn et al., Proc. Natl. Acad. Sci. USA, 2013, 110:5145-5150; Gramer et al., mAbs, 2013, 5:962-972; and Labrijn et al., Nature Protocols, 2014, 9:2450-2463; each of which is incorporated by reference in its entirety. Methods of making antibodies comprising scFvs fused to the C-terminus of the C_{H3} from an IgG are described in Coloma and Morrison, Nature Biotechnol., 1997, 15:159-163, incorporated by reference in its entirety. Methods of making antibodies in which a Fab molecule is attached to the constant region of an immunoglobulin are described in Miler et al., J. Immunol., 2003, 170:4854-4861, incorporated by reference in its entirety. Methods of making CovX-Bodies are described in Doppalapudi et al., Proc. Natl. Acad. Sci. USA, 2010, 107:22611-22616, incorporated by reference in its entirety. Methods of making Fcab antibodies are described in Wozniak-Knopp et al., Protein Eng. Des. Sel., 2010, 23:289-297, incorporated by reference in its entirety. Methods of making TandAb® antibodies are described in Kipriyanov et al., J. Mol. Biol., 1999, 293:41-56 and Zhukovsky et al., Blood, 2013, 122:5116, each of which is incorporated by reference in its entirety. Methods of making tandem Fabs are described in WO 2015/103072, incorporated by reference in its entirety. Methods of making Zybodies™ are described in LaFleur et al., mAbs, 2013, 5:208-218, incorporated by reference in its entirety.

4.9. Methods of Making Variants

In some embodiments, an antibody provided herein is an affinity matured variant of a parent antibody, which may be generated, for example, using phage display-based affinity maturation techniques. Briefly, one or more CDR residues may be mutated and the variant antibodies, or portions thereof, displayed on phage and screened for affinity. Such alterations may be made in CDR “hotspots,” or residues encoded by codons that undergo mutation at high frequency during the somatic maturation process (see Chowdhury, Methods Mol. Biol., 2008, 207:179-196, incorporated by reference in its entirety), and/or residues that contact the antigen.

Any suitable method can be used to introduce variability into a polynucleotide sequence(s) encoding an antibody, including error-prone PCR, chain shuffling, and oligonucleotide-directed mutagenesis such as trinucleotide-directed mutagenesis (TRIM). In some aspects, several CDR residues (e.g., 4-6 residues at a time) are randomized. CDR residues involved in antigen binding may be specifically identified, for example, using alanine scanning mutagenesis or modeling. CDR-H3 and CDR-L3 in particular are often targeted for mutation.

The introduction of diversity into the variable regions and/or CDRs can be used to produce a secondary library. The secondary library is then screened to identify antibody variants with improved affinity. Affinity maturation by constructing and reselecting from secondary libraries has been described, for example, in Hoogenboom et al., Methods in Molecular Biology, 2001, 178:1-37, incorporated by reference in its entirety.

4.10. Vectors, Host Cells, and Recombinant Methods

Also provided are isolated nucleic acids encoding TF antibodies, vectors comprising the nucleic acids, and host cells comprising the vectors and nucleic acids, as well as recombinant techniques for the production of the antibodies.

For recombinant production of an antibody, the nucleic acid(s) encoding it may be isolated and inserted into a replicable vector for further cloning (i.e., amplification of the DNA) or expression. In some aspects, the nucleic acid may be produced by homologous recombination, for example as described in U.S. Pat. No. 5,204,244, incorporated by reference in its entirety.

Many different vectors are known in the art. The vector components generally include one or more of the following: a signal sequence, an origin of replication, one or more marker genes, an enhancer element, a promoter, and a transcription termination sequence, for example as described in U.S. Pat. No. 5,534,615, incorporated by reference in its entirety.

Illustrative examples of suitable host cells are provided below. These host cells are not meant to be limiting, and any suitable host cell may be used to produce the antibodies provided herein.

Suitable host cells include any prokaryotic (e.g., bacterial), lower eukaryotic (e.g., yeast), or higher eukaryotic (e.g., mammalian) cells. Suitable prokaryotes include eubacteria, such as Gram-negative or Gram-positive organisms, for example, Enterobacteriaceae such as Escherichia (E. coli), Enterobacter, Erwinia, Klebsiella, Proteus, Salmonella (S. typhimurium), Serratia (S. marcescans), Shigella, Bacilli (B. subtilis and B. licheniformis), Pseudomonas (P. aeruginosa), and Streptomyces. One useful E. coli cloning host is E. coli 294, although other strains such as E. coli B, E. coli X1776, and E. coli W3110 are also suitable.

In addition to prokaryotes, eukaryotic microbes such as filamentous fungi or yeast are also suitable cloning or expression hosts for TF antibody-encoding vectors. Saccharomyces cerevisiae, or common baker's yeast, is a commonly used lower eukaryotic host microorganism. However, a number of other genera, species, and strains are available and useful, such as Schizosaccharomyces pombe, Kluyveromyces (K. lactis, K. fragilis, K. bulgaricus K. wickeramii, K. waltii, K. drosophilarum, K. thermotolerans, and K. marxianus), Yarrowia, Pichia pastoris, Candida (C. albicans), Trichoderma reesia, Neurospora crassa, Schwanniomyces (S. occidentalis), and filamentous fungi such as, for example Penicillium, Tolypocladium, and Aspergillus (A. nidulans and A. niger).

Useful mammalian host cells include COS-7 cells, HEK293 cells, baby hamster kidney (BHK) cells, Chinese hamster ovary (CHO), mouse sertoli cells, African green monkey kidney cells (VERO-76), and the like.

The host cells used to produce the TF antibody of this invention may be cultured in a variety of media. Commercially available media such as, for example, Ham's F10, Minimal Essential Medium (MEM), RPMI-1640, and Dulbecco's Modified Eagle's Medium (DMEM) are suitable for culturing the host cells. In addition, any of the media described in Ham et al., Meth. Enz., 1979, 58:44; Barnes et al., Anal. Biochem., 1980, 102:255; and U.S. Pat. Nos. 4,767,704, 4,657,866, 4,927,762, 4,560,655, and 5,122,469; or WO 90/03430 and WO 87/00195 may be used. Each of the foregoing references is incorporated by reference in its entirety.

Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics, trace elements (defined as inorganic compounds usually present at final concentrations in the micromolar range), and glucose or an equivalent energy source. Any other necessary supplements may also be included at appropriate concentrations that would be known to those skilled in the art.

The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. For example, Carter et al. (Bio/Technology, 1992, 10:163-167, incorporated by reference in its entirety) describes a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation.

In some embodiments, the antibody is produced in a cell-free system. In some aspects, the cell-free system is an in vitro transcription and translation system as described in Yin et al., mAbs, 2012, 4:217-225, incorporated by reference in its entirety. In some aspects, the cell-free system utilizes a cell-free extract from a eukaryotic cell or from a prokaryotic cell. In some aspects, the prokaryotic cell is E. coli. Cell-free expression of the antibody may be useful, for example, where the antibody accumulates in a cell as an insoluble aggregate, or where yields from periplasmic expression are low.

Where the antibody is secreted into the medium, supernatants from such expression systems are generally first concentrated using a commercially available protein concentration filter, for example, an Amicon® or Millipore® Pellcon® ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being a particularly useful purification technique. The suitability of protein A as an affinity ligand depends on the species and isotype of any immunoglobulin Fc domain that is present in the antibody. Protein A can be used to purify antibodies that comprise human γ1, γ2, or γ4 heavy chains (Lindmark et al., J. Immunol. Meth., 1983, 62:1-13, incorporated by reference in its entirety). Protein G is useful for all mouse isotypes and for human γ3 (Guss et al., EMBO J., 1986, 5:1567-1575, incorporated by reference in its entirety).

The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H3} domain, the BakerBond ABX® resin is useful for purification.

Other techniques for protein purification, such as fractionation on an ion-exchange column, ethanol precipitation, Reverse Phase HPLC, chromatography on silica, chromatography on heparin Sepharose, chromatofocusing, SDS-PAGE, and ammonium sulfate precipitation are also available, and can be applied by one of skill in the art.

Following any preliminary purification step(s), the mixture comprising the antibody of interest and contaminants may be subjected to low pH hydrophobic interaction chromatography using an elution buffer at a pH between about 2.5 to about 4.5, generally performed at low salt concentrations (e.g., from about 0 to about 0.25 M salt).

### 5. Cytotoxic Agents

In some embodiments, ADCs provided herein comprise a cytotoxic agent. The cytotoxic agents provided herein include various anti-tumor or anti-cancer agents known in the art. In some embodiments, the cytotoxic agents cause destruction of cancer cells. In some embodiments, the cytotoxic agents inhibit the growth or proliferation of cancer cells.

Suitable cytotoxic agents include anti-angiogenic agents, pro-apoptotic agents, anti-mitotic agents, anti-kinase agents, alkylating agents, hormones, hormone agonists, hormone antagonists, chemokines, drugs, prodrugs, toxins, enzymes, antimetabolites, antibiotics, alkaloids, and radioactive isotopes.

In some embodiments, the cytotoxic agent comprises at least one of: calicheamycin, camptothecin, carboplatin, irinotecan, SN-38, cyclophosphamide, cytarabine, dacarbazine, docetaxel, dactinomycin, daunorubicin, doxorubicin, etoposide, idarubicin, topotecan, vinca alkaloid, maytansinoid, maytansinoid analog, pyrrolobenzodiazepine, taxoid, duocarmycin, dolastatin, auristatin and derivatives thereof.

In certain embodiments, the cytotoxic agent is an auristatin derivative. In certain embodiments, the auristatin derivative is monomethyl auristatin E moiety (MMAE). In certain embodiments, the auristatin derivative is monomethyl auristatin F (MMAF). In some embodiments, the auristatin derivative is one of the auristatin derivatives described in International Patent Application Publication No. WO 2016/041082. In some embodiments, the auristatin derivative is a moiety derived from a compound of general Formula I:

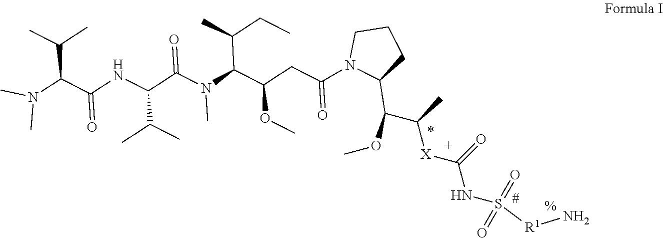

wherein: X is *—C(O)NHCH(CH₂(R²))—⁺, wherein * and + represent the respective points of attachment as indicated in Formula I, or X is absent; R¹ is selected from the group consisting of:

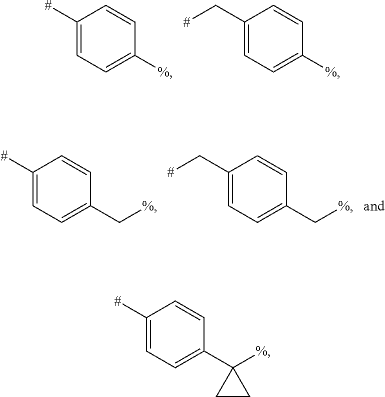

wherein # and % represent the respective points of attachment as indicated in Formula I; and R² is phenyl.

In some embodiments, in compounds of general Formula I, R¹ is selected from the group consisting of:

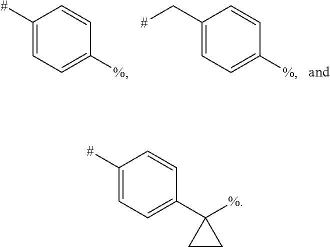

In some embodiments, in compounds of general Formula I, the compound is represented by Formula II:

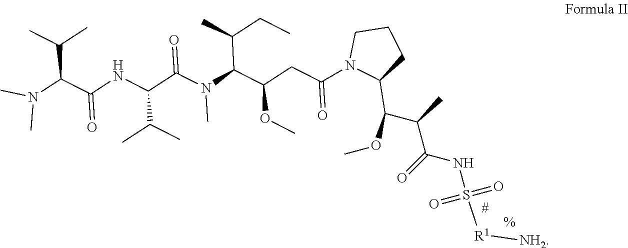

In some embodiments, in compounds of general Formula II, R¹ is selected from the group consisting of:

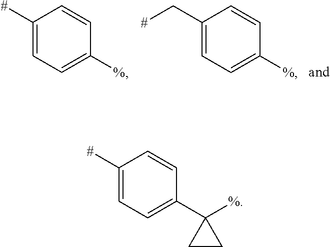

In some embodiments, in compounds of general Formula II, R¹ is selected from the group consisting of:

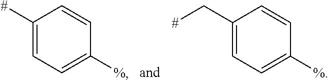

In some embodiments, in compounds of general Formula II, R¹ is:

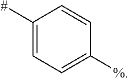

In some embodiments, in compounds of general Formula I, the compound is represented by Formula III:

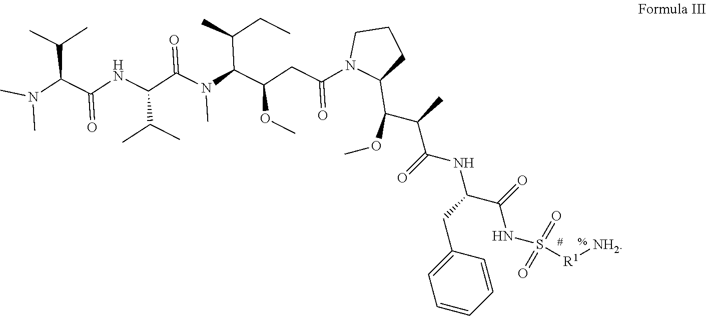

In some embodiments, in compounds of general Formula III, R¹ is selected from the group consisting of:

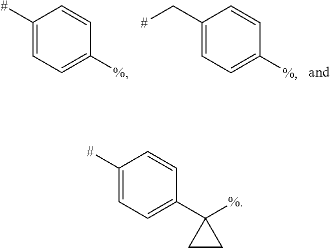

In some embodiments, in compounds of general Formula III, R¹ is selected from the group consisting of:

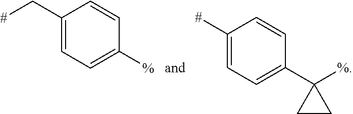

In some embodiments, in compounds of general Formula III, R¹ is:

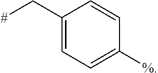

In certain embodiments, the compound of Formula I is Compound 9:

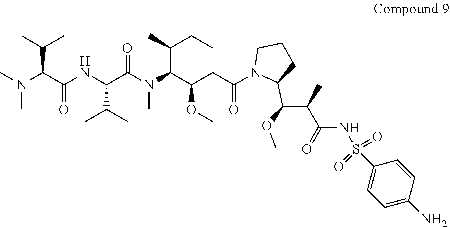 [SMILES: CC[C@H](C)[C@@H]([C@@H](CC(=O)N1CCC[C@H]1[C@H](OC)[C@@H](C)C(=O)NS(=O)(=O)c1ccc(N)cc1)OC)N(C)C(=O)[C@@H](NC(=O)[C@H](C(C)C)N(C)C)C(C)C]

It is to be understood that reference to compounds of general Formula I throughout the remainder of this disclosure includes, in various embodiments, compounds of general Formula II and general Formula III, to the same extent as if embodiments reciting each of these formulae individually were specifically recited.

In some embodiments, the cytotoxic agent is a diagnostic agent, such as a radioactive isotope, a metal chelator, an enzyme, a fluorescent compound, a bioluminescent compound, or a chemiluminescent compound.

In some embodiments, the cytotoxic agent is a cytotoxic payload improved safety profile, for example XMT-1267 and other cytotoxic payloads described in Trail et al., Pharmacol Ther, 2018, 181:126-142.

In certain embodiments, the ADC of the present disclosure comprises a TF antibody conjugated to an auristatin derivative (toxin) via a linker (L). In certain embodiments, the ADC comprises: (a) an antigen binding protein (Ab) which binds to the extracellular domain of human Tissue Factor (TF), wherein the Ab comprises a VH-CDR1, a VH-CDR2, a VH-CDR3, a VL-CDR1, a VL-CDR2, and a VL-CDR3, wherein (i) the VH-CDR1 comprises SEQ ID NO: 872, the VH-CDR2 comprises SEQ ID NO: 873, the VH-CDR3 comprises SEQ ID NO: 874, the VL-CDR1 comprises SEQ ID NO: 875, the VL-CDR2 comprises SEQ ID NO: 876, and the VL-CDR3 comprises SEQ ID NO: 877, (ii) the VH-CDR1, VH-CDR2, VH-CDR3, VL-CDR1, VL-CDR2, and VL-CDR3 are from the antibody designated 25A3, (iii) the VH-CDR1, VH-CDR2, VH-CDR3, VL-CDR1, VL-CDR2, and VL-CDR3 are from the antibody designated 25A, (i) the VH-CDR1, VH-CDR2, VH-CDR3, VL-CDR1, VL-CDR2, and VL-CDR3 are from the antibody designated 25A5, (v) the VH-CDR1, VH-CDR2, VH-CDR3, VL-CDR1, VL-CDR2, and VL-CDR3 are from the antibody designated 25A5-T, or (vi) the VH-CDR1, VH-CDR2, VH-CDR3, VL-CDR1, VL-CDR2, and VL-CDR3 are from the antibody designated 25G1; and (b) one or more linker-toxin moieties represented by Formula IV:

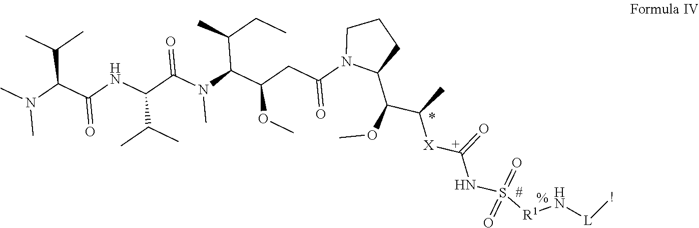

wherein: X is *—C(O)NHCH(CH₂(R²))—⁺, wherein * and + represent the respective points of attachment as indicated in Formula IV, or X is absent; L is a linker; ! represents the point of attachment of L to the Ab, where L is attached to the Ab through a covalent bond; R¹ is selected from the group consisting of:

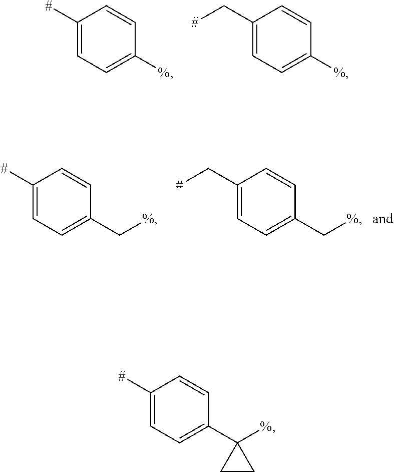

wherein # and % represent the respective points of attachment as indicated in Formula IV; and R² is phenyl.

In some embodiments, in the linker-toxin moiety of general Formula IV, X is absent.

In some embodiments, in the linker-toxin moiety of general Formula IV, L is a cleavable linker.

In some embodiments, in the linker-toxin moiety of general Formula IV, L is a peptide-containing linker.

In some embodiments, the linker-toxin moiety of general Formula IV is represented by general Formula V:

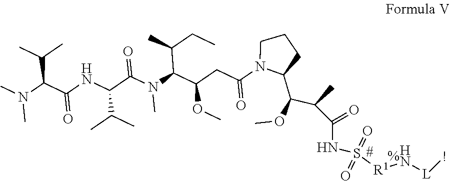

wherein R¹, L and ! are as defined above for general Formula IV.

In some embodiments, in the linker-toxin moiety of general Formula V, R¹ is selected from the group consisting of:

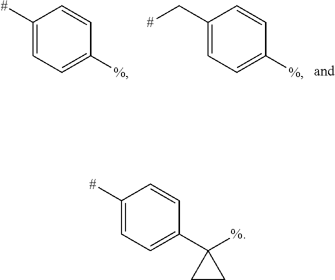

In some embodiments, in the linker-toxin moiety of general Formula V, R¹ is selected from the group consisting of:

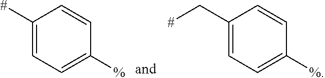

In some embodiments, in the linker-toxin moiety of general Formula V, R¹ is:

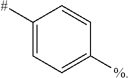

In some embodiments, in the linker-toxin moiety of general Formula V, L is a cleavable linker.

In some embodiments, in the linker-toxin moiety of general Formula V, L is a peptide-containing linker.

In some embodiments, in the linker-toxin moiety of general Formula V, L is a protease-cleavable linker.

In some embodiments, in the linker-toxin moiety of general Formula IV or Formula V, L is a linker selected from one of N-(β-maleimidopropyloxy)-N-hydroxysuccinimide ester (BMPS), N-(ε-maleimidocaproyloxy)succinimide ester (EMCS), N-[γ-maleimidobutyryloxy]succinimide ester (GMBS), 1,6-hexane-bis-vinylsulfone (HBVS), succinimidyl 4-(N-maleimidomethyl)cyclohexane-1-carboxy-(6-amidocaproate) (LC-SMCC), m-maleimidobenzoyl-N-hydroxysuccinimide ester (MBS), 4-(4-N-Maleimidophenyl)butyric acid hydrazide (MPBH), succinimidyl 3-(bromoacetamido)propionate (SBAP), succinimidyl iodoacetate (SIA), succinimidyl (4-iodoacetyl)aminobenzoate (STAB), N-succinimidyl-3-(2-pyridyldithio) propionate (SPDP), N-succinimidyl-4-(2-pyridylthio)pentanoate (SPP), succinimidyl 4-(N-maleimidomethyl)cyclohexane-1-carboxylate (SMCC), succinimidyl 4-(p-maleimidophenyl)butyrate (SMPB), succinimidyl 6-[(β-maleimidopropionamido)hexanoate] (SMPH), iminothiolane (IT), sulfo-EMCS, sulfo-GMBS, sulfo-KMUS, sulfo-MBS, sulfo-SIAB, sulfo-SMCC, sulfo-SMPB, and succinimidyl-(4-vinylsulfone)benzoate (SVSB).

In some embodiments, in the linker-toxin moiety of general Formula IV or Formula V, L comprises a poly(ethylene)glycol chain of the formula:

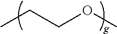

wherein g is an integer from 1-20.

In some embodiments, in the linker-toxin moiety of general Formula IV or Formula V, g is 3.

Also contemplated herein, are ADCs comprising a TF antibody conjugated to a linker-toxin of general Formula IV or Formula V, in which the linker has general Formula VIII or general Formula IX as described below.

In certain embodiments, the ADC of the present disclosure comprising a tissue factor (TF) antibody conjugated to an auristatin derivative (toxin) via a linker (L) has general Formula VI:

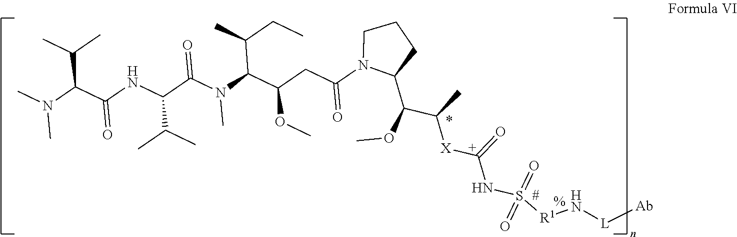

wherein: Ab represents the TF antibody; n is an integer greater than or equal to 1; X is *—C(O)NHCH(CH₂(R²))—⁺, wherein * and + represent the respective points of attachment as indicated in Formula VI, or X is absent; L is a linker, where L is attached to the Ab through a covalent bond; R¹ is selected from the group consisting of:

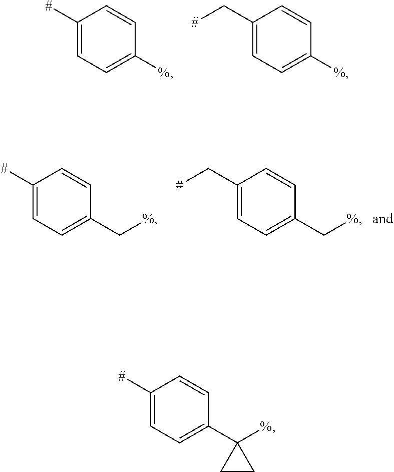

wherein # and % represent the respective points of attachment as indicated in Formula VI; and R² is phenyl.

In some embodiments, in the ADC of general Formula VI, n is an integer from 1 to 10. In some embodiments, in the ADC of general Formula VI, n is an integer selected from the group consisting of 1, 2, 3, 4, and 5. In some embodiments, in the ADC of general Formula VI, n is an integer selected from the group consisting of 2, 3, and 4.

In some embodiments, in the ADC of general Formula VI, R¹ is selected from the group consisting of:

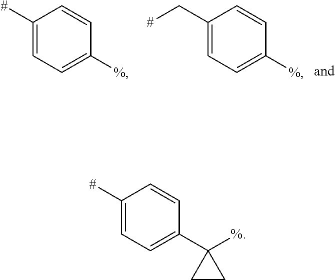

In some embodiments, in the ADC of general Formula VI, X is absent.

In some embodiments, in the ADC of general Formula VI, R¹ is selected from the group consisting of:

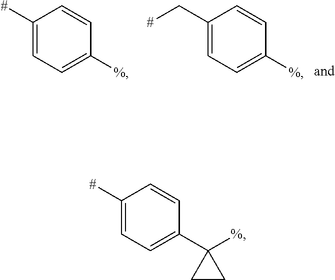

and X is absent.

In some embodiments, in the ADC of general Formula VI, R¹ is selected from the group consisting of:

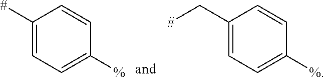

In some embodiments, in the ADC of general Formula VI, R¹ is selected from the group consisting of:

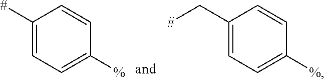

and X is absent.

In some embodiments, in the ADC of general Formula VI, R¹ is:

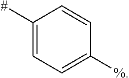

In some embodiments, in the ADC of general Formula VI, R¹ is:

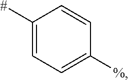

and X is absent.

In some embodiments, in the ADC of general Formula VI, L is a cleavable linker. In some embodiments, in the ADC of general Formula VI, L is a peptide-containing linker. In some embodiments, in the ADC of general Formula VI, L is a protease-cleavable linker. In some embodiments, in the ADC of general Formula VI, L is a linker selected from one of N-(β-maleimidopropyloxy)-N-hydroxysuccinimide ester (BMPS), N-(ε-maleimidocaproyloxy) succinimide ester (EMCS), N-[γ-maleimidobutyryloxy]succinimide ester (GMBS), 1,6-hexane-bis-vinylsulfone (HBVS), succinimidyl 4-(N-maleimidomethyl)cyclohexane-1-carboxy-(6-amidocaproate) (LC-SMCC), m-maleimidobenzoyl-N-hydroxysuccinimide ester (MBS), 4-(4-N-Maleimidophenyl)butyric acid hydrazide (MPBH), succinimidyl 3-(bromoacetamido)propionate (SBAP), succinimidyl iodoacetate (SIA), succinimidyl (4-iodoacetyl)aminobenzoate (SIAB), N-succinimidyl-3-(2-pyridyldithio) propionate (SPDP), N-succinimidyl-4-(2-pyridylthio)pentanoate (SPP), succinimidyl 4-(N-maleimidomethyl)cyclohexane-1-carboxylate (SMCC), succinimidyl 4-(p-maleimidophenyl)butyrate (SMPB), succinimidyl 6-[β-maleimidopropionamido)hexanoate] (SMPH), iminothiolane (IT), sulfo-EMCS, sulfo-GMBS, sulfo-KMUS, sulfo-MBS, sulfo-SIAB, sulfo-SMCC, sulfo-SMPB and succinimidyl-(4-vinylsulfone)benzoate (SVSB).

In some embodiments, in the ADC of general Formula VI, L comprises a poly(ethylene)glycol chain of the formula:

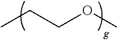

wherein g is an integer from 1-20.

In some embodiments, in the ADC of general Formula VI, g is 3.

In certain embodiments of the ADC of general formula VI, L is represented by a linker of general Formula VII:

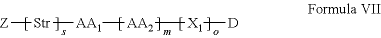

wherein: Z represents a functional group that binds to a target group (e.g., the thiol of a cysteine or primary amine of a lysine group) of the TF antibody; D represents the point of attachment to the amino group as indicated in Formula VI; Str is a stretcher; AA₁ and AA₂ are each independently an amino acid, wherein AA₁-[AA₂]ₘ forms a protease cleavage site; X₁ is a self-immolative group; s is an integer selected from 0 and 1; m is an integer selected from the group consisting of 1, 2, 3, and 4; and o is an integer selected from 0, 1, and 2.

In some embodiments, in the ADC of general Formula VI, where L is a linker of general Formula VII, [Str]ₛ is selected from the group consisting of alkylene, stretchers based on aliphatic acids, stretchers based on aliphatic diacids, stretchers based on aliphatic amines and stretchers based on aliphatic diamines.

In some embodiments, in the ADC of general Formula VI, where L is a linker of general Formula VII, [Str]ₛ is selected from the group consisting of diglycolate-based stretchers, malonate-based stretchers, caproate-based stretchers and caproamide-based stretchers.

In some embodiments, in the ADC of general Formula VI, where L is a linker of general Formula VII, [Str]ₛ is selected from the group consisting of glycine-based stretchers, polyethylene glycol-based stretchers, and monomethoxy polyethylene glycol-based stretchers.

In some embodiments, in the ADC of general Formula VI, where L is a linker of general Formula VII, [Str]ₛ is:

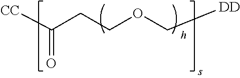

wherein h is an integer from 1-20, CC refers to the point of attachment to AA₁; and DD refers to the point of attachment to Z.

In some embodiments, in the ADC of general Formula VI, where L is a linker of general Formula VII, [Str]ₛ is selected from:

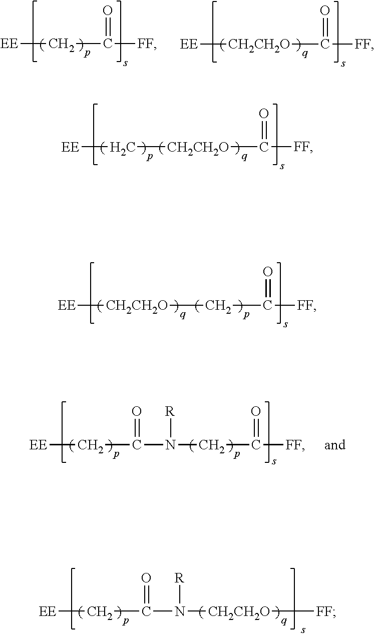

wherein: EE and FF represent the points of attachment to Z and AA₁, respectively; R is selected from hydrogen and C₁-C₆ alkyl; each occurrence of p is independently an integer from 2 to 10; and each occurrence of q is independently an integer from 1 to 10.

In some embodiments, in the ADC of general Formula VI, where L is a linker of general Formula VII, [Str]ₛ is selected from the group consisting of:

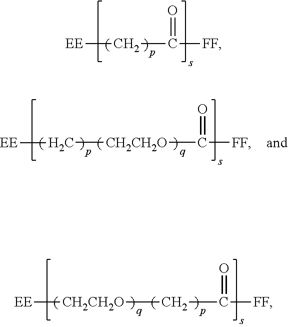

wherein: EE and FF represent the points of attachment to Z and AA₁, respectively; each occurrence of p is independently an integer from 2 to 10; and each occurrence of q is independently an integer from 1 to 10.

In some embodiments, in the ADC of general Formula VI, where L is a linker of general Formula VII, [Str]ₛ is selected from:

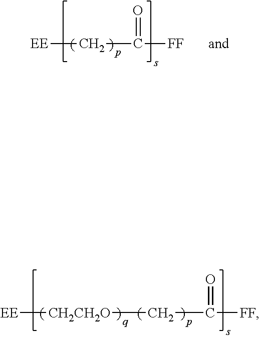

wherein: EE and FF represent the points of attachment to Z and AA₁, respectively; each occurrence of p is independently an integer from 2 to 6, and q is an integer from 2 to 8.

In some embodiments, in the ADC of general Formula VI, where L is a linker of general Formula VII, AA₁-[AA₂]ₘ is selected from Val-Lys, Ala-Lys, Phe-Lys, Val-Cit, Phe-Cit, Leu-Cit, Ile-Cit, Trp-Cit, Phe-Arg, Ala-Phe, Val-Ala, Met-Lys, Asn-Lys, Ile-Pro, Ile-Val, Asp-Val, His-Val, Met-(D)Lys, Asn-(D)Lys, Val-(D)Asp, NorVal-(D)Asp, Ala-(D)Asp, Me₃Lys-Pro, PhenylGly-(D)Lys, Met-(D)Lys, Asn-(D)Lys, Pro-(D)Lys, Met-(D)Lys, Met-Cit-Val, Gly-Cit-Val, (D)Phe-Phe-Lys, (D)Ala-Phe-Lys, Gly-Phe-Leu-Gly, and Ala-Leu-Ala-Leu.

In some embodiments, in the ADC of general Formula VI, where L is a linker of general Formula VII, s is 1.

In some embodiments, in the ADC of general Formula VI, where L is a linker of general Formula VII, o is 0.

In some embodiments, in the ADC of general Formula VI, where L is a linker of general Formula VII, m is selected from 1, 2 and 3.

In some embodiments, in the ADC of general Formula VI, where L is a linker of general Formula VII, m is 1.

In some embodiments, in the ADC of general Formula VI, where L is a linker of general Formula VII, AA₁-[AA₂]ₘ is a dipeptide selected from Val-Lys, Ala-Lys, Phe-Lys, Val-Cit, Phe-Cit, Leu-Cit, Be-Cit and Trp-Cit.

In some embodiments, in the ADC of general Formula VI, where L is a linker of general Formula VII, each X₁ is independently selected from p-aminobenzyloxycarbonyl (PABC), p-aminobenzyl ether (PABE) and methylated ethylene diamine (MED).

In some embodiments, in the ADC of general Formula VI, where L is a linker of general Formula VII, and [Str]ₛ is:

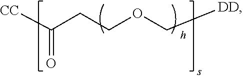

s is 1 and h is 3.

In certain embodiments, the ADC comprises a linker-toxin moiety having the structure of Formula VIII:

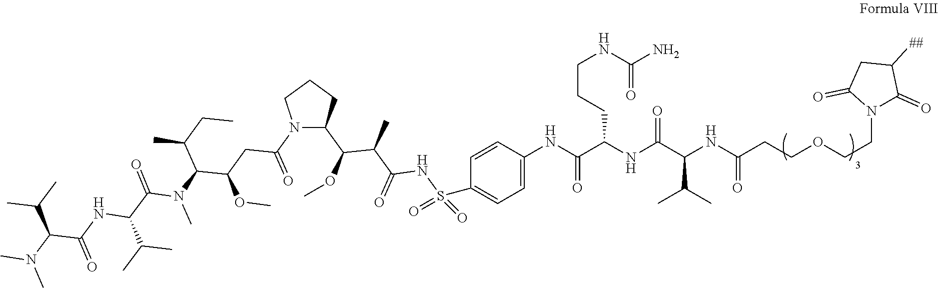

wherein ## represents the point of attachment of the linker-toxin moiety to the TF antibody and the linker-toxin moiety is attached to the TF antibody through a covalent bond.

In some embodiments, provided herein is an antibody-drug conjugate of Formula IX:

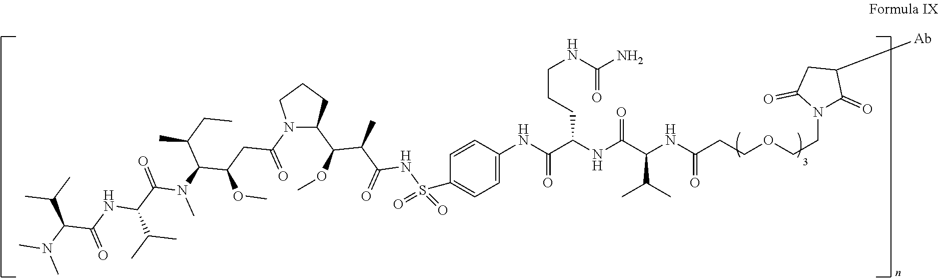

wherein:

Ab is a tissue factor (TF) antibody, and n is an integer greater than or equal to 1. In some embodiments, in the ADC of Formula IX, n is an integer from 1 to 10. In some embodiments of the ADC of Formula IX, n is selected from the group consisting of 1, 2, 3, 4, and 5. In some embodiments, in the ADC of Formula IX, n is an integer selected from the group consisting of 2, 3, and 4. In some embodiments, in the ADC of Formula IX, the succinimidyl group is attached to the Ab through a covalent bond.

In some embodiments of the ADC of Formula IX, the Ab comprises a VH-CDR1, a VH-CDR2, a VH-CDR3, a VL-CDR1, a VL-CDR2, and a VL-CDR3, wherein
i. the VH-CDR1 comprises SEQ ID NO: 872, the VH-CDR2 comprises SEQ ID NO: 873, the VH-CDR3 comprises SEQ ID NO: 874, the VL-CDR1 comprises SEQ ID NO: 875, the VL-CDR2 comprises SEQ ID NO: 876, and the VL-CDR3 comprises SEQ ID NO: 877,
ii. the VH-CDR1, VH-CDR2, VH-CDR3, VL-CDR1, VL-CDR2, and VL-CDR3 are from the antibody designated 25A3,
iii. the VH-CDR1, VH-CDR2, VH-CDR3, VL-CDR1, VL-CDR2, and VL-CDR3 are from the antibody designated 25A,
iv. the VH-CDR1, VH-CDR2, VH-CDR3, VL-CDR1, VL-CDR2, and VL-CDR3 are from the antibody designated 25A5,
v. the VH-CDR1, VH-CDR2, VH-CDR3, VL-CDR1, VL-CDR2, and VL-CDR3 are from the antibody designated 25A5-T, or
vi. the VH-CDR1, VH-CDR2, VH-CDR3, VL-CDR1, VL-CDR2, and VL-CDR3 are from the antibody designated 25G1, n is an integer greater than or equal to 1.

In some embodiments of the ADC of Formula IX, the Ab comprises a VH-CDR1, a VH-CDR2, a VH-CDR3, a VL-CDR1, a VL-CDR2, and a VL-CDR3 from the antibody designated 25A3.

In an embodiment, the ADCs described herein comprise an antibody that comprises:

a full heavy chain sequence that is

QVQLVQSGAEVKKPGASVKVSCKASGYTFDx[V/A]YGISWVRQAPGQGLE
WMGWIAPYx[N/S]GNTNYAQKLQGRVTMTTDTSTSTAYMELRSLRSDDTA
VYYCARDAGTYSPFGYGMDVWGQGTTVTVSSASTKGPSVFPLAPSSKSTSG
GTAALGCLVKDYFPEPVTVSWNSGALTSGVHTFPAVLQSSGLYSLSSVVTV
PSSSLGTQTYICNVNHKPSNTKVDKRVEPKSCDKTHTCPPCPAPELLGGPS
VFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLSL
SPG

and a light chain sequence that is

DIQMTQSPSTLSASVGDRVTITCx[R/Q]ASx[Q/E]SIx[S/N]x[S/
N]WLAWYQQKPGKAPKLLIYKAx[S/Y]x[S/N]LEx[S/Y]GVPSRFS
GSGSGTEFTLTISSLQPDDFATYYCQx[Q/L]FQx[S/K]LPPFTFGGG
TKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQ
GLSSPVTKSFNRGEC,

a heavy chain sequence that is

QVQLVQSGAEVKKPGASVKVSCKASGYTFDVYGISWVRQAPGQGLEWMGW
IAPYSGNTNYAQKLQGRVTMTTDTSTSTAYMELRSLRSDDTAVYYCARDA
GTYSPFGYGMDVWGQGTTVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PG

and a light chain sequence that is

DIQMTQSPSTLSASVGDRVTITCQASQSINNWLAWYQQKPGKAPKLLIYK
AYNLESGVPSRFSGSGSGTEFTLTISSLQPDDFATYYCQLFQSLPPFTFG
GGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC,

a heavy chain sequence that is

QVQLVQSGAEVKKPGASVKVSCKASGYTFDVYGISWVRQAPGQGLEWMGW
IAPYSGNTNYAQKLQGRVTMTTDTSTSTAYMELRSLRSDDTAVYYCARDA
GTYSPFGYGMDVWGQGTTVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PG

and a light chain sequence that is

DIQMTQSPSTLSASVGDRVTITCRASQSISSWLAWYQQKPGKAPKLLIYK
ASSLESGVPSRFSGSGSGTEFTLTISSLQPDDFATYYCQQFQSLPPFTFG
GGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC,

a heavy chain sequence that is

QVQLVQSGAEVKKPGASVKVSCKASGYTFDAYGISWVRQAPGQGLEWMGW
IAPYSGNTNYAQKLQGRVTMTTDTSTSTAYMELRSLRSDDTAVYYCARDA
GTYSPFGYGMDVWGQGTTVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PG

and a light chain sequence that is

DIQMTQSPSTLSASVGDRVTITCRASESISNWLAWYQQKPGKAPKLLIYK
AYSLEYGVPSRFSGSGSGTEFTLTISSLQPDDFATYYCQQFQKLPPFTFG
GGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC,

a heavy chain sequence that is

QVQLVQSGAEVKKPGASVKVSCKASGYTFRSYGISWVRQAPGQGLEWMGW
VAPYSGNTNYAQKLQGRVTMTTDTSTSTAYMELRSLRSDDTAVYYCARDA
GTYSPYGYGMDVWGQGTTVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PG

and a light chain sequence that is

DIQMTQSPSTLSASVGDRVTITCRASHSIDSWLAWYQQKPGKAPKLLIYK
ASYLESGVPSRFSGSGSGTEFTLTISSLQPDDFATYYCQLFQSLPPFTFG
GGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC,

or
a full heavy chain sequence that is

QVQLVQSGAEVKKPGASVKVSCKASGYTFDVYGISWVRQAPGQGLEWMGW
IAPYSGNTNYAQKLQGRVTMTTDTSTSTAYMELRSLRSDDTAVYYCARDA
GTYSPFGYGMDVWGQGTTVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PG

and a light chain sequence that is

DIQMTQSPSTLSASVGDRVTITCRASESISNWLAWYQQKPGKAPKLLIYK
AYSLEYGVPSRFSGSGSGTEFTLTISSLQPDDFATYYCQQFQKLPPFTFG
GGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC.

In an embodiment, the ADCs described herein comprise an antibody that comprises:

a heavy chain sequence that is

QVQLVQSGAEVKKPGASVKVSCKASGYTFDVYGISWVRQAPGQGLEWMGW
IAPYSGNTNYAQKLQGRVTMTTDTSTSTAYMELRSLRSDDTAVYYCARDA
GTYSPFGYGMDVWGQGTTVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PG

and a light chain sequence that is

DIQMTQSPSTLSASVGDRVTITCQASQSINNWLAWYQQKPGKAPKLLIYK
AYNLESGVPSRFSGSGSGTEFTLTISSLQPDDFATYYCQLFQSLPPFTFG
GGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC.

In an embodiment, described herein is an antibody-drug conjugate comprising an antibody (Ab) and one or more linker-toxins of the following structure of Formula VIII:

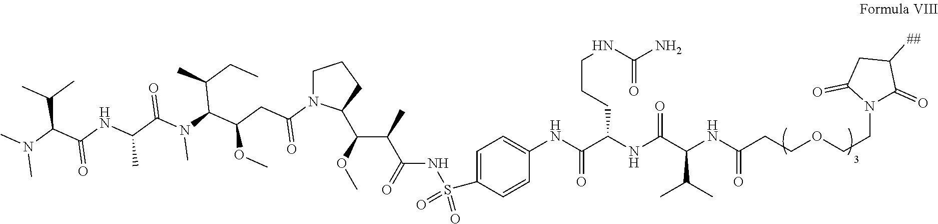

wherein: Ab is a tissue factor (TF) antibody, wherein the Ab comprises a VH-CDR1, a VH-CDR2, a VH-CDR3, a VL-CDR1, a VL-CDR2, and a VL-CDR3 from the antibody designated 25A3; the one or more linker-toxins are attached to the Ab through a covalent bond; and ## represents a point of attachment of the linker-toxin to the Ab.

In some embodiments, provided herein is a composition comprising an ADC comprising an antibody (Ab) and one or more linker-toxins of Formula VIII. In an embodiment, the composition comprises a multiplicity of drug-antibody ratio (DAR) species. In some embodiments, the average DAR of the composition is 2-4.

In an embodiments, provided herein is an antibody-drug conjugate comprising an antibody (Ab) and one or more linker-toxins of the following structure of Formula VIII:

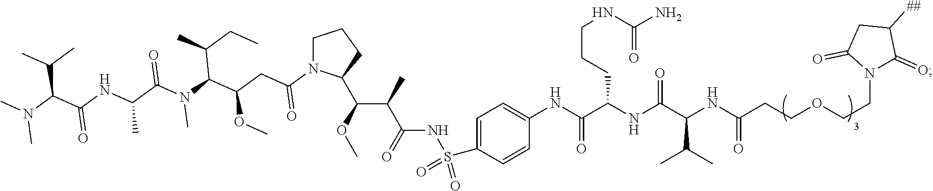

wherein:
Ab is a tissue factor (TF) antibody, wherein the Ab comprises a heavy chain sequence that is

QVQLVQSGAEVKKPGASVKVSCKASGYTFDVYGISWVRQAPGQGLEWMGW
IAPYSGNTNYAQKLQGRVTMTTDTSTSTAYMELRSLRSDDTAVYYCARDA
GTYSPFGYGMDVWGQGTTVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PG

and a light chain sequence that is

DIQMTQSPSTLSASVGDRVTITCQASQSINNWLAWYQQKPGKAPKLLIYK
AYNLESGVPSRFSGSGSGTEFTLTISSLQPDDFATYYCQLFQSLPPFTFG
GGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC,

the one or more linker-toxins are attached to the Ab through a covalent bond; and ## represents a point of attachment of the linker-toxin to the Ab.

In another embodiment, described herein is an antibody-drug conjugate composition comprising an ADC of the present disclosure, wherein the composition comprises a multiplicity of drug-antibody ratio (DAR) species, wherein the average DAR of the composition is 2-4.

### Linkers

In some embodiments, ADCs provided herein comprise a linker. In some embodiments, an unbound linker comprises two reactive termini: an antibody conjugation reactive terminus and an cytotoxic agent conjugation reactive terminus. For example, the linker can be conjugated to the antibody through a cysteine thiol or lysine amine group on the antibody, in which case, the antibody conjugation reactive terminus is typically a thiol-reactive group such as a double bond, a leaving group such as a chloro, bromo or iodo, an R-sulfanyl group or sulfonyl group, or an amine-reactive group such as a carboxyl group. The cytotoxic agent conjugation reactive terminus of the linker can be conjugated to the cytotoxic agent, for example, through formation of an amide bond with a basic amine or carboxyl group on the cytotoxin.

In some embodiments, the linker is a non-cleavable linker. In some embodiments, the linker is a cleavable linker. In some embodiments, the cytotoxic agent is released from the ADC in a cell.

Suitable linkers of ADCs include labile linkers, acid labile linkers (e.g., hydrazone linkers), photolabile linkers, charged linkers, disulfide-containing linkers, peptidase-sensitive linkers (e.g., peptide linkers comprising amino acids, for example, valine and/or citrulline such as citrulline-valine or phenylalanine-lysine), β-glucuronide-linkers (See e.g., Graaf et al., Curr Pharm Des, 2002, 8:1391-1403), dimethyl linkers (See e.g., Chari et al., Cancer Research, 1992, 52:127-131; U.S. Pat. No. 5,208,020), thio-ether linkers, or hydrophilic linkers (See e.g., Kovtun et al., Cancer Res., 2010, 70:2528-2537).

Other linkers include those having a functional group that allows for bridging of two interchain cysteines on the antibody, such as a ThioBridge™ linker (Badescu et al., Bioconjug. Chem., 25:1124-1136 (2014)), a dithiomaleimide (DTM) linker (Behrens et al., Mol. Pharm., 12:3986-3998 (2015)), a dithioaryl(TCEP)pyridazinedione based linker (Lee et al., Chem. Sci., 7:799-802 (2016)), a dibromopyridazinedione based linker (Maruani et al., Nat. Commun., 6:6645 (2015)) and others known in the art.

A linker may comprise one or more linker components. Typically, a linker will comprise two or more linker components. Exemplary linker components include functional groups for reaction with the antibody, functional groups for reaction with the toxin, stretchers, peptide components, self-immolative groups, self-elimination groups, hydrophilic moieties, and the like. Various linker components are known in the art, some of which are described below.

Certain useful linker components can be obtained from various commercial sources, such as Pierce Biotechnology, Inc. (now Thermo Fisher Scientific, Waltham, Mass.) and Molecular Biosciences Inc. (Boulder, Colo.), or may be synthesized in accordance with procedures described in the art (see, for example, Toki et al., J. Org. Chem., 67:1866-1872 (2002); Dubowchik, et al., Tetrahedron Letters, 38:5257-60 (1997); Walker, M. A., J. Org. Chem., 60:5352-5355 (1995); Frisch, et al., Bioconjugate Chem., 7:180-186 (1996); U.S. Pat. Nos. 6,214,345 and 7,553,816, and International Patent Application Publication No. WO 02/088172).

Examples of linker components include, but are not limited to, N-(β-maleimidopropyloxy)-N-hydroxysuccinimide ester (BMPS), N-(ε-maleimidocaproyloxy) succinimide ester (EMCS), N-[γ-maleimidobutyryloxy]succinimide ester (GMBS), 1,6-hexane-bis-vinylsulfone (HBVS), succinimidyl 4-(N-maleimidomethyl)cyclohexane-1-carboxy-(6-amidocaproate) (LC-SMCC), m-maleimidobenzoyl-N-hydroxysuccinimide ester (MBS), 4-(4-N-Maleimidophenyl)butyric acid hydrazide (MPBH), succinimidyl 3-(bromoacetamido)propionate (SBAP), succinimidyl iodoacetate (SIA), succinimidyl (4-iodoacetyl)aminobenzoate (SIAB), N-succinimidyl-3-(2-pyridyldithio) propionate (SPDP), N-succinimidyl-4-(2-pyridylthio)pentanoate (SPP), succinimidyl 4-(N-maleimidomethyl)cyclohexane-1-carboxylate (SMCC), succinimidyl 4-(p-maleimidophenyl)butyrate (SMPB), succinimidyl 6-[(β-maleimidopropionamido)hexanoate] (SMPH), iminothiolane (IT), sulfo-EMCS, sulfo-GMBS, sulfo-KMUS, sulfo-MBS, sulfo-SIAB, sulfo-SMCC, sulfo-SMPB and succinimidyl-(4-vinylsulfone)benzoate (SVSB).

Additional examples include bis-maleimide reagents such as dithiobismaleimidoethane (DTME), bis-maleimido-trioxyethylene glycol (BMPEO), 1,4-bismaleimidobutane (BMB), 1,4 bismaleimidyl-2,3-dihydroxybutane (BMDB), bismaleimidohexane (BMH), bismaleimidoethane (BMOE), BM(PEG)₂ and BM(PEG)₃; bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate) and bis-active fluorine compounds (such as 1,5-difluoro-2,4-dinitrobenzene).

In certain embodiments, the linker comprises a poly(ethylene)glycol chain of the formula:

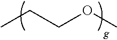

wherein g is an integer from 1-20. In some embodiments, g is 3.

In certain embodiments, the linker is a cleavable linker comprising a peptide component that includes two or more amino acids and is cleavable by an intracellular protease, such as lysosomal protease or an endosomal protease. A peptide component may comprise amino acid residues that occur naturally and/or minor amino acids and/or non-naturally occurring amino acid analogues, such as citrulline. Peptide components may be designed and optimized for enzymatic cleavage by a particular enzyme, for example, a tumor-associated protease, cathepsin B, C or D, or a plasmin protease.

In certain embodiments, the linker included in the ADCs may be a dipeptide-containing linker, such as a linker containing valine-citrulline (Val-Cit) or phenylalanine-lysine (Phe-Lys). Other examples of suitable dipeptides for inclusion in linkers include Val-Lys, Ala-Lys, Me-Val-Cit, Phe-homoLys, Phe-Cit, Leu-Cit, Ile-Cit, Trp-Cit, Phe-Arg, Ala-Phe, Val-Ala, Met-Lys, Asn-Lys, Ile-Pro, Ile-Val, Asp-Val, His-Val, Met-(D)Lys, Asn-(D)Lys, Val-(D)Asp, NorVal-(D)Asp, Ala-(D)Asp, Me₃Lys-Pro, PhenylGly-(D)Lys, Met-(D)Lys, Asn-(D)Lys, Pro-(D)Lys and Met-(D)Lys. Cleavable linkers may also include longer peptide components such as tripeptides, tetrapeptides or pentapeptides. Examples include, but are not limited to, the tripeptides Met-Cit-Val, Gly-Cit-Val, (D)Phe-Phe-Lys and (D)Ala-Phe-Lys, and the tetrapeptides Gly-Phe-Leu-Gly and Ala-Leu-Ala-Leu.

In certain embodiments, the cytotoxic agent is conjugated to the antibody using a linker comprising valine-citrulline (vc).

Cleavable linkers may optionally further comprise one or more additional components such as self-immolative and self-elimination groups, stretchers or hydrophilic moieties.

Self-immolative and self-elimination groups that find use in linkers include, for example, p-aminobenzyloxycarbonyl (PABC) and p-aminobenzyl ether (PABE) groups, and methylated ethylene diamine (MED). Other examples of self-immolative groups include, but are not limited to, aromatic compounds that are electronically similar to the PABC or PABE group such as heterocyclic derivatives, for example 2-aminoimidazol-5-methanol derivatives as described in U.S. Pat. No. 7,375,078. Other examples include groups that undergo cyclization upon amide bond hydrolysis, such as substituted and unsubstituted 4-aminobutyric acid amides (Rodrigues et al., Chemistry Biology, 2:223-227 (1995)) and 2-aminophenylpropionic acid amides (Amsberry, et al., J. Org. Chem., 55:5867-5877 (1990)).

Stretchers that find use in linkers for ADCs include, for example, alkylene groups and stretchers based on aliphatic acids, diacids, amines or diamines, such as diglycolate, malonate, caproate and caproamide. Other stretchers include, for example, glycine-based stretchers, polyethylene glycol (PEG) stretchers and monomethoxy polyethylene glycol (mPEG) stretchers. PEG and mPEG stretchers also function as hydrophilic moieties.

Examples of components commonly found in cleavable linkers that may find use in the ADCs of the present disclosure in some embodiments include, but are not limited to, SPBD, sulfo-SPBD, hydrazone, Val-Cit, maleidocaproyl (MC or mc), mc-Val-Cit, mc-Val-Cit-PABC, Phe-Lys, mc-Phe-Lys, mc-Phe-Lys-PABC, maleimido triethylene glycolate (MT), MT-Val-Cit, MT-Phe-Lys and adipate (AD).

In certain embodiments, the linker included in the ADCs of the present disclosure are peptide-based linkers having general Formula VII:

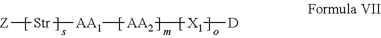

wherein: Str is a stretcher; AA₁ and AA₂ are each independently an amino acid, wherein AA₁-[AA₂]ₘ forms a protease cleavage site; X₁ is a self-immolative group; Z is the point of attachment to a functional group that binds with a target group (e.g., the thiol of a cysteine or primary amine of a lysine group) on the antibody; D is the point of attachment to the cytotoxic agent; s is 0 or 1; m is an integer between 1 and 4, and o is 0, 1 or 2.

In some embodiments, in the linker of general Formula VII, Z is:

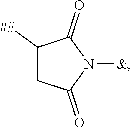

wherein ## represents the point of attachment of the succinimidyl group to the TF antibody and the succinimidyl group is attached to the TF antibody through a covalent bond, and & represents the point of attachment to [Str]ₛ.

In some embodiments, in the linker of general Formula VII, [Str]ₛ is selected from the group consisting of alkylene, stretchers based on aliphatic acids, stretchers based on aliphatic diacids, stretchers based on aliphatic amines and stretchers based on aliphatic diamines.

In some embodiments, in the linker of general Formula VII, [Str]ₛ is selected from the group consisting of diglycolate-based stretchers, malonate-based stretchers, caproate-based stretchers and caproamide-based stretchers.

In some embodiments, in the linker of general Formula VII, [Str]ₛ is selected from the group consisting of glycine-based stretchers, polyethylene glycol-based stretchers, and monomethoxy polyethylene glycol-based stretchers.

In some embodiments, in the linker of general Formula VII, [Str]ₛ is:

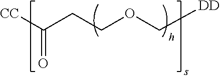

wherein h is an integer from 1-20, CC refers to the point of attachment to AA₁; and DD refers to the point of attachment to Z.

In some embodiments, in the linker of general Formula VII, [Str]ₛ is selected from:

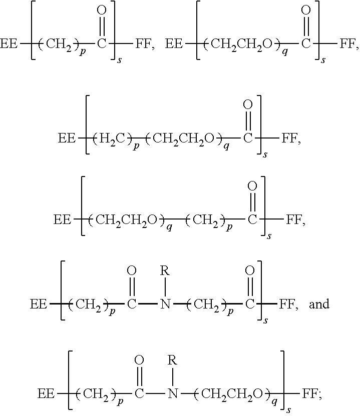

wherein: EE and FF represent the points of attachment to Z and AA₁, respectively; R is selected from hydrogen and C₁-C₆ alkyl; each occurrence of p is independently an integer from 2 to 10; and each occurrence of q is independently an integer from 1 to 10.

In some embodiments, in the linker of general Formula VII, [Str]ₛ is selected from the group consisting of:

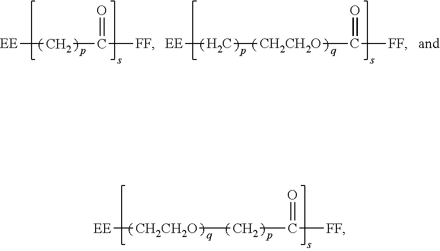

wherein: EE and FF represent the points of attachment to Z and AA₁, respectively; each occurrence of p is independently an integer from 2 to 10; and each occurrence of q is independently an integer from 1 to 10.

In some embodiments, in the linker of general Formula VII, [Str]ₛ is selected from:

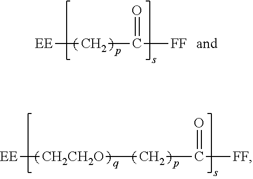

wherein: EE and FF represent the points of attachment to Z and AA₁, respectively; each occurrence of p is independently an integer from 2 to 6, and q is an integer from 2 to 8.

In some embodiments, in the linker of general Formula VII, AA₁-[AA₂]ₘ is selected from Val-Lys, Ala-Lys, Phe-Lys, Val-Cit, Phe-Cit, Leu-Cit, Ile-Cit, Trp-Cit, Phe-Arg, Ala-Phe, Val-Ala, Met-Lys, Asn-Lys, Ile-Pro, Ile-Val, Asp-Val, His-Val, Met-(D)Lys, Asn-(D)Lys, Val-(D)Asp, NorVal-(D)Asp, Ala-(D)Asp, Me₃Lys-Pro, PhenylGly-(D)Lys, Met-(D)Lys, Asn-(D)Lys, Pro-(D)Lys, Met-(D)Lys, Met-Cit-Val, Gly-Cit-Val, (D)Phe-Phe-Lys, (D)Ala-Phe-Lys, Gly-Phe-Leu-Gly and Ala-Leu-Ala-Leu.

In some embodiments, in the linker of general Formula VII, m is 1 (i.e. AA₁-[AA₂]ₘ is a dipeptide).

In some embodiments, in the linker of general Formula VII, AA₁-[AA₂]ₘ is a dipeptide selected from Val-Lys, Ala-Lys, Phe-Lys, Val-Cit, Phe-Cit, Leu-Cit, Ile-Cit and Trp-Cit.

In some embodiments, in the linker of general Formula VII, each X₁ is independently selected from p-aminobenzyloxycarbonyl (PABC), p-aminobenzyl ether (PABE) and methylated ethylene diamine (MED).

In some embodiments, in the linker of general Formula VII, m is 1, 2 or 3.

In some embodiments, in the linker of general Formula VII, s is 1.

In some embodiments, in the linker of general Formula VII, o is 0.

In some embodiments, in the linker of general Formula VII:

### Z is

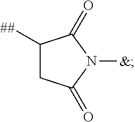

wherein ## represents the point of attachment of the succinimidyl group the TF antibody and the succinimidyl group is attached the TF antibody through a covalent bond, & represents the point of attachment to [Str]ₛ;
[Str]ₛ is selected from

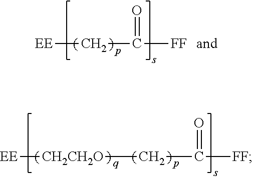

EE and FF represent the points of attachment to Z and AA₁, respectively; p is an integer between 2 and 6; q is an integer between 2 and 8; m is 1; AA₁-AA₂ is a dipeptide selected from the group consisting of Val-Lys, Ala-Lys, Phe-Lys, Val-Cit, Phe-Cit, Leu-Cit, Ile-Cit and Trp-Cit; s is 1; and o is 0.

In certain embodiments, the linker included in the ADCs of the present disclosure has general Formula X:

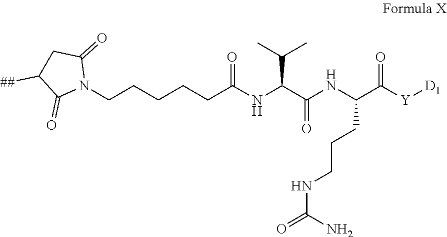

wherein: ## is the point of attachment to the antibody and the succinimidyl group is attached to the antibody through a covalent bond; Y is one or more additional linker components, or is absent, and D₁ is the point of attachment to a cytotoxic agent. In some embodiments of general Formula X, Ab comprises a VH-CDR1, a VH-CDR2, a VH-CDR3, a VL-CDR1, a VL-CDR2, and a VL-CDR3, wherein

i. the VH-CDR1 comprises SEQ ID NO: 872, the VH-CDR2 comprises SEQ ID NO: 873, the VH-CDR3 comprises SEQ ID NO: 874, the VL-CDR1 comprises SEQ ID NO: 875, the VL-CDR2 comprises SEQ ID NO: 876, and the VL-CDR3 comprises SEQ ID NO: 877,

ii. the VH-CDR1, VH-CDR2, VH-CDR3, VL-CDR1, VL-CDR2, and VL-CDR3 are from the antibody designated 25A3,

iii. the VH-CDR1, VH-CDR2, VH-CDR3, VL-CDR1, VL-CDR2, and VL-CDR3 are from the antibody designated 25A,

iv. the VH-CDR1, VH-CDR2, VH-CDR3, VL-CDR1, VL-CDR2, and VL-CDR3 are from the antibody designated 25A5,

v. the VH-CDR1, VH-CDR2, VH-CDR3, VL-CDR1, VL-CDR2, and VL-CDR3 are from the antibody designated 25A5-T, or

vi. the VH-CDR1, VH-CDR2, VH-CDR3, VL-CDR1, VL-CDR2, and VL-CDR3 are from the antibody designated 25G1.

In some embodiments, in general Formula X, Y is [X₁]ₒ, wherein X₁ is a self-immolative group and o is an integer selected from 1 and 2. In some embodiments, in general Formula X, each X₁ is selected from the group consisting of p-aminobenzyloxycarbonyl (PABC), p-aminobenzyl ether (PABE) and methylated ethylene diamine (MED). In some embodiments, in general Formula X, Y is absent.

In certain embodiments, the linker included in the ADCs of the present disclosure has general Formula XI:

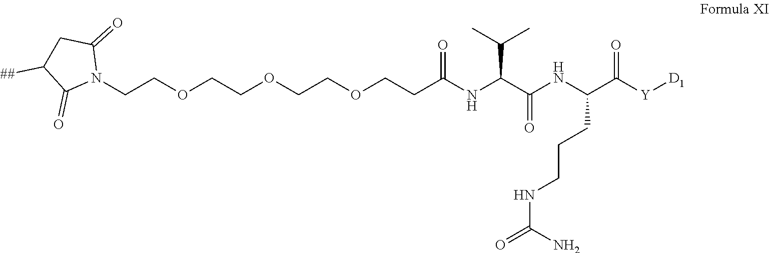

wherein: ## is the point of attachment to the antibody and the succinimidyl group is attached to the antibody through a covalent bond; Y is one or more additional linker components, or is absent, and D₁ is the point of attachment to a cytotoxic agent. In some embodiments, the ADC comprises the linker of general Formula XI, and Ab comprises a VH-CDR1, a VH-CDR2, a VH-CDR3, a VL-CDR1, a VL-CDR2, and a VL-CDR3, wherein
i. the VH-CDR1 comprises SEQ ID NO: 872, the VH-CDR2 comprises SEQ ID NO: 873, the VH-CDR3 comprises SEQ ID NO: 874, the VL-CDR1 comprises SEQ ID NO: 875, the VL-CDR2 comprises SEQ ID NO: 876, and the VL-CDR3 comprises SEQ ID NO: 877,
ii. the VH-CDR1, VH-CDR2, VH-CDR3, VL-CDR1, VL-CDR2, and VL-CDR3 are from the antibody designated 25A3,
iii. the VH-CDR1, VH-CDR2, VH-CDR3, VL-CDR1, VL-CDR2, and VL-CDR3 are from the antibody designated 25A,
iv. the VH-CDR1, VH-CDR2, VH-CDR3, VL-CDR1, VL-CDR2, and VL-CDR3 are from the antibody designated 25A5,
v. the VH-CDR1, VH-CDR2, VH-CDR3, VL-CDR1, VL-CDR2, and VL-CDR3 are from the antibody designated 25A5-T, or
vi. the VH-CDR1, VH-CDR2, VH-CDR3, VL-CDR1, VL-CDR2, and VL-CDR3 are from the antibody designated 25G1.

In some embodiments, Y is [X₁]ₒ, wherein X₁ is a self-immolative group and o is an integer selected from 1 and 2. In some embodiments, each X₁ is selected from the group consisting of p-aminobenzyloxycarbonyl (PABC), p-aminobenzyl ether (PABE) and methylated ethylene diamine (MED). In some embodiments, Y is absent. In some embodiments of the linker of Formula X or Formula XI, the cytotoxic agent is selected from the group consisting of a diagnostic agent, a metal chelator, an enzyme, a fluorescent compound, a bioluminescent compound, or a chemiluminescent compound.

In some embodiments of the linker of Formula X or Formula XI, the cytotoxic agent is a cytotoxic payload having an improved safety profile.

In another embodiment, a compound comprising a linker of general Formula XII is capable of chemically binding with a target group (e.g., the thiol of a cysteine or primary amine of a lysine group) on a tissue factor (TF) antibody to form an ADC of the present disclosure:

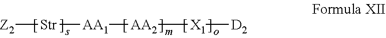

wherein: Str is a stretcher; AA₁ and AA₂ are each independently an amino acid, wherein AA₁-[AA₂]ₘ forms a protease cleavage site; X₁ is a self-immolative group; D₂ is the point of attachment to a cytotoxic agent; Z₂ is a functional group capable of reacting with a target group on a TF antibody to form a bond with the TF antibody; s is 0 or 1; m is an integer between 1 and 4, and o is 0, 1 or 2.

In some embodiments, in the linker of general Formula XII, Z₂ is:

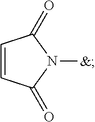

and & represents the point of attachment to [Str]ₛ.

In some embodiments, in the linker of general Formula XII, [Str]ₛ is selected from the group consisting of alkylene, stretchers based on aliphatic acids, stretchers based on aliphatic diacids, stretchers based on aliphatic amines and stretchers based on aliphatic diamines.

In some embodiments, in the linker of general Formula XII, [Str]ₛ is selected from the group consisting of diglycolate-based stretchers, malonate-based stretchers, caproate-based stretchers and caproamide-based stretchers.

In some embodiments, in the linker of general Formula XII, [Str]ₛ is selected from the group consisting of glycine-based stretchers, polyethylene glycol-based stretchers, and monomethoxy polyethylene glycol-based stretchers.

In some embodiments, in the linker of general Formula XII, [Str] is:

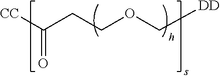

wherein h is an integer from 1-20, CC refers to the point of attachment to AA₁; and DD refers to the point of attachment to Z.

In some embodiments, in the linker of general Formula XII, [Str]ₛ is selected

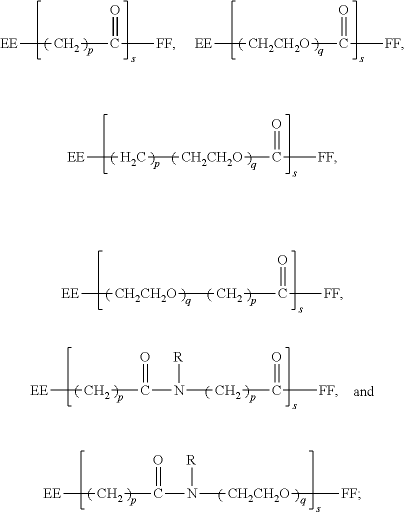

wherein: EE and FF represent the points of attachment to Z and AA₁, respectively; R is selected from hydrogen and C₁-C₆ alkyl; each occurrence of p is independently an integer from 2 to 10; and each occurrence of q is independently an integer from 1 to 10.

In some embodiments, in the linker of general Formula XII, [Str]ₛ is selected from the group consisting of:

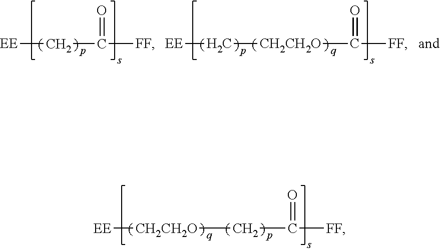

wherein: EE and FF represent the points of attachment to Z and AA₁, respectively; each occurrence of p is independently an integer from 2 to 10; and each occurrence of q is independently an integer from 1 to 10.

In some embodiments, in the linker of general Formula XII, [Str]ₛ is selected from:

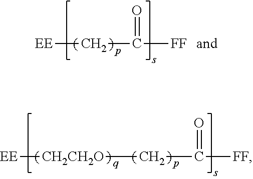

wherein: EE and FF represent the points of attachment to Z and AA₁, respectively; each occurrence of p is independently an integer from 2 to 6, and q is an integer from 2 to 8.

In some embodiments, in the linker of general Formula XII, AA₁-[AA₂]ₘ is selected from Val-Lys, Ala-Lys, Phe-Lys, Val-Cit, Phe-Cit, Leu-Cit, Ile-Cit, Trp-Cit, Phe-Arg, Ala-Phe, Val-Ala, Met-Lys, Asn-Lys, Ile-Pro, Ile-Val, Asp-Val, His-Val, Met-(D)Lys, Asn-(D)Lys, Val-(D)Asp, NorVal-(D)Asp, Ala-(D)Asp, Me₃Lys-Pro, PhenylGly-(D)Lys, Met-(D)Lys, Asn-(D)Lys, Pro-(D)Lys, Met-(D)Lys, Met-Cit-Val, Gly-Cit-Val, (D)Phe-Phe-Lys, (D)Ala-Phe-Lys, Gly-Phe-Leu-Gly and Ala-Leu-Ala-Leu.

In some embodiments, in the linker of general Formula XII, m is 1 (i.e. AA₁-[AA₂]ₘ is a dipeptide).

In some embodiments, in the linker of general Formula XII, AA₁-[AA₂]ₘ is a dipeptide selected from Val-Lys, Ala-Lys, Phe-Lys, Val-Cit, Phe-Cit, Leu-Cit, Ile-Cit and Trp-Cit.

In some embodiments, in the linker of general Formula XII, each X₁ is independently selected from p-aminobenzyloxycarbonyl (PABC), p-aminobenzyl ether (PABE) and methylated ethylene diamine (MED).

In some embodiments, in the linker-toxin compound of general Formula XII, m is 1, 2 or 3.

In some embodiments, in the linker of general Formula XII, s is 1.

In some embodiments, in the linker of general Formula XII, o is 0.

In some embodiments, in the linker of general Formula XII:

### Z is

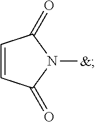

& represents the point of attachment to [Str]ₛ;
[Str]ₛ is selected from

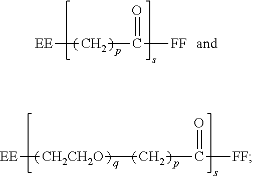

EE and FF represent the points of attachment to Z and AA₁, respectively; p is an integer between 2 and 6; q is an integer between 2 and 8; m is 1; AA₁-AA₂ is a dipeptide selected from the group consisting of Val-Lys, Ala-Lys, Phe-Lys, Val-Cit, Phe-Cit, Leu-Cit, Ile-Cit and Trp-Cit; s is 1; and o is 0.

In certain embodiments, the compound comprising the linker of general Formula XII has the following structure:

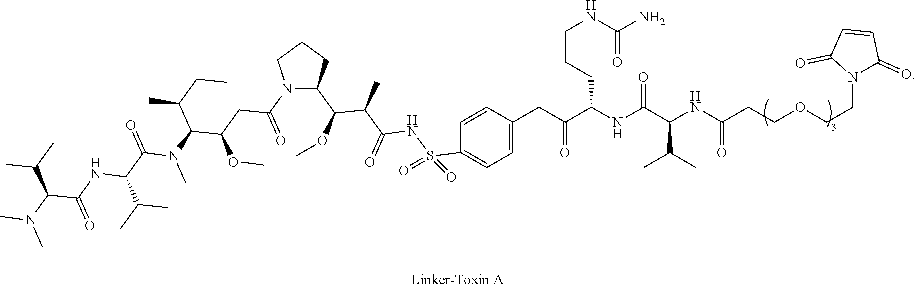

### Methods for Making Antibody-Drug Conjugates

The ADCs can be prepared using any suitable methods as disclosed in the art employing organic chemistry reactions, conditions, and reagents known to those skilled in the art, see e.g., Bioconjugate Techniques, 2nd Ed., G. T. Hermanson, ed., Elsevier, San Francisco, 2008.

For example, conjugation may be achieved by (1) reaction of a nucleophilic group or an electrophilic group of the antibody with a bifunctional linker to form an antibody-linker intermediate Ab-L, via a covalent bond, followed by reaction with the activated cytotoxic agent (D), or (2) reaction of a nucleophilic group or an electrophilic group of the cytotoxic agent with a bifunctional linker to form linker-toxin D-L, via a covalent bond, followed by reaction with the nucleophilic group or an electrophilic group of the antibody.

In certain embodiments, described herein is a process for preparing an antibody-drug conjugate, the process comprising: (A) reacting a nucleophilic or an electrophilic group on an antigen binding protein (Ab) which binds to the extracellular domain of human Tissue Factor (TF) (SEQ ID NO:810) with a bifunctional linker to form an Ab-linker intermediate, and reacting the Ab-linker intermediate with the —NH₂ group on the auristatin derivative of general Formula I

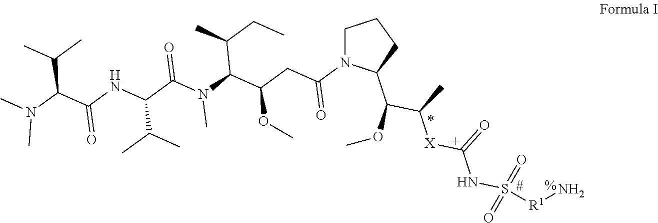

wherein: X is *—C(O)NHCH(CH₂(R²))—⁺, wherein * and + represent the respective points of attachment as indicated in Formula I, or X is absent; R¹ is selected from the group consisting of:

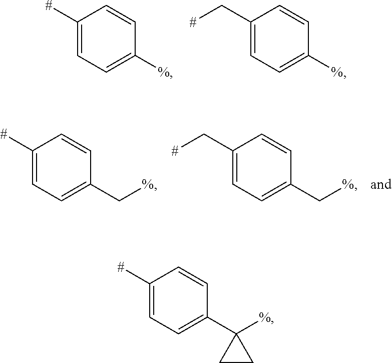

wherein # and % represent the respective points of attachment as indicated in Formula I; and R² is phenyl, to provide the antibody drug conjugate; or (B) reacting the —NH₂ group on the auristatin derivative of general Formula I with a bifunctional linker to form a linker-toxin intermediate, and reacting the linker-toxin intermediate with a nucleophilic or an electrophilic group on an antigen binding protein (Ab) which binds to the extracellular domain of human Tissue Factor (TF) (SEQ ID NO: 810) to provide the antibody-drug conjugate, wherein, in (A) or (B), (a) the Ab comprises a VH-CDR1, a VH-CDR2, a VH-CDR3, a VL-CDR1, a VL-CDR2, and a VL-CDR3, wherein

i. the VH-CDR1 comprises SEQ ID NO: 872, the VH-CDR2 comprises SEQ ID NO: 873, the VH-CDR3 comprises SEQ ID NO: 874, the VL-CDR1 comprises SEQ ID NO: 875, the VL-CDR2 comprises SEQ ID NO: 876, and the VL-CDR3 comprises SEQ ID NO: 877,

ii. the VH-CDR1, VH-CDR2, VH-CDR3, VL-CDR1, VL-CDR2, and VL-CDR3 are from the antibody designated 25A3,

iii. the VH-CDR1, VH-CDR2, VH-CDR3, VL-CDR1, VL-CDR2, and VL-CDR3 are from the antibody designated 25A,

iv. the VH-CDR1, VH-CDR2, VH-CDR3, VL-CDR1, VL-CDR2, and VL-CDR3 are from the antibody designated 25A5,

v. the VH-CDR1, VH-CDR2, VH-CDR3, VL-CDR1, VL-CDR2, and VL-CDR3 are from the antibody designated 25A5-T, or

vi. the VH-CDR1, VH-CDR2, VH-CDR3, VL-CDR1, VL-CDR2, and VL-CDR3 are from the antibody designated 25G1; and
(b) the antibody-drug conjugate comprises one or more moieties represented by Formula IV:

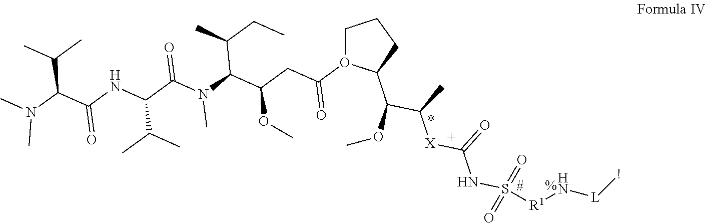

wherein: X is *—C(O)NHCH(CH₂(R²))—⁺, wherein * and + represent the respective points of attachment as indicated in Formula IV, or X is absent; L is a linker; ! represents the point of attachment of L to the Ab, where L is attached to the Ab through a covalent bond; R¹ is selected from the group consisting of:

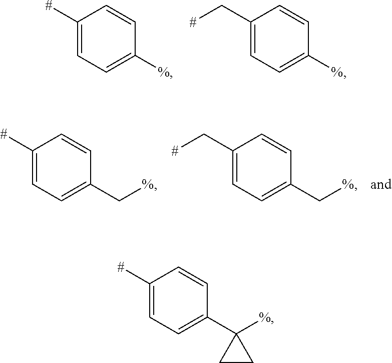

wherein # and % represent the respective points of attachment as indicated in Formula IV; and R² is phenyl.

In certain embodiments, described herein is a process for preparing an antibody-drug conjugate, the process comprising: (A) reacting a nucleophilic or an electrophilic group on an antigen binding protein (Ab) which binds to the extracellular domain of human Tissue Factor (TF) (SEQ ID NO:810) with a first linker component of a bifunctional linker that comprises two or more linker components followed by sequential addition of the remaining linker component(s) to form an Ab-linker intermediate, and reacting the Ab-linker intermediate with the —NH₂ group on the auristatin derivative of general Formula I:

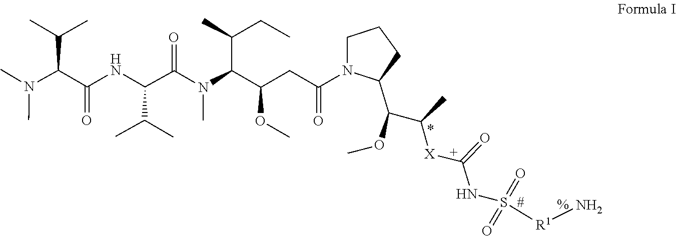

wherein: X is *—C(O)NHCH(CH₂(R²))—⁺, wherein * and + represent the respective points of attachment as indicated in Formula I, or X is absent; R¹ is selected from the group consisting of:

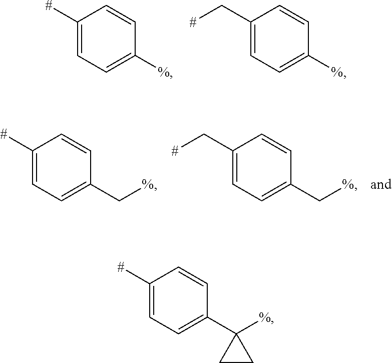

wherein # and % represent the respective points of attachment as indicated in Formula I; and R² is phenyl, to provide the antibody drug conjugate; or (B) reacting the —NH₂ group on the auristatin derivative of general Formula I with a first linker component of a bifunctional linker that comprises two or more linker components followed by sequential addition of the remaining linker component(s) to form a linker-toxin intermediate, and reacting the linker-toxin intermediate with a nucleophilic or an electrophilic group on an antigen binding protein (Ab) which binds to the extracellular domain of human Tissue Factor (TF) (SEQ ID NO: 810) to provide the antibody-drug conjugate, wherein, in (A) or (B), (a) the Ab comprises a VH-CDR1, a VH-CDR2, a VH-CDR3, a VL-CDR1, a VL-CDR2, and a VL-CDR3, wherein

vii. the VH-CDR1 comprises SEQ ID NO: 872, the VH-CDR2 comprises SEQ ID NO: 873, the VH-CDR3 comprises SEQ ID NO: 874, the VL-CDR1 comprises SEQ ID NO: 875, the VL-CDR2 comprises SEQ ID NO: 876, and the VL-CDR3 comprises SEQ ID NO: 877,

viii. the VH-CDR1, VH-CDR2, VH-CDR3, VL-CDR1, VL-CDR2, and VL-CDR3 are from the antibody designated 25A3,

ix. the VH-CDR1, VH-CDR2, VH-CDR3, VL-CDR1, VL-CDR2, and VL-CDR3 are from the antibody designated 25A,

x. the VH-CDR1, VH-CDR2, VH-CDR3, VL-CDR1, VL-CDR2, and VL-CDR3 are from the antibody designated 25A5,

xi. the VH-CDR1, VH-CDR2, VH-CDR3, VL-CDR1, VL-CDR2, and VL-CDR3 are from the antibody designated 25A5-T, or

xii. the VH-CDR1, VH-CDR2, VH-CDR3, VL-CDR1, VL-CDR2, and VL-CDR3 are from the antibody designated 25G1; and
(b) the antibody-drug conjugate comprises one or more moieties represented by Formula IV:

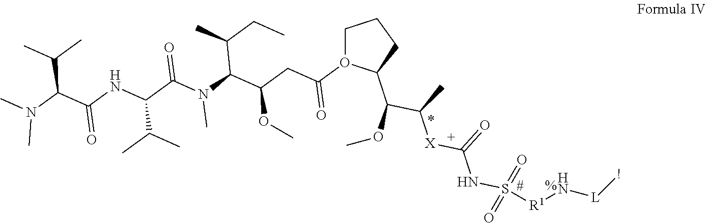

wherein: X is *—C(O)NHCH(CH₂(R²))—⁺, wherein * and + represent the respective points of attachment as indicated in Formula IV, or X is absent; L is a linker; ! represents the point of attachment of L to the Ab, where L is attached to the Ab through a covalent bond; R¹ is selected from the group consisting of:

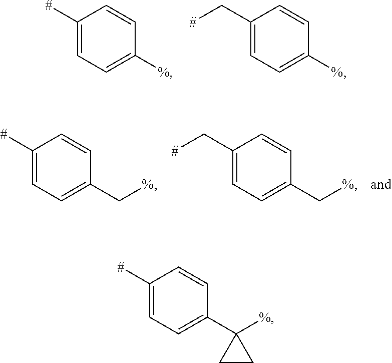

wherein # and % represent the respective points of attachment as indicated in Formula IV; and R² is phenyl.

In certain embodiments, the nucleophilic or electrophilic group on the Ab is a thiol or an amine. In certain embodiments of the process for preparing the ADC, the process further comprises treating the Ab with a reducing agent to reduce one or more disulfide linkages in the Ab to provide the nucleophilic thiol group. In certain embodiments of the process for preparing the ADC, L is represented by Formula VII:

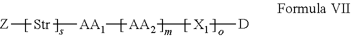

wherein: Z represents a functional group that binds to a target group of the Ab; D represents the point of attachment to the amino group as indicated in Formula I; Str is a stretcher; AA₁ and AA₂ are each independently an amino acid, wherein AA₁-[AA₂]ₘ forms a protease cleavage site; X₁ is a self-immolative group; s is an integer selected from 0 and 1; m is an integer selected from the group consisting of 1, 2, 3, and 4; and o is an integer selected from 0, 1, and 2.

In certain embodiments in which the cytotoxic agent is a compound of general Formula I, the ADCs may be prepared by a method comprising (A) (i) reacting a nucleophilic or electrophilic group on the antibody with a bifunctional linker to form an antibody-linker intermediate, or (ii) reacting a nucleophilic or electrophilic group on the antibody with a first linker component of a bifunctional linker that comprises two or more linker components followed by sequential addition of the remaining linker component(s) to form an antibody-linker intermediate, and (B) reacting the antibody-linker intermediate with the —NH₂ group on the compound of general Formula I to provide the ADC.

In certain embodiments in which the cytotoxic agent is a compound of general Formula I, the ADCs may be prepared by a method comprising (A) (i) reacting the NH₂ group on the compound of general Formula I with a bifunctional linker to form a linker-toxin intermediate, or (ii) reacting the NH₂ group on the compound of general Formula I with a first linker component of a bifunctional linker that comprises two or more linker components followed by sequential addition of the remaining linker component(s) to form a linker-toxin intermediate, and (B) reacting the linker-toxin intermediate with a nucleophilic or electrophilic group on the antibody to provide the antibody-drug conjugate.

In some embodiments, the electrophilic or nucleophilic group on the antibody is a thiol (for example from a cysteine residue on the antibody), or an amine (for example from a lysine residue on the antibody). In some embodiments, the bifunctional linker has general Formula VII, general Formula X or general Formula XI. Compounds of general Formula I and linker-toxins comprising compounds of general Formula I may be prepared by standard synthetic organic chemistry protocols from commercially available starting materials. Exemplary methods are provided in International Patent Application Publication No. WO 2016/041082 and in the Examples section below.

In certain embodiments, the ADCs of the present disclosure are prepared by conjugating the linker-cytotoxic agent to cysteine residues that have been liberated by reducing one or more interchain disulfide linkages on the antibody. Suitable reducing agents are known in the art and include, for example, dithiothreitol (DTT), tris(2-carboxyethyl)phosphine (TCEP), 2-mercaptoethanol, cysteamine and a number of water soluble phosphines.

In some embodiments, the ADCs are made with site-specific conjugation techniques, resulting in homogeneous drug loading and avoiding ADC subpopulations with altered antigen-binding or pharmacokinetics. In some embodiments, “thiomabs” comprising cysteine substitutions at positions on the heavy and light chains are engineered to provide reactive thiol groups that do not disrupt immunoglobulin folding and assembly or alter antigen binding (Junutula et al., J. Immunol. Meth., 2008, 332: 41-52; Junutula et al., Nat. Biotechnol., 2008, 26: 925-932). In some embodiments, selenocysteine is co-translationally inserted into an antibody sequence by recoding the stop codon UGA from termination to selenocysteine insertion, allowing site specific covalent conjugation at the nucleophilic selenol group of selenocysteine in the presence of the other natural amino acids (See e.g., Hofer et al., Proc. Natl. Acad. Sci. USA, 2008, 105:12451-12456; Hofer et al., Biochemistry, 2009, 48(50):12047-12057). Alternatively, the antibody may be modified to include other non-natural amino acids that provide reactive handles, such as p-acetylphenylalanine, formylglycine or p-azidomethyl-L-phenylalanine (see, for example, Axup et al., PNAS, 109:16101-16106 (2012); Wu et al., PNAS, 106:3000-3005 (2009); Zimmerman et al., Bioconj. Chem., 25:351-361 (2014)). In certain embodiments, ADCs were synthesized as described in Behrens et al., Mol Pharm, 2015, 12:3986-98.

### 6. Assays

A variety of assays known in the art may be used to identify and characterize anti-TF antibodies and anti-TF ADCs provided herein.

6.1. Binding, Competition, and Epitope Mapping Assays

Specific antigen-binding activity of the antibodies provided herein may be evaluated by any suitable method, including using SPR, BLI, RIA and MSD-SET, as described elsewhere in this disclosure. Additionally, antigen-binding activity may be evaluated by ELISA assays and Western blot assays.

Assays for measuring competition between two antibodies, or an antibody and another molecule (e.g., one or more ligands of TF) are described elsewhere in this disclosure and, for example, in Harlow and Lane, Antibodies: A Laboratory Manual ch. 14, 1988, Cold Spring Harbor Laboratory, Cold Spring Harbor, N.Y, incorporated by reference in its entirety.

Assays for mapping the epitopes to which the antibodies provided herein bind are described, for example, in Morris “Epitope Mapping Protocols,” in Methods in Molecular Biology vol. 66, 1996, Humana Press, Totowa, N.J., incorporated by reference in its entirety. In some embodiments, the epitope is determined by peptide competition. In some embodiments, the epitope is determined by mass spectrometry. In some embodiments, the epitope is determined by crystallography.

6.2. Thrombin Generation, FXa Conversion, and TF Signaling Assays

Thrombin generation in the presence of the antibodies provided herein can be determined by the Thrombin Generation Assay (TGA), as described elsewhere in this disclosure.

Assays for measuring FXa conversion in the presence of the antibodies provided herein are described elsewhere in this disclosure.

Inhibition of TF signaling can be determined by measuring the production of a cytokine regulated by the TF signaling, such as IL8 and GM-CSF. Assays for determining the IL8 and/or GM-CSF level are provided elsewhere in this disclosure and, for example, in Hjortoe et al., Blood, 2004, 103:3029-3037.

6.3. Assays for Effector Functions

Effector function following treatment with the antibodies provided herein may be evaluated using a variety of in vitro and in vivo assays known in the art, including those described in Ravetch and Kinet, Annu. Rev. Immunol., 1991, 9:457-492; U.S. Pat. Nos. 5,500,362, 5,821,337; Hellstrom et al., Proc. Nat'l Acad. Sci. USA, 1986, 83:7059-7063; Hellstrom et al., Proc. Nat'l Acad. Sci. USA, 1985, 82:1499-1502; Bruggemann et al., J. Exp. Med., 1987, 166:1351-1361; Clynes et al., Proc. Nat'l Acad. Sci. USA, 1998, 95:652-656; WO 2006/029879; WO 2005/100402; Gazzano-Santoro et al., J. Immunol. Methods, 1996, 202:163-171; Cragg et al., Blood, 2003, 101:1045-1052; Cragg et al. Blood, 2004, 103:2738-2743; and Petkova et al., Int'l. Immunol., 2006, 18:1759-1769; each of which is incorporated by reference in its entirety.

6.4. Cytotoxicity Assays and In Vivo Studies

Assays for evaluating cytotoxicity of the antibody-drug conjugates (ADCs) provided herein are described elsewhere in this disclosure.

Xenograft studies in immune compromised mice for evaluating the in vivo efficacy of the ADCs provided herein are described elsewhere in this disclosure.

Syngeneic studies in immune competent mice for evaluating the in vivo efficacy of the ADCs are included in this disclosure.

6.5. Immunohistochemistry (IHC) Assays

Immunohistochemistry (IHC) assays for evaluating the TF expression in patient samples are described elsewhere in this disclosure.

6.6. Chimeric Construct Mapping and Epitope Binning Assays

Epitope binding differences between the anti-human TF antibodies provided herein can be determined by the chimeric TF construct mapping experiments and the epitope binning assays, as described elsewhere in this disclosure.

### 7. Pharmaceutical Compositions

The antibodies or ADCs provided herein can be formulated in any appropriate pharmaceutical composition and administered by any suitable route of administration. Suitable routes of administration include, but are not limited to, the intravitreal, intraarterial, intradermal, intramuscular, intraperitoneal, intravenous, nasal, parenteral, pulmonary, and subcutaneous routes.

The pharmaceutical composition may comprise one or more pharmaceutical excipients. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

7.1. Parenteral Dosage Forms

In certain embodiments, the antibodies or ADCs provided herein are formulated as parenteral dosage forms. Parenteral dosage forms can be administered to subjects by various routes including, but not limited to, subcutaneous, intravenous (including infusions and bolus injections), intramuscular, and intraarterial. Because their administration typically bypasses subjects' natural defenses against contaminants, parenteral dosage forms are typically, sterile or capable of being sterilized prior to administration to a subject. Examples of parenteral dosage forms include, but are not limited to, solutions ready for injection, dry (e.g., lyophilized) products ready to be dissolved or suspended in a pharmaceutically acceptable vehicle for injection, suspensions ready for injection, and emulsions.

### 8. Dosage and Unit Dosage Forms

In human therapeutics, the doctor will determine the posology which he considers most appropriate according to a preventive or curative treatment and according to the age, weight, condition and other factors specific to the subject to be treated.

In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic antibodies or ADCs.

The amount of the antibody/ADC or composition which will be effective in the prevention or treatment of a disorder or one or more symptoms thereof can vary with the nature and severity of the disease or condition, and the route by which the antibody/ADC is administered. The frequency and dosage can also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the antibodies or ADCs provided herein are also encompassed by the dosage amounts and dose frequency schedules provided herein. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

As discussed in more detail elsewhere in this disclosure, an antibody or ADC provided herein may optionally be administered with one or more additional agents useful to prevent or treat a disease or disorder. The effective amount of such additional agents may depend on the amount of ADC present in the formulation, the type of disorder or treatment, and the other factors known in the art or described herein.

### 9. Therapeutic Applications

For therapeutic applications, the antibodies or ADCs of the invention are administered to a mammal, generally a human, in a pharmaceutically acceptable dosage form such as those known in the art and those discussed above. For example, the antibodies or ADCs of the invention may be administered to a human intravenously as a bolus or by continuous infusion over a period of time, by intravitreal, intramuscular, intraperitoneal, intra-cerebrospinal, subcutaneous, intra-articular, intrasynovial, intrathecal, or intratumoral routes. The antibodies or ADCs also are suitably administered by peritumoral, intralesional, or perilesional routes, to exert local as well as systemic therapeutic effects. The intraperitoneal route may be particularly useful, for example, in the treatment of ovarian tumors.

The antibodies or ADCs provided herein may be useful for the treatment of any disease or condition involving TF. In some embodiments, the disease or condition is a disease or condition that can benefit from treatment with an anti-TF antibody or ADC.

In some embodiments, the antibodies or ADCs provided herein are provided for use as a medicament. In some embodiments, the antibodies or ADCs provided herein are provided for use in the manufacture or preparation of a medicament. In some embodiments, the medicament is for the treatment of a disease or condition that can benefit from an anti-TF antibody or ADC.

In some embodiments, provided herein is a method of treating a disease or condition in a subject in need thereof by administering an effective amount of an anti-TF antibody or ADC provided herein to the subject.

In some embodiments, the disease or condition that can benefit from treatment with an anti-TF antibody or ADC is cancer. In some embodiments, the anti-TF antibodies or ADCs provided herein are provided for use as a medicament for the treatment of cancer. In some embodiments, the anti-TF antibodies or ADCs provided herein are provided for use in the manufacture or preparation of a medicament for the treatment of cancer. In some embodiments, provided herein is a method of treating cancer in a subject in need thereof by administering an effective amount of an anti-TF antibody or ADC provided herein to the subject.

TF is involved in thrombosis, metastasis, tumor growth, and/or tumor angiogenesis of various types of cancers, such as ovarian cancer (See Sakurai et al., Int J Gynecol Cancer, 2017, 27:37-43; Koizume et al., Biomark Cancer, 2015, 7:1-13; each of which is incorporated by reference in its entirety), cervical cancer (See Cocco et al., BMC Cancer, 2011, 11:263, incorporated by reference in its entirety), head and neck cancer (See Christensen et al., BMC Cancer, 2017, 17:572, incorporated by reference in its entirety), prostate cancer (See Yao et al., Cancer Invest., 2009, 27:430-434; Abdulkadir et al., Hum Pathol., 2009, 31:443-447; each of which is incorporated by reference in its entirety), pancreatic cancer (See Zhang et al., Oncotarget, 2017, 8:59086-59102, incorporated by reference in its entirety), triple negative breast cancer (See Zhang et al., Oncotarget, 2017, 8:59086-59102, incorporated by reference in its entirety), glioblastoma (See Guan et al., Clin Biochem., 2002, 35:321-325; Carneiro-Lobo et al., J Thromb Haemost, 2009, 7:1855-1864; each of which is incorporated by reference in its entirety), lung cancer (See Yeh et al., PLoS One, 2013, 8:e75287; Regina et al., Clin Chem., 2009, 55:1834-42; each of which is incorporated by reference in its entirety), gastric cancer (See Lo et al., Br J Cancer., 2012, 107:1125-1130, incorporated by reference in its entirety), esophageal cancer (See Chen et al., Acta Histochem., 2010, 3:233-239, incorporated by reference in its entirety), bladder cancer (See Patry et al., Int J Cancer., 2008, 122:1592-1597, incorporated by reference in its entirety), melanoma (See Bromberg et al., Proc Natl Acad Sci USA., 1995, 92:8205-8209, incorporated by reference in its entirety), and kidney cancer (See Silva et al., Int Braz J Urol., 2014, 40:499-506, incorporated by reference in its entirety).

Any suitable cancer may be treated with the antibodies or ADCs provided herein. In some embodiments, the cancer is head and neck cancer. In some embodiments, the cancer is ovarian cancer. In some embodiments, the cancer is gastric cancer. In some embodiments, the cancer is esophageal cancer. In some embodiments, the cancer is cervical cancer. In some embodiments, the cancer is prostate cancer. In some embodiments, the cancer is pancreatic cancer. In some embodiments, the cancer is estrogen receptors negative (ER−), progesterone receptors negative (PR−), and HER2 negative (HER2−) triple negative breast cancer. In some embodiments, the cancer is glioblastoma. In some embodiments, the cancer is lung cancer. In some embodiments, the cancer is bladder cancer. In some embodiments, the cancer is melanoma. In some embodiments, the cancer is kidney cancer. In some embodiments, the cancer is ocular melanoma. Additional information on the types of cancers that can be treated with anti-TF antibodies or ADCs is provided in van den Berg et al., Blood, 2012, 119:924-932, which is incorporated by reference in its entirety.

In some embodiments, provided herein is a method of delaying the onset of a cancer in a subject in need thereof by administering an effective amount of an antibody or ADC provided herein to the subject. In some embodiments, provided herein is a method for late intervention treatment of cancer in a subject in need thereof. For example, the ADC can reduce the size of a tumor (e.g., tumor volume) in a subject in need thereof or inhibit the growth of a tumor in a subject in need thereof.

In some embodiments, provided herein is a method of preventing the onset of a cancer in a subject in need thereof by administering an effective amount of an antibody or ADC provided herein to the subject.

In some embodiments, provided herein is a method of reducing the size of a tumor (e.g., tumor volume) in a subject in need thereof by administering an effective amount of an antibody or ADC provided herein to the subject. In some embodiments, an ADC provided herein reduces tumor size (e.g. tumor volume) by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99%. In some embodiments, an ADC provided herein inhibits tumor growth by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99%.

In some embodiments, provided herein is a method of reducing the number of metastases in a subject in need thereof by administering an effective amount of an antibody or ADC provided herein to the subject.

In some embodiments, provided herein is a method for extending the period of overall survival, median survival time, or progression-free survival in a subject in need thereof by administering an effective amount of an antibody or ADC provided herein to the subject.

In some embodiments, provided herein is a method for treating a subject who has become resistant to a standard of care therapeutic by administering an effective amount of an antibody or ADC provided herein to the subject.

In some embodiments, the disease or condition that can benefit from treatment with an anti-TF antibody or ADC is a disease or condition involving neovascularization. In certain embodiments, the disease or condition involving neovascularization is cancer. In some embodiments, the disease or condition that can benefit from treatment with an anti-TF antibody or ADC is a disease or condition involving vascular inflammation.

In some embodiments, the anti-TF antibodies and ADCs provided herein are provided for use as a medicament for the treatment of a disease or condition involving neovascularization. In some embodiments, the anti-TF antibodies and ADCs provided herein are provided for use in the manufacture or preparation of a medicament for the treatment of a disease or condition involving neovascularization. In certain embodiments, the disease or condition involving neovascularization is cancer. In some embodiments, the anti-TF antibodies and ADCs provided herein are provided for use as a medicament for the treatment of a disease or condition involving vascular inflammation. In some embodiments, the anti-TF antibodies and ADCs provided herein are provided for use in the manufacture or preparation of a medicament for the treatment of a disease or condition involving vascular inflammation.

In some embodiments, provided herein is a method of treating a disease or condition involving neovascularization in a subject in need thereof by administering an effective amount of an anti-TF antibody or ADC provided herein to the subject. In certain embodiments, the disease or condition involving neovascularization is cancer. In some embodiments, provided herein is a method of treating a disease or condition involving vascular inflammation in a subject in need thereof by administering an effective amount of an anti-TF antibody or ADC provided herein to the subject.

In some embodiments, provided herein is a method of delaying the onset of a disease or condition involving neovascularization in a subject in need thereof by administering an effective amount of an antibody or ADC provided herein to the subject.

In some embodiments, provided herein is a method of preventing the onset of a disease or condition involving neovascularization in a subject in need thereof by administering an effective amount of an antibody or ADC provided herein to the subject.

In some embodiments, provided herein is a method of delaying the onset of a disease or condition involving vascular inflammation in a subject in need thereof by administering an effective amount of an antibody or ADC provided herein to the subject.

In some embodiments, provided herein is a method of preventing the onset of a disease or condition involving vascular inflammation in a subject in need thereof by administering an effective amount of an antibody or ADC provided herein to the subject.

In some embodiments, an ADC provided herein, upon administration to a subject, is well tolerated by the subject. In some embodiments, an ADC provided herein, upon administration to a subject, has better tolerability relative to other anti-TF-ADCs such as clone 25A3 linked to MMAE. For example, the ADC may result in reduced skin toxicity, e.g., relative to the other anti-TF-ADCs. Indicators of skin toxicity include, without limitation, skin irritation, skin ulceration, skin rash, skin inflammation, itching, scratching, cracking, soreness, increases sensitivity to light or sun exposure, numbness, burning sensation, tingling, bumps, blisters, hives, peeling and pain.

In some embodiments, one or more ADCs provided herein, upon administration to a subject, does not require administration of one or more anti-inflammatory agents (e.g., a steroid—e.g., either topical or systemic). In some aspects one or more ADCs provided herein, upon administration to a subject, result in a reduced need for administration of one or more anti-inflammatory agents (e.g., a steroid—e.g., either topical or systemic) relative to other anti-TF-ADCs such as clone 25A3 linked to MMAE.

In some embodiments, an ADC provided herein, upon administration to a subject, results in low or absent liver toxicity, e.g., relative to baseline or relative to a different anti-TF ADC. In some embodiments, an ADC provided herein, upon administration to a subject, results in reduced liver toxicity relative to other anti-TF ADCs such as clone 25A3 linked to MMAE. This may be evaluated, e.g., using markers for hepatic damage. Non-limiting examples of markers for hepatic damage include albumin, bilirubin, globulin, gamma glutamyl transferase (γGT or GGT), glutamate pyruvate transaminase (GPT), alkaline phosphatase (ALP), alanine aminotransferase (ALT), Aspartate aminotransferase (AST), AST to platelet ratio index (APRI), Enhanced liver fibrosis (ELF), Fibrosis-4 (FIB-4), and Fibro index. For example, a reduction of hepatic damage or a reduction of the progression of hepatic damage is measured by a reduction in serum levels of ALP, AST, ALT, γGT or bilirubin.

In some embodiments, an ADC provided herein, upon administration to a subject, the antibody-drug conjugate does not increase aspartate aminotransferase (AST) levels in the subject relative to baseline levels. In some embodiments, an ADC provided herein, upon administration to a subject, results in reduced aspartate aminotransferase (AST) levels in the subject relative to baseline levels or relative to a different anti-TF ADC. In some embodiments, an ADC provided herein, upon administration to a subject, does not increase alanine transaminase levels in the subject, relative to baseline levels. In some embodiments, an ADC provided herein, upon administration to a subject, results in reduced alanine transaminase levels in the subject relative to baseline levels or relative to a different anti-TF ADC.

In some embodiments, an ADC provided herein, upon administration to a subject, results in reduced, low, or an absence of neutropenia, e.g., relative to baseline. This can be relative to a different anti-TF antibody-drug conjugate such as clone 25A3 linked to MMAE.

In some embodiments, an ADC provided herein, upon administration to a subject, does not alter, increase or decrease the number of monocytes in a subject, e.g., relative to baseline. This can be relative to a different anti-TF antibody-drug conjugate such as clone 25A3 linked to MMAE.

Non-limiting examples of anti-inflammatory agents include non-steroidal anti-inflammatory drugs (NSAIDs), steroidal anti-inflammatory drugs, beta-agonists, anticholinergic agents, antihistamines (e.g., ethanolamines, ethylenediamines, piperazines, and phenothiazine), and methyl xanthines. Examples of NSAIDs include, but are not limited to, aspirin, ibuprofen, salicylates, acetominophen, celecoxib, diclofenac, etodolac, fenoprofen, indomethacin, ketoralac, oxaprozin, nabumentone, sulindac, tolmentin, rofecoxib, naproxen, ketoprofen and nabumetone. Such NSAIDs function by inhibiting a cyclooxgenase enzyme (e.g., COX-1 and/or COX-2). Examples of steroidal anti-inflammatory drugs include, but are not limited to, glucocorticoids, dexamethasone, cortisone, hydrocortisone, prednisone, prednisolone, triamcinolone, azulfidine, and eicosanoids such as prostaglandins, thromboxanes, and leukotrienes. These anti-inflammatory agents may be topical or systemic.

Anti-inflammatory agents and their dosages, routes of administration and recommended usage are known in the art and have been described in such literature as the Physician's Desk Reference (60^{th} ed., 2006).

### 10. Combination Therapies

In some embodiments, an antibody or ADC provided herein is administered with at least one additional therapeutic agent. Any suitable additional therapeutic agent may be administered with an antibody or ADC provided herein. In some aspects, the additional therapeutic agent is selected from radiation, a cytotoxic agent, a chemotherapeutic agent, a cytostatic agent, an anti-hormonal agent, an immunostimulatory agent, an anti-angiogenic agent, and combinations thereof.

The additional therapeutic agent may be administered by any suitable means. In some embodiments, an antibody or ADC provided herein and the additional therapeutic agent are included in the same pharmaceutical composition. In some embodiments, an antibody or ADC provided herein and the additional therapeutic agent are included in different pharmaceutical compositions.

In embodiments where an antibody or ADC provided herein and the additional therapeutic agent are included in different pharmaceutical compositions, administration of the antibody or ADC can occur prior to, simultaneously, and/or following, administration of the additional therapeutic agent.

### 11. Diagnostic Methods

Also provided are methods for detecting the presence of TF on cells from a subject. Such methods may be used, for example, to predict and evaluate responsiveness to treatment with an antibody or ADC provided herein.

In some embodiments, the method can be used to detect TF in a subject having or suspected of having a disease or condition. In some embodiments, the methods comprise (a) receiving a sample from the subject; and (b) detecting the presence or the level of TF in the sample by contacting the sample with the antibody provided herein. In some embodiments, the methods comprise (a) administering to the subject the antibody provided herein; and (b) detecting the presence or the level of TF in the subject. In some embodiments, the disease or condition is a cancer. In some embodiments, the cancer is head and neck cancer. In some embodiments, the cancer is ovarian cancer. In some embodiments, the cancer is gastric cancer. In some embodiments, the cancer is esophageal cancer. In some embodiments, the cancer is cervical cancer. In some embodiments, the cancer is prostate cancer. In some embodiments, the cancer is pancreatic cancer. In some embodiments, the cancer is estrogen receptors negative (ER−), progesterone receptors negative (PR−), and HER2 negative (HER2−) triple negative breast cancer. In some embodiments, the cancer is glioblastoma. In some embodiments, the cancer is lung cancer. In some embodiments, the cancer is bladder cancer. In some embodiments, the cancer is melanoma. In some embodiments, the cancer is kidney cancer. In some embodiments, the disease or condition involves neovascularization. In certain embodiments, the disease or condition involving neovascularization is cancer. In some embodiments, the disease or condition involves vascular inflammation.

In some embodiments, the methods comprise (a) administering to the subject the ADC provided herein; and (b) detecting the presence or the level of TF in the subject. In some embodiments, the disease or condition is a cancer. In some embodiments, the cancer is head and neck cancer. In some embodiments, the cancer is ovarian cancer. In some embodiments, the cancer is gastric cancer. In some embodiments, the cancer is esophageal cancer. In some embodiments, the cancer is cervical cancer. In some embodiments, the cancer is prostate cancer. In some embodiments, the cancer is pancreatic cancer. In some embodiments, the cancer is estrogen receptors negative (ER−), progesterone receptors negative (PR−), and HER2 negative (HER2−) triple negative breast cancer. In some embodiments, the cancer is glioblastoma. In some embodiments, the cancer is lung cancer. In some embodiments, the cancer is bladder cancer. In some embodiments, the cancer is melanoma. In some embodiments, the cancer is kidney cancer.

In some embodiments, the antibody provided herein is conjugated with a fluorescent label. In some embodiments, the antibody provided herein is conjugated with a radioactive label. In some embodiments, the antibody provided herein is conjugated with an enzyme label.

In some embodiments, the ADC provided herein comprises a fluorescent label. In some embodiments, the ADC provided herein comprises a radioactive label. In some embodiments, the ADC provided herein comprises an enzyme label.

In some embodiments, the relative amount of TF expressed by such cells is determined. The fraction of cells expressing TF and the relative amount of TF expressed by such cells can be determined by any suitable method. In some embodiments, flow cytometry is used to make such measurements. In some embodiments, fluorescence assisted cell sorting (FACS) is used to make such measurement.

### 12. Kits

Also provided are kits comprising the antibodies or ADCs provided herein. The kits may be used for the treatment, prevention, and/or diagnosis of a disease or disorder, as described herein.

In some embodiments, the kit comprises a container and a label or package insert on or associated with the container. Suitable containers include, for example, bottles, vials, syringes, and IV solution bags. The containers may be formed from a variety of materials, such as glass or plastic. The container holds a composition that is by itself, or when combined with another composition, effective for treating, preventing and/or diagnosing a disease or disorder. The container may have a sterile access port. For example, if the container is an intravenous solution bag or a vial, it may have a port that can be pierced by a needle. At least one active agent in the composition is an antibody or ADC provided herein. The label or package insert indicates that the composition is used for treating the selected condition.

In some embodiments, the kit comprises (a) a first container with a first composition contained therein, wherein the first composition comprises an antibody or ADC provided herein; and (b) a second container with a second composition contained therein, wherein the second composition comprises a further therapeutic agent. The kit in this embodiment of the invention may further comprise a package insert indicating that the compositions can be used to treat a particular condition.

Alternatively, or additionally, the kit may further comprise a second (or third) container comprising a pharmaceutically-acceptable excipient. In some aspects, the excipient is a buffer. The kit may further include other materials desirable from a commercial and user standpoint, including filters, needles, and syringes.

### EXAMPLES

The following are examples of methods and compositions of the invention. It is understood that various other embodiments may be practiced, given the general description provided herein.

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

### Example 1: Synthesis of Antibody-Drug Conjugates (ADCs)

Antibody-Drug Conjugates (ADCs) of anti-TF antibodies and Linker-Toxin A (also referred to herein as “LT-A”) were prepared as described below. The structure of Linker-Toxin A, unlinked, is shown in FIG. 1. Anti-TF antibodies are described in PCT/US2019/12427, filed on Jan. 4, 2019; herein incorporated by reference in its entirety for all purposes.

Briefly, 5 to 10 mg/mL of 25A3 antibody (see Table 8 for CDR and V region sequences of clone 25A3) in phosphate-buffered saline (PBS), pH 7.4 was reduced by the addition of Tris(2-carboxyethyl)phosphine (2.0-2.5 or 3.2 molar equivalents) and a final concentration of 0.8 mM diethylenetriamine-pentaacetic acid. After 2 hr at 37° C., the partially reduced antibody was cooled on ice for 10 minutes, then conjugated for 1 h with 8 molar equivalents of Linker-Toxin A on ice. The reaction was quenched with an excess of N-acetyl-L-cysteine. The quenched reaction was allowed to sit on ice for 30 minutes prior to purification. ADCs were purified through two rounds of 40 kDa MWCO Zeba™ Spin Desalting Columns (10 mL Columns, Product #8772, Lot # RL240689) each, as per the manufacturer's protocol. Prior to purification, both sets of columns was primed with sterile PBS. The ADC was purified through one set of PBS primed columns first, the sample was then collected and purified a second time through the other set. After the second purification, the ADC was pooled back together and sterile filtered and frozen at −80° C.

Drug-antibody ratio (DAR) may be measured by UV/vis spectroscopy, hydrophobic interaction chromatography (HIC), and/or reverse phase liquid chromatography separation with time-of-flight detection and mass characterization {RP-UPLC/Mass spectrometry), as described in WO 2016/041082. Distribution of drug-linked forms (for example, the fraction of DAR0, DAR1, DAR2, etc. species) may also be analyzed by various techniques known in the art, including MS (with or without an accompanying chromatographic separation step), hydrophobic interaction chromatography, reverse-phase I-IPLC or iso-electric focusing gel electrophoresis (IEF), as also described in WO 2016/041082.

For this example, the drug-antibody ratio (DAR) of the resulting ADCs was ˜3. The DAR was determined by hydrophobic interaction chromatography: Average DAR=(0×(DAR0 Area %)+2×(DAR2 Area %)+4×(DAR4 Area %)+6×(DAR6 Area %)+8×(DAR8 Area %)/100. Size exclusion chromatography was used to ensure the ADC preparation was at least 95% monomeric.

A depiction of an ADC comprising LT-A is shown in FIG. 2. ADCs comprising 25A3 and LT-A, e.g. as prepared in this example, were used in the assays and studies of Examples 2-8 below.

### Example 2: Cytotoxicity Assays of Antibody-Drug Conjugates (ADCs)

To evaluate cytotoxicity of ADCs, TF-positive A431 cells were plated in 384-well plates (Greiner Bio-One, Monroe, N.C., USA) at 4×10³ cells per well in 40 μL of media. An ADC comprising the anti-TF antibody 25A3 or an isotype control antibody conjugated to Linker-Toxin A were prepared as described in Example 1, then serially diluted starting at 5 nM. Cells were incubated with the ADCs for 4 h, followed by a washout and another 68 hr of culture in fresh medium, or incubated with the ADCs for 3 days. Cell viability was subsequently assessed by lysis in CellTiter-Glo (CTG) assay reagent (Promega, Madison, Wis., USA). CTG luminescence was measured on an Envision plate reader and the mean and standard deviation of 4 replicates were graphed in Prism. For each ADC, the IC₅₀ were calculated in Prism using a 4-parameter binding model.

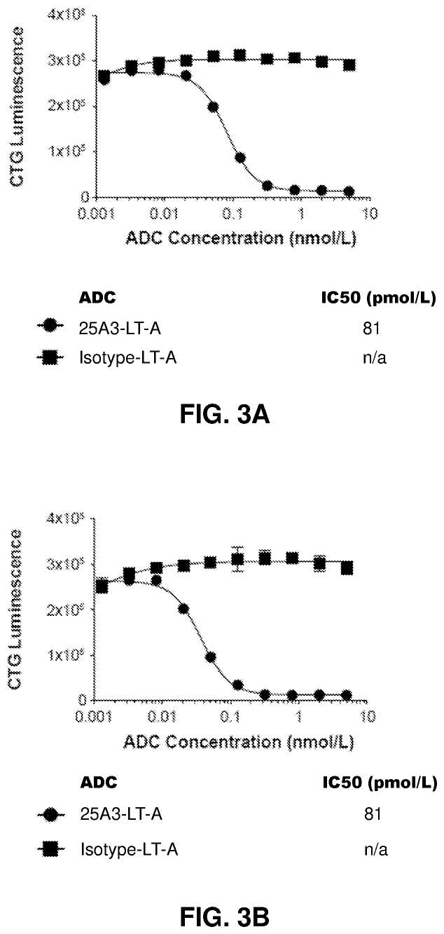

FIG. 3A shows the cell viability as indicated by CTG luminescence and the calculated IC₅₀ in TF-positive A431 cells after a 4 h incubation with isotype control or 25A3-LT-A, followed by washout and 68 h of culture. FIG. 3B shows the cell viability as indicated by CTG luminescence and the calculated IC₅₀ in TF-positive A431 cells after a 3-day incubation with either isotype control or 25A3-LT-A. Only the anti-TF ADC resulted in cytotoxicity in TF-positive A431 cells.

These data indicate that anti-TF antibody-drug conjugates reduced the viability of TF-positive cells in vitro.

### Example 3: Effect of Anti-TF ADC in MDA MB213 Xenograft Model

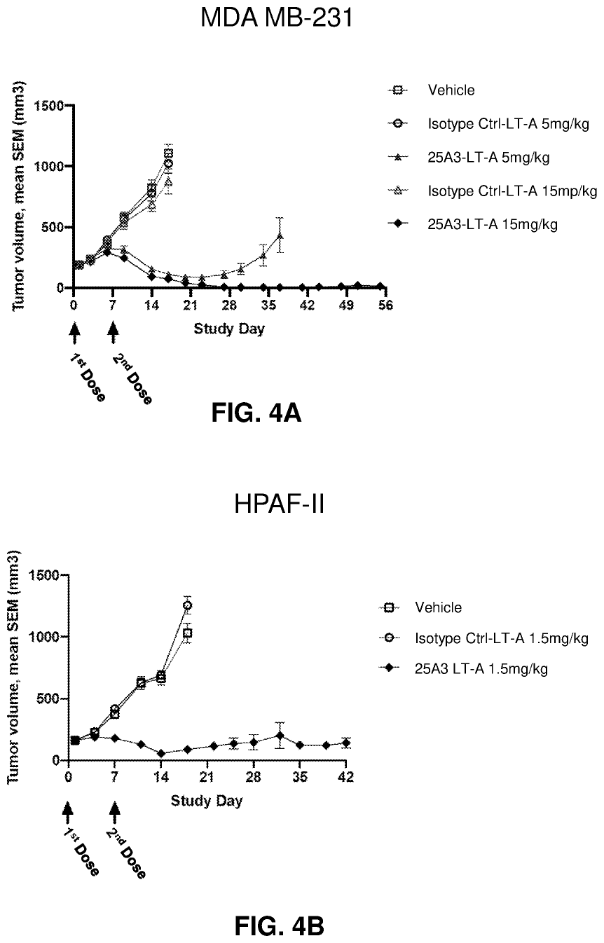

Xenograft studies in immune compromised mice were performed to evaluate the efficacy of the ADCs in vivo. The TF-positive MDA-MB231 triple-negative breast carcinoma cell line was implanted subcutaneously in the flank of athymic nude mice (Charles River Laboratories, Wilmington, Mass.). Animals were randomized when tumors reached an average size of 150-200 mm3 and treated with the indicated dose of the anti-TF antibody-drug conjugate 25A3-LT-A, prepared as described in Example 1, isotype control-LT-A or vehicle (PBS) intraperitoneally (i.p.) once weekly for 2 weeks. Body weight and tumor size assessments were performed bi-weekly. Animals were removed from study and euthanized once tumor size reached 1200 mm³ or skin ulceration was evident. Results are depicted in FIG. 4A. Treatment with 5 mg/kg 25A3-LT-A reduced tumor volume and delayed tumor growth, as compared to either dose of the isotype control LT-A. Treatment with 15 mg/kg 25A3-LT-A reduced tumor volume and prevented tumor growth, as compared to either dose of the isotype control LT-A. These data indicate that anti-TF antibody-drug conjugate 25A3-LT-A was effective in reducing the tumor size in vivo.

### Example 4: Effect of Anti-TF ADC in HPAF-II Xenograft Model

Xenograft studies in immune compromised mice were performed to evaluate the efficacy of the ADCs in vivo. TF-positive HPAF-II pancreatic carcinoma cells were implanted subcutaneously in the flank of athymic nude mice (Charles River Laboratories, Wilmington, Mass.). Animals were randomized to treatment groups when tumors reached an average size of 150-200 mm³ and treated with the indicated dose of the anti-TF antibody-drug conjugate 25A3-LT-A, prepared as described in Example 1, isotype control-LT-A or vehicle (PBS) intraperitoneally (i.p.) once weekly for 2 weeks. Body weight and tumor size assessments were performed bi-weekly. Animals were removed from study and euthanized once tumor size reached 1200 mm³ or skin ulceration was evident. Results are depicted in FIG. 4B. ADCs comprising anti-TF antibody 25A3 conjugated to LT-A decreased the tumor size compared to the vehicle-treated or Isotype control LT-A-treated groups. These data indicate that anti-TF antibody-drug conjugate 25A3-LT-A was effective in reducing the tumor size in vivo.

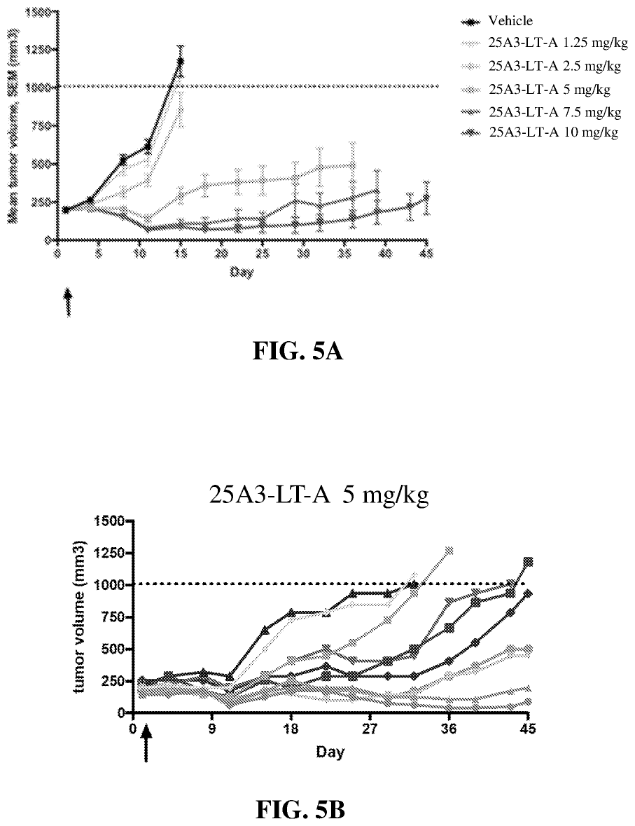

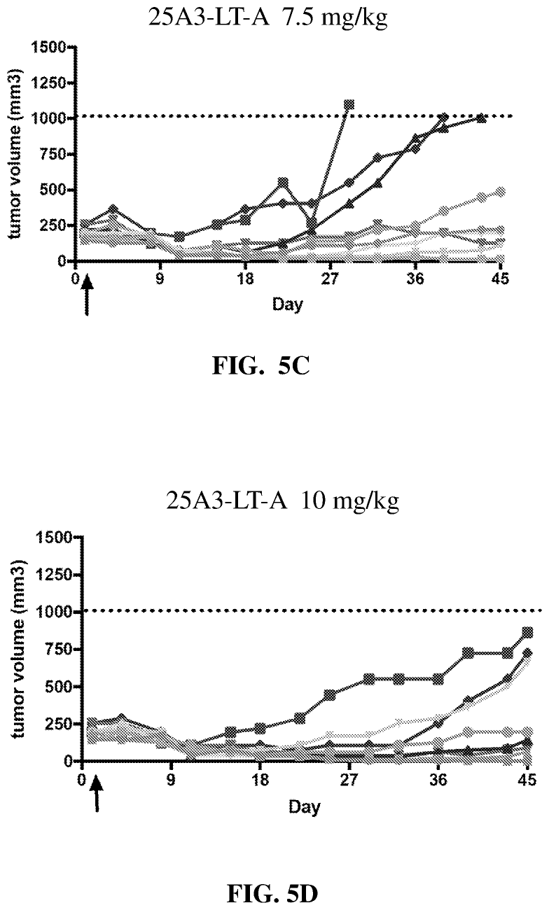

For a dose response study, the indicated dose of the anti-TF antibody-drug conjugate 25A3-LT-A was administered i.p. once, when the tumors reached a size of 200 mm³. FIGS. 5A-5D show the effects on tumor volume of 25A3-LT-A administered at doses ranging from 1.25 mg/kg to 10 mg/kg. Mice treated with 1.25 mg/kg or the vehicle had tumors that exceeded 1000 mm³ within 15 days of receiving treatment. In contrast, mice treated with 5 mg/kg, 7.5 mg/kg or 10 mg/kg 25A3-LT-A showed slower tumor growth over the first five weeks following treatment.

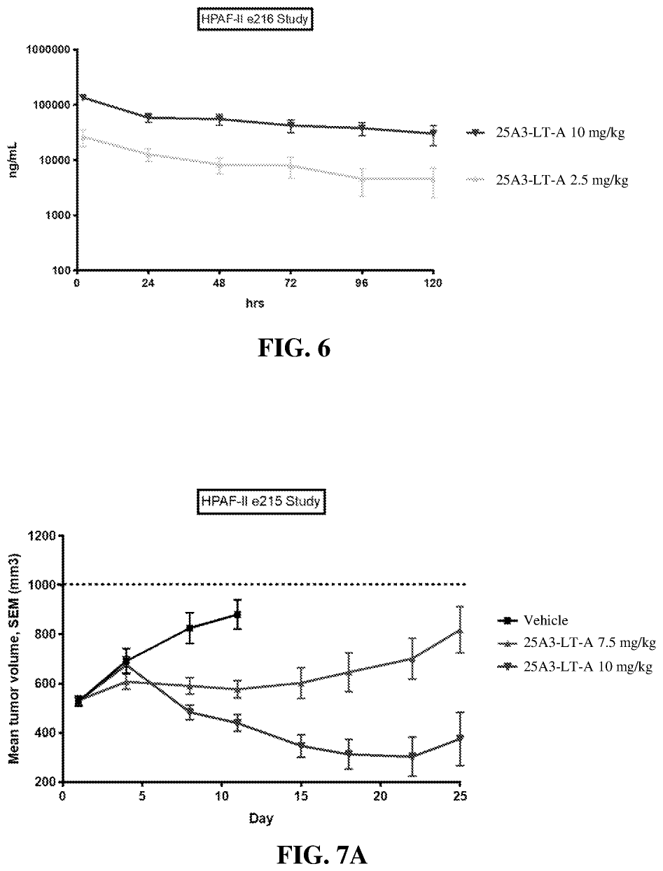

For a pharmacokinetic (PK) study, the mice were treated i.p. once with either 2.5 mg/kg or 10 mg/kg of the anti-TF antibody-drug conjugate 25A3-LT-A, starting when the tumors reached a size of 200 mm³. Briefly, samples were collected every 24 hours for 5 days by mandibular bleeds (0.1 mL). The concentration of 25A3-LT-A was measured in a PK assay where hTF was the coating reagent and a secondary anti-hIgG was the detector. The results of the PK assay are shown in FIG. 6 and Table 23. The data revealed that the anti-TF antibody-drug conjugate 25A3-LT-A had linear pharmacokinetics.

TABLE 23
Results from the PK assay for mice treated with 25A3-LT-A.

| | | | AUC₀₋ₜ | AUCinf | | CL | Vss |
|---|---|---|---|---|---|---|---|
| | cₘₐₓ | tₘₐₓ | (μg/mL | (μg/mL | T1/2 | (mL/kg/ | (mL/ |
| Dose | (μg/mL) | (hr) | *hr) | *hr) | (hr) | hr) | kg) |
| 2.5 mg/kg | 26.3 | 2 | 1190 | 1670 | 71.7 | 1.50 | 139 |
| 25A3-LT-A | | | | | | | |
| 10 mg/kg | 136 | 2 | 6740 | 10500 | 86.3 | 0.951 | 111 |
| 25A3-LT-A | | | | | | | |

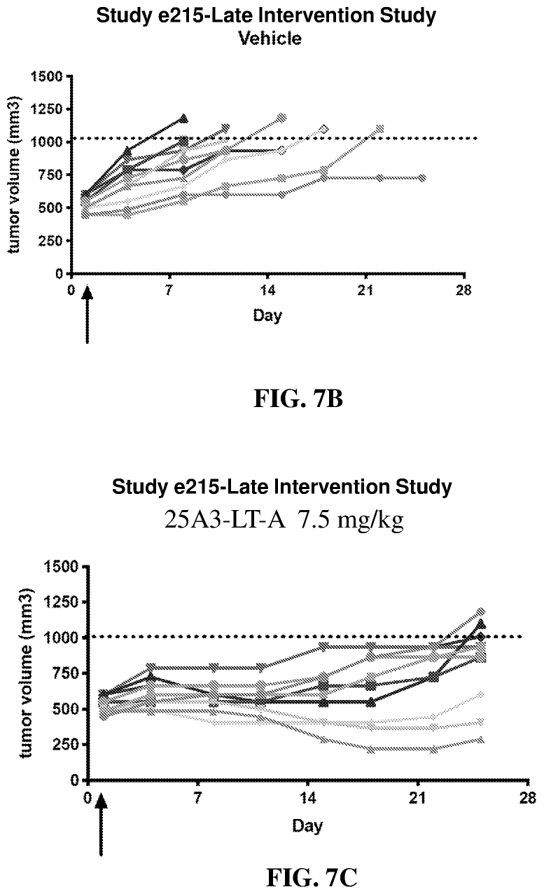

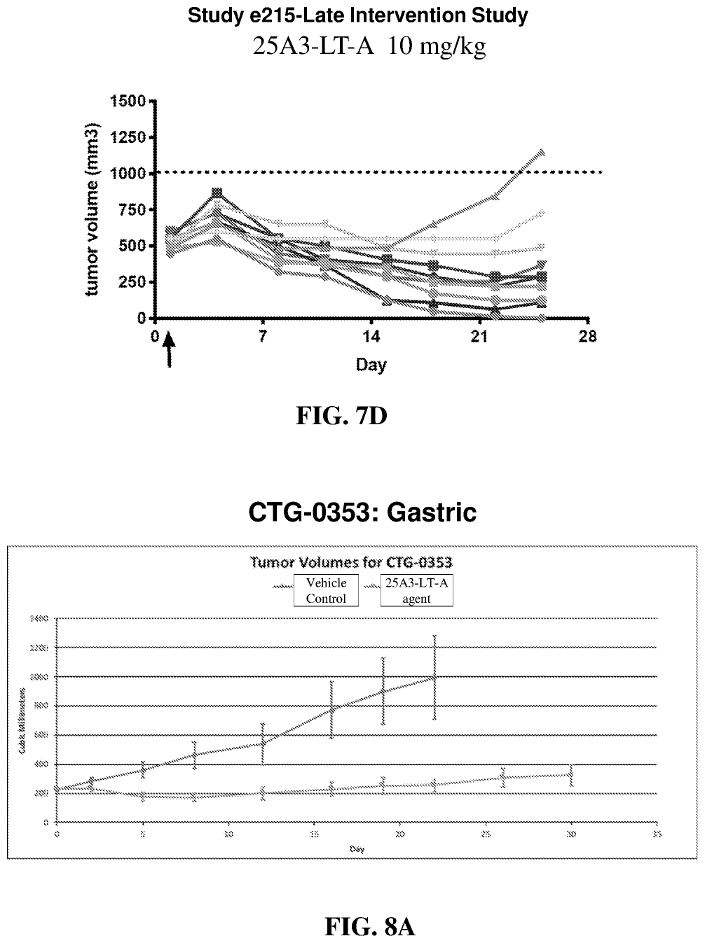

To evaluate the effects of the anti-TF ADC administered during late intervention, mice were treated i.p. with either 7.5 mg/kg or 10 mg/kg of the anti-TF antibody-drug conjugate 25A3-LT-A, once starting when the tumors reached a size of 500 mm³. The results are shown in FIGS. 7A-7D. Mice treated with 7.5 mg/kg of 25A3-LT-A showed reduced tumor growth relative to mice in the control group (vehicle). The data also showed negative tumor growth (i.e., a reduction in tumor volume) for mice that were treated with the higher dose of 10 mg/kg relative to mice in the control group (vehicle).

### Example 5: Effect of Anti-TF ADC in Various Patient Derived Xenograft Models

Patient-derived xenograft (PDX) studies in athymic nude mice (Envigo, Indianapolis, Ind.) were performed to evaluate the efficacy of the 25A3-LT-A ADCs in vivo. Briefly, tumors were passaged in stock animals and harvested for re-implantation. Study animals were implanted unilaterally on the left flank with tumor fragments and were randomized to treatment groups when their tumors reached an average size of 150-200 mm³. Animals were treated i.p. with 10 mg/kg of 25A3-LT-A or the vehicle control (PBS) once. Body weight and tumor volume measurements were performed bi-weekly. Animals were removed from the study and euthanized after 30 days, once tumor size reached 1200 mm³ or when skin ulceration was evident. Mean tumor volume (MTV) with the standard error of the mean (SEM) was plotted over time. Treatment efficacy was determined by calculating tumor growth inhibition (% TGI=100%×[1−(final MTV−initial MTV of a treated group)/(final MTV−initial MTV of the control group)]) before any of the animals in the vehicle arm were euthanized due to a tumor size≥1200 mm³.

The immunohistochemical (IHC) analysis was used for detection of TF expression and cellular localization (membranous vs cytoplasmic). Tissues from untreated mice were pretreated using Rip Tide (Mosaic Laboratories, Lake Forest, Calif.) for 40 min at 95-97° C. in a water bath, cooled for 10 min on the bench, rinsed 3 times with distilled water, and rinsed for 5 min with Splash-T Buffer (Mosaic Laboratories). Tissue sections were blocked in EnVision Peroxidase-Blocking Reagent (EnVision+ Mouse HRP Detection Kit, Agilent, Carpinteria, Calif.) for 5 min, followed by 2 rinses in Splash-T Buffer for 5 min each. Next, the tissue sections were stained with an anti-TF antibody (mouse clone HTF-1) or a mouse negative control reagent for 30 min, followed by 2 rinses in Splash-T Buffer for 5 min each. The second staining step of the tissue sections was carried out for 30 min with EnVision+ Mouse HRP (EnVision+ Mouse HRP Detection Kit), followed by 2 rinses in Splash-T Buffer for 5 min each. To visualize the anti-TF staining, tissue sections were developed with DAB chromogen (EnVision+ Mouse HRP Detection Kit) for 5 min, followed by 10 dips and a 5 min rinse in distilled water. Tissue sections were counterstained with Hematoxylin for 5 min followed by 3 rinses in distilled water.

Staining intensity was scored on a semi-quantitative integer scale from 0 (negative) to 3 (or “3+”) by a certified anatomic pathologist. The percentage of cells staining positively at each intensity level was recorded. Scoring was based on localization of TF to the cell membrane. The H score combines components of staining intensity with the percentage of positive cells. It has a value between 0 and 300 and is defined as: 1×(percentage of cells staining at 1+ intensity)+2×(percentage of cells staining at 2+ intensity)+3×(percentage of cells staining at 3+ intensity)=H score. 3+ is the strongly stained, 2+ is moderately stained, 1+ is weakly stained and 0 is no stain.

PDX Study 1

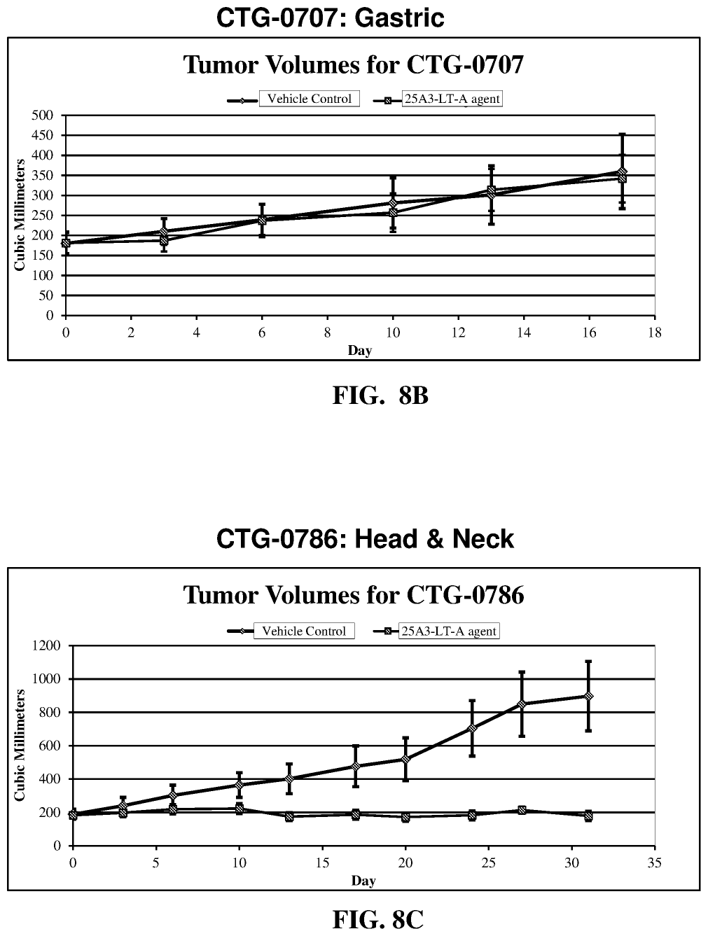

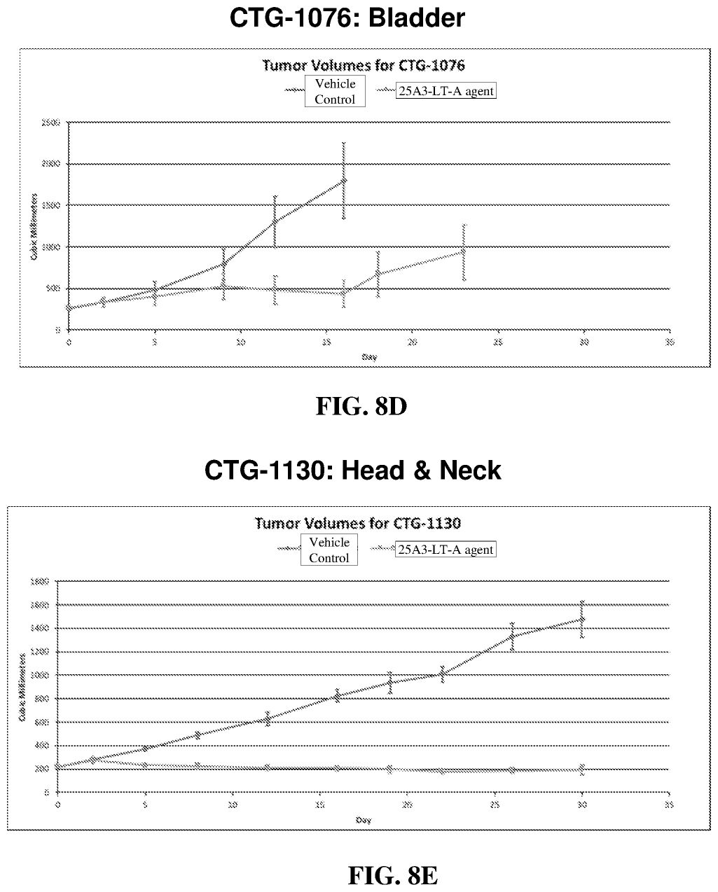

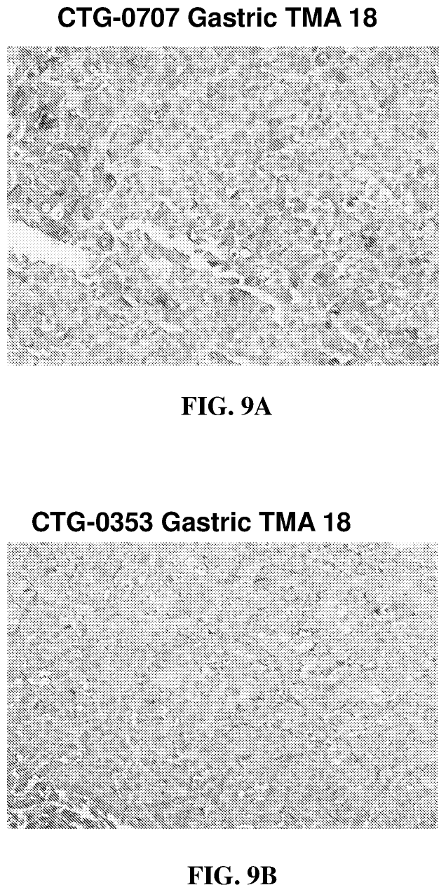

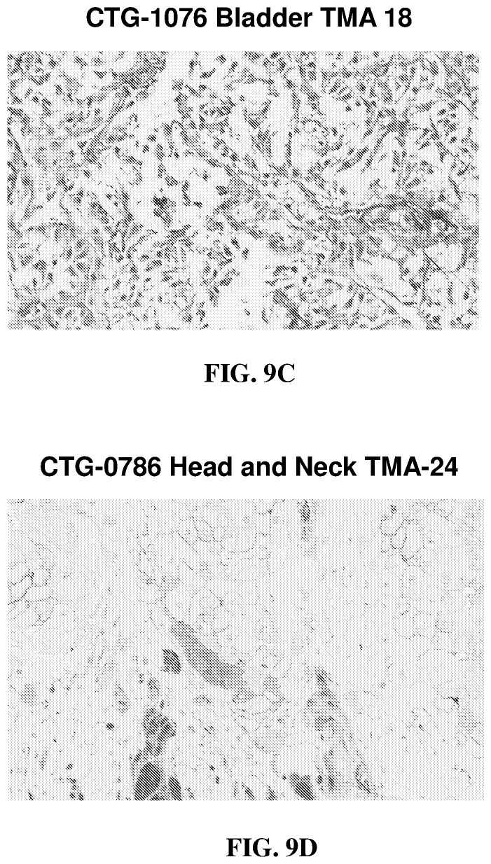

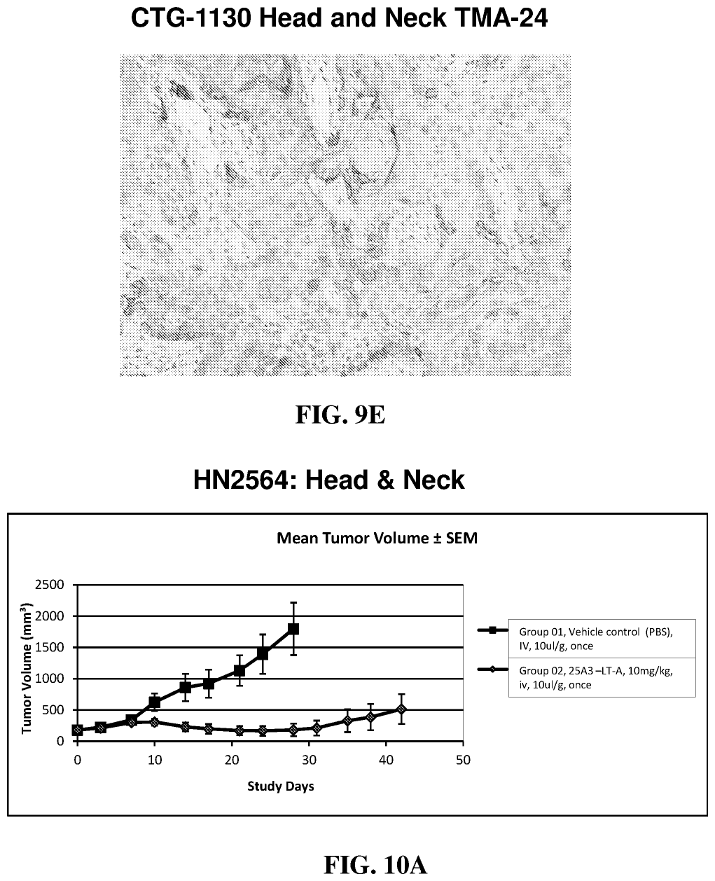

Using the methods disclosed above, five mice (models) were evaluated for efficacy of the 25A3-LT-A ADCs. The results are shown in Tables 24-29 and FIGS. 8A-8E. As shown, four out of the five models exhibited significant tumor growth inhibition. These data suggest high efficacy in a variety of tumor types and TF-expressing cancers. IHC analysis of the models revealed comparable H-scores between different tumor models (all between 100 and 200), indicating comparable levels of TF expression, and heterogenous distribution of TF (FIGS. 9A-9E).

TABLE 24
Tumor growth inhibition reported in PDX study 1.

| Model# | Tumor Type | Tumor Growth Inhibition |
|---|---|---|
| CTG-0353 | Gastric | 97% |
| CTG-0707 | Gastric | 10% |
| CTG-0786 | Head and neck | 100% |
| CTG-1076 | Bladder | 88% |
| CTG-1130 | Head and neck | 101% |

FIG. 9A and Table 25 show the results from the IHC analysis of the CTG-0707 gastric model. In the following tables, SCL=subcellular localization. M=membrane staining; C=cytoplasmic staining; MC=membrane/cytoplasmic staining CM=cytoplasmic/membrane staining.

TABLE 25
H-score determination for the CTG-0707 gastric model.

| % Cells Staining at Each Intensity | | | | | | | Percent | |
|---|---|---|---|---|---|---|---|---|
| 3+ | SCL | 2+ | SCL | 1+ | SCL | 0 | Positive | H-Score |
| 0 | | 60 | CM | 20 | CM | 20 | 80 | 140 |

FIG. 9B and Table 26 show the results from the IHC analysis of the CTG-0353 gastric model.

TABLE 26
H-score determination for the CTG-0353 gastric model.

| % Cells Staining at Each Intensity | | | | | | | Percent | |
|---|---|---|---|---|---|---|---|---|
| 3+ | SCL | 2+ | SCL | 1+ | SCL | 0 | Positive | H-Score |
| 10 | MC | 40 | MC | 20 | MC | 30 | 70 | 130 |

FIG. 9C and Table 27 show the results from the IHC analysis of the CTG-1076 bladder model.

TABLE 27
H-score determination for the CTG-1076 bladder model

| % Cells Staining at Each Intensity | | | | | | | Percent | |
|---|---|---|---|---|---|---|---|---|
| 3+ | SCL | 2+ | SCL | 1+ | SCL | 0 | Positive | H-Score |
| 0 | | 70 | MC | 10 | MC | 20 | 80 | 150 |

FIG. 9D and Table 28 show the results from the IHC analysis of the CTG-0786 head and neck model.

TABLE 28
H-score determination for the CTG-0786 head and neck model.

| % Cells Staining at Each Intensity | | | | | | | Percent | |
|---|---|---|---|---|---|---|---|---|
| 3+ | SCL | 2+ | SCL | 1+ | SCL | 0 | Positive | H-Score |
| 0 | | 50 | MC | 30 | MC | 20 | 80 | 130 |

FIG. 9E and Table 29 show the results from the IHC analysis of the CTG-1130 head and neck model.

TABLE 29
H-score determination for the CTG-1130 head and neck model.

| % Cells Staining at Each Intensity | | | | | | | Percent | |
|---|---|---|---|---|---|---|---|---|
| 3+ | SCL | 2+ | SCL | 1+ | SCL | 0 | Positive | H-Score |
| 20 | MC | 30 | MC | 40 | MC | 10 | 90 | 160 |

PDX Study 2

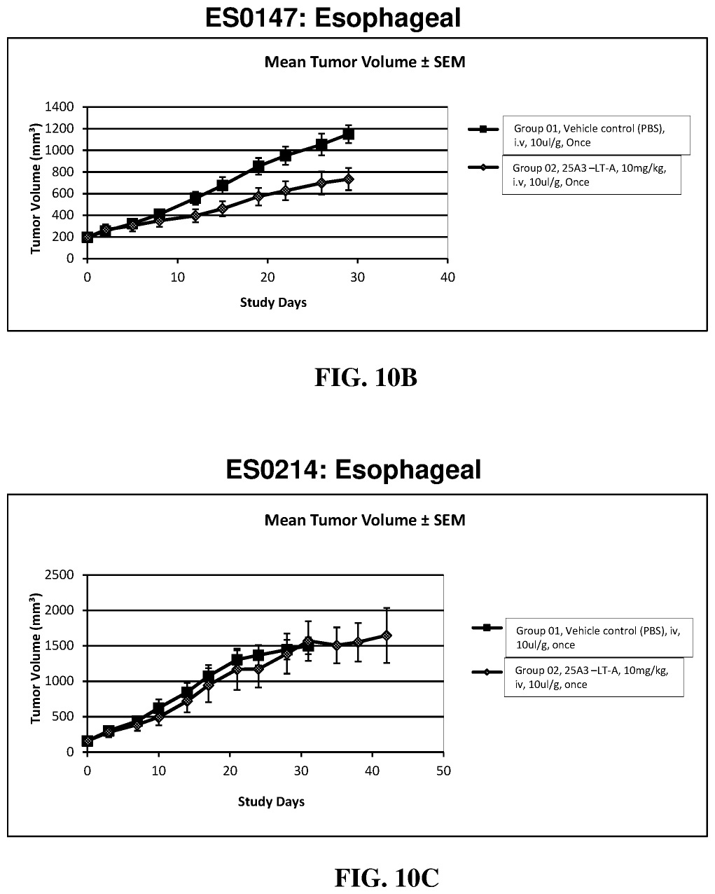

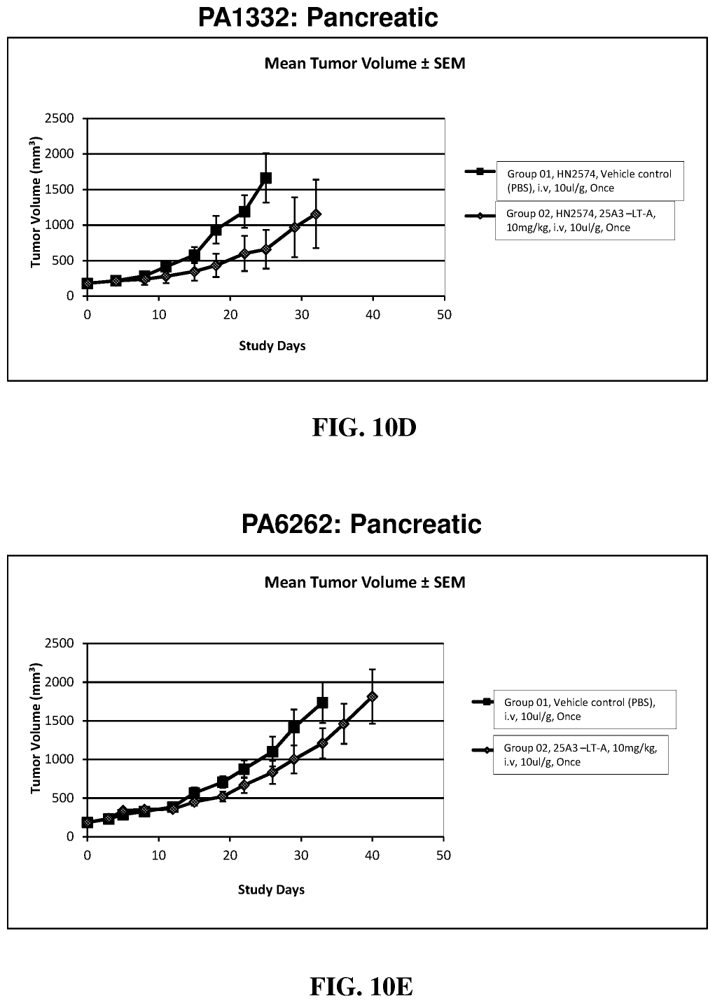

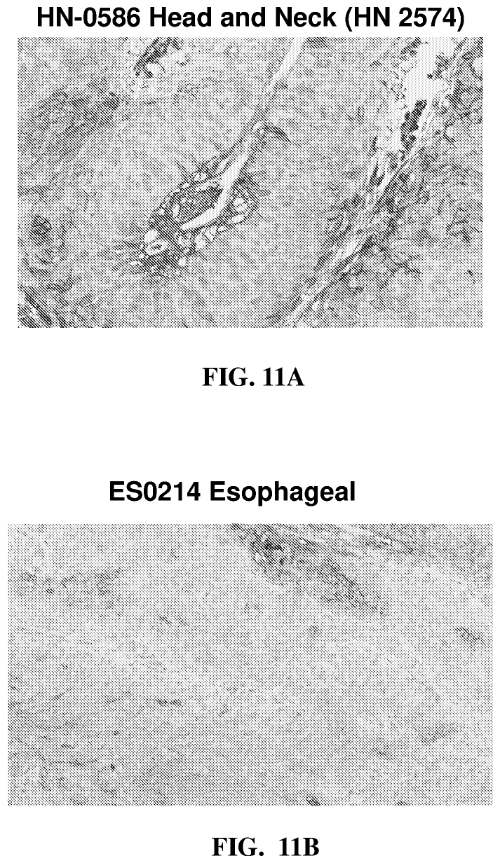

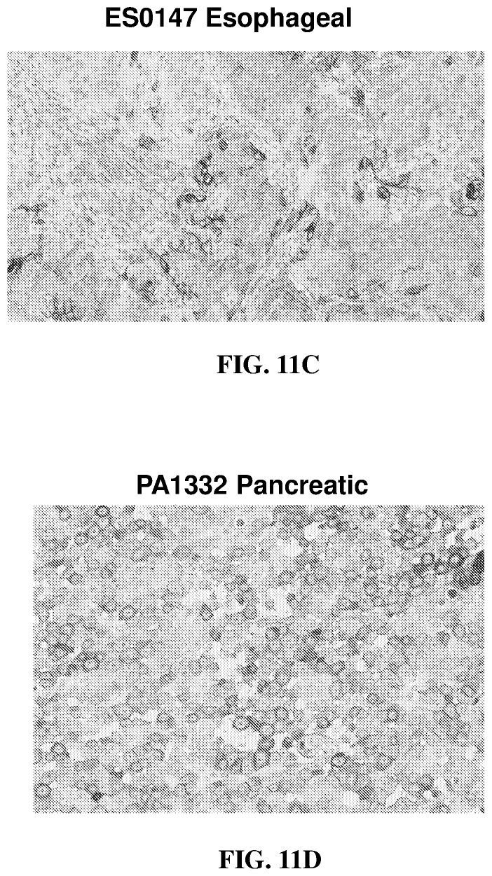

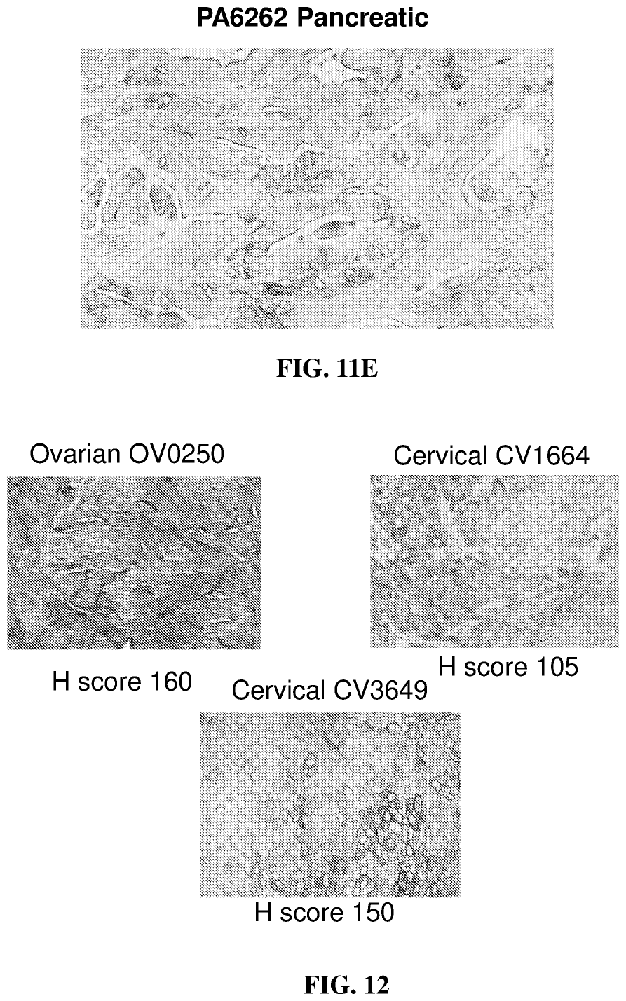

Using the methods disclosed above, five mice were evaluated for efficacy of the 25A3-LT-A ADCs. The results are shown in Tables 30-35 below and FIGS. 10A-10E. As shown, two out of the five models exhibited significant tumor growth inhibition. There was greater variation between models tested in PDX Study 2 relative to the models tested in PDX Study 1, which were conducted by different third party vendors. In particular the two esophageal cancer models and one pancreatic cancer model (PA6262) exhibited low tumor growth inhibition. Some potential explanations for the low tumor growth inhibition observed in these models include:
Low or absent TF expression relative to the other models, post-implantation; and/or
Necrosis within the tumors; and/or
Experimental variation or error.
   IHC analysis of the models revealed comparable H-scores between different tumor models (indicating comparable levels of TF expression) and heterogenous distribution of TF (FIGS. 11A-12).

TABLE 30
Tumor growth inhibition reported in PDX study 2.

| Model# | Tumor Type | Tumor Growth Inhibition |
|---|---|---|
| HN2574 | HN—Head and Neck Cancer | 90% |
| ES0147 | ES—Esophageal Cancer | 36% |
| ES0214 | ES—Esophageal Cancer | −4% |
| PA1332 | PA—Pancreatic Cancer | 60% |
| PA6262 | PA—Pancreatic Cancer | 30% |

FIG. 11A and Table 31 show the results from the IHC analysis of the HN 2574 head and neck model.

TABLE 31
H-score determination for the HN 2574 head and neck model.

| % Cells Staining at Each Intensity | | | | | | | Percent | |
|---|---|---|---|---|---|---|---|---|
| 3+ | SCL | 2+ | SCL | 1+ | SCL | 0 | Positive | H-Score |
| 10 | MC | 30 | MC | 50 | MC | 10 | 90 | 140 |

FIG. 11B and Table 32 show the results from the IHC analysis of the ES0214 esophageal model.

TABLE 32
H-score determination for the ES0214 esophageal model.

| % Cells Staining at Each Intensity | | | | | | | Percent | |
|---|---|---|---|---|---|---|---|---|
| 3+ | SCL | 2+ | SCL | 1+ | SCL | 0 | Positive | H-Score |
| 10 | CM | 20 | CM | 40 | MC | 30 | 70 | 110 |

FIG. 11C and Table 33 show the results from the IHC analysis of the ES0147 esophageal model.

TABLE 33
H-score determination for the ES0147 esophageal model.

| % Cells Staining at Each Intensity | | | | | | | Percent | |
|---|---|---|---|---|---|---|---|---|
| 3+ | SCL | 2+ | SCL | 1+ | SCL | 0 | Positive | H-Score |
| 15 | MC | 15 | MC | 30 | MC | 40 | 60 | 105 |

FIG. 11D and Table 34 show the results from the IHC analysis of the PA1332 pancreatic model.

TABLE 34
H-score determination for the PA1332 pancreatic model.

| % Cells Staining at Each Intensity | | | | | | | Percent | |
|---|---|---|---|---|---|---|---|---|
| 3+ | SCL | 2+ | SCL | 1+ | SCL | 0 | Positive | H-Score |
| 10 | CM | 60 | CM | 25 | CM | 5 | 95 | 175 |

FIG. 11E and Table 35 show the results from the IHC analysis of the PA6262 pancreatic model.

TABLE 35
H-score determination for the PA6262 pancreatic model.

| % Cells Staining at Each Intensity | | | | | | | Percent | |
|---|---|---|---|---|---|---|---|---|
| 3+ | SCL | 2+ | SCL | 1+ | SCL | 0 | Positive | H-Score |
| 10 | MC | 30 | CM | 50 | MC | 10 | 90 | 140 |

FIG. 12 shows immunostains from three additional mouse models that were implanted with ovarian or cervical cancer tumor patient-derived xenografts and underwent IHC analysis using the methods disclosed above in Example 5.

### Example 6: Effect of Anti-TF ADC in Gastric Patient Derived Xenograft Model

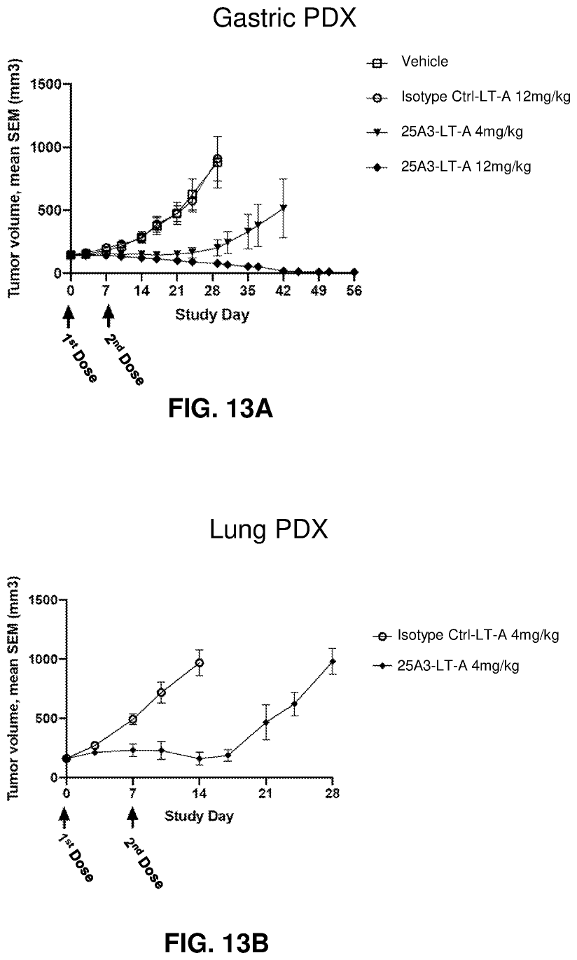

Xenograft studies in immune compromised mice were performed to evaluate the efficacy of the ADCs in vivo. The TF-positive gastric patient derived xenograft was implanted subcutaneously in the flank of athymic nude mice (Envigo, Indianapolis, Ind.). Animals were randomized when tumors reached an average size of 150-200 mm3 and treated with the indicated dose of the anti-TF antibody-drug conjugate 25A3-LT-A, prepared as described in Example 1, isotype control-LT-A or vehicle (PBS) intraperitoneally (i.p.) once weekly for 2 weeks. Body weight and tumor size assessments were performed bi-weekly. Animals were removed from study and euthanized once tumor size reached 1200 mm³ or skin ulceration was evident. Results are depicted in FIG. 13A. ADCs comprising anti-TF antibody 25A3 conjugated to LT-A (25A3-LT-A) decreased the tumor size compared to the vehicle-treated or Isotype control LT-A-treated groups. Treatment with 4 mg/kg 25A3-LT-A reduced tumor volume and delayed tumor growth, as compared to 12 mg/kg of the isotype control LT-A. Treatment with 12 mg/kg 25A3-LT-A reduced tumor volume and prevented tumor growth, as compared to 12 mg/kg isotype control LT-A. These data indicate that anti-TF antibody-drug conjugate 25A3-LT-A was effective in reducing the tumor size in vivo.

### Example 7: Effect of Anti-TF ADC in Lung Patient Derived Xenograft Model

Xenograft studies in immune compromised mice were performed to evaluate the efficacy of the ADCs in vivo. The TF-positive lung patient derived xenograft was implanted subcutaneously in the flank of NSG™ (NOD.Cg-Prkdc^{scid} Il2rgt^{m1Wijl}/SzJ) mice (Jackson Laboratories, Sacramento, Calif.). Animals were randomized when tumors reached an average size of 150-200 mm3 and treated with the indicated dose of the anti-TF antibody drug conjugate 25A3-LT-A, prepared as described in Example 1, or isotype control-LT-A intraperitoneally (i.p.) once weekly for 2 weeks. Body weight and tumor size assessments were performed bi-weekly. Animals were removed from study and euthanized once tumor size reached 1200 mm³ or skin ulceration was evident. Results are depicted in FIG. 13B. ADCs comprising anti-TF antibody 25A3 conjugated to LT-A delayed tumor growth as compared to the Isotype control LT-A-treated group. These data indicate that anti-TF antibody-drug conjugate 25A3-LT-A was effective in delaying tumor progression in vivo.

### Example 8: Pilot Cyno Toxicology Study: 25A3-LT-A vs. 25A3-MMAE

The objective of this study was to assess toxicity parameters of the ADC 25A3-LT-A relative to the anti-TF antibody-drug conjugate 25A3-MMAE and to determine whether the former exhibits similar if not better on-target toxicology relative to publicly available data for another anti-TF antibody drug conjugate comprising MMAE, (tisotumab vedotin (Genmab)). Tisotumab vedotin is an anti-TF fully human monoclonal antibody conjugated to MMAE with a protease-cleavable linker. (Chenard-Poirier et al., Annals of Oncology 28.suppl_5 (2017)). Tisotumab vedotin has been shown in previous studies to cause dose limiting toxicity (e.g., neutropenia) when administered at the dose of 2.2 mg/kg. (de Bono et al., The Lancet Oncology 20.3 (2019): 383-393). Neutropenia and skin toxicity have also been observed at a dose of 3 mg/kg with tisotumab vedotin. It has resulted in dose limiting toxicities at 6 mg/kg, at which the subject(s) display grade 4 neutropenia and severe skin irritation and skin ulceration. (Parren, P., Advancing Towards the Clinic As Soon As Possible: Pre-Clinical Development of a Therapeutic ADC Targeting Tissue Factor. World ADC Conference, Oct. 16, 2013; Geoij, B. E. C. G. Antibody-drug conjugates in cancer. Diss. Faculty of Medicine, Leiden University Medical Center (LUMC), Leiden University, 2016.) Previous studies using a HER-2-targeted antibody conjugated to LT-A revealed that the HER-2-LT-A did not result in significant neutropenia up to 18 mg/kg. Additionally, transient ALT and AST elevations have been reported for tisotumab vedotin.

To conduct the current pilot toxicology study, female cynomolgus (“cyno”) monkeys (n=3 per group) were treated (by intravenous injection) with 25A3-LT-A or 25A3-MMAE and received the indicated doses on days 1, 22, and 36 of the study. Animals treated with 25A3-MMAE received 1.5 mg/kg, 3 mg/kg, or 6 mg/kg per dose, while animals treated with 25A3-LT-A received 3 mg/kg, 6 mg/kg or 18 mg/kg per dose. All monkeys that survived until day 43 of the study underwent scheduled euthanasia.

Clinical Observations: Skin Toxicity

Table 36 provides qualitative data relating to skin toxicity in various treatment groups by the end of the study. As shown, there was more severe skin irritation in animals treated with 25A3-MMAE than in animals treated with 25A3-LT-A. For example, only one of three animals required topical steroidal treatment in the 6.0 mg/kg 25A3-LT-A, while two of three animals in the 6.0 mg/kg 25A3-MMAE group required topical and systemic steroids to counter skin irritation. Among all of the 25A3 LT-A treatment groups (n=12), only one animal required systemic steroids and that animal had received the highest tested dose of 18.0 mg/kg, which was 3× the highest dose tested in the 25A3-MMAE cohort. These data indicate greater skin toxicity (and lower tolerability) when using an MMAE-based anti-TF ADC compared to a counterpart LT-A-based anti-TF antibody-ADC.

TABLE 36
Clinical observations relating to skin toxicity.

| Group | Observations | Treatment |
|---|---|---|
| 1.5 mg/kg | Red skin in inguinal zone | |
| 25A3-MMAE | after first dose in all | |
| | animals and up to day 43 | |
| | in 1 animal | |
| 3.0 mg/kg | Red skin in inguinal zone, | |
| 25A3-MMAE | hind limbs and muzzle | |
| | after first dose | |
| 6.0 mg/kg | Extensive red and dry skin | Two animals: topical and |
| 25A3-MMAE | after first dose, more | systemic steroids |
| | limited after second dose | |
| 3.0 mg/kg | Red skin in inguinal region | |
| 25A3-LT-A | and hind limbs | |
| 6.0 mg/kg | Red skin extended to more | One animal: topical |
| 25A3-LT-A | regions | steroid |
| | Excessive scratching in | |
| | one animal | |
| 12.0 mg/kg | Red skin extended to more | One animal: topical |
| 25A3-LT-A | regions | steroid |
| | Excessive scratching | |
| 18.0 mg/kg | Red skin extended to more | One animal: topical and |
| 25A3-LT-A | regions | systemic steroids |
| | Conjunctivitis/eye infection | |
| | in 1 animal | |

Clinical Chemistry: Liver Toxicity

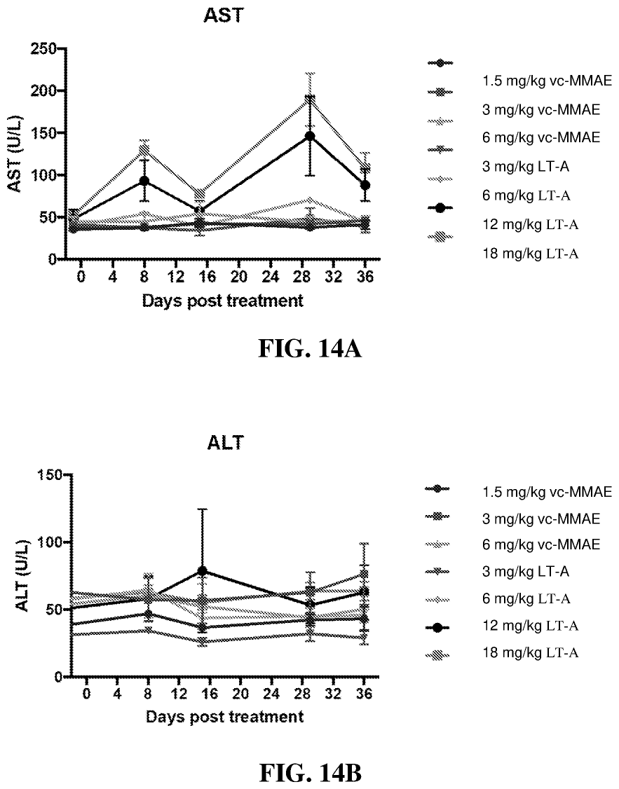

To evaluate liver toxicity parameters associated with 25A3-MMAE and 25A3-LT-A treatment, globulin, albumin, alanine aminotransferase (ALT), and aspartate aminotransferase (AST) were measured. Blood samples were collected from each of the monkeys on days 0 (pretreatment), 8, 15, 29, and 36 of the study and before euthanasia. FIGS. 14A and 14B show the AST and ALT levels for the indicated treatment groups, respectively. These data showed a transient increase in AST over the course of the study in monkeys treated with 12 mg/kg 25A3-LT-A and 18 mg/kg 25A3-LT-A. Both of these treatment groups exhibited Grade 2 AST elevation. Additionally, the 12 mg/kg 25A3-LT-A and 18 mg/kg 25A3-LT-A treatment groups exhibited slight increases in globulin and slight increases in albumin. Neither 25A3-MMAE nor 25A3-LT-A resulted in transient ALT elevation.

Hematology

To further evaluate potential off-target effects of 25A3-MMAE and 25A3-LT-A, neutrophils and monocytes in the monkeys were measured. Blood samples were collected from each of the monkeys on days 0 (pretreatment), 4, 8, 15, 25, 29, 36, and 43 of the study and before euthanasia. The samples were used to complete a blood count, which determined hematology parameters including, but not limited to, monocyte count and neutrophil count.

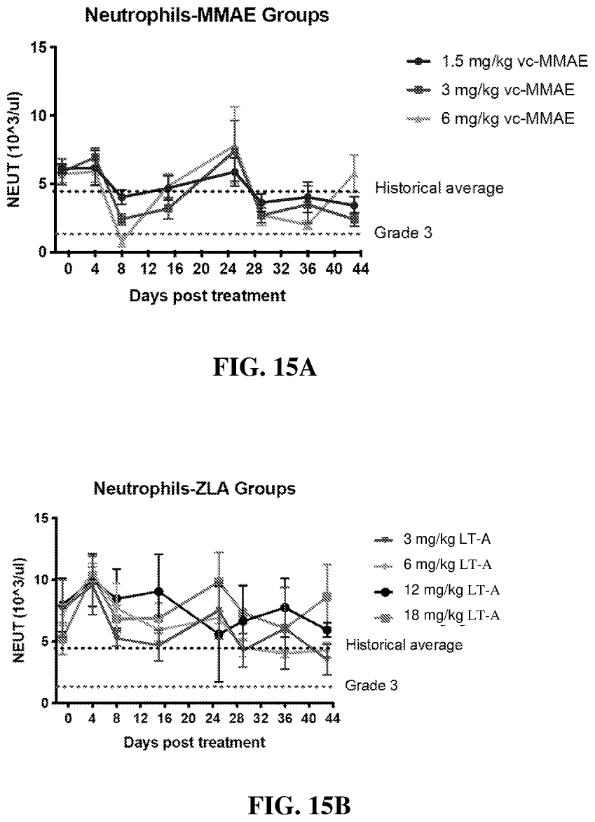

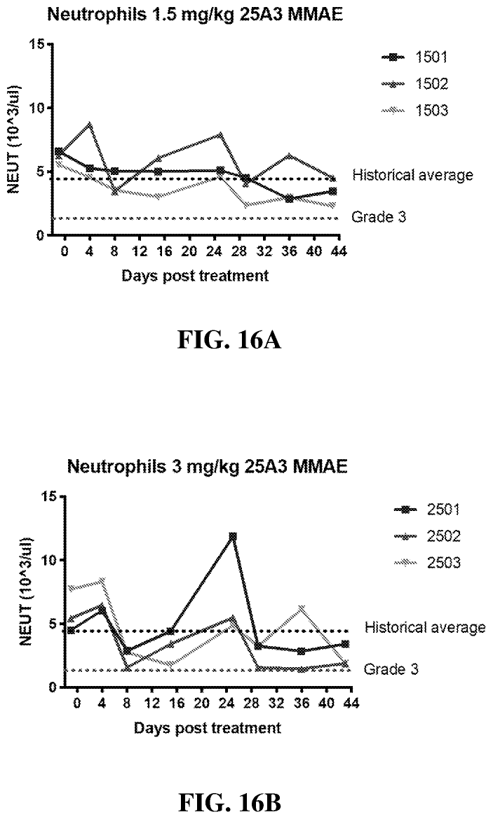

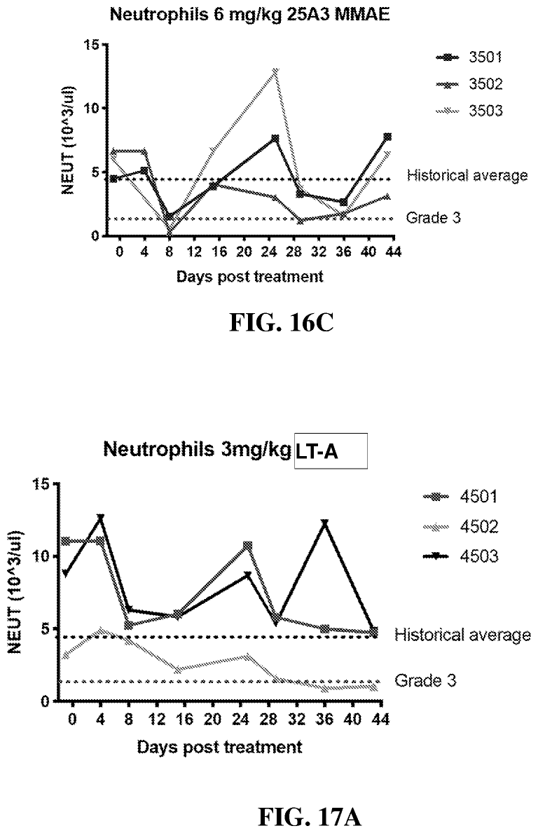

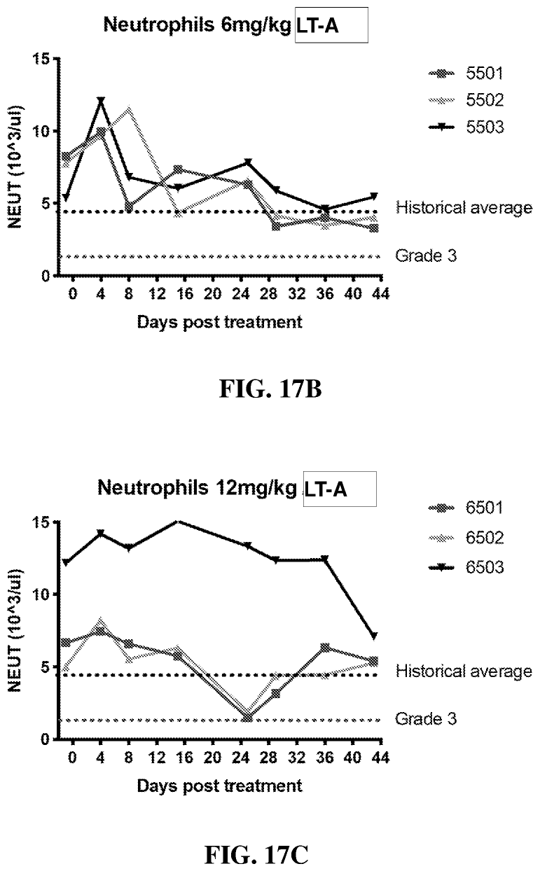

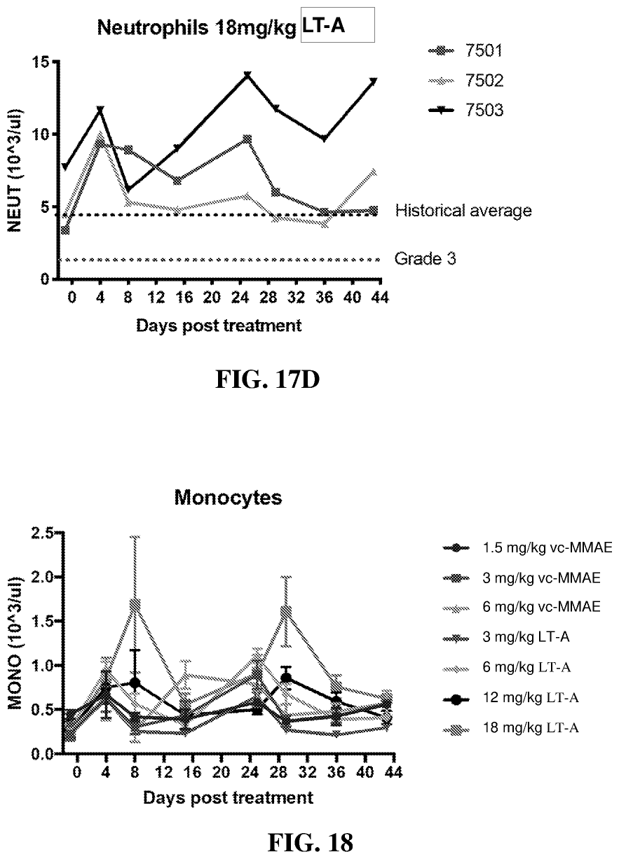

FIGS. 15A-17D show the neutrophil levels over the course of the study. There was a marked decrease in neutrophils for all animals in the 3 mg/kg 25A3-MMAE and 6 mg/kg 25A3-MMAE groups. In contrast, most monkeys in the 25A3-LT-A groups stayed above historical averages or had a single drop. The one exception was monkey 4502; however it should be noted that monkey 4502 started with a very low neutrophil count (FIG. 17A). The data showed grade 3 and 4 neutropenia in the monkeys treated with 6 mg/kg 25A3-MMAE and no neutropenia for monkeys in the 25A3-LT-A treatment groups. This indicates dose-limiting neutropenia at 6 mg/kg when treating with 25A3-MMAE but not when treating with 25A3-LT-A.

The monocyte levels were comparable across 25A3-MMAE and 25A3-LT-A treatment groups, except for the 18 mg/kg 25A3-LT-A treatment group (FIG. 18). Within the 18 mg/kg 25A3-LT-A treatment group, the transient monocyte elevation was attributed to monkey 7503, which had an eye infection.

PK and Immunogenicity

To examine ADC PK, blood samples were collected from the monkeys after administration of the first and second doses and evaluated using a monoclonal antibody (mAB) assay and an intact ADC Assay. The mAB assay used hTF as the coating reagent and a secondary anti-hIgG as the detector. The intact ADC assay used an anti-toxin mAb as the coating reagents and anti-IgG as the detector. Tables 37 and 38 shows the results from the mAb assay and the intact ADC Assay.

TABLE 37
Mean mAB assay results with standard deviation.

| | Dose | 1^{st} Dose | | | 2^{nd} Dose | | |
|---|---|---|---|---|---|---|---|
| | Level | | AUC-0₀₋₂₁ | | | AUC₂₁₋₄₂ | |
| Mole- | (mg/ | Cₘₐₓ | (μg*d/ | t_{1/2} | | (μg*d/ | t_{1/2} |
| cule | kg) | (μg/mL) | mL) | (days) | Cₘₐₓ | mL) | (days) |
| 25A3- | 1.5 | 33.7 | 53.0 | 1.15 | 14.0 | 17.1 | 0.679 |
| MMAE | | (2.79) | (10.4) | (0.166) | (15.6) | (23.5) | (0.371) |
| 25A3- | 3 | 66.2 | 137 | 1.45 | 36.7 | 21.7 | 0.462 |
| MMAE | | (5.10) | (16.3) | (0.166) | (12.3) | (12.5) | (0.202) |
| 25A3- | 6 | 139 | 358 | 1.67 | 95.3 | 146 | 1.04 |
| MMAE | | (6.40) | (64.2) | (0.164) | (55.4) | (137) | (0.779) |
| 25A3- | 3 | 62.9 | 200 | 1.67 | 54.7 | 137 | 1.15 |
| LT-A | | (9.17) | (19.1) | (0.230) | (31.6) | (97.8) | (0.282) |
| 25A3- | 6 | 162 | 504 | 1.81 | 142 | 259 | 1.62 |
| LT-A | | (23.7) | (74.1) | (0.220) | (26.8) | (59.1) | (0.536) |
| 25A3- | 12 | 291 | 1290 | 2.67 | 273 | 748 | 1.67 |
| LT-A | | (15.2) | (84.2) | (0.562) | (140) | (117) | (0.139) |
| 25A3- | 18 | 466 | 1970 | 4.73 | 375 | 2000 | 3.97 |
| LT-A | | (37.6) | (207) | (0.423) | (82.2) | (145) | (0.229) |

TABLE 38
Mean Intact ADC Assay results with standard deviation

| | Dose | 1^{st} Dose | | | 2^{nd} Dose | | |
|---|---|---|---|---|---|---|---|
| | Level | Cₘₐₓ | AUC-0₀₋₂₁ | t_{1/2} | | AUC₂₁₋₄₂ | t_{1/2} |
| Molecule | (mg/kg) | (μg/mL) | (μg*d/mL) | (days) | Cₘₐₓ | (μg*d/mL) | (days) |
| 25A3- | 1.5 | 31.6 | 38.4 | 1.12 | 15.8 | 16.4 | 0.820 |
| MMAE | | (1.63) | (5.87) | (0.169) | (14.1) | (15.8) | (0.201) |
| 25A3- | 3 | 57.1 | 88.9 | 1.34 | 39.2 | 26.4 | 0.582 |
| MMAE | | (7.17) | (11.5) | (0.161) | (14.0) | (16.1) | (0.234) |
| 25A3- | 6 | 157 | 265 | 1.64 | 99.2 | 103 | 0.957 |
| MMAE | | (15.7) | (45.0) | (0.172) | (56.7) | (86.4) | (0.681) |
| 25A3-LT-A | 3 | 68.1 | 214 | 1.91 | 59.4 | 170 | 1.39 |
| | | (6.23) | (27.4) | (0.250) | (32.3) | (110) | (0.369) |
| 25A3-LT-A | 6 | 165 | 511 | 2.23 | 152 | 302 | 2.26 |
| | | (10.7) | (84.2) | (0.436) | (13.6) | (35.2) | (0.823) |
| 25A3-LT-A | 12 | 281 | 1250 | 3.03 | 278 | 805 | 2.12 |
| | | (18.7) | (86.7) | (0.589) | (22.4) | (118) | (0.179) |
| 25A3-LT-A | 18 | 464 | 2000 | 5.10 | 378 | 2100 | 4.14 |
| | | (24.5) | (212) | (0.816) | (90.4) | (205) | (0.410) |

For both 25A3-MMAE and 25A3-LT-A, time concentrations profiles were similar, as determined with the mAB and intact ADC assays. This suggests that each of the ADCs do not degrade rapidly.

The findings from analyzing the PK and immunogenicity data are summarized in Table 39. In the case of 25A3-MMAE, the PK increased approximately linearly with the first dose, and the concentration following the second dose administration was markedly lower than after the first dose administration. In the case of 25A3-LT-A, across groups, the concentrations after the second dose administration were similar to those observed after the first administration (except at the 3 mg/kg dose). The data suggest slight target-mediated drug disposition, which decreases with increasing dose. The data also showed faster clearance of 25A3-MMAE compared to 25A3-LT-A at 3 and 6 mg/kg, suggesting non-target mediated uptake.

As shown in Table 39, all of the monkeys developed anti-drug antibodies (ADA). While the immunogenicity was observed, generally, ADA in cynomolgus monkeys is not predictive of ADA in humans.

TABLE 39
The PK and Immunogenicity findings.

| Group | PK | Immunogenicity |
|---|---|---|
| 1.5 mg/kg | Significant decrease in | All animals positive at |
| 25A3-MMAE | exposure between first and | day 22 (titers 105-2080) |
| | second dose | and day 43 (titers 30-7385) |
| 3.0 mg/kg | Significant decrease in | All animals positive at |
| 25A3-MMAE | exposure between first and | day 22 (titers 410-1429) |
| | second dose | and day 43 (titers 1176- |
| | | 1387) |
| 6.0 mg/kg | Significant decrease in | All animals positive at |
| 25A3-MMAE | exposure between first and | day 22 (titers 301-6066) |
| | second dose | and day 43 (titers 27-2177) |
| 3.0 mg/kg | Moderate decrease in | All animals positive at |
| 25A3-LT-A | exposure between first and | day 22 (titers 5-874) and |
| | second dose | 2 on day 43 (titers 301- |
| | | 1341) |
| 6.0 mg/kg | Moderate decrease in | All animals positive at |
| 25A3-LT-A | exposure between first and | day 22 (titers 219-395) |
| | second dose | and day 43 (titers 198- |
| | | 1432) |
| 12.0 mg/kg | Slight decrease in exposure | All animals positive at |
| 25A3-LT-A | between first and second | day 22 (titers 63-92) and |
| | dose | day 43 (titers 26-53) |
| 18.0 mg/kg | Slight decrease in exposure | All animals positive at |
| 25A3-LT-A | between first and second | day 22 (titers 4-46) and |
| | dose | day 43 (titers 2-32) |

### Example 9: Synthesis of Linker-Toxin A

The following example describes the preparation of an exemplary linker-toxin (Linker-Toxin A, also referred to as LT-A) that comprises the auristatin derivative, Compound 9:

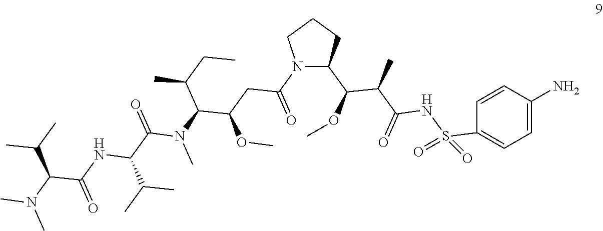 [SMILES: CC[C@H](C)[C@@H]([C@@H](CC(=O)N1CCC[C@H]1[C@H](OC)[C@@H](C)C(=O)NS(=O)(=O)c1ccc(N)cc1)OC)N(C)C(=O)[C@@H](NC(=O)[C@H](C(C)C)N(C)C)C(C)C]

Similar protocols may be employed to prepare linker-toxins comprising other auristatin derivatives of general Formula I as described herein (see also International Patent Application Publication No. WO 2016/041082).

### 7.1 Ethyl (2R,3R)-3-methoxy-2-methyl-3-((S)-pyrrolidin-2-yl)propanoate (Compound 1)

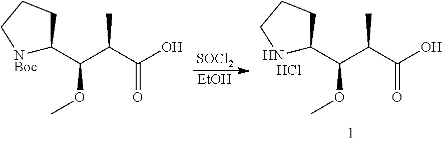

To a stirred solution of (2R,3R)-3-((S)-1-(tert-butoxycarbonyl)pyrrolidin-2-yl)-3-methoxy-2-methylpropanoic acid (Boc-Dap-OH, 4.31 g, 15.0 mmol) in absolute ethanol (27.0 mL) at 0° C. was added thionyl chloride (3.0 mL) in a dropwise fashion. The resulting solution was allowed to warm to room temperature and progress was monitored by HPLC-MS. After 18 h, no remaining starting material was detected and the solution was concentrated to dryness under reduced pressure. The resulting oil was suspended in toluene (10 mL) and concentrated under reduced pressure two times, then suspended in diethyl ether (5 mL) and concentrated under reduced pressure two times to afford the title compound as a white solid foam (3.78 g, quant yield %). MS m/z obs.=216.5 (M+1).

### 7.2 (3R,4S,5S)-4-((S)-2-(((benzyloxy)carbonyl)amino)-N,3-dimethylbutanamido)-3-methoxy-5-methylheptanoic Acid (Compound 3)

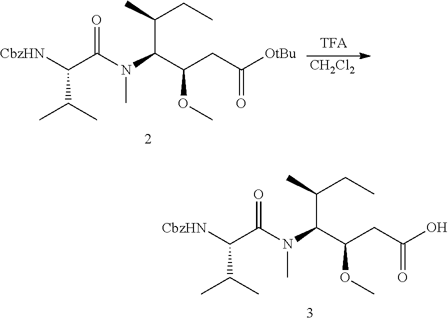

Compound 2 was prepared as described in International Patent Application Publication No. WO 2016/041082.

To a stirred solution of Compound 2 (6.965 g, 14.14 mmol) in dichloromethane (20 mL) was added trifluoroacetic acid (5.0 mL). The reaction was monitored for completion by HPLC-MS and after 40 h no starting material remained. The reaction was concentrated under reduced pressure, co-evaporated with toluene (2×10 mL) and dichloromethane (2×10 mL) to obtain a foamy white solid (6.2 g, quant yield with residual TFA). This material was dissolved in 200 mL of hot 1:3 EtOAc:hexanes and allowed to cool to room temperature. During cooling, a precipitate formed as well as some small crystals. 5 mL EtOAc was added and the suspension was heated once again to fully dissolve the precipitate. More crystals formed on cooling to room temperature and the flask was placed at −30° C. overnight. The following morning the mother liquor was decanted and the crystals rinsed with 2×50 mL hexanes and dried under high vacuum. Recovered 5.67 g of the title compound as a crystalline product. MS m/z obs.=405.7 (M+1).

### 7.3 Ethyl (2R,3R)-3-((S)-1-((3R,4S,5S)-4-((S)-2-(((benzyloxy)carbonyl)amino)-N,3-dimethylbutanamido)-3-methoxy-5-methylheptanoyl)pyrrolidin-2-yl)-3-methoxy-2-methylpropanoate (Compound 4)

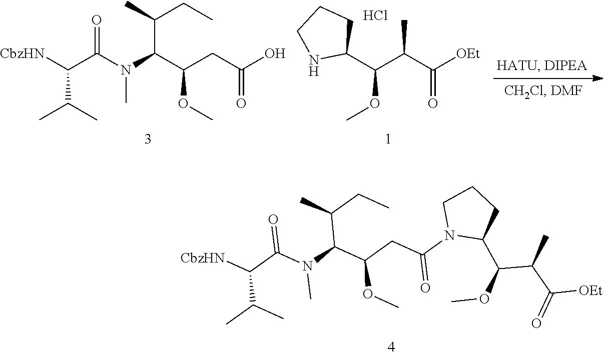

To a stirred solution of Compound 3 (6.711 g, 15.37 mmol, 1.025 equiv) in a mixture of dichloromethane (5.0 mL) and N,N-dimethylformamide (5.0 mL) at room temperature was added HATU (5.732 g, 15.07 mmol, 1.005 equiv) and N,N-diisopropylethylamine (7.84 mL, 3 equiv). After stirring for 30 minutes at room temperature, a solution of Compound 1 (3.776 g, 15.00 mmol, 1.0 equiv) in a mixture of dichloromethane (1.0 mL) and N,N-dimethylformamide (1.0 mL) was added dropwise and rinsed in residual Compound 1 with an additional 3 mL of 1:1 dichloromethane:N,N-dimethylformamide. The reaction was monitored by HPLC-MS and no remaining Compound 1 was observed after 15 minutes. The reaction was concentrated under reduced pressure, diluted with ethyl acetate (˜125 mL) and the organic phase was extracted with 1 M HCl (2×50 mL), 1×dH₂O (1×50 mL), saturated NaHCO₃ (3×50 mL), brine (25 mL). Acidic and basic aqueous layers were both washed with 25 mL EtOAc. All organics were then pooled and dried over MgSO₄, filtered and concentrated to give a red oil. The residue was dissolved in a minimal amount of dichloromethane (˜10 mL), loaded on to a Biotage® SNAP Ultra 360 g silica gel column (Isolera™ Flash System; Biotage AB, Sweden) for purification (20-100% EtOAc in hexanes over 10 column volumes). Fractions containing pure product were pooled to recover 7.9 g of foamy white solid. Impure fractions were subjected to a second purification on a Biotage® SNAP Ultra 100 g silica gel column and pooled with pure product to recover the title compound as a white foam solid (8.390 g, 88.3%). MS m/z obs.=634.7 (M+1).

### 7.4 (2R,3R)-3-((S)-1-((3R,4S,5S)-4-((S)-2-(((benzyloxy)carbonyl)amino)-N,3-dimethyl butanamido)-3-methoxy-5-methylheptanoyl)pyrrolidin-2-yl)-3-methoxy-2-methylpropanoic Acid (Compound 5)

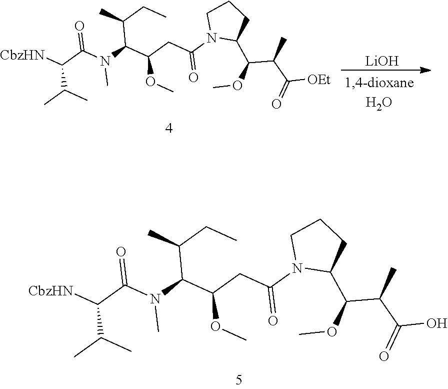 [SMILES: CCOC(=O)[C@H](C)[C@@H](OC)[C@@H]1CCCN1C(=O)C[C@@H](OC)[C@H]([C@@H](C)CC)N(C)C(=O)[C@@H](NC(=O)OCc1ccccc1)C(C)C ; "LiOH" ; CC[C@H](C)[C@@H]([C@@H](CC(=O)N1CCC[C@H]1[C@H](OC)[C@@H](C)C(=O)O)OC)N(C)C(=O)[C@@H](NC(=O)OCc1ccccc1)C(C)C]

To a stirred solution of Compound 4 (8.390 g, 13.24 mmol) in 1,4-dioxane (158 mL) was added dH₂O (39.7 ml) and lithium hydroxide monohydrate (1 M in H₂O, 39.7 mL, 3 equiv). The reaction was stirred at 4° C. and monitored by HPLC-MS for consumption of starting material, which took 3 days until only trace Compound 4 remained. During the course of the reaction, a new product, corresponding to loss of methanol (β-elimination, <2%) formed in small percentages in addition to the desired material. The reaction was acidified with the addition of 1 M aqueous HCl (50 mL) and concentrated under reduced pressure to remove the dioxane. The remaining reaction mixture was extracted with ethyl acetate (4×50 mL) and the organic phase was pooled, washed with brine (15 mL+2 mL 2 M HCl), dried over MgSO₄, filtered and concentrated under reduced pressure to yield a light colored oil. The oil was re-dissolved in diethyl ether (˜50 mL) and concentrated under reduced pressure (3×) to facilitate the removal of residual dioxane, affording the title product as a stiff oil (7.81 g 97% yield with some residual dioxane and Compound 4). MS m/z obs.=606.7 (M+1).

### 7.5 Benzyl ((S)-1-(a3R,4S,5S)-3-methoxy-1-((S)-2-((1R,2R)-1-methoxy-2-methyl-3-oxo-3-((4-(2,2,2-trifluoroacetamido)phenyl)sulfonamido)propyl)pyrrolidin-1-yl)-5-methyl-1-oxoheptan-4-yl)(methyl)amino)-3-methyl-1-oxobutan-2-yl)carbamate (Compound 7)

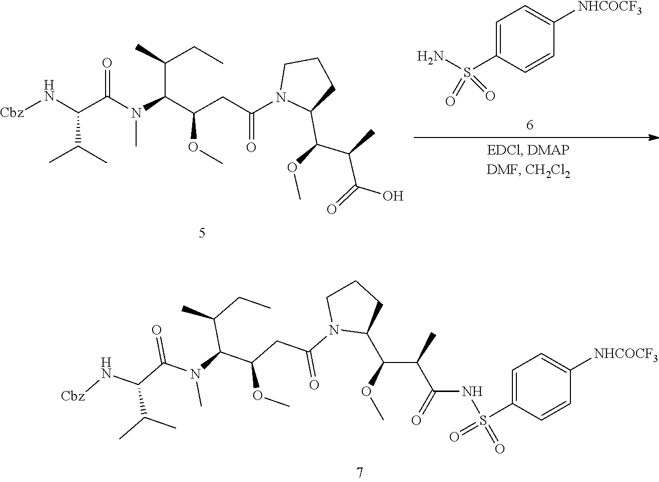

Compound 6 was prepared as described in International Patent Application Publication No. WO 2016/041082.

To a stirred solution of Compound 5 (7.12 g, 11.754 mmol) in dichloromethane (20 mL) was added 2,2,2-trifluoro-N-(4-sulfamoylphenyl)acetamide (Compound 6, 4.095 g, 1.3 equiv, dissolved in 3 mL DMF), N,N-dimethylpyridine (1.867 g, 1.3 equiv) and N,N-dimethylformamide (1.5 mL) to generate a light yellow suspension. Further addition of 5 mL of DMF did not clarify the solution. N-(3-Dimethylaminopropyl)-N′-ethylcarbodiimide hydrochloride (EDCI) (2.817 g, 1.25 equiv) was added in a single portion and the reaction was monitored by HPLC-MS. After 48 hr, reaction was no longer progressing and an additional 400 mg of EDCI was added. After 18 hr, no remaining starting material was observed and the reaction was concentrated under reduced pressure to give a yellow oil. The oil was dissolved in ethyl acetate (˜150 mL) and 1 M HCl (20 mL), and the organic phase was washed with cold 2 M HCl (2×10 mL), saturated NaHCO₃ (1×10 mL), brine (20 mL+5 mL 2 M HCl). Acidic and basic aqueous fractions were extracted with EtOAc (1×20 mL), all organic fractions were pooled, dried over MgSO₄ and concentrated under reduced pressure to yield an oily crude solid (13 g). The residue was dissolved in dichloromethane (˜10 mL), loaded on to a Biotage® SNAP Ultra 360 g silica gel column and purified under a 10-100% EtOAc (2% AcOH) in hexanes gradient over 12 column volumes with a 3-column volume plateau at 50% EtOAc. Fractions containing the pure product were pooled, concentrated under reduced pressure, dissolved and concentrated from toluene (2×10 mL) and diethyl ether (2×10 mL) to afford the desired product, 7.1 g of white foam solid. Impure fractions were subjected to repeat purification under shallower gradient conditions using a Biotage® SNAP Ultra 100 g silica gel column on an Isolera™ instrument. All pure fractions were pooled to recover pure product (the title compound) as a white foam solid (8.60 g, 86%). MS m/z obs.=856.7 (M+1).

### 7.6 (S)-2-amino-N-((3R,4S,5S)-3-methoxy-1-((S)-2-((1R,2R)-1-methoxy-2-methyl-3-oxo-3-((4-(2,2,2-trifluoroacetamido)phenyl)sulfonamido)propyl)pyrrolidin-1-yl)-5-methyl-1-oxoheptan-4-yl)-N,3-dimethylbutanamide (Compound 7a)

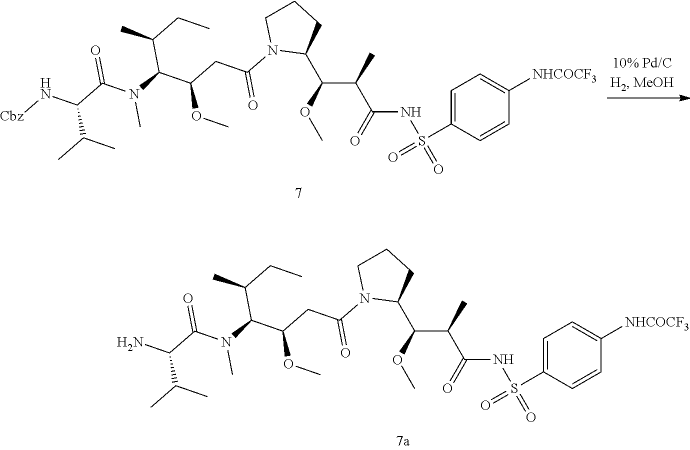

Compound 7 (3.71 g, 4.33 mmol) was dissolved in 10% N,N-dimethylformamide in ethyl acetate (30 mL) in a round bottom flask containing a magnetic stirrer and fitted with a 3-way gas line adapter. The vessel was twice evacuated under reduced pressure and charged with nitrogen gas. 10% palladium on carbon (0.461 g, 0.1 equiv) was added in a single portion, the 3-way adapter was fitted to the flask, a hydrogen balloon was fitted to the adapter and the vessel twice evacuated under reduced pressure and charged with hydrogen. The reaction was allowed to stir for 2 days, over which time the hydrogen balloon was occasionally recharged. After approximately 48 h, HPLC-MS analysis indicated that no starting material remained. The reaction was diluted with methanol (20 mL) and filtered through a plug of celite. The celite was washed with methanol (2×50 mL). All filtrates were pooled and concentrated under reduced pressure and the resulting oil dissolved and concentrated from dichloromethane. After drying under reduced pressure, the title compound was isolated as a colorless powder (3.10 g, 99%). MS m/z obs.=722.6 (M+1).

### 7.7 (S)-2-((S)-2-(dimethylamino)-3-methylbutanamido)-N-((3R,4S,5S)-3-methoxy-1-((S)-2-((1R,2R)-1-methoxy-2-methyl-3-oxo-3-((4-(2,2,2-trifluoroacetamido)phenyl)sulfonamido) propyl)pyrrolidin-1-yl)-5-methyl-1-oxoheptan-4-yl)-N,3-dimethylbutanamide (Compound 8)

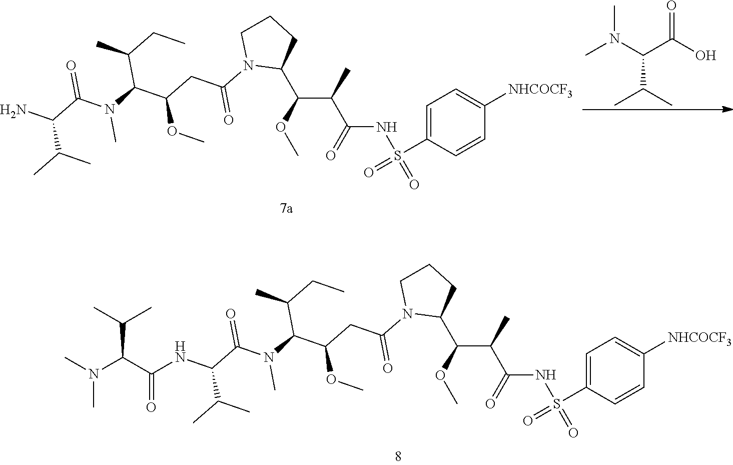

To a stirred solution of N,N-(L)-dimethylvaline (1.696 g, 9.35 mmol) in N,N-dimethylformamide (10 mL) was added HATU (3.216 g, 8.46 mmol) and di-isopropylethylamine (3.10 mL, 17.8 mmol). A clear yellow solution resulted after 5 minutes. Stirring was continued for an additional 10 minutes, then Compound 7a (3.213 g, 4.45 mmol) was added in a single portion. After an additional 1 h of stirring, HPLC-MS indicated that trace amounts of Compound 7a remained and the reaction was for 16 h. The reaction was then concentrated under reduced pressure, diluted with ethyl acetate (120 mL) and 40 mL 1:1 NaHCO₃ (sat.): 5% LiCl and transferred to a separating funnel. The aqueous layer was removed and the organic phase was washed with LiCl (1×20 mL), NaHCO₃ (sat., 2×20 mL). Aqueous layers were pooled and extracted with EtOAc (3×50 mL). Organic layers were pooled and washed with brine (1×20 mL), dried over sodium sulfate, filtered and concentrated to give a DMF-laden oil which was concentrated via rotary evaporator to remove residual DMF, yielding 7 g of crude straw colored oil. The oil was dissolved in a minimal amount of 10% methanol in dichloromethane (˜11 mL) and loaded onto a Biotage® SNAP Ultra 360 g silica gel column for purification (2-20% MeOH in CH₂Cl₂ over 15 column volumes, product eluting around 10-13%). The fractions containing the desired product were pooled and concentrated under reduced pressure to afford the title compound as a colorless foam. Impure fractions were combined, evaporated and subjected to repeat purification on a Biotage® SNAP Ultra 100 g silica gel column on an Isolera™ instrument and combined with the pure product from the first column to yield a colorless foam solid (3.78 g). MS m/z obs.=850.6 (M+1).

### 7.8 (S)—N-((3R,4S,5R)-1-((S)-2-((1R,2R)-3-((4-aminophenyl)sulfonamido)-1-methoxy-2-methyl-3-oxopropyl)pyrrolidin-1-yl)-3-methoxy-5-methyl-1-oxoheptan-4-yl)-2-((S)-2-(dimethylamino)-3-methylbutanamido)-N,3-dimethylbutanamide (Compound 9)

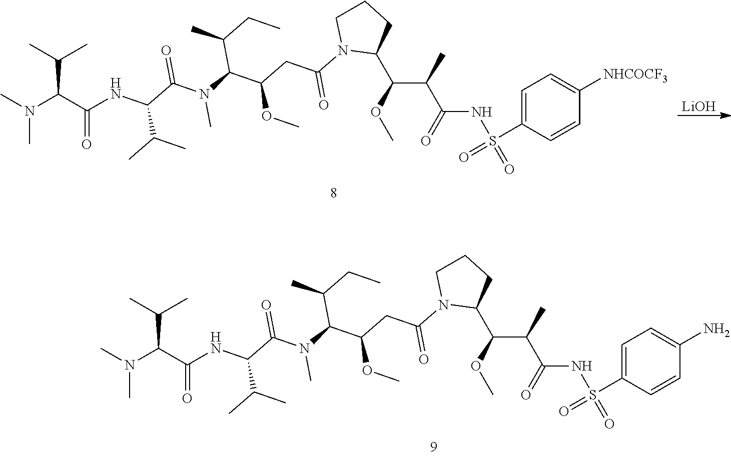

To a stirred solution of Compound 8 (0.980 g, 1.154 mmol) in 1,4-dioxanes (15 mL) was added water (3.5 mL) and 1 M lithium hydroxide monohydrate (3 equiv., 3.46 mL). The resulting light suspension was allowed to stir at 4° C. and was monitored by HPLC-MS for consumption of the starting material. When the conversion was complete (˜5 days), the reaction was neutralized with 3.46 mL of 1 M HCl and concentrated under reduced pressure to remove dioxane. The resulting aqueous phase was diluted with 60 mL EtOAc and 5 mL brine, then extracted with ethyl acetate (2×30 mL). The organic fractions were pooled, dried over Na₂SO₄, filtered and evaporated to yield the title compound as a tan solid (0.930 g). R_{f}=0.5 (8% MeOH in CH₂Cl₂). MS m/z obs.=753.7 (M+1).

### 7.9 2,3,5,6-tetrafluorophenyl 3-(2-(2-(2-(2,5-dioxo-2,5-dihydro-1H-pyrrol-1-yl)ethoxy)ethoxy)ethoxy)propanoate (Compound 15)

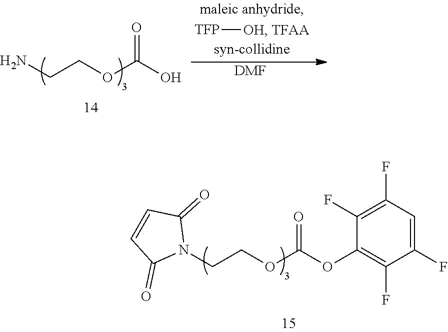

In a dried 50 mL conical flask, 3-(2-(2-(2-aminoethoxy)ethoxy)ethoxy)propanoic acid (Compound 14, 1.000 g, 4.52 mmol) and maleic anhydride (0.443 g, 4.52 mmol) were dissolved in anhydrous N,N-dimethylformamide (5 mL). The reaction was stirred at room temperature for 6 hr under N₂, then cooled to 0° C. and syn-collidine (1.263 mL, 2.1 eq) was added dropwise. In a separate dried 50 mL conical flask, tetrafluorophenol (3.002 g, 4 eq) was dissolved in anhydrous N,N-dimethylformamide (10 mL). The flask was cooled to 0° C. in an ice bath and trifluoroacetic anhydride (2.548 mL, 4 eq) was added dropwise. After stirring for 15 minutes, syn-collidine (2.407 mL, 4 eq) was added dropwise. The flask was allowed to stir for another 15 minutes, and then the contents were added to the first flask dropwise, via syringe. The reaction was allowed to warm to room temperature and stirring was continued under N₂. The reaction was monitored by HPLC-MS for the consumption of starting materials. After 6 days, the reaction was complete with the total consumption of Compound 14, leaving only Compound 15 and a small amount (˜5%) of the bis-TFP maleic amide intermediate. The reaction was transferred to a separating funnel, diluted with diethyl ether (75 ml) and washed with 5% LiCl (1×20 mL), 1 M HCl (2×20 mL), sat. NaHCO₃ (5×20 mL) and brine (1×20 mL). The organic layer was dried over Na₂SO₄, filtered and evaporated to give brown crude oil with residual DMF. Crude oil was dissolved in 8 mL of 1:1 DMF:H₂O+0.1% TFA, loaded onto a 60 g Biotage® SNAP Ultra C18 column (Biotage AB, Uppsala, Sweden) and purified under a linear 30-100% gradient of ACN/H₂O+0.1% TFA over 8 column volumes. Pure fractions were pooled and diluted with brine (20 mL), then extracted 3×50 mL Et₂O. Pooled organics were dried over MgSO₄, filtered and evaporated to recover the title compound as a light-yellow oil (1.34 g, 66% yield).

### 7.10 Tert-butyl ((S)-1-(((S)-1-((4-(N-((2R,3R)-3-((S)-1-((3R,4S,5S)-4-((S)-2-((S)-2-(dimethylamino)-3-methylbutanamido)-N,3-dimethylbutanamido)-3-methoxy-5-methylheptanoyl)pyrrolidin-2-yl)-3-methoxy-2-methylpropanoyl)sulfamoyl)phenyl)amino)-1-oxo-5-ureidopentan-2-yl)amino)-3-methyl-1-oxobutan-2-yl)carbamate (Compound 12)

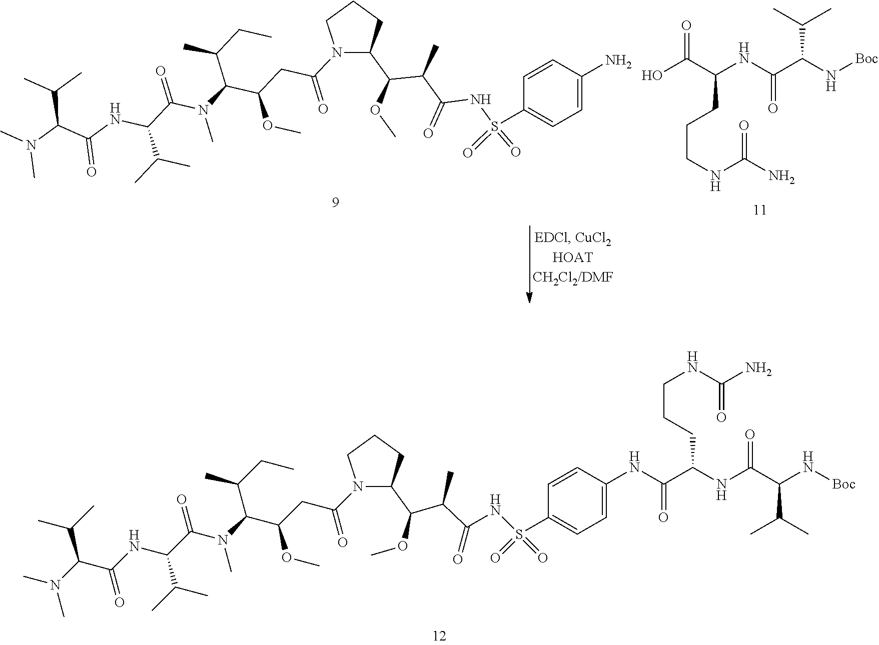 [SMILES: CC[C@H](C)[C@@H]([C@@H](CC(=O)N1CCC[C@H]1[C@H](OC)[C@@H](C)C(=O)NS(=O)(=O)c1ccc(N)cc1)OC)N(C)C(=O)[C@@H](NC(=O)[C@H](C(C)C)N(C)C)C(C)C ; CC(C)[C@H](NC(=O)OC(C)(C)C)C(=O)N[C@@H](CCCNC(N)=O)C(=O)O ; CC[C@H](C)[C@@H]([C@@H](CC(=O)N1CCC[C@H]1[C@H](OC)[C@@H](C)C(=O)NS(=O)(=O)c1ccc(NC(=O)[C@H](CCCNC(N)=O)NC(=O)[C@@H](NC(=O)OC(C)(C)C)C(C)C)cc1)OC)N(C)C(=O)[C@@H](NC(=O)[C@H](C(C)C)N(C)C)C(C)C]

Compound 11 was prepared as described in International Patent Application Publication No. WO 2016/041082.

To an empty 25 mL pear shaped flask, was added Compound 11 (1.342 g, 3.58 mmol, 3.0 equiv), 1-ethyl-3-(3-dimethylaminopropyl)carbodiimide hydrochloride (0.664 g, 3.46 mmol, 2.9 equiv) and 7-hydroxy-azabenzotriazole (HOAT) (0.472 g, 3.46 mmol, 2.9 equiv). These solids were dissolved in a mixture of N,N-dimethylformamide (0.5 mL) and dichloromethane (4.5 mL) with stirring at room temperature over 30 minutes. Separately, Compound 9 (0.900 g, 1.20 mmol) was dissolved in a mixture of N,N-dimethylformamide (0.2 mL) and dichloromethane (1.8 mL) and added to the pear shaped flask, rinsing with dichloromethane (1.0 mL). Stirring rate was increased to 1000 rpm, producing a vortex. Within 2 minutes of adding Compound 9, copper (II) chloride (0.514 g, 3.83 mmol, 3.2 equiv) was added in one portion directly into the center of the vortex through a narrow powder funnel. The initially light-yellow solution turned to a dark-brown suspension which changed over 10 minutes to a dark-green suspension. The reaction was monitored for completion by HPLC-MS and no change to reaction progress was observed between the samples taken at 30 minutes and 1 h (˜95% complete). The reaction was allowed to stir overnight at room temperature, then 2-(2-aminoethylamino)ethanol (0.483 mL, 4.781 mmol, 4 equiv), EtOAc (10 mL) and dH₂O (5 mL) were added to the stirred suspension, which underwent a color change to deep blue. The suspension was stirred vigorously for 4 hr as the suspended solids gradually dissolved into the biphasic mixture. This mixture was transferred to a separating funnel and diluted with EtOAc (100 mL) and brine (10 mL), and the aqueous layer was extracted using 10% IpOH/EtOAc (4×50 mL). The organic layers were pooled and washed with brine (10 mL), dried over Na₂SO₄, and evaporated to yield a faintly blue crude solid. This crude solid was dissolved in a mixture of methanol (0.5 mL) and dichloromethane (6 mL) and purified on a Biotage® SNAP Ultra 100 g silica gel column (2-20% MeOH in CH₂Cl₂ over 10 column volumes, followed by an 8-column volume plateau at 20% MeOH). The product eluted as a broad peak after 1-2 column volumes at ˜20% MeOH in CH₂Cl₂. Fractions containing the desired material were pooled and concentrated under reduced pressure to give the title compound as a white solid (1.105 g, 83%). MS m/z obs.=555.9 ((M+2)/2), 1109.8 (M+1).

### 7.11 (S)-2-((S)-2-amino-3-methylbutanamido)-N-(4-(N-((2R,3R)-3-((S)-1-((3R,4S,5R)-4-((S)-2-((S)-2-(dimethylamino)-3-methylbutanamido)-N,3-dimethylbutanamido)-3-methoxy-5-methylheptanoyl)pyrrolidin-2-yl)-3-methoxy-2-methylpropanoyl)sulfamoyl)phenyl)-5-ureidopentanamide (Compound 13)

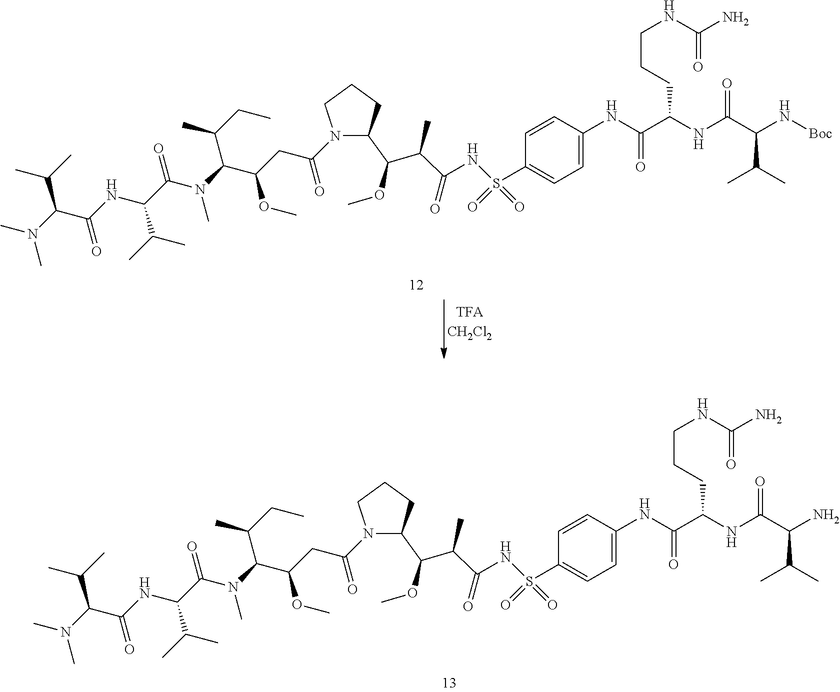 [SMILES: CC[C@H](C)[C@@H]([C@@H](CC(=O)N1CCC[C@H]1[C@H](OC)[C@@H](C)C(=O)NS(=O)(=O)c1ccc(NC(=O)[C@H](CCCNC(N)=O)NC(=O)[C@@H](NC(=O)OC(C)(C)C)C(C)C)cc1)OC)N(C)C(=O)[C@@H](NC(=O)[C@H](C(C)C)N(C)C)C(C)C ; CC[C@H](C)[C@@H]([C@@H](CC(=O)N1CCC[C@H]1[C@H](OC)[C@@H](C)C(=O)NS(=O)(=O)c1ccc(NC(=O)[C@H](CCCNC(N)=O)NC(=O)[C@@H](N)C(C)C)cc1)OC)N(C)C(=O)[C@@H](NC(=O)[C@H](C(C)C)N(C)C)C(C)C]

To a solution of Compound 12 (0.926 g, 0.834 mmol) was added a mixture of dichloromethane (10 mL) and trifluoroacetic acid (2.0 mL). The reaction was monitored by HPLC-MS for consumption of starting material (˜45 minutes). The reaction was co-evaporated with acetonitrile (2×10 mL) and dichloromethane (2×10 mL) under reduced pressure to remove excess trifluoroacetic acid. The resulting residue was dissolved in a minimal amount of dichloromethane and methanol (3:1, v/v, ˜2 mL), and added to a stirred solution of diethyl ether (200 mL) and hexanes (100 mL) dropwise via pipette, producing a suspension of light white solids. The solids were filtered and dried under vacuum to afford the title compound in the form of a white powder, as the trifluoroacetate salt (1.04 g, quantitative yield with some residual solvents). MS m/z obs.=505.8 ((M+2)/2).

### 7.12 (S)—N-(4-(N-((2R,3R)-3-((S)-1-((3R,4S,5R)-4-((S)-2-((S)-2-(dimethylamino)-3-methylbutanamido)-N,3-dimethylbutanamido)-3-methoxy-5-methylheptanoyl)pyrrolidin-2-yl)-3-methoxy-2-methylpropanoyl)sulfamoyl)phenyl)-2-((S)-1-(2,5-dioxo-2,5-dihydro-1H-pyrrol-1-yl)-14-isopropyl-12-oxo-3,6,9-trioxa-13-azapentadecanamido)-5-ureidopentanamide (Linker-Toxin A)

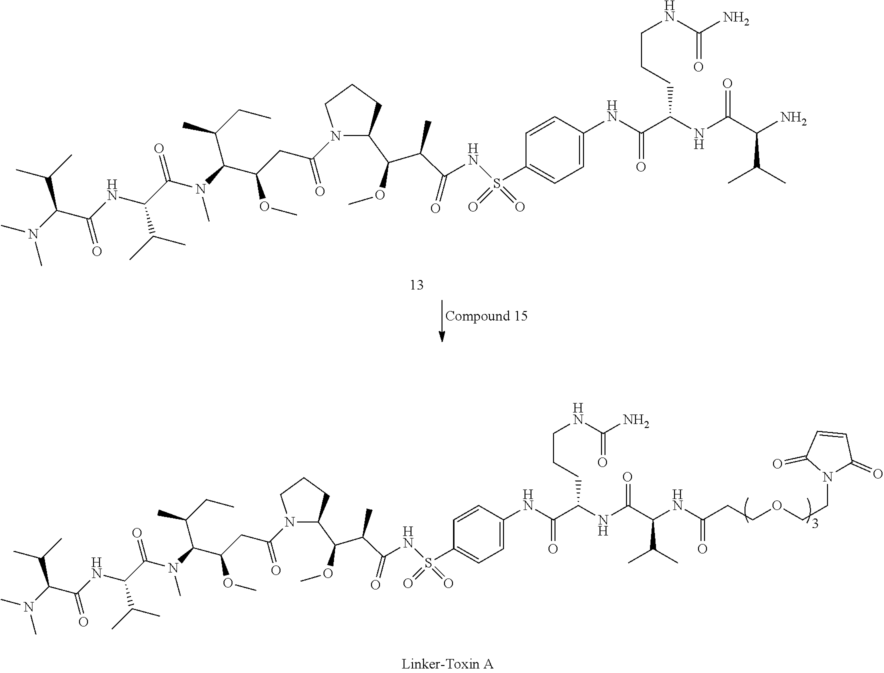

To a stirred solution of Compound 13 (0.722 g, 0.584 mmol) in N,N-dimethylformamide (4 mL) was added Compound 15 (0.314 g, 1.2 equiv) and diisopropylethylamine (0.305 mL, 3.0 equiv). HPLC-MS analysis at 2 h indicated no remaining starting material. The reaction was acidified with TFA (300 μL) and then diluted with diH₂O+0.1% TFA (9 mL). The resultant solution was loaded onto a 120 g Biotage® SNAP Ultra C18 column (Biotage, Uppsala, Sweden) and purified under an ACN/H₂O+0.1% TFA gradient: 20-60% ACN over 10 column volumes, 60-100% ACN over 5 column volumes. Product eluted near 40% ACN. Pure fractions as identified by LCMS were pooled and lyophilized. A white powder solid was recovered from the lyophilizer. The lyophilization was repeated at higher concentration (approx. 50 mg/mL in 2:1 H₂O/ACN) into a vial to produce the title compound as a denser, less flocculant lyophilized solid (754.2 mg, 91%). MS m/z obs.=647.4 ((M+2)/2), 1292.8 (M+1).

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### Sequences

TABLE 5
Variable region sequences

| Clone | VH Domains (SEQ ID NO) | VL Domains (SEQ ID NO) |
|---|---|---|
| 25A | QVQLVQSGAEVKKPGASVKVSCKASGYTF | DIQMTQSPSTLSASVGDRVTITCRASQSISS |
| | DVYGISWVRQAPGQGLEWMGWIAPYNGN | WLAWYQQKPGKAPKLLIYKASSLESGVPS |
| | TNYAQKLQGRVTMTTDTSTSTAYMELRSL | RFSGSGSGTEFTLTISSLQPDDFATYYCQQF |
| | RSDDTAVYYCARDAGTYSPFGYGMDVWG | QSLPPFTFGGGTKVEIK (SEQ ID NO: 114) |
| | QGTTVTVSS (SEQ ID NO: 113) | |
| 25A3 | QVQLVQSGAEVKKPGASVKVSCKASGYTF | DIQMTQSPSTLSASVGDRVTITCQASQSINN |
| | DVYGISWVRQAPGQGLEWMGWIAPYSGN | WLAWYQQKPGKAPKLLIYKAYNLESGVPS |
| | TNYAQKLQGRVTMTTDTSTSTAYMELRSL | RFSGSGSGTEFTLTISSLQPDDFATYYCQLF |
| | RSDDTAVYYCARDAGTYSPFGYGMDVWG | QSLPPFTFGGGTKVEIK (SEQ ID NO: 152) |
| | QGTTVTVSS (SEQ ID NO: 151) | |
| 25A5 | QVQLVQSGAEVKKPGASVKVSCKASGYTF | DIQMTQSPSTLSASVGDRVTITCRASESISN |
| | DVYGISWVRQAPGQGLEWMGWIAPYSGN | WLAWYQQKPGKAPKLLIYKAYSLEYGVPS |
| | TNYAQKLQGRVTMTTDTSTSTAYMELRSL | RFSGSGSGTEFTLTISSLQPDDFATYYCQQF |
| | RSDDTAVYYCARDAGTYSPFGYGMDVWG | QKLPPFTFGGGTKVEIK (SEQ ID NO: 190) |
| | QGTTVTVSS (SEQ ID NO: 189) | |
| 25A5- | QVQLVQSGAEVKKPGASVKVSCKASGYTF | DIQMTQSPSTLSASVGDRVTITCRASESISN |
| T | DAYGISWVRQAPGQGLEWMGWIAPYSGN | WLAWYQQKPGKAPKLLIYKAYSLEYGVPS |
| | TNYAQKLQGRVTMTTDTSTSTAYMELRSL | RFSGSGSGTEFTLTISSLQPDDFATYYCQQF |
| | RSDDTAVYYCARDAGTYSPFGYGMDVWG | QKLPPFTFGGGTKVEIK (SEQ ID NO: 837) |
| | QGTTVTVSS (SEQ ID NO: 836) | |
| 25G | QVQLVQSGAEVKKPGASVKVSCKASGYTF | DIQMTQSPSTLSASVGDRVTITCRASQSISS |
| | RSYGISWVRQAPGQGLEWMGWVAPYNG | WLAWYQQKPGKAPKLLIYKASSLESGVPS |
| | NTNYAQKLQGRVTMTTDTSTSTAYMELRS | RFSGSGSGTEFTLTISSLQPDDFATYYCQQF |
| | LRSDDTAVYYCARDAGTYSPYGYGMDVW | QSLPPFTFGGGTKVEIK (SEQ ID NO: 228) |
| | GQGTTVTVSS (SEQ ID NO: 227) | |
| 25G1 | QVQLVQSGAEVKKPGASVKVSCKASGYTF | DIQMTQSPSTLSASVGDRVTITCRASHSIDS |
| | RSYGISWVRQAPGQGLEWMGWVAPYSGN | WLAWYQQKPGKAPKLLIYKASYLESGVPS |
| | TNYAQKLQGRVTMTTDTSTSTAYMELRSL | RFSGSGSGTEFTLTISSLQPDDFATYYCQLF |
| | RSDDTAVYYCARDAGTYSPYGYGMDVW | QSLPPFTFGGGTKVEIK (SEQ ID NO: 266) |
| | GQGTTVTVSS (SEQ ID NO: 265) | |
| 25G9 | QVQLVQSGAEVKKPGASVKVSCKASGYTF | DIQMTQSPSTLSASVGDRVTITCQASQSIDS |
| | RSYGISWVRQAPGQGLEWMGWVAPYSGN | WLAWYQQKPGKAPKLLIYSASYLESGVPS |
| | TNYAQKLQGRVTMTTDTSTSTAYMELRSL | RFSGSGSGTEFTLTISSLQPDDFATYYCQRF |
| | RSDDTAVYYCARDAGTYSPYGYGMDVW | QSLPPFTFGGGTKVEIK (SEQ ID NO: 304) |
| | GQGTTVTVSS (SEQ ID NO: 303) | |

TABLE 6
Variable region sequence consensus

| Group | VH Domain Consensus (SEQ ID NO) | VL Domain Consensus (SEQ ID NO) |
|---|---|---|
| 25 | QVQLVQSGAEVKKPGASVKVSCKASGYT | DIQMTQSPSTLSASVGDRVTITCx[R/Q]ASx |
| | Fx[D/R]x[S/V/A]YGISWVRQAPGQGLEWM | [Q/E/H]SIx[S/D/N]x[S/N]WLAWYQQKPGK |
| | GWx[I/V]APYx[S/N]GNTNYAQKLQGRVT | APKLLIYx[K/S]Ax[S/Y]x[S/Y/N]LEx[S/Y]G |
| | MTTDTSTSTAYMELRSLRSDDTAVYYCAR | VPSRFSGSGSGTEFTLTISSLQPDDFATYYC |
| | DAGTYSPx[F/Y]GYGMDVWGQGTTVTVSS | Qx[Q/L/R]FQx[S/K]LPPFTFGGGTKVEIK |
| | (SEQ ID NO: 763) | (SEQ ID NO: 764) |

TABLE 7
Antibody 25A-CDR Sequences

| | | Exemplary* | Kabat | Chothia | AbM | Contact | IMGT |
|---|---|---|---|---|---|---|---|
| VH | VH | GYTFDVYGIS | VYGIS | GYTFDVY | GYTFDVYGIS | DVYGIS | GYTFDVYG |
| CDR | CDR1 | (SEQ ID NO: 77) | (SEQ ID NO: 83) | (SEQ ID NO: 89) | (SEQ ID NO: 95) | (SEQ ID NO: 101) | (SEQ ID NO: 107) |
| Seq. | VH | WIAPYNGNTNYA | WIAPYNGNTNYA | PYNG | WIAPYNGNTN | WMGWIAPYNGNT | IAPYNGNT |
| | CDR2 | QKLQG | QKLQG | (SEQ ID NO: 90) | (SEQ ID NO: 96) | N | (SEQ ID NO: 108) |
| | | (SEQ ID NO: 78) | (SEQ ID NO: 84) | | | (SEQ ID NO: 102) | |
| | VH | DAGTYSPFGYGM | DAGTYSPFGYGM | AGTYSPFGYGMD | DAGTYSPFGYGM | ARDAGTYSPFGYG | ARDAGTYSPFGTG |
| | CDR3 | DV | DV | (SEQ ID NO: 91) | DV | MD | MDV |
| | | (SEQ ID NO: 79) | (SEQ ID NO: 85) | | (SEQ ID NO: 97) | (SEQ ID NO: 103) | (SEQ ID NO: 109) |
| VL | VL | RASQSISSWLA | RASQSISSWLA | SQSISSW | RASQSISSWLA | SSWLAWY | QSISSW |
| CDR | CDR1 | (SEQ ID NO: 80) | (SEQ ID NO: 86) | (SEQ ID NO: 92) | (SEQ ID NO: 98) | (SEQ ID NO: 104) | (SEQ ID NO: 110) |
| Seq. | VL | KASSLES | KASSLES | KAS | KASSLES | LLIYKASSLE | KAS |
| | CDR2 | (SEQ ID NO: 81) | (SEQ ID NO: 87) | (SEQ ID NO: 93) | (SEQ ID NO: 99) | (SEQ ID NO: 105) | (SEQ ID NO: 111) |
| | VL | QQFQSLPPFT | QQFQSLPPFT | FQSLPPF | QQFQSLPPFT | QQFQSLPPF | QQFQSLPPFT |
| | CDR3 | (SEQ ID NO: 82) | (SEQ ID NO: 88) | (SEQ ID NO: 94) | (SEQ ID NO: 100) | (SEQ ID NO: 106) | (SEQ ID NO: 112) |

VH Sequence*:
QVQLVQSGAEVKKPGASVKVSCKASGYTFDVYGISWVRQAPGQGLEWMGWIAPYNGNTNYAQKLQGRVTMTTDTSTSTAYMELRSLRSDDTAVYYCARD
AGTYSPFGYGMDVWGQGTTVTVSS (SEQ ID NO: 113)
VL Sequence*:
DIQMTQSPSTLSASVGDRVTITCRASQSISSWLAWYQQKPGKAPKLLIYKASSLESGVPSRFSGSGSGTEFTLTISSLQPDDFATYYCQQFQSLPPFTF
GGGTKVEIK (SEQ ID NO: 114)
*Exemplary CDR sequences encompass amino acids as determined by Kabat plus Chothia

TABLE 8
Antibody 25A3-CDR Sequences

| | | Exemplary* | Kabat | Chothia | AbM | Contact | IMGT |
|---|---|---|---|---|---|---|---|
| VH | VH | GYTFDVYGIS | VYGIS | GYTFDVY | GYTFDVYGIS | DVYGIS | GYTFDVYG |
| CDR | CDR1 | (SEQ ID NO: 115) | (SEQ ID NO: 121) | (SEQ ID NO: 127) | (SEQ ID NO: 133) | (SEQ ID NO: 139) | (SEQ ID NO: |
| Seq. | | | | | | | 145) |
| | VH | WIAPYSGNTNY | WIAPYSGNTNY | PYSG | WIAPYSGNTN | WMGWIAPYSG | IAPYSGNT |
| | CDR2 | AQKLQG | AQKLQG | (SEQ ID NO: 128) | (SEQ ID NO: 134) | NTN | (SEQ ID NO: |
| | | (SEQ ID NO: 116) | (SEQ ID NO: 122) | | | (SEQ ID NO: 140) | 146) |
| | VH | DAGTYSPFGYG | DAGTYSPFGYG | AGTYSPFGYG | DAGTYSPFGYG | ARDAGTYSPFG | ARDAGTYSPFG |
| | CDR3 | MDV | MDV | MD | MDV | YGMD | TGMDV |
| | | (SEQ ID NO: 117) | (SEQ ID NO: 123) | (SEQ ID NO: 129) | (SEQ ID NO: 135) | (SEQ ID NO: 141) | (SEQ ID NO: |
| | | | | | | | 147) |
| VL | VL | QASQSINNWLA | QASQSINNWLA | SQSINNW | QASQSINNWLA | NNWLAWY | QSINNW |
| CDR | CDR1 | (SEQ ID NO: 118) | (SEQ ID NO: 124) | (SEQ ID NO: 130) | (SEQ ID NO: 136) | (SEQ ID NO: 142) | (SEQ ID NO: |
| Seq. | | | | | | | 148) |
| | VL | KAYNLES | KAYNLES | KAY | KAYNLES | LLIYKAYNLE | KAY |
| | CDR2 | (SEQ ID NO: 119) | (SEQ ID NO: 125) | (SEQ ID NO: 131) | (SEQ ID NO: 137) | (SEQ ID NO: 143) | (SEQ ID NO: |
| | | | | | | | 149) |
| | VL | QLFQSLPPFT | QLFQSLPPFT | FQSLPPF | QLFQSLPPFT | QLFQSLPPF | QLFQSLPPFT |
| | CDR3 | (SEQ ID NO: 120) | (SEQ ID NO: 126) | (SEQ ID NO: 132) | (SEQ ID NO: 138) | (SEQ ID NO: 144) | (SEQ ID NO: |
| | | | | | | | 150) |

VH Sequence*:
QVQLVQSGAEVKKPGASVKVSCKASGYTFDVYGISWVRQAPGQGLEWMGWIAPYSGNTNYAQKLQGRVTMTTDTSTSTAYME
LRSLRSDDTAVYYCARDAGTYSPFGYGMDVWGQGTTVTVSS (SEQ ID NO: 151)
VL Sequence*:
DIQMTQSPSTLSASVGDRVTITCQASQSINNWLAWYQQKPGKAPKLLIYKAYNLESGVPSRFSGSGSGTEFTLTISSLQPDDFATYY
CQLFQSLPPFTFGGGTKVEIK (SEQ ID NO: 152)
*Exemplary CDR sequences encompass amino acids as determined by Kabat plus Chothia

TABLE 9
Antibody 25A5-CDR Sequences

| | | Exemplary* | Kabat | Chothia | AbM | Contact | IMGT |
|---|---|---|---|---|---|---|---|
| VH | VH | GYTFDVYGIS | VYGIS | GYTFDVY | GYTFDVYGIS | DVYGIS | GYTFDVYG |
| CDR | CDR2 | (SEQ ID NO: 153) | (SEQ ID NO: 159) | (SEQ ID NO: 165) | (SEQ ID NO: 171) | (SEQ ID NO: 177) | (SEQ ID NO: |
| Seq. | | | | | | | 183) |
| | VH | WIAPYSGNTNY | WIAPYSGNTNY | PYSG | WIAPYSGNTN | WMGWIAPYSG | IAPYSGNT |
| | CDR2 | AQKLQG | AQKLQG | (SEQ ID NO: 166) | (SEQ ID NO: 172) | NTN | (SEQ ID NO: |
| | | (SEQ ID NO: 154) | (SEQ ID NO: 160) | | | (SEQ ID NO: 178) | 184) |
| | VH | DAGTYSPFGYG | DAGTYSPFGYG | AGTYSPFGYG | DAGTYSPFGYG | ARDAGTYSPFG | ARDAGTYSPFG |
| | CDR3 | MDV | MDV | MD | MDV | YGMD | TGMDV |
| | | (SEQ ID NO: 155) | (SEQ ID NO: 161) | (SEQ ID NO: 167) | (SEQ ID NO: 173) | (SEQ ID NO: 179) | (SEQ ID NO: |
| | | | | | | | 185) |
| VL | VL | RASESISNWLA | RASESISNWLA | SESISNW | RASESISNWLA | SNWLAWY | ESISNW |
| CDR | CDR1 | (SEQ ID NO: 156) | (SEQ ID NO: 162) | (SEQ ID NO: 168) | (SEQ ID NO: 174) | (SEQ ID NO: 180) | (SEQ ID NO: |
| Seq. | | | | | | | 186) |
| | VL | KAYSLEY | KAYSLEY | KAY | KAYSLEY | LLIYKAYSLE | KAY |
| | CDR2 | (SEQ ID NO: 157) | (SEQ ID NO: 163) | (SEQ ID NO: 169) | (SEQ ID NO: 175) | (SEQ ID NO: 181) | (SEQ ID NO: |
| | | | | | | | 187) |
| | VL | QQFQKLPPFT | QQFQKLPPFT | FQKLPPF | QQFQKLPPFT | QQFQKLPPF | QQFQKLPPFT |
| | CDR3 | (SEQ ID NO: 158) | (SEQ ID NO: 164) | (SEQ ID NO: 170) | (SEQ ID NO: 176) | (SEQ ID NO: 182) | (SEQ ID NO: |
| | | | | | | | 188) |

VH Sequence*:
QVQLVQSGAEVKKPGASVKVSCKASGYTFDVYGISWVRQAPGQGLEWMGWIAPYSGNTNYAQKLQGRVTMTTDTSTSTAYME
LRSLRSDDTAVYYCARDAGTYSPFGYGMDVWGQGTTVTVSS (SEQ ID NO: 189)
VL Sequence*:
DIQMTQSPSTLSASVGDRVTITCRASESISNWLAWYQQKPGKAPKLLIYKAYSLEYGVPSRFSGSGSGTEFTLTISSLQPDDFATYYC
QQFQKLPPFTFGGGTKVEIK (SEQ ID NO: 190)
*Exemplary CDR sequences encompass amino acids as determined by Kabat plus Chothia

TABLE 10
Antibody 25A5-T-CDR Sequences

| | | Exemplary* | Kabat | Chothia | AbM | Contact | IMGT |
|---|---|---|---|---|---|---|---|
| VH | VH | GYTFDAYGIS | AYGIS | GYTFDAY | GYTFDAYGIS | DAYGIS | GYTFDAYG |
| CDR | CDR1 | (SEQ ID NO: 884) | (SEQ ID NO: 890) | (SEQ ID NO: 896) | (SEQ ID NO: 902) | (SEQ ID NO: 908) | (SEQ ID NO: |
| Seq. | | | | | | | 914) |
| | VH | WIAPYSGNTNY | WIAPYSGNTNY | PYSG | WIAPYSGNTN | WMGWIAPYSG | IAPYSGNT |
| | CDR2 | AQKLQG | AQKLQG | (SEQ ID NO: 897) | (SEQ ID NO: 903) | NTN | (SEQ ID NO: |
| | | (SEQ ID NO: 885) | (SEQ ID NO: 891) | | | (SEQ ID NO: 909) | 915) |
| | VH | DAGTYSPFGYG | DAGTYSPFGYG | AGTYSPFGYG | DAGTYSPFGYG | ARDAGTYSPFG | ARDAGTYSPFG |
| | CDR3 | MDV | MDV | MD | MDV | YGMD | TGMDV |
| | | (SEQ ID NO: 886) | (SEQ ID NO: 892) | (SEQ ID NO: 898) | (SEQ ID NO: 904) | (SEQ ID NO: 910) | (SEQ ID NO: |
| | | | | | | | 916) |
| VL | VL | RASESISNWLA | RASESISNWLA | SESISNW | RASESISNWLA | SNWLAWY | ESISNW |
| CDR | CDR1 | (SEQ ID NO: 887) | (SEQ ID NO: 893) | (SEQ ID NO: 899) | (SEQ ID NO: 905) | (SEQ ID NO: 911) | (SEQ ID NO: |
| Seq. | | | | | | | 917) |
| | VL | KAYSLEY | KAYSLEY | KAY | KAYSLEY | LLIYKAYSLE | KAY |
| | CDR2 | (SEQ ID NO: 888) | (SEQ ID NO: 894) | (SEQ ID NO: 900) | (SEQ ID NO: 906) | (SEQ ID NO: 912) | (SEQ ID NO: |
| | | | | | | | 918) |
| | VL | QQFQKLPPFT | QQFQKLPPFT | FQKLPPF | QQFQKLPPFT | QQFQKLPPF | QQFQKLPPFT |
| | CDR3 | (SEQ ID NO: 889) | (SEQ ID NO: 895) | (SEQ ID NO: 901) | (SEQ ID NO: 907) | (SEQ ID NO: 913) | (SEQ ID NO: |
| | | | | | | | 919) |

VH Sequence*:
QVQLVQSGAEVKKPGASVKVSCKASGYTFDAYGISWVRQAPGQGLEWMGWIAPYSGNTNYAQKLQGRVTMTTDTSTSTAYME
LRSLRSDDTAVYYCARDAGTYSPFGYGMDVWGQGTTVTVSS (SEQ ID NO: 836)
VL Sequence*:
DIQMTQSPSTLSASVGDRVTITCRASESISNWLAWYQQKPGKAPKLLIYKAYSLEYGVPSRFSGSGSGTEFTLTISSLQPDDFATYYC
QQFQKLPPFTFGGGTKVEIK (SEQ ID NO: 837)
*Exemplary CDR sequences encompass amino acids as determined by Kabat plus Chothia

TABLE 11
Antibody 25G-CDR Sequences

| | | Exemplary* | Kabat | Chothia | AbM | Contact | IMGT |
|---|---|---|---|---|---|---|---|
| VH | VH | GYTFRSYGIS | SYGIS | GYTFRSY | GYTFRSYGIS | RSYGIS | GYTFRSYG |
| CDR | CDR1 | (SEQ ID NO: 191) | (SEQ ID NO: 197) | (SEQ ID NO: 203) | (SEQ ID NO: 209) | (SEQ ID NO: 215) | (SEQ ID NO: |
| Seq. | | | | | | | 221) |
| | VH | WVAPYNGNTN | WVAPYNGNTN | PYNG | WVAPYNGNTN | WMGWVAPYN | VAPYNGNT |
| | CDR2 | YAQKLQG | YAQKLQG | (SEQ ID NO: 204) | (SEQ ID NO: 210) | GNTN | (SEQ ID NO: |
| | | (SEQ ID NO: 192) | (SEQ ID NO: 198) | | | (SEQ ID NO: 216) | 222) |
| | VH | DAGTYSPYGY | DAGTYSPYGY | AGTYSPYGYG | DAGTYSPYGY | ARDAGTYSPY | ARDAGTYSPY |
| | CDR3 | GMDV | GMDV | MD | GMDV | GYGMD | GYGMDV |
| | | (SEQ ID NO: 193) | (SEQ ID NO: 199) | (SEQ ID NO: 205) | (SEQ ID NO: 211) | (SEQ ID NO: 217) | (SEQ ID NO: |
| | | | | | | | 223) |
| VL | VL | RASQSISSWLA | RASQSISSWLA | SQSISSW | RASQSISSWLA | SSWLAWY | QSISSW |
| CDR | CDR1 | (SEQ ID NO: 194) | (SEQ ID NO: 200) | (SEQ ID NO: 206) | (SEQ ID NO: 212) | (SEQ ID NO: 218) | (SEQ ID NO: |
| Seq. | | | | | | | 224) |
| | VL | KASSLES | KASSLES | KAS | KASSLES | LLIYKASSLE | KAS |
| | CDR2 | (SEQ ID NO: 195) | (SEQ ID NO: 201) | (SEQ ID NO: 207) | (SEQ ID NO: 213) | (SEQ ID NO: 219) | (SEQ ID NO: |
| | | | | | | | 225) |
| | VL | QQFQSLPPFT | QQFQSLPPFT | FQSLPPF | QQFQSLPPFT | QQFQSLPPF | QQFQSLPPFT |
| | CDR3 | (SEQ ID NO: 196) | (SEQ ID NO: 202) | (SEQ ID NO: 208) | (SEQ ID NO: 214) | (SEQ ID NO: 220) | (SEQ ID NO: |
| | | | | | | | 226) |

VH Sequence*:
QVQLVQSGAEVKKPGASVKVSCKASGYTFRSYGISWVRQAPGQGLEWMGWVAPYNGNTNYAQKLQGRVTMTTDTSTSTAYM
ELRSLRSDDTAVYYCARDAGTYSPYGYGMDVWGQGTTVTVSS (SEQ ID NO: 227)
VL Sequence*:
DIQMTQSPSTLSASVGDRVTITCRASQSISSWLAWYQQKPGKAPKLLIYKASSLESGVPSRFSGSGSGTEFTLTISSLQPDDFATYYC
QQFQSLPPFTFGGGTKVEIK (SEQ ID NO: 228)
*Exemplary CDR sequences encompass amino acids as determined by Kabat plus Chothia

TABLE 12
Antibody 25G1-CDR Sequences

| | | Exemplary* | Kabat | Chothia | AbM | Contact | IMGT |
|---|---|---|---|---|---|---|---|
| VH | VH | GYTFRSYGIS | SYGIS | GYTFRSY | GYTFRSYGIS | RSYGIS | GYTFRSYG |
| CDR | CDR1 | (SEQ ID NO: 229) | (SEQ ID NO: 235) | (SEQ ID NO: 241) | (SEQ ID NO: 247) | (SEQ ID NO: 253) | (SEQ ID NO: |
| Seq. | | | | | | | 259) |
| | VH | WVAPYSGNTN | WVAPYSGNTN | PYSG | WVAPYSGNT | WMGWVAPYS | VAPYSGNT |
| | CDR2 | YAQKLQG | YAQKLQG | (SEQ ID NO: 242) | N | GNTN | (SEQ ID NO: |
| | | (SEQ ID NO: 230) | (SEQ ID NO: 236) | | (SEQ ID NO: 248) | (SEQ ID NO: 254) | 260) |
| | VH | DAGTYSPYGY | DAGTYSPYGY | AGTYSPYGYG | DAGTYSPYGY | ARDAGTYSPY | ARDAGTYSPY |
| | CDR3 | GMDV | GMDV | MD | GMDV | GYGMD | GYGMDV |
| | | (SEQ ID NO: 231) | (SEQ ID NO: 237) | (SEQ ID NO: 243) | (SEQ ID NO: 249) | (SEQ ID NO: 255) | (SEQ ID NO: |
| | | | | | | | 261) |
| VL | VL | RASHSIDSWLA | RASHSIDSWLA | SHSIDSW | RASHSIDSWLA | DSWLAWY | HS IDSW |
| CDR | CDR1 | (SEQ ID NO: 232) | (SEQ ID NO: 238) | (SEQ ID NO: 244) | (SEQ ID NO: 250) | (SEQ ID NO: 256) | (SEQ ID NO: |
| Seq. | | | | | | | 262) |
| | VL | KASYLES | KASYLES | KAS | KASYLES | LLIY KASYLE | KAS |
| | CDR2 | (SEQ ID NO: 233) | (SEQ ID NO: 239) | (SEQ ID NO: 245) | (SEQ ID NO: 251) | (SEQ ID NO: 257) | (SEQ ID NO: |
| | | | | | | | 263) |
| | VL | QLFQSLPPFT | QLFQSLPPFT | FQSLPPF | QLFQSLPPFT | QLFQSLPPF | QLFQSLPPFT |
| | CDR3 | (SEQ ID NO: 234) | (SEQ ID NO: 240) | (SEQ ID NO: 246) | (SEQ ID NO: 252) | (SEQ ID NO: 258) | (SEQ ID NO: |
| | | | | | | | 264) |

VH Sequence*:
QVQLVQSGAEVKKPGASVKVSCKASGYTFRSYGISWVRQAPGQGLEWMGWVAPYSGNTNYAQKLQGRVTMTTDTSTSTAYME
LRSLRSDDTAVYYCARDAGTYSPYGYGMDVWGQGTTVTVSS (SEQ ID NO: 265)
VL Sequence*:
DIQMTQSPSTLSASVGDRVTITCRASHSIDSWLAWYQQKPGKAPKLLIYKASYLESGVPSRFSGSGSGTEFTLTISSLQPDDFATYYC
QLFQSLPPFTFGGGTKVEIK (SEQ ID NO: 266)
*Exemplary CDR sequences encompass amino acids as determined by Kabat plus Chothia

TABLE 13
Antibody 25G9-CDR Sequences

| | | Exemplary* | Kabat | Chothia | AbM | Contact | IMGT |
|---|---|---|---|---|---|---|---|
| VH | VH | GYTFRSYGIS | SYGIS | GYTFRSY | GYTFRSYGIS | RSYGIS | GYTFRSYG |
| CDR | CDR1 | (SEQ ID NO: 267) | (SEQ ID NO: 273) | (SEQ ID NO: 279) | (SEQ ID NO: 285) | (SEQ ID NO: 291) | (SEQ ID NO: |
| Seq. | | | | | | | 297) |
| | VH | WVAPYSGNT | WVAPYSGNTN | PYSG | WVAPYSGNTN | WMGWVAPYS | VAPYSGNT |
| | CDR2 | NYAQKLQG | YAQKLQG | (SEQ ID NO: 280) | (SEQ ID NO: 286) | GNTN | (SEQ ID NO: |
| | | (SEQ ID NO: 268) | (SEQ ID NO: 274) | | | (SEQ ID NO: 292) | 298) |
| | VH | DAGTYSPYGY | DAGTYSPYGY | AGTYSPYGYG | DAGTYSPYGY | ARDAGTYSPYG | ARDAGTYSPYG |
| | CDR3 | GMDV | GMDV | MD | GMDV | YGMD | YGMDV |
| | | (SEQ ID NO: 269) | (SEQ ID NO: 275) | (SEQ ID NO: 281) | (SEQ ID NO: 287) | (SEQ ID NO: 293) | (SEQ ID NO: |
| | | | | | | | 299) |
| VL | VL | QASQSIDSWLA | QASQSIDSWLA | SQSIDSW | QASQSIDSWLA | DSWLAWY | QSIDSW |
| CDR | CDR1 | (SEQ ID NO: 270) | (SEQ ID NO: 276) | (SEQ ID NO: 282) | (SEQ ID NO: 288) | (SEQ ID NO: 294) | (SEQ ID NO: |
| Seq. | | | | | | | 300) |
| | VL | SASYLES | SASYLES | SAS | SASYLES | LLIYSASYLE | SAS |
| | CDR2 | (SEQ ID NO: 271) | (SEQ ID NO: 277) | (SEQ ID NO: 283) | (SEQ ID NO: 289) | (SEQ ID NO: 295) | (SEQ ID NO: |
| | | | | | | | 301) |
| | VL | QRFQSLPPFT | QRFQSLPPFT | FQSLPPF | QRFQSLPPFT | QRFQSLPPF | QRFQSLPPFT |
| | CDR3 | (SEQ ID NO: 272) | (SEQ ID NO: 278) | (SEQ ID NO: 284) | (SEQ ID NO: 290) | (SEQ ID NO: 296) | (SEQ ID NO: |
| | | | | | | | 302) |

VH Sequence*:
QVQLVQSGAEVKKPGASVKVSCKASGYTFRSYGISWVRQAPGQGLEWMGWVAPYSGNTNYAQKLQGRVTMTTDTSTSTAYME
LRSLRSDDTAVYYCARDAGTYSPYGYGMDVWGQGTTVTVSS (SEQ ID NO: 303)
VL Sequence*:
DIQMTQSPSTLSASVGDRVTITCQASQSIDSWLAWYQQKPGKAPKLLIYSASYLESGVPSRFSGSGSGTEFTLTISSLQPDDFATYYC
QRFQSLPPFTFGGGTKVEIK (SEQ ID NO: 304)
*Exemplary CDR sequences encompass amino acids as determined by Kabat plus Chothia

TABLE 14
Consensus CDRs

| Antibody | | | | | | | |
|---|---|---|---|---|---|---|---|
| Group | | 1 | 25 | 29 | 39 | 43 | 54 |
| VH | VH | GFTFSx[D/S]YA | GYTFx[D/R]x[S/ | GFTFx[H/R]Sx[R/ | GGTFSSNAIG | GGSx[I/L]SSGx | GYTFANYYMH |
| CDR | CDR1 | Mx[A/G] | V]YGIS | Y]GMH | (SEQ ID NO: 791) | [Q/Y]YWS | (SEQ ID NO: |
| Seq.* | | (SEQ ID NO: 773) | (SEQ ID NO: 779) | (SEQ ID NO: 785) | | (SEQ ID NO: 797) | 803) |
| | VH | x[A/T]ISGSGGL | Wx[I/V]APYx[S/ | VITYDGINKYY | SIIPIIGFANYAQ | EIx[Y/G]x[Y/A]S | IINPSGGITVYA |
| | CDR2 | TYYADSVKG | N]GNTNYAQKL | ADSVEG | KFQG | GSTRYNPSLKS | QKFQG |
| | | (SEQ ID NO: 774) | QG (SEQ ID | (SEQ ID NO: 786) | (SEQ ID NO: 792) | (SEQ ID NO: 798) | (SEQ ID NO: |
| | | | NO: 780) | | | | 804) |
| | VH | APYGYYMDV | DAGTYSPx[F/Y] | DGVYYGVYDY | DSGYYYGASSF | Dx[T/A]PYYYx | GGSKVAALAF |
| | CDR3 | (SEQ ID NO: 775) | GYGMDV | (SEQ ID NO: 787) | GMDV | [E/G/D]GGYYYY | DI |
| | | | (SEQ ID NO: 781) | | (SEQ ID NO: 793) | MDV | (SEQ ID NO: |
| | | | | | | (SEQ ID NO: 799) | 805) |
| VL | VL | RASQSISSWLA | x[R/Q]ASx[Q/E/ | KSSQSVLFSSN | RASQSVSSNLA | RASx[Q/E/D]SV | QASQDISNSLN |
| CDR | CDR1 | (SEQ ID NO: 776) | H]SIx[S/D/N]x[S/ | NKNYLA | (SEQ ID NO: 794) | x[S/D]SSx[Y/F]L | (SEQ ID NO: |
| Seq.* | | | N]WLA | (SEQ ID NO: 788) | | A (SEQ ID | 806) |
| | | | (SEQ ID NO: 782) | | | NO: 800) | |
| | VL | KASSLES | x[K/S]Ax[S/Y]x | WASTRES | GASTRAT | GAx[S/D/F/Y]x[S/ | DASNLET |
| | CDR2 | (SEQ ID NO: 777) | [S/Y/N]LEx[S/Y] | (SEQ ID NO: 789) | (SEQ ID NO: 795) | T]Rx[A/Q]x[T/N] | (SEQ ID NO: |
| | | | (SEQ ID NO: 783) | | | (SEQ ID | 807) |
| | | | | | | NO: 801) | |
| | VL | QQYKSYIT | Qx[Q/L/R]FQx[S/ | QQFHSYPLT | EQYNNLPLT | QQx[V/A/D]GV | QQYNFHPLT |
| | CDR3 | (SEQ ID NO: 778) | K]LPPFT | (SEQ ID NO: 790) | (SEQ ID NO: 796) | VPYT | (SEQ ID NO: |
| | | | (SEQ ID NO: 784) | | | (SEQ ID NO: 802) | 808) |

*Exemplary CDR sequences encompass amino acids as determined by Kabat plus Chothia

TABLE 15
Human, Cynomolgus Monkey,and Mouse TF Sequences

| | | Cynomolgus Monkey | |
|---|---|---|---|
| Species | Human (Homo sapiens) | (Macaca fascicularis) | Mouse (Mus musculus) |
| Full-length | METPAWPRVPRPETAV | METPAWPRVPRPETAV | MAILVRPRLLAALAPTF |
| sequence | ARTLLLGWVFAQVAGA | ARTLLLGWVFAQVAGA | LGCLLLQVTAGAGIPEK |
| [signal | SGTTNTVAAYNLTWKS | SGTTNTVAAYNLTWKS | AFNLTWISTDFKTILEW |
| sequence | TNFKTILEWEPKPVNQV | TNFKTILEWEPKPINQV | QPKPTNYTYTVQISDRS |
| underlined] | YTVQISTKSGDWKSKCF | YTVQISTKSGDWKSKCF | RNWKNKCFSTTDTECD |
| | YTTDTECDLTDEIVKDV | YTADTECDLTDEIVKDV | LTDEIVKDVTWAYEAK |
| | KQTYLARVFSYPAGNV | KQTYLARVFSYPAGHV | VLSVPRRNSVHGDGDQ |
| | ESTGSAGEPLYENSPEFT | ESTGSTEEPPYENSPEFT | LVIHGEEPPFTNAPKFLP |
| | PYLETNLGQPTIQSFEQV | PYLETNLGQPTIQSFEQV | YRDTNLGQPVIQQFEQD |
| | GTKVNVTVEDERTLVR | GTKVNVTVQDEWTLVR | GRKLNVVVKDSLTLVR |
| | RNNTFLSLRDVFGKDLI | RNDTFLSLRDVFGKDLI | KNGTFLTLRQVFGKDLG |
| | YTLYYWKSSSSGKKTA | YTLYYWKSSSSGKKTA | YIITYRKGSSTGKKTNIT |
| | KTNTNEFLIDVDKGENY | KTNTNEFLIDVDKGENY | NTNEFSIDVEEGVSYCFF |
| | CFSVQAVIPSRTVNRKS | CFSVQAVIPSRRTANRK | VQAMIFSRKTNQNSPGS |
| | TDSPVECMGQEKGEFRE | STDSPVECMGHEKGESR | STVCTEQWKSFLGETLII |
| | IFYIIGAVVFVVIILVIILA | EIFYIIGAVVFVVIILVIIL | VGAVVLLATIFIILLSISL |
| | ISLHKCRKAGVGQSWK | AISLHKCKKARVGRSW | CKRRKNRAGQKGKNTP |
| | ENSPLNVS (SEQ ID | KENSPLNVA (SEQ ID | SRLA (SEQ ID NO: 817) |
| | NO: 809) | NO: 813) | |
| Extracellular | SGTTNTVAAYNLTWKS | SGTTNTVAAYNLTWKS | AGIPEKAFNLTWISTDFK |
| domain (ECD) | TNFKTILEWEPKPVNQV | TNFKTILEWEPKPINQV | TILEWQPKPTNYTYTVQ |
| | YTVQISTKSGDWKSKCF | YTVQISTKSGDWKSKCF | ISDRSRNWKNKCFSTTD |
| | YTTDTECDLTDEIVKDV | YTADTECDLTDEIVKDV | TECDLTDEIVKDVTWA |
| | KQTYLARVFSYPAGNV | KQTYLARVFSYPAGHV | YEAKVLSVPRRNSVHG |
| | ESTGSAGEPLYENSPEFT | ESTGSTEEPPYENSPEFT | DGDQLVIHGEEPPFTNA |
| | PYLETNLGQPTIQSFEQV | PYLETNLGQPTIQSFEQV | PKFLPYRDTNLGQPVIQ |
| | GTKVNVTVEDERTLVR | GTKVNVTVQDEWTLVR | QFEQDGRKLNVVVKDS |
| | RNNTFLSLRDVFGKDLI | RNDTFLSLRDVFGKDLI | LTLVRKNGTFLTLRQVF |
| | YTLYYWKSSSSGKKTA | YTLYYWKSSSSGKKTA | GKDLGYIITYRKGSSTG |
| | KTNTNEFLIDVDKGENY | KTNTNEFLIDVDKGENY | KKTNITNTNEFSIDVEEG |
| | CFSVQAVIPSRTVNRKS | CFSVQAVIPSRRTANRK | VSYCFFVQAMIFSRKTN |
| | TDSPVECMGQEKGEFRE | STDSPVECMGHEKGESR | QNSPGSSTVCTEQWKSF |
| | (SEQ ID NO: 810) | E (SEQ ID NO: 814) | LGE (SEQ ID NO: 818) |
| Sequence of TF | SGTTNTVAAYNLTWKS | SGTTNTVAAYNLTWKS | AGIPEKAFNLTWISTDFK |
| ECD-His (TF- | TNFKTILEWEPKPVNQV | TNFKTILEWEPKPINQV | TILEWQPKPTNYTYTVQ |
| His) protein | YTVQISTKSGDWKSKCF | YTVQISTKSGDWKSKCF | ISDRSRNWKNKCFSTTD |
| | YTTDTECDLTDEIVKDV | YTADTECDLTDEIVKDV | TECDLTDEIVKDVTWA |
| | KQTYLARVFSYPAGNV | KQTYLARVFSYPAGHV | YEAKVLSVPRRNSVHG |
| | ESTGSAGEPLYENSPEFT | ESTGSTEEPPYENSPEFT | DGDQLVIHGEEPPFTNA |
| | PYLETNLGQPTIQSFEQV | PYLETNLGQPTIQSFEQV | PKFLPYRDTNLGQPVIQ |
| | GTKVNVTVEDERTLVR | GTKVNVTVQDEWTLVR | QFEQDGRKLNVVVKDS |
| | RNNTFLSLRDVFGKDLI | RNDTFLSLRDVFGKDLI | LTLVRKNGTFLTLRQVF |
| | YTLYYWKSSSSGKKTA | YTLYYWKSSSSGKKTA | GKDLGYIITYRKGSSTG |
| | KTNTNEFLIDVDKGENY | KTNTNEFLIDVDKGENY | KKTNITNTNEFSIDVEEG |
| | CFSVQAVIPSRTVNRKS | CFSVQAVIPSRRTANRK | VSYCFFVQAMIFSRKTN |
| | TDSPVECMGQEKGEFRE | STDSPVECMGHEKGESR | QNSPGSSTVCTEQWKSF |
| | TGHHHHHH (SEQ ID | ETGHHHHHH (SEQ ID | LGETGHHHHHH (SEQ |
| | NO: 811) | NO: 815) | ID NO: 819) |
| Sequence of TF | SGTTNTVAAYNLTWKS | SGTTNTVAAYNLTWKS | AGIPEKAFNLTWISTDFK |
| ECD-Fc (TF- | TNFKTILEWEPKPVNQV | TNFKTILEWEPKPINQV | TILEWQPKPTNYTYTVQ |
| Fc) fusion | YTVQISTKSGDWKSKCF | YTVQISTKSGDWKSKCF | ISDRSRNWKNKCFSTTD |
| protein | YTTDTECDLTDEIVKDV | YTADTECDLTDEIVKDV | TECDLTDEIVKDVTWA |
| | KQTYLARVFSYPAGNV | KQTYLARVFSYPAGHV | YEAKVLSVPRRNSVHG |
| | ESTGSAGEPLYENSPEFT | ESTGSTEEPPYENSPEFT | DGDQLVIHGEEPPFTNA |
| | PYLETNLGQPTIQSFEQV | PYLETNLGQPTIQSFEQV | PKFLPYRDTNLGQPVIQ |
| | GTKVNVTVEDERTLVR | GTKVNVTVQDEWTLVR | QFEQDGRKLNVVVKDS |
| | RNNTFLSLRDVFGKDLI | RNDTFLSLRDVFGKDLI | LTLVRKNGTFLTLRQVF |
| | YTLYYWKSSSSGKKTA | YTLYYWKSSSSGKKTA | GKDLGYIITYRKGSSTG |
| | KTNTNEFLIDVDKGENY | KTNTNEFLIDVDKGENY | KKTNITNTNEFSIDVEEG |
| | CFSVQAVIPSRTVNRKS | CFSVQAVIPSRRTANRK | VSYCFFVQAMIFSRKTN |
| | TDSPVECMGQEKGEFRE | STDSPVECMGHEKGESR | QNSPGSSTVCTEQWKSF |
| | TGENLYFQGDKTHTCPP | ETGENLYFQGDKTHTCP | LGETGENLYFQGDKTHT |
| | CPAPELLGGPSVFLFPPK | PCPAPELLGGPSVFLFPP | CPPCPAPELLGGPSVFLF |
| | PKDTLMISRTPEVTCVV | KPKDTLMISRTPEVTCV | PPKPKDTLMISRTPEVTC |
| | VDVSHEDPEVKFNWYV | VVDVSHEDPEVKFNWY | VVVDVSHEDPEVKFNW |
| | DGVEVHNAKTKPREEQ | VDGVEVHNAKTKPREE | YVDGVEVHNAKTKPRE |
| | YNSTYRVVSVLTVLHQ | QYNSTYRVVSVLTVLH | EQYNSTYRVVSVLTVLH |
| | DWLNGKEYKCKVSNKA | QDWLNGKEYKCKVSNK | QDWLNGKEYKCKVSNK |
| | LPAPIEKTISKAKGQPRE | ALPAPIEKTISKAKGQPR | ALPAPIEKTISKAKGQPR |
| | PQVYTLPPSREEMTKNQ | EPQVYTLPPSREEMTKN | EPQVYTLPPSREEMTKN |
| | VSLTCLVKGFYPSDIAV | QVSLTCLVKGFYPSDIA | QVSLTCLVKGFYPSDIA |
| | EWESNGQPENNYKTTPP | VEWESNGQPENNYKTT | VEWESNGQPENNYKTT |
| | VLDSDGSFFLYSKLTVD | PPVLDSDGSFFLYSKLT | PPVLDSDGSFFLYSKLT |
| | KSRWQQGNVFSCSVMH | VDKSRWQQGNVFSCSV | VDKSRWQQGNVFSCSV |
| | EALHNHYTQKSLSLSPG | MHEALHNHYTQKSLSL | MHEALHNHYTQKSLSL |
| | K (SEQ ID NO: 812) | SPGK (SEQ ID NO: 816) | SPGK (SEQ ID NO: 820) |

TABLE 16
Sequences of Anti-TF Antibodies

| Antibody | VH domain | VL domain |
|---|---|---|
| 10H10 | EVQLVQSGAEVKKPGESLRISCKGSGYT | DIVMTQTPLSLPVTPGEPASISCKSSQSL |
| (M1593) | FAPYWIEWVRQMPGKGLEWMGDILPG | LSSGNQKNYLTWYLQKPGQSPQLLIYW |
| | TGFTTYSPSFQGHVTISADKSISTAYLQW | ASTRESGVPDRFSGSGSGTDFTLKISRVE |
| | SSLKASDTAMYYCARSGYYGNSGFAY | AEDVGVYYCQNDYTYPLTFGQGTKLEI |
| | WGQGTLVTVSS (SEQ ID NO: 821) | K (SEQ ID NO: 822) |
| TF-011 | EVQLLESGGGLVQPGGSLRLSCAASGFT | DIQMTQSPPSLSASAGDRVTITCRASQGI |
| | FSNYAMSWVRQAPGKGLEWVSSISGSG | SSRLAWYQQKPEKAPKSLIYAASSLQSG |
| | DYTYYTDSVKGRFTISRDNSKNTLYLQ | VPSRFSGSGSGTDFTLTISSLQPEDFATY |
| | MNSLRAEDTAVYYCARSPWGYYLDSW | YCQQYNSYPYTFGQGTKLEIK (SEQ ID |
| | GQGTLVTVSS (SEQ ID NO: 828) | NO: 829) |
| 5G9 | QVQLVESGGGVVQPGRSLRLSCKASGF | DIQMTQSPSSLSASVGDRVTITCKASQDI |
| (humanized | NIKDYYMHWVRQAPGKGLEWIGLIDPE | RKYLNWYQQKPGKAPKLLIYYATSLAD |
| TF8-5G9, | NGNTIYDPKFQGRFTISADNSKNTLFLQ | GVPSRFSGSGSGTDYTFTISSLQPEDIAT |
| CNTO 860) | MDSLRPEDTAVYYCARDNSYYFDYWG | YYCLQHGESPYTFGQGTKLEIT (SEQ ID |
| | QGTPVTVSS (SEQ ID NO: 830) | NO: 831) |

TABLE 17
Pig TF sequences

| Species | Pig (Sus scrofa) |
|---|---|
| Full-length sequence [signal sequence | MATPTGPPVSCPKAAVARALLLGWVLVQVAGATGTTDVIV |
| underlined] | AYNLTWKSTNFKTILEWEPKPINYVYTVQISPRLGDWKNKC |
| | FHTTDTECDVTDEIMRNVKETYVARVLSYPADTVLTAQEPPF |
| | TNSPPFTPYLDTNLGQPVIQSFEQVGTKLNVTVEAARTLVRV |
| | NGTFLRLRDVFGKDLNYTLYYWRASSTGKKKATTNTNEFLI |
| | DVDKGENYCFSVQAVIPSRRVNQKSPESRIECTSQEKAVSRE |
| | LFLIVGAVVFAVIVFVLVLSVSLYKCRKERAGPSGKENAPLN |
| | VA (SEQ ID NO: 824) |
| Extracellular domain (ECD) | TGTTDVIVAYNLTWKSTNFKTILEWEPKPINYVYTVQISPRL |
| | GDWKNKCFHTTDTECDVTDEIMRNVKETYVARVLSYPADT |
| | VLTAQEPPFTNSPPFTPYLDTNLGQPVIQSFEQVGTKLNVTVE |
| | AARTLVRVNGTFLRLRDVFGKDLNYTLYYWRASSTGKKKA |
| | TTNTNEFLIDVDKGENYCFSVQAVIPSRRVNQKSPESRIECTS |
| | QEKAVSRE (SEQ ID NO: 825) |
| Sequence of TF ECD-His (TF-His) | TGTTDVIVAYNLTWKSTNFKTILEWEPKPINYVYTVQISPRL |
| protein | GDWKNKCFHTTDTECDVTDEIMRNVKETYVARVLSYPADT |
| | VLTAQEPPFTNSPPFTPYLDTNLGQPVIQSFEQVGTKLNVTVE |
| | AARTLVRVNGTFLRLRDVFGKDLNYTLYYWRASSTGKKKA |
| | TTNTNEFLIDVDKGENYCFSVQAVIPSRRVNQKSPESRIECTS |
| | QEKAVSRETGHHHHHH (SEQ ID NO: 826) |
| Sequence of TF ECD-Fc (TF-Fc) | TGTTDVIVAYNLTWKSTNFKTILEWEPKPINYVYTVQISPRL |
| fusion protein | GDWKNKCFHTTDTECDVTDEIMRNVKETYVARVLSYPADT |
| | VLTAQEPPFTNSPPFTPYLDTNLGQPVIQSFEQVGTKLNVTVE |
| | AARTLVRVNGTFLRLRDVFGKDLNYTLYYWRASSTGKKKA |
| | TTNTNEFLIDVDKGENYCFSVQAVIPSRRVNQKSPESRIECTS |
| | QEKAVSRETGENLYFQGDKTHTCPPCPAPELLGGPSVFLFPP |
| | KPKDTLMISRTPEVTCVVVDVSHEDPEVKFNWYVDGVEVH |
| | NAKTKPREEQYNSTYRVVSVLTVLHQDWLNGKEYKCKVSN |
| | KALPAPIEKTISKAKGQPREPQVYTLPPSREEMTKNQVSLTCL |
| | VKGFYPSDIAVEWESNGQPENNYKTTPPVLDSDGSFFLYSKL |
| | TVDKSRWQQGNVFSCSVMHEALHNHYTQKSLSLSPGK (SEQ |
| | ID NO: 827) |

TABLE 18
Rabbit TF sequences

| Species | Rabbit (Oryctolagus cuniculus) |
|---|---|
| Full-length sequence [signal sequence | MAPPTRLQVPRPGTAVPYTVLLGWLLAQVARAADTTGRAY |
| underlined] | NLTWKSTNFKTILEWEPKSIDHVYTVQISTRLENWKSKCFLT |
| | AETECDLTDEVVKDVGQTYMARVLSYPARNGNTTGFPEEPP |
| | FRNSPEFTPYLDTNLGQPTIQSFEQVGTKLNVTVQDARTLVR |
| | RNGTFLSLRAVFGKDLNYTLYYWRASSTGKKTATTNTNEFL |
| | IDVDKGENYCFSVQAVIPSRKRKQRSPESLTECTSREQGRAR |
| | EMFFIIGAVVVVALLIIVLSVTVYKCRKARAGPSGKESSPLNI |
| | A (SEQ ID NO: 832) |
| Extracellular domain (ECD) | ADTTGRAYNLTWKSTNFKTILEWEPKSIDHVYTVQISTRLEN |
| | WKSKCFLTAETECDLTDEVVKDVGQTYMARVLSYPARNGN |
| | TTGFPEEPPFRNSPEFTPYLDTNLGQPTIQSFEQVGTKLNVTV |
| | QDARTLVRRNGTFLSLRAVFGKDLNYTLYYWRASSTGKKT |
| | ATTNTNEFLIDVDKGENYCFSVQAVIPSRKRKQRSPESLTECT |
| | SREQGRAREM (SEQ ID NO: 833) |
| Sequence of TF ECD-His (TF-His) | ADTTGRAYNLTWKSTNFKTILEWEPKSIDHVYTVQISTRLEN |
| protein | WKSKCFLTAETECDLTDEVVKDVGQTYMARVLSYPARNGN |
| | TTGFPEEPPFRNSPEFTPYLDTNLGQPTIQSFEQVGTKLNVTV |
| | QDARTLVRRNGTFLSLRAVFGKDLNYTLYYWRASSTGKKT |
| | ATTNTNEFLIDVDKGENYCFSVQAVIPSRKRKQRSPESLTECT |
| | SREQGRAREMTGHHHHHH (SEQ ID NO: 834) |
| Sequence of TF ECD-Fc (TF-Fc) | ADTTGRAYNLTWKSTNFKTILEWEPKSIDHVYTVQISTRLEN |
| fusion protein | WKSKCFLTAETECDLTDEVVKDVGQTYMARVLSYPARNGN |
| | TTGFPEEPPFRNSPEFTPYLDTNLGQPTIQSFEQVGTKLNVTV |
| | QDARTLVRRNGTFLSLRAVFGKDLNYTLYYWRASSTGKKT |
| | ATTNTNEFLIDVDKGENYCFSVQAVIPSRKRKQRSPESLTECT |
| | SREQGRAREMENLYFQGDKTHTCPPCPAPELLGGPSVFLFPP |
| | KPKDTLMISRTPEVTCVVVDVSHEDPEVKFNWYVDGVEVH |
| | NAKTKPREEQYNSTYRVVSVLTVLHQDWLNGKEYKCKVSN |
| | KALPAPIEKTISKAKGQPREPQVYTEPPSREEMTKNQVSLTCL |
| | VKGFYPSDIAVEWESNGQPENNYKTTPPVLDSDGSFFLYSKL |
| | TVDKSRWQQGNVFSCSVMHEALHNHYTQKSLSLSPGK (SEQ |
| | ID NO: 835) |

TABLE 19
Rat TF ECD and chimeric construct ECD sequences

| Rat/Chimeric construct | Extracellular domain (ECD) sequence |
|---|---|
| rTF (rat TF) | AGTPPGKAFNLTWISTDFKTILEWQPKPTNYTYTVQISDRSRNWKYKC |
| | TGTTDTECDLTDEIVKDVNWTYEARVLSVPWRNSTHGKETLFGTHGE |
| | EPPFTNARKFLPYRDTKIGQPVIQKYEQGGTKLKVTVKDSFTLVRKNG |
| | TFLTLRQVFGNDLGYILTYRKDSSTGRKTNTTHTNEFLIDVEKGVSYCF |
| | FAQAVIFSRKTNHKSPESITKCTEQWKSVLGE (SEQ ID NO: 838) |
| h1-107_r | AGTPPGKAFNLTWISTDFKTILEWQPKPTNYTYTVQISDRSRNWKYKC |
| | TGTTDTECDLTDEIVKDVNWTYEARVLSVPWRNSTHGKETLFGTHGE |
| | EPPFTNARKFLPYRDTKLGQPTIQSFEQVGTKVNVTVEDERTLVRRNN |
| | TFLSLRDVFGKDLIYTLYYWKSSSSGKKTAKTNTNEFLIDVDKGENYC |
| | FSVQAVIPSRTVNRKSTDSPVECMGQEKGEFRE (SEQ ID NO: 839) |
| h1-77_r | AGTPPGKAFNLTWISTDFKTILEWQPKPTNYTYTVQISDRSRNWKYKC |
| | TGTTDTECDLTDEIVKDVNWTYEARVLSYPAGNVESTGSAGEPLYENS |
| | PEFTPYLETNLGQPTIQSFEQVGTKVNVTVEDERTLVRRNNTFLSLRDV |
| | FGKDLIYTLYYWKSSSSGKKTAKTNTNEFLIDVDKGENYCFSVQAVIP |
| | SRTVNRKSTDSPVECMGQEKGEFRE (SEQ ID NO: 840) |
| h1-38_r | AGTPPGKAFNLTWISTDFKTILEWQPKPTNYTYTVQISTKSGDWKSKC |
| | FYTTDTECDLTDEIVKDVKQTYLARVFSYPAGNVESTGSAGEPLYENS |
| | PEFTPYLETNLGQPTIQSFEQVGTKVNVTVEDERTLVRRNNTFLSLRDV |
| | FGKDLIYTLYYWKSSSSGKKTAKTNTNEFLIDVDKGENYCFSVQAVIP |
| | SRTVNRKSTDSPVECMGQEKGEFRE (SEQ ID NO: 841) |
| h39-77_r | SGTTNTVAAYNLTWKSTNFKTILEWEPKPVNQVYTVQISDRSRNWKY |
| | KCTGTTDTECDLTDEIVKDVNWTYEARVLSYPAGNVESTGSAGEPLY |
| | ENSPEFTPYLETNLGQPTIQSFEQVGTKVNVTVEDERTLVRRNNTFLSL |
| | RDVFGKDLIYTLYYWKSSSSGKKTAKTNTNEFLIDVDKGENYCFSVQ |
| | AVIPSRTVNRKSTDSPVECMGQEKGEFRE (SEQ ID NO: 842) |
| h78-107_r | SGTTNTVAAYNLTWKSTNFKTILEWEPKPVNQVYTVQISTKSGDWKS |
| | KCFYTTDTECDLTDEIVKDVKQTYLARVFSVPWRNSTHGTHGEEPPFT |
| | NARKFLPYRDTKLGQPTIQSFEQVGTKVNVTVEDERTLVRRNNTFLSL |
| | RDVFGKDLIYTLYYWKSSSSGKKTAKTNTNEFLIDVDKGENYCFSVQ |
| | AVIPSRTVNRKSTDSPVECMGQEKGEFRE (SEQ ID NO: 843) |
| h78-107_r.v2 | SGTTNTVAAYNLTWKSTNFKTILEWEPKPVNQVYTVQISTKSGDWKS |
| | KCFYTTDTECDLTDEIVKDVKQTYLARVFSVPWRNSTHGKETLFGTH |
| | GEEPPFTNARKFLPYRDTKLGQPTIQSFEQVGTKVNVTVEDERTLVRR |
| | NNTFLSLRDVFGKDLIYTLYYWKSSSSGKKTAKTNTNEFLIDVDKGEN |
| | YCFSVQAVIPSRTVNRKSTDSPVECMGQEKGEFRE (SEQ ID NO: 844) |
| h78-93_r | SGTTNTVAAYNLTWKSTNFKTILEWEPKPVNQVYTVQISTKSGDWKS |
| | KCFYTTDTECDLTDEIVKDVKQTYLARVFSVPWRNSTHGKETLFGTH |
| | GEEPPYENSPEFTPYLETNLGQPTIQSFEQVGTKVNVTVEDERTLVRRN |
| | NTFLSLRDVFGKDLIYTLYYWKSSSSGKKTAKTNTNEFLIDVDKGENY |
| | CFSVQAVIPSRTVNRKSTDSPVECMGQEKGEFRE (SEQ ID NO: 845) |
| h94-107_r | SGTTNTVAAYNLTWKSTNFKTILEWEPKPVNQVYTVQISTKSGDWKS |
| | KCFYTTDTECDLTDEIVKDVKQTYLARVFSYPAGNVESTGSAGEPLFT |
| | NARKFLPYRDTKLGQPTIQSFEQVGTKVNVTVEDERTLVRRNNTFLSL |
| | RDVFGKDLIYTLYYWKSSSSGKKTAKTNTNEFLIDVDKGENYCFSVQ |
| | AVIPSRTVNRKSTDSPVECMGQEKGEFRE (SEQ ID NO: 846) |
| h108-219_r | SGTTNTVAAYNLTWKSTNFKTILEWEPKPVNQVYTVQISTKSGDWKS |
| | KCFYTTDTECDLTDEIVKDVKQTYLARVFSYPAGNVESTGSAGEPLYE |
| | NSPEFTPYLETNIGQPVIQKYEQGGTKLKVTVKDSFTLVRKNGTFLTLR |
| | QVFGNDLGYILTYRKDSSTGRKTNTTHTNEFLIDVEKGVSYCFFAQAV |
| | IFSRKTNHKSPESITKCTEQWKSVLGE (SEQ ID NO: 847) |
| h108-158_r | SGTTNTVAAYNLTWKSTNFKTILEWEPKPVNQVYTVQISTKSGDWKS |
| | KCFYTTDTECDLTDEIVKDVKQTYLARVFSYPAGNVESTGSAGEPLYE |
| | NSPEFTPYLETNIGQPVIQKYEQGGTKLKVTVKDSFTLVRKNGTFLTLR |
| | QVFGNDLGYILTYRKSSSSGKKTAKTNTNEFLIDVDKGENYCFSVQAV |
| | IPSRTVNRKSTDSPVECMGQEKGEFRE (SEQ ID NO: 848) |
| h108-132_r | SGTTNTVAAYNLTWKSTNFKTILEWEPKPVNQVYTVQISTKSGDWKS |
| | KCFYTTDTECDLTDEIVKDVKQTYLARVFSYPAGNVESTGSAGEPLYE |
| | NSPEFTPYLETNIGQPVIQKYEQGGTKLKVTVKDSFTLVRRNNTFLSLR |
| | DVFGKDLIYTLYYWKSSSSGKKTAKTNTNEFLIDVDKGENYCFSVQA |
| | VIPSRTVNRKSTDSPVECMGQEKGEFRE (SEQ ID NO: 849) |
| h133-158_r | SGTTNTVAAYNLTWKSTNFKTILEWEPKPVNQVYTVQISTKSGDWKS |
| | KCFYTTDTECDLTDEIVKDVKQTYLARVFSYPAGNVESTGSAGEPLYE |
| | NSPEFTPYLETNLGQPTIQSFEQVGTKVNVTVEDERTLVRKNGTFLTLR |
| | QVFGNDLGYILTYRKSSSSGKKTAKTNTNEFLIDVDKGENYCFSVQAV |
| | IPSRTVNRKSTDSPVECMGQEKGEFRE (SEQ ID NO: 850) |
| h133-145_r | SGTTNTVAAYNLTWKSTNFKTILEWEPKPVNQVYTVQISTKSGDWKS |
| | KCFYTTDTECDLTDEIVKDVKQTYLARVFSYPAGNVESTGSAGEPLYE |
| | NSPEFTPYLETNLGQPTIQSFEQVGTKVNVTVEDERTLVRKNGTFLTLR |
| | QVFGKDLIYTLYYWKSSSSGKKTAKTNTNEFLIDVDKGENYCFSVQA |
| | VIPSRTVNRKSTDSPVECMGQEKGEFRE (SEQ ID NO: 851) |
| h133-139_r | SGTTNTVAAYNLTWKSTNFKTILEWEPKPVNQVYTVQISTKSGDWKS |
| | KCFYTTDTECDLTDEIVKDVKQTYLARVFSYPAGNVESTGSAGEPLYE |
| | NSPEFTPYLETNLGQPTIQSFEQVGTKVNVTVEDERTLVRKNGTFLSLR |
| | DVFGKDLIYTLYYWKSSSSGKKTAKTNTNEFLIDVDKGENYCFSVQA |
| | VIPSRTVNRKSTDSPVECMGQEKGEFRE (SEQ ID NO: 852) |
| h140-145_r | SGTTNTVAAYNLTWKSTNFKTILEWEPKPVNQVYTVQISTKSGDWKS |
| | KCFYTTDTECDLTDEIVKDVKQTYLARVFSYPAGNVESTGSAGEPLYE |
| | NSPEFTPYLETNLGQPTIQSFEQVGTKVNVTVEDERTLVRRNNTFLTLR |
| | QVFGKDLIYTLYYWKSSSSGKKTAKTNTNEFLIDVDKGENYCFSVQA |
| | VIPSRTVNRKSTDSPVECMGQEKGEFRE (SEQ ID NO: 853) |
| h146-158_r | SGTTNTVAAYNLTWKSTNFKTILEWEPKPVNQVYTVQISTKSGDWKS |
| | KCFYTTDTECDLTDEIVKDVKQTYLARVFSYPAGNVESTGSAGEPLYE |
| | NSPEFTPYLETNLGQPTIQSFEQVGTKVNVTVEDERTLVRRNNTFLSLR |
| | DVFGNDLGYILTYRKSSSSGKKTAKTNTNEFLIDVDKGENYCFSVQAV |
| | IPSRTVNRKSTDSPVECMGQEKGEFRE (SEQ ID NO: 854) |
| h146-151_r | SGTTNTVAAYNLTWKSTNFKTILEWEPKPVNQVYTVQISTKSGDWKS |
| | KCFYTTDTECDLTDEIVKDVKQTYLARVFSYPAGNVESTGSAGEPLYE |
| | NSPEFTPYLETNLGQPTIQSFEQVGTKVNVTVEDERTLVRRNNTFLSLR |
| | DVFGNDLIYTLYYWKSSSSGKKTAKTNTNEFLIDVDKGENYCFSVQA |
| | VIPSRTVNRKSTDSPVECMGQEKGEFRE (SEQ ID NO: 855) |
| h152-158_r | SGTTNTVAAYNLTWKSTNFKTILEWEPKPVNQVYTVQISTKSGDWKS |
| | KCFYTTDTECDLTDEIVKDVKQTYLARVFSYPAGNVESTGSAGEPLYE |
| | NSPEFTPYLETNLGQPTIQSFEQVGTKVNVTVEDERTLVRRNNTFLSLR |
| | DVFGKDLGYILTYRKSSSSGKKTAKTNTNEFLIDVDKGENYCFSVQAV |
| | IPSRTVNRKSTDSPVECMGQEKGEFRE (SEQ ID NO: 856) |
| h159-219_r | SGTTNTVAAYNLTWKSTNFKTILEWEPKPVNQVYTVQISTKSGDWKS |
| | KCFYTTDTECDLTDEIVKDVKQTYLARVFSYPAGNVESTGSAGEPLYE |
| | NSPEFTPYLETNLGQPTIQSFEQVGTKVNVTVEDERTLVRRNNTFLSLR |
| | DVFGKDLIYTLYYWKDSSTGRKTNTTHTNEFLIDVEKGVSYCFFAQAV |
| | IFSRKTNHKSPESITKCTEQWKSVLGE (SEQ ID NO: 857) |
| h159-189_r | SGTTNTVAAYNLTWKSTNFKTILEWEPKPVNQVYTVQISTKSGDWKS |
| | KCFYTTDTECDLTDEIVKDVKQTYLARVFSYPAGNVESTGSAGEPLYE |
| | NSPEFTPYLETNLGQPTIQSFEQVGTKVNVTVEDERTLVRRNNTFLSLR |
| | DVFGKDLIYTLYYWKDSSTGRKTNTTHTNEFLIDVEKGVSYCFFAQAV |
| | IPSRTVNRKSTDSPVECMGQEKGEFRE (SEQ ID NO: 858) |
| h159-174_r | SGTTNTVAAYNLTWKSTNFKTILEWEPKPVNQVYTVQISTKSGDWKS |
| | KCFYTTDTECDLTDEIVKDVKQTYLARVFSYPAGNVESTGSAGEPLYE |
| | NSPEFTPYLETNLGQPTIQSFEQVGTKVNVTVEDERTLVRRNNTFLSLR |
| | DVFGKDLIYTLYYWKDSSTGRKTNTTHTNEFLIDVDKGENYCFSVQA |
| | VIPSRTVNRKSTDSPVECMGQEKGEFRE (SEQ ID NO: 859) |
| h159-166_r | SGTTNTVAAYNLTWKSTNFKTILEWEPKPVNQVYTVQISTKSGDWKS |
| | KCFYTTDTECDLTDEIVKDVKQTYLARVFSYPAGNVESTGSAGEPLYE |
| | NSPEFTPYLETNLGQPTIQSFEQVGTKVNVTVEDERTLVRRNNTFLSLR |
| | DVFGKDLIYTLYYWKDSSTGRKTAKTNTNEFLIDVDKGENYCFSVQA |
| | VIPSRTVNRKSTDSPVECMGQEKGEFRE (SEQ ID NO: 860) |
| h167-174_r | SGTTNTVAAYNLTWKSTNFKTILEWEPKPVNQVYTVQISTKSGDWKS |
| | KCFYTTDTECDLTDEIVKDVKQTYLARVFSYPAGNVESTGSAGEPLYE |
| | NSPEFTPYLETNLGQPTIQSFEQVGTKVNVTVEDERTLVRRNNTFLSLR |
| | DVFGKDLIYTLYYWKSSSSGKKTNTTHTNEFLIDVDKGENYCFSVQAV |
| | IPSRTVNRKSTDSPVECMGQEKGEFRE (SEQ ID NO: 861) |
| h175-189_r | SGTTNTVAAYNLTWKSTNFKTILEWEPKPVNQVYTVQISTKSGDWKS |
| | KCFYTTDTECDLTDEIVKDVKQTYLARVFSYPAGNVESTGSAGEPLYE |
| | NSPEFTPYLETNLGQPTIQSFEQVGTKVNVTVEDERTLVRRNNTFLSLR |
| | DVFGKDLIYTLYYWKSSSSGKKTAKTNTNEFLIDVEKGVSYCFFAQAV |
| | IPSRTVNRKSTDSPVECMGQEKGEFRE (SEQ ID NO: 862) |
| h190-219_r | SGTTNTVAAYNLTWKSTNFKTILEWEPKPVNQVYTVQISTKSGDWKS |
| | KCFYTTDTECDLTDEIVKDVKQTYLARVFSYPAGNVESTGSAGEPLYE |
| | NSPEFTPYLETNLGQPTIQSFEQVGTKVNVTVEDERTLVRRNNTFLSLR |
| | DVFGKDLIYTLYYWKSSSSGKKTAKTNTNEFLIDVDKGENYCFSVQA |
| | VIFSRKTNHKSPESITKCTEQWKSVLGE (SEQ ID NO: 863) |
| hTF_K68N | SGTTNTVAAYNLTWKSTNFKTILEWEPKPVNQVYTVQISTKSGDWKS |
| | KCFYTTDTECDLTDEIVKDVNQTYLARVFSYPAGNVESTGSAGEPLYE |
| | NSPEFTPYLETNLGQPTIQSFEQVGTKVNVTVEDERTLVRRNNTFLSLR |
| | DVFGKDLIYTLYYWKSSSSGKKTAKTNTNEFLIDVDKGENYCFSVQA |
| | VIPSRTVNRKSTDSPVECMGQEKGEFRE (SEQ ID NO: 865) |
| hTF_K149N | SGTTNTVAAYNLTWKSTNFKTILEWEPKPVNQVYTVQISTKSGDWKS |
| | KCFYTTDTECDLTDEIVKDVKQTYLARVFSYPAGNVESTGSAGEPLYE |
| | NSPEFTPYLETNLGQPTIQSFEQVGTKVNVTVEDERTLVRRNNTFLSLR |
| | DVFGNDLIYTLYYWKSSSSGKKTAKTNTNEFLIDVDKGENYCFSVQA |
| | VIPSRTVNRKSTDSPVECMGQEKGEFRE (SEQ ID NO: 866) |
| hTF_N171H_T197K | SGTTNTVAAYNLTWKSTNFKTILEWEPKPVNQVYTVQISTKSGDWKS |
| | KCFYTTDTECDLTDEIVKDVKQTYLARVFSYPAGNVESTGSAGEPLYE |
| | NSPEFTPYLETNLGQPTIQSFEQVGTKVNVTVEDERTLVRRNNTFLSLR |
| | DVFGKDLIYTLYYWKSSSSGKKTAKTHTNEFLIDVDKGENYCFSVQA |
| | VIPSRKVNRKSTDSPVECMGQEKGEFRE (SEQ ID NO :867) |
| r141-194_h | AGTPPGKAFNLTWISTDFKTILEWQPKPTNYTYTVQISDRSRNWKYKC |
| | TGTTDTECDLTDEIVKDVNWTYEARVLSVPWRNSTHGKETLFGTHGE |
| | EPPFTNARKFLPYRDTKIGQPVIQKYEQGGTKLKVTVKDSFTLVRRNN |
| | TFLSLRDVFGKDLIYTLYYWKSSSSGKKTAKTNTNEFLIDVDKGENYC |
| | FSVQAVIFSRKTNHKSPESITKCTEQWKSVLGE (SEQ ID NO: 864) |

TABLE 20
Variable region sequence consensus

| Group | VH Domain Consensus (SEQ ID NO) | VL Domain Consensus (SEQ ID NO) |
|---|---|---|
| Lineage | QVQLVQSGAEVKKPGASVKVSCKASGYT | DIQMTQSPSTLSASVGDRVTITCx[R/Q]ASx |
| 25A | FDx[V/A]YGISWVRQAPGQGLEWMGWIAP | [Q/E]SIx[S/N]x[S/N]WLAWYQQKPGKAPKL |
| | Yx[N/S]GNTNYAQKLQGRVTMTTDTSTST | LIYKAx[S/Y]x[S/N]LEx[S/Y]GVPSRFSGSGS |
| | AYMELRSLRSDDTAVYYCARDAGTYSPF | GTEFTLTISSLQPDDFATYYCQx[Q/L]FQx[S/ |
| | GYGMDVWGQGTTVTVSS (SEQ ID | K]LPPFTFGGGTKVEIK (SEQ ID NO: 869) |
| | NO: 868) | |
| Lineage | QVQLVQSGAEVKKPGASVKVSCKASGYT | DIQMTQSPSTLSASVGDRVTITCx[R/Q]ASx |
| 25G | FRSYGISWVRQAPGQGLEWMGWVAPYx | [Q/H]SIx[S/D]SWLAWYQQKPGKAPKLLIY |
| | [N/S]GNTNYAQKLQGRVTMTTDTSTSTAY | x[K/S]ASx[S/Y]LESGVPSRFSGSGSGTEFTL |
| | MELRSLRSDDTAVYYCARDAGTYSPYGY | TISSLQPDDFATYYCQx[Q/L/R]FQSLPPFTF |
| | GMDVWGQGTTVTVSS (SEQ ID NO: 870) | GGGTKVEIK (SEQ ID NO: 871) |

TABLE 21
Consensus CDRs

| Antibody Group | | Lineage 25A | Lineage 25G |
|---|---|---|---|
| VH CDR | VH CDR1 | GYTFDx[V/A]YGIS | GYTFRSYGIS (SEQ ID NO: 878) |
| Seq.* | | (SEQ ID NO: 872) | |
| | VH CDR2 | WIAPYx[N/S]GNTNYAQKLQG | WVAPYx[N/S]GNTNYAQKLQG |
| | | (SEQ ID NO: 873) | (SEQ ID NO: 879) |
| | VH CDR3 | DAGTYSPFGYGMDV | DAGTYSPYGYGMDV |
| | | (SEQ ID NO: 874) | (SEQ ID NO: 880) |
| VL CDR | VL CDR1 | x[R/Q]ASx[Q/E]SIx[S/N]x[S/N]WLA | x[R/Q]ASx[Q/H]SIx[S/D]SWLA |
| Seq.* | | (SEQ ID NO: 875) | (SEQ ID NO: 881) |
| | VL CDR2 | KAx[S/Y]x[S/N]LEx[S/Y] | x[K/S]ASx[S/Y]LES |
| | | (SEQ ID NO: 876) | (SEQ ID NO: 882) |
| | VL CDR3 | Qx[Q/L]FQx[S/K]LPPFT | Qx[Q/L/R]FQSLPPFT |
| | | (SEQ ID NO: 877) | (SEQ ID NO: 883) |

*Exemplary CDR sequences encompass amino acids as determined by Kabat plus Chothia

TABLE 22
Antibody sequences

| Clone | HEAVY CHAIN | LIGHT CHAIN |
|---|---|---|
| 25A | QVQLVQSGAEVKKPGASVKVSCKASGY | DIQMTQSPSTLSASVGDRVTITCRASQSIS |
| | TFDVYGISWVRQAPGQGLEWMGWIAP | SWLAWYQQKPGKAPKLLIYKASSLESG |
| | YSGNTNYAQKLQGRVTMTTDTSTSTAY | VPSRFSGSGSGTEFTLTISSLQPDDFATYY |
| | MELRSLRSDDTAVYYCARDAGTYSPFG | CQQFQSLPPFTFGGGTKVEIKRTVAAPSV |
| | YGMDVWGQGTTVTVSSASTKGPSVFPL | FIFPPSDEQLKSGTASVVCLLNNFYPREAK |
| | APSSKSTSGGTAALGCLVKDYFPEPVTVS | VQWKVDNALQSGNSQESVTEQDSKDSTYS |
| | WNSGALTSGVHTFPAVLQSSGLYSLSSVV | LSSTLTLSKADYEKHKVYACEVTHQGLSSP |
| | TVPSSSLGTQTYICNVNHKPSNTKVDKRV | VTKSFNRGEC |
| | EPKSCDKTHTCPPCPAPELLGGPSVFLFPP | |
| | KPKDTLMISRTPEVTCVVVDVSHEDPEVK | |
| | FNWYVDGVEVHNAKTKPREEQYNSTYR | |
| | VVSVLTVLHQDWLNGKEYKCKVSNKAL | |
| | PAPIEKTISKAKGQPREPQVYTLPPSREEM | |
| | TKNQVSLTCLVKGFYPSDIAVEWESNGQP | |
| | ENNYKTTPPVLDSDGSFFLYSKLTVDKSR | |
| | WQQGNVFSCSVMHEALHNHYTQKSLSLS | |
| | PG | |
| 25A3 | QVQLVQSGAEVKKPGASVKVSCKASGY | DIQMTQSPSTLSASVGDRVTITCQASQSI |
| | TFDVYGISWVRQAPGQGLEWMGWIAP | NNWLAWYQQKPGKAPKLLIYKAYNLES |
| | YSGNTNYAQKLQGRVTMTTDTSTSTAY | GVPSRFSGSGSGTEFTLTISSLQPDDFAT |
| | MELRSLRSDDTAVYYCARDAGTYSPFG | YYCQLFQSLPPFTFGGGTKVEIKRTVAA |
| | YGMDVWGQGTTVTVSSASTKGPSVFPL | PSVFIFPPSDEQLKSGTASVVCLLNNFYPRE |
| | APSSKSTSGGTAALGCLVKDYFPEPVTVS | AKVQWKVDNALQSGNSQESVTEQDSKDS |
| | WNSGALTSGVHTFPAVLQSSGLYSLSSVV | TYSLSSTLTLSKADYEKHKVYACEVTHQG |
| | TVPSSSLGTQTYICNVNHKPSNTKVDKRV | LSSPVTKSFNRGEC |
| | EPKSCDKTHTCPPCPAPELLGGPSVFLFPP | |
| | KPKDTLMISRTPEVTCVVVDVSHEDPEVK | |
| | FNWYVDGVEVHNAKTKPREEQYNSTYR | |
| | VVSVLTVLHQDWLNGKEYKCKVSNKAL | |
| | PAPIEKTISKAKGQPREPQVYTLPPSREEM | |
| | TKNQVSLTCLVKGFYPSDIAVEWESNGQP | |
| | ENNYKTTPPVLDSDGSFFLYSKLTVDKSR | |
| | WQQGNVFSCSVMHEALHNHYTQKSLSLS | |
| | PG | |
| 25A5 | QVQLVQSGAEVKKPGASVKVSCKASGY | DIQMTQSPSTLSASVGDRVTITCRASESIS |
| | TFDVYGISWVRQAPGQGLEWMGWIAP | NWLAWYQQKPGKAPKLLIYKAYSLEYG |
| | YSGNTNYAQKLQGRVTMTTDTSTSTAY | VPSRFSGSGSGTEFTLTISSLQPDDFATYY |
| | MELRSLRSDDTAVYYCARDAGTYSPFG | CQQFQKLPPFTFGGGTKVEIKRTVAAPS |
| | YGMDVWGQGTTVTVSSASTKGPSVFPL | VFIFPPSDEQLKSGTASVVCLLNNFYPREA |
| | APSSKSTSGGTAALGCLVKDYFPEPVTVS | KVQWKVDNALQSGNSQESVTEQDSKDST |
| | WNSGALTSGVHTFPAVLQSSGLYSLSSVV | YSLSSTLTLSKADYEKHKVYACEVTHQGL |
| | TVPSSSLGTQTYICNVNHKPSNTKVDKRV | SSPVTKSFNRGEC |
| | EPKSCDKTHTCPPCPAPELLGGPSVFLFPP | |
| | KPKDTLMISRTPEVTCVVVDVSHEDPEVK | |
| | FNWYVDGVEVHNAKTKPREEQYNSTYR | |
| | VVSVLTVLHQDWLNGKEYKCKVSNKAL | |
| | PAPIEKTISKAKGQPREPQVYTLPPSREEM | |
| | TKNQVSLTCLVKGFYPSDIAVEWESNGQP | |
| | ENNYKTTPPVLDSDGSFFLYSKLTVDKSR | |
| | WQQGNVFSCSVMHEALHNHYTQKSLSLS | |
| | PG | |
| 25A5T | QVQLVQSGAEVKKPGASVKVSCKASGY | DIQMTQSPSTLSASVGDRVTITCRASESIS |
| | TFDAYGISWVRQAPGQGLEWMGWIAP | NWLAWYQQKPGKAPKLLIYKAYSLEYG |
| | YSGNTNYAQKLQGRVTMTTDTSTSTAY | VPSRFSGSGSGTEFTLTISSLQPDDFATYY |
| | MELRSLRSDDTAVYYCARDAGTYSPFG | CQQFQKLPPFTFGGGTKVEIKRTVAAPS |
| | YGMDVWGQGTTVTVSSASTKGPSVFPL | VFIFPPSDEQLKSGTASVVCLLNNFYPREA |
| | APSSKSTSGGTAALGCLVKDYFPEPVTVS | KVQWKVDNALQSGNSQESVTEQDSKDST |
| | WNSGALTSGVHTFPAVLQSSGLYSLSSVV | YSLSSTLTLSKADYEKHKVYACEVTHQGL |
| | TVPSSSLGTQTYICNVNHKPSNTKVDKRV | SSPVTKSFNRGEC |
| | EPKSCDKTHTCPPCPAPELLGGPSVFLFPP | |
| | KPKDTLMISRTPEVTCVVVDVSHEDPEVK | |
| | FNWYVDGVEVHNAKTKPREEQYNSTYR | |
| | VVSVLTVLHQDWLNGKEYKCKVSNKAL | |
| | PAPIEKTISKAKGQPREPQVYTLPPSREEM | |
| | TKNQVSLTCLVKGFYPSDIAVEWESNGQP | |
| | ENNYKTTPPVLDSDGSFFLYSKLTVDKSR | |
| | WQQGNVFSCSVMHEALHNHYTQKSLSLS | |
| | PG | |
| 25G | QVQLVQSGAEVKKPGASVKVSCKASGY | DIQMTQSPSTLSASVGDRVTITCRASQSIS |
| | TFRSYGISWVRQAPGQGLEWMGWVAP | SWLAWYQQKPGKAPKLLIYKASSLESG |
| | YNGNTNYAQKLQGRVTMTTDTSTSTA | VPSRFSGSGSGTEFTLTISSLQPDDFATYY |
| | YMELRSLRSDDTAVYYCARDAGTYSPY | CQQFQSLPPFTFGGGTKVEIKRTVAAPSV |
| | GYGMDVWGQGTTVTVSSASTKGPSVFP | FIFPPSDEQLKSGTASVVCLLNNFYPREAK |
| | LAPSSKSTSGGTAALGCLVKDYFPEPVTV | VQWKVDNALQSGNSQESVTEQDSKDSTYS |
| | SWNSGALTSGVHTFPAVLQSSGLYSLSSV | LSSTLTLSKADYEKHKVYACEVTHQGLSSP |
| | VTVPSSSLGTQTYICNVNHKPSNTKVDKR | VTKSFNRGEC |
| | VEPKSCDKTHTCPPCPAPELLGGPSVFLFP | |
| | PKPKDTLMISRTPEVTCVVVDVSHEDPEV | |
| | KFNWYVDGVEVHNAKTKPREEQYNSTY | |
| | RVVSVLTVLHQDWLNGKEYKCKVSNKA | |
| | LPAPIEKTISKAKGQPREPQVYTLPPSREE | |
| | MTKNQVSLTCLVKGFYPSDIAVEWESNG | |
| | QPENNYKTTPPVLDSDGSFFLYSKLTVDK | |
| | SRWQQGNVFSCSVMHEALHNHYTQKSLS | |
| | LSPG | |
| 25G1 | QVQLVQSGAEVKKPGASVKVSCKASGY | DIQMTQSPSTLSASVGDRVTITCRASHSI |
| | TFRSYGISWVRQAPGQGLEWMGWVAP | DSWLAWYQQKPGKAPKLLIYKASYLES |
| | YSGNTNYAQKLQGRVTMTTDTSTSTAY | GVPSRFSGSGSGTEFTLTISSLQPDDFAT |
| | MELRSLRSDDTAVYYCARDAGTYSPYG | YYCQLFQSLPPFTFGGGTKVEIKRTVAA |
| | YGMDVWGQGTTVTVSSASTKGPSVFPL | PSVFIFPPSDEQLKSGTASVVCLLNNFYPRE |
| | APSSKSTSGGTAALGCLVKDYFPEPVTVS | AKVQWKVDNALQSGNSQESVTEQDSKDS |
| | WNSGALTSGVHTFPAVLQSSGLYSLSSVV | TYSLSSTLTLSKADYEKHKVYACEVTHQG |
| | TVPSSSLGTQTYICNVNHKPSNTKVDKRV | LSSPVTKSFNRGEC |
| | EPKSCDKTHTCPPCPAPELLGGPSVFLFPP | |
| | KPKDTLMISRTPEVTCVVVDVSHEDPEVK | |
| | FNWYVDGVEVHNAKTKPREEQYNSTYR | |
| | VVSVLTVLHQDWLNGKEYKCKVSNKAL | |
| | PAPIEKTISKAKGQPREPQVYTLPPSREEM | |
| | TKNQVSLTCLVKGFYPSDIAVEWESNGQP | |
| | ENNYKTTPPVLDSDGSFFLYSKLTVDKSR | |
| | WQQGNVFSCSVMHEALHNHYTQKSLSLS | |
| | PG | |
| 25G9 | QVQLVQSGAEVKKPGASVKVSCKASGY | DIQMTQSPSTLSASVGDRVTITCQASQSI |
| | TFRSYGISWVRQAPGQGLEWMGWVAP | DSWLAWYQQKPGKAPKLLIYSASYLES |
| | YSGNTNYAQKLQGRVTMTTDTSTSTAY | GVPSRFSGSGSGTEFTLTISSLQPDDFAT |
| | MELRSLRSDDTAVYYCARDAGTYSPYG | YYCQRFQSLPPFTFGGGTKVEIKRTVAA |
| | YGMDVWGQGTTVTVSSASTKGPSVFPL | PSVFIFPPSDEQLKSGTASVVCLLNNFYPRE |
| | APSSKSTSGGTAALGCLVKDYFPEPVTVS | AKVQWKVDNALQSGNSQESVTEQDSKDS |
| | WNSGALTSGVHTFPAVLQSSGLYSLSSVV | TYSLSSTLTLSKADYEKHKVYACEVTHQG |
| | TVPSSSLGTQTYICNVNHKPSNTKVDKRV | LSSPVTKSFNRGEC |
| | EPKSCDKTHTCPPCPAPELLGGPSVFLFPP | |
| | KPKDTLMISRTPEVTCVVVDVSHEDPEVK | |
| | FNWYVDGVEVHNAKTKPREEQYNSTYR | |
| | VVSVLTVLHQDWLNGKEYKCKVSNKAL | |
| | PAPIEKTISKAKGQPREPQVYTLPPSREEM | |
| | TKNQVSLTCLVKGFYPSDIAVEWESNGQP | |
| | ENNYKTTPPVLDSDGSFFLYSKLTVDKSR | |
| | WQQGNVFSCSVMHEALHNHYTQKSLSLS | |
| | PG | |
| Lineage | QVQLVQSGAEVKKPGASVKVSCKASGY | DIQMTQSPSTLSASVGDRVTITCx[R/Q]AS |
| 25A | TFDx[V/A]YGISWVRQAPGQGLEWMG | x[Q/E]SIx[S/N]x[S/N]WLAWYQQKPGKAP |
| consens | WIAPYx[N/S]GNTNYAQKLQGRVTMTT | KLLIYKAx[S/Y]x[S/N]LEx[S/Y]GVPSRFSG |
| us | DTSTSTAYMELRSLRSDDTAVYYCARD | SGSGTEFTLTISSLQPDDFATYYCQx[Q/L] |
| | AGTYSPFGYGMDVWGQGTTVTVSSAST | FQx[S/K]LPPFTFGGGTKVEIKRTVAAPSV |
| | KGPSVFPLAPSSKSTSGGTAALGCLVKDY | FIFPPSDEQLKSGTASVVCLLNNFYPREAK |
| | FPEPVTVSWNSGALTSGVHTFPAVLQSSG | VQWKVDNALQSGNSQESVTEQDSKDSTYS |
| | LYSLSSVVTVPSSSLGTQTYICNVNHKPSN | LSSTLTLSKADYEKHKVYACEVTHQGLSSP |
| | TKVDKRVEPKSCDKTHTCPPCPAPELLGG | VTKSFNRGEC |
| | PSVFLFPPKPKDTLMISRTPEVTCVVVDVS | |
| | HEDPEVKFNWYVDGVEVHNAKTKPREE | |
| | QYNSTYRVVSVLTVLHQDWLNGKEYKC | |
| | KVSNKALPAPIEKTISKAKGQPREPQVYTL | |
| | PPSREEMTKNQVSLTCLVKGFYPSDIAVE | |
| | WESNGQPENNYKTTPPVLDSDGSFFLYSK | |
| | LTVDKSRWQQGNVFSCSVMHEALHNHY | |
| | TQKSLSLSPG | |

variable regions in bold; cysteines involved in drug conjugation underlined

1. An antibody-drug conjugate comprising:
a. an antigen binding protein (Ab) which binds to the extracellular domain of human Tissue Factor (TF) (SEQ ID NO:810), wherein the Ab comprises a VH-CDR1, a VH-CDR2, a VH-CDR3, a VL-CDR1, a VL-CDR2, and a VL-CDR3, wherein
i. the VH-CDR1 comprises SEQ ID NO: 872, the VH-CDR2 comprises SEQ ID NO: 873, the VH-CDR3 comprises SEQ ID NO: 874, the VL-CDR1 comprises SEQ ID NO: 875, the VL-CDR2 comprises SEQ ID NO: 876, and the VL-CDR3 comprises SEQ ID NO: 877,
ii. the VH-CDR1, VH-CDR2, VH-CDR3, VL-CDR1, VL-CDR2, and VL-CDR3 are from the antibody designated 25A3,
iii. the VH-CDR1, VH-CDR2, VH-CDR3, VL-CDR1, VL-CDR2, and VL-CDR3 are from the antibody designated 25A,
iv. the VH-CDR1, VH-CDR2, VH-CDR3, VL-CDR1, VL-CDR2, and VL-CDR3 are from the antibody designated 25A5,
v. the VH-CDR1, VH-CDR2, VH-CDR3, VL-CDR1, VL-CDR2, and VL-CDR3 are from the antibody designated 25A5-T, or
vi. the VH-CDR1, VH-CDR2, VH-CDR3, VL-CDR1, VL-CDR2, and VL-CDR3 are from the antibody designated 25G1;
and
b. one or more linker-toxin moieties represented by Formula IV: 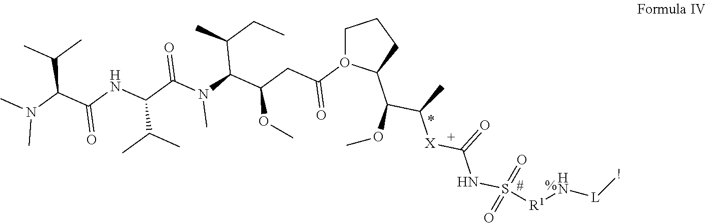
wherein:
X is *—C(O)NHCH(CH₂(R²))—⁺, wherein * and + represent the respective points of attachment as indicated in Formula IV, or X is absent;
L is a linker;
! represents the point of attachment of L to the Ab, where L is attached to the Ab through a covalent bond;
R¹ is selected from the group consisting of: 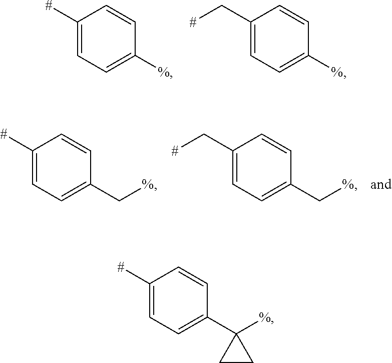
wherein # and % represent the respective points of attachment as indicated in Formula IV; and
R² is phenyl.

2. The antibody-drug conjugate of claim 1, wherein R¹ is selected from the group consisting of:

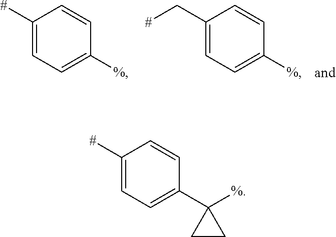

3. The antibody-drug conjugate of claim 1 or claim 2, wherein X is absent.

4. The antibody-drug conjugate of any one of claims 1-3, wherein the linker-toxin moiety of Formula IV is represented by Formula V:

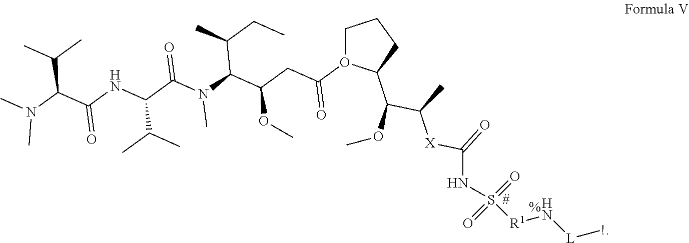

5. The antibody-drug conjugate of claim 4, wherein R¹ is selected from the group consisting of:

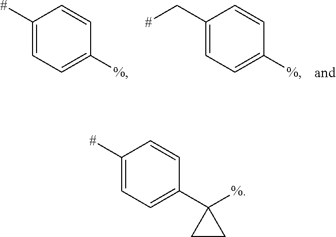

6. The antibody-drug conjugate of claim 4 or claim 5, wherein R¹ is selected from the group consisting of:

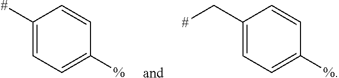

7. The antibody-drug conjugate of any one of claims 4-6, wherein R¹ is:

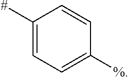

8. The antibody-drug conjugate of any one of the preceding claims, wherein L is a cleavable linker.

9. The antibody-drug conjugate of any one of the preceding claims, wherein L is a peptide-containing linker.

10. The antibody-drug conjugate of any one the preceding claims, wherein L is a protease-cleavable linker.

11. The antibody-drug conjugate of any one of claims 1-7, wherein L is a linker selected from one of N-(β-maleimidopropyloxy)-N-hydroxy succinimide ester (BMPS), N-(ε-maleimidocaproyloxy) succinimide ester (EMCS), N-[γ-maleimidobutyryloxy]succinimide ester (GMBS), 1,6-hexane-bis-vinylsulfone (HBVS), succinimidyl 4-(N-maleimidomethyl)cyclohexane-1-carboxy-(6-amidocaproate) (LC-SMCC), m-maleimidobenzoyl-N-hydroxysuccinimide ester (MBS), 4-(4-N-Maleimidophenyl)butyric acid hydrazide (MPBH), succinimidyl 3-(bromoacetamido)propionate (SBAP), succinimidyl iodoacetate (SIA), succinimidyl (4-iodoacetyl)aminobenzoate (SIAB), N-succinimidyl-3-(2-pyridyldithio) propionate (SPDP), N-succinimidyl-4-(2-pyridylthio)pentanoate (SPP), succinimidyl 4-(N-maleimidomethyl)cyclohexane-1-carboxylate (SMCC), succinimidyl 4-(p-maleimidophenyl)butyrate (SMPB), succinimidyl 6-[(β-maleimidopropionamido)hexanoate] (SMPH), iminothiolane (IT), sulfo-EMCS, sulfo-GMBS, sulfo-KMUS, sulfo-MBS, sulfo-SIAB, sulfo-SMCC, sulfo-SMPB, and succinimidyl-(4-vinylsulfone)benzoate (SVSB).

12. The antibody-drug conjugate of any one of claims 1-7, wherein L comprises a poly(ethylene)glycol chain of the formula: 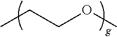
wherein g is an integer from 1-20.

13. The antibody-drug conjugate of claim 12, wherein g is 3.

14. An antibody-drug conjugate of Formula VI: 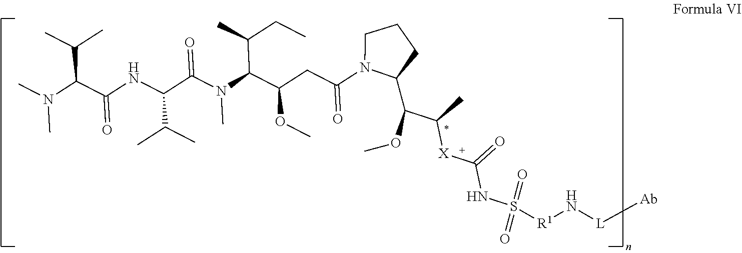
wherein:
Ab represents a tissue factor (TF) antibody;
n is an integer greater than or equal to 1;
X is *—C(O)NHCH(CH₂(R²))—⁺, wherein * and + represent the respective points of attachment as indicated in Formula VI, or X is absent;
L is a linker;
R¹ is selected from the group consisting of: 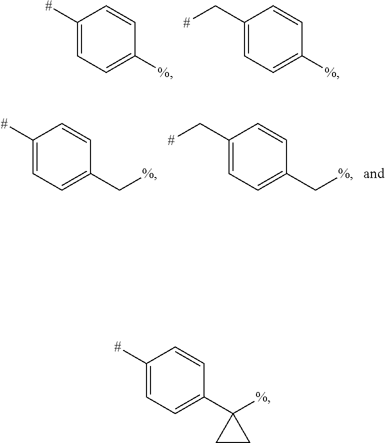
wherein # and % represent the respective points of attachment as indicated in Formula VI; and
R² is phenyl; and wherein
the Ab comprises a VH-CDR1, a VH-CDR2, a VH-CDR3, a VL-CDR1, a VL-CDR2, and a VL-CDR3, wherein
i. the VH-CDR1 comprises SEQ ID NO: 872, the VH-CDR2 comprises SEQ ID NO: 873, the VH-CDR3 comprises SEQ ID NO: 874, the VL-CDR1 comprises SEQ ID NO: 875, the VL-CDR2 comprises SEQ ID NO: 876, and the VL-CDR3 comprises SEQ ID NO: 877,
ii. the VH-CDR1, VH-CDR2, VH-CDR3, VL-CDR1, VL-CDR2, and VL-CDR3 are from the antibody designated 25A3,
iii. the VH-CDR1, VH-CDR2, VH-CDR3, VL-CDR1, VL-CDR2, and VL-CDR3 are from the antibody designated 25A,
iv. the VH-CDR1, VH-CDR2, VH-CDR3, VL-CDR1, VL-CDR2, and VL-CDR3 are from the antibody designated 25A5,
v. the VH-CDR1, VH-CDR2, VH-CDR3, VL-CDR1, VL-CDR2, and VL-CDR3 are from the antibody designated 25A5-T, or
vi. the VH-CDR1, VH-CDR2, VH-CDR3, VL-CDR1, VL-CDR2, and VL-CDR3 are from the antibody designated 25G1.

15. The antibody-drug conjugate of claim 14, wherein R¹ is selected from the group consisting of:

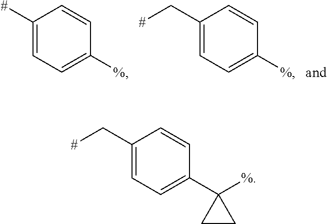

16. The antibody-drug conjugate of claim 14 or 15, wherein X is absent.

17. The antibody-drug conjugate of claim 15 or claim 16, wherein R¹ is selected from the group consisting of:

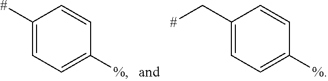

18. The antibody-drug conjugate of any one of claims 15-17, wherein R¹ is:

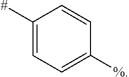

19. The antibody-drug conjugate of any one of claims 16-17, wherein L is a cleavable linker.

20. The antibody-drug conjugate of any one of claims 14-19, wherein L is a peptide-containing linker.

21. The antibody-drug conjugate of any one of claims 14-19, wherein L is a protease-cleavable linker.

22. The antibody-drug conjugate of any one of claims 14-19, wherein L is a linker selected from one of N-(β-maleimidopropyloxy)-N-hydroxy succinimide ester (BMPS), N-(ε-maleimidocaproyloxy) succinimide ester (EMCS), N-[γ-maleimidobutyryloxy]succinimide ester (GMBS), 1,6-hexane-bis-vinylsulfone (HBVS), succinimidyl 4-(N-maleimidomethyl)cyclohexane-1-carboxy-(6-amidocaproate) (LC-SMCC), m-maleimidobenzoyl-N-hydroxysuccinimide ester (MBS), 4-(4-N-Maleimidophenyl)butyric acid hydrazide (MPBH), succinimidyl 3-(bromoacetamido)propionate (SBAP), succinimidyl iodoacetate (SIA), succinimidyl (4-iodoacetyl)aminobenzoate (SIAB), N-succinimidyl-3-(2-pyridyldithio) propionate (SPDP), N-succinimidyl-4-(2-pyridylthio)pentanoate (SPP), succinimidyl 4-(N-maleimidomethyl)cyclohexane-1-carboxylate (SMCC), succinimidyl 4-(p-maleimidophenyl)butyrate (SMPB), succinimidyl 6-[(β-maleimidopropionamido)hexanoate] (SMPH), iminothiolane (IT), sulfo-EMCS, sulfo-GMBS, sulfo-KMUS, sulfo-MBS, sulfo-SIAB, sulfo-SMCC, sulfo-SMPB, and succinimidyl-(4-vinylsulfone)benzoate (SVSB).

23. The antibody-drug conjugate of any one of claims 14-19, wherein L comprises a poly(ethylene)glycol chain of the formula: 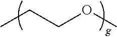
wherein g is an integer from 1-20.

24. The antibody-drug conjugate of claim 23, wherein g is 3.

25. The antibody-drug conjugate of claim 14, wherein L is represented by Formula VII: 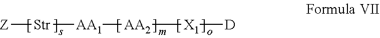
wherein:
Z represents a functional group that binds to a target group of the TF antibody;
D represents the point of attachment to the amino group as indicated in Formula VI;
Str is a stretcher;
AA₁ and AA₂ are each independently an amino acid, wherein AA₁-[AA₂]ₘ forms a protease cleavage site;
X₁ is a self-immolative group;
s is an integer selected from 0 and 1;
m is an integer selected from the group consisting of 1, 2, 3, and 4;
o is an integer selected from 0, 1, and 2.

26. The antibody-drug conjugate of claim 25, wherein n is an integer selected from the group consisting of 1, 2, 3, 4, and 5.

27. The antibody-drug conjugate of claim 25 or claim 26, wherein [Str]ₛ is selected from the group consisting of alkylene, stretchers based on aliphatic acids, stretchers based on aliphatic diacids, stretchers based on aliphatic amines and stretchers based on aliphatic diamines.

28. The antibody-drug conjugate of any one of claims 25-27, wherein [Str]ₛ is selected from the group consisting of diglycolate-based stretchers, malonate-based stretchers, caproate-based stretchers and caproamide-based stretchers.

29. The antibody-drug conjugate of claim 25 or claim 26, wherein [Str]ₛ is selected from the group consisting of glycine-based stretchers, polyethylene glycol-based stretchers, and monomethoxy polyethylene glycol-based stretchers.

30. The antibody-drug conjugate of claim 25 or claim 26, wherein [Str]ₛ is: 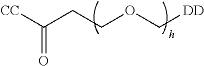
wherein
h is an integer from 1-20,
CC refers to the point of attachment to AA₁; and
DD refers to the point of attachment to Z.

31. The antibody-drug conjugate of claim 25 or claim 26, wherein [Str]ₛ is selected from: 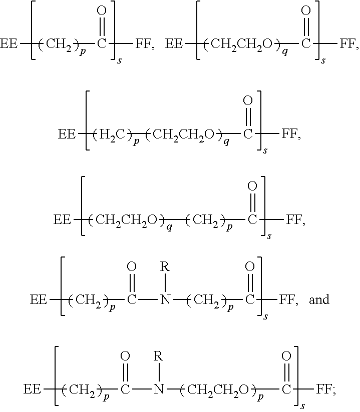
wherein:
EE and FF represent the points of attachment to Z and AA₁, respectively;
R is selected from hydrogen and C₁-C₆ alkyl;
each occurrence of p is independently an integer from 2 to 10; and
each occurrence of q is independently an integer from 1 to 10.

32. The antibody-drug conjugate of claim 25, claim 26, or claim 31, wherein [Str]ₛ is selected from the group consisting of: 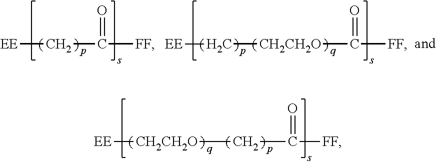
wherein:
EE and FF represent the points of attachment to Z and AA₁, respectively;
each occurrence of p is independently an integer from 2 to 10; and
each occurrence of q is independently an integer from 1 to 10.

33. The antibody-drug conjugate of claim 25, claim 26, claim 31, or claim 32, wherein [Str]ₛ is selected from: 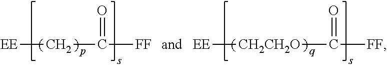
wherein:
EE and FF represent the points of attachment to Z and AA₁, respectively;
each occurrence of p is independently an integer from 2 to 6, and q is an integer from 2 to 8.

34. The antibody-drug conjugate of any one of claims 25-33, wherein AA₁-[AA₂]ₘ is selected from Val-Lys, Ala-Lys, Phe-Lys, Val-Cit, Phe-Cit, Leu-Cit, Ile-Cit, Trp-Cit, Phe-Arg, Ala-Phe, Val-Ala, Met-Lys, Asn-Lys, Ile-Pro, Ile-Val, Asp-Val, His-Val, Met-(D)Lys, Asn-(D)Lys, Val-(D)Asp, NorVal-(D)Asp, Ala-(D)Asp, Me₃Lys-Pro, PhenylGly-(D)Lys, Met-(D)Lys, Asn-(D)Lys, Pro-(D)Lys, Met-(D)Lys, Met-Cit-Val, Gly-Cit-Val, (D)Phe-Phe-Lys, (D)Ala-Phe-Lys, Gly-Phe-Leu-Gly, and Ala-Leu-Ala-Leu.

35. The antibody-drug conjugate of any one of claims 25-34, wherein m is selected from 1, 2 and 3.

36. The antibody-drug conjugate of any one of claims 25-35, wherein m is 1.

37. The antibody-drug conjugate of any one of claims 25-36, wherein AA₁-[AA₂]ₘ is a dipeptide selected from Val-Lys, Ala-Lys, Phe-Lys, Val-Cit, Phe-Cit, Leu-Cit, Ile-Cit and Trp-Cit.

38. The antibody-drug conjugate of any one of claims 25-37, wherein each X₁ is independently selected from p-aminobenzyloxycarbonyl (PABC), p-aminobenzyl ether (PABE) and methylated ethylene diamine (MED).

39. The antibody-drug conjugate of claim 30, wherein s is 1 and h is 3.

40. The antibody-drug conjugate of any one of claims 25-39, wherein s is 1.

41. The antibody-drug conjugate of any one of claims 25-40, wherein o is 0.

42. An antibody drug-conjugate comprising a linker-toxin moiety of the Formula VIII: 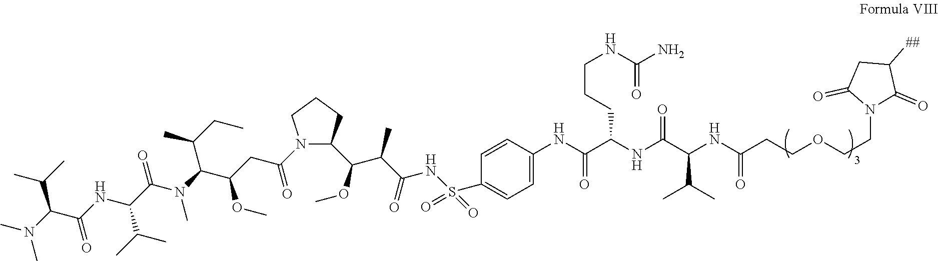
wherein ## represents the point of attachment of the linker-toxin moiety to the TF antibody and the linker-toxin moiety is attached to the TF antibody through a covalent bond.

43. An antibody-drug conjugate of Formula IX: 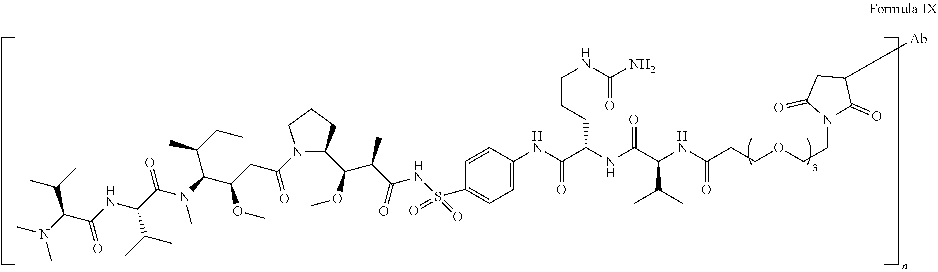
wherein:
Ab is a tissue factor (TF) antibody, wherein the Ab comprises a VH-CDR1, a VH-CDR2, a VH-CDR3, a VL-CDR1, a VL-CDR2, and a VL-CDR3, wherein
i. the VH-CDR1 comprises SEQ ID NO: 872, the VH-CDR2 comprises SEQ ID NO: 873, the VH-CDR3 comprises SEQ ID NO: 874, the VL-CDR1 comprises SEQ ID NO: 875, the VL-CDR2 comprises SEQ ID NO: 876, and the VL-CDR3 comprises SEQ ID NO: 877,
ii. the VH-CDR1, VH-CDR2, VH-CDR3, VL-CDR1, VL-CDR2, and VL-CDR3 are from the antibody designated 25A3,
iii. the VH-CDR1, VH-CDR2, VH-CDR3, VL-CDR1, VL-CDR2, and VL-CDR3 are from the antibody designated 25A,
iv. the VH-CDR1, VH-CDR2, VH-CDR3, VL-CDR1, VL-CDR2, and VL-CDR3 are from the antibody designated 25A5,
v. the VH-CDR1, VH-CDR2, VH-CDR3, VL-CDR1, VL-CDR2, and VL-CDR3 are from the antibody designated 25A5-T, or
vi. the VH-CDR1, VH-CDR2, VH-CDR3, VL-CDR1, VL-CDR2, and VL-CDR3 are from the antibody designated 25G1,
n is an integer greater than or equal to 1, and
the succinimidyl group is attached to the Ab through a covalent bond.

44. The antibody-drug conjugate of claim 43, wherein n is selected from the group consisting of 1, 2, 3, 4, and 5.

45. The antibody-drug conjugate of claim 43 or 44, wherein n is selected from the group consisting of 2, 3, and 4.

46. An antibody-drug conjugate comprising a linker as represented by Formula X: 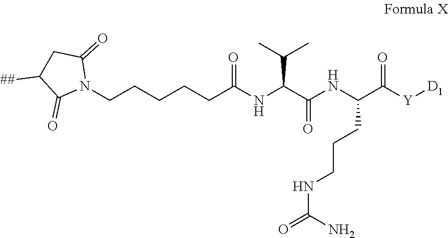
wherein:
## is the point of attachment to the antibody and the succinimidyl group is attached to the antibody through a covalent bond;
Y is one or more additional linker components, or is absent; and
D₁ is the point of attachment to a cytotoxic agent, and wherein
the Ab comprises a VH-CDR1, a VH-CDR2, a VH-CDR3, a VL-CDR1, a VL-CDR2, and a VL-CDR3, wherein
i. the VH-CDR1 comprises SEQ ID NO: 872, the VH-CDR2 comprises SEQ ID NO: 873, the VH-CDR3 comprises SEQ ID NO: 874, the VL-CDR1 comprises SEQ ID NO: 875, the VL-CDR2 comprises SEQ ID NO: 876, and the VL-CDR3 comprises SEQ ID NO: 877,
ii. the VH-CDR1, VH-CDR2, VH-CDR3, VL-CDR1, VL-CDR2, and VL-CDR3 are from the antibody designated 25A3,
iii. the VH-CDR1, VH-CDR2, VH-CDR3, VL-CDR1, VL-CDR2, and VL-CDR3 are from the antibody designated 25A,
iv. the VH-CDR1, VH-CDR2, VH-CDR3, VL-CDR1, VL-CDR2, and VL-CDR3 are from the antibody designated 25A5,
v. the VH-CDR1, VH-CDR2, VH-CDR3, VL-CDR1, VL-CDR2, and VL-CDR3 are from the antibody designated 25A5-T, or
vi. the VH-CDR1, VH-CDR2, VH-CDR3, VL-CDR1, VL-CDR2, and VL-CDR3 are from the antibody designated 25G1.

47. An antibody-drug conjugate comprising a linker as represented by Formula XI: 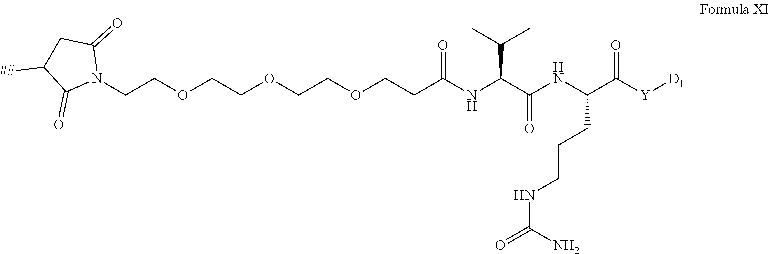
wherein:
## is the point of attachment to the antibody and the succinimidyl group is attached to the antibody through a covalent bond;
Y is one or more additional linker components, or is absent; and
D₁ is the point of attachment to a cytotoxic agent, and wherein
the Ab comprises a VH-CDR1, a VH-CDR2, a VH-CDR3, a VL-CDR1, a VL-CDR2, and a VL-CDR3, wherein
i. the VH-CDR1 comprises SEQ ID NO: 872, the VH-CDR2 comprises SEQ ID NO: 873, the VH-CDR3 comprises SEQ ID NO: 874, the VL-CDR1 comprises SEQ ID NO: 875, the VL-CDR2 comprises SEQ ID NO: 876, and the VL-CDR3 comprises SEQ ID NO: 877,
ii. the VH-CDR1, VH-CDR2, VH-CDR3, VL-CDR1, VL-CDR2, and VL-CDR3 are from the antibody designated 25A3,
iii. the VH-CDR1, VH-CDR2, VH-CDR3, VL-CDR1, VL-CDR2, and VL-CDR3 are from the antibody designated 25A,
iv. the VH-CDR1, VH-CDR2, VH-CDR3, VL-CDR1, VL-CDR2, and VL-CDR3 are from the antibody designated 25A5,
v. the VH-CDR1, VH-CDR2, VH-CDR3, VL-CDR1, VL-CDR2, and VL-CDR3 are from the antibody designated 25A5-T, or
vi. the VH-CDR1, VH-CDR2, VH-CDR3, VL-CDR1, VL-CDR2, and VL-CDR3 are from the antibody designated 25G1.

48. The antibody-drug conjugate of claim 46 or claim 47, wherein the cytotoxic agent is selected from the group consisting of a diagnostic agent, a metal chelator, an enzyme, a fluorescent compound, a bioluminescent compound, or a chemiluminescent compound.

49. The antibody-drug conjugate of claim 46 or claim 47, wherein the cytotoxic agent is a cytotoxic payload having an improved safety profile.

50. The antibody-drug conjugate of any one of the preceding claims, wherein the Ab comprises:
a. a VH sequence that is SEQ ID NO: 868 and a VL sequence that is SEQ ID NO: 869,
b. a VH that is SEQ ID NO: 151 and a VL sequence that is SEQ ID NO: 152,
c. a VH sequence that is SEQ ID NO: 113 and a VL sequence that is SEQ ID NO: 114,
d. a VH sequence that is SEQ ID NO: 189 and a VL sequence that is SEQ ID NO: 190,
e. a VH sequence that is SEQ ID NO: 836 and a VL sequence that is SEQ ID NO: 837, or
f. a VH sequence that is SEQ ID NO: 265 and a VL sequence that is SEQ ID NO: 266.

51. The antibody-drug conjugate of any one of the preceding claims, wherein the Ab comprises:
a. a heavy chain sequence that is
QVQLVQSGAEVKKPGASVKVSCKASGYTFDx[V/A]YGISWVRQAPGQ
GLEWMGWIAPYx[N/S]GNTNYAQKLQGRVTMTTDTSTSTAYMELRSL
RSDDTAVYYCARDAGTYSPFGYGMDVWGQGTTVTVSSASTKGPSVF
PLAPSSKSTSGGTAALGCLVKDYFPEPVTVSWNSGALTSGVHTFPAVL
QSSGLYSLSSVVTVPSSSLGTQTYICNVNHKPSNTKVDKRVEPKSCDK
THTCPPCPAPELLGGPSVFLFPPKPKDTLMISRTPEVTCVVVDVSHEDP
EVKFNWYVDGVEVHNAKTKPREEQYNSTYRVVSVLTVLHQDWLNG
KEYKCKVSNKALPAPIEKTISKAKGQPREPQVYTLPPSREEMTKNQVS
LTCLVKGFYPSDIAVEWESNGQPENNYKTTPPVLDSDGSFFLYSKLTV
DKSRWQQGNVFSCSVMHEALHNHYTQKSLSLSPG
and a light chain sequence that is
DIQMTQSPSTLSASVGDRVTITCx[R/Q]ASx[Q/E]SIx[S/N]x[S/
N]WLAWYQQKPGKAPKLLIYKAx[S/Y]x[S/N]LEx[S/Y]GVPSRFS
GSGSGTEFTLTISSLQPDDFATYYCQx[Q/L]FQx[S/K]LPPFTFGGG
TKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQ
GLSSPVTKSFNRGEC,
b. a heavy chain sequence that is
QVQLVQSGAEVKKPGASVKVSCKASGYTFDVYGISWVRQAPGQGLE
WMGWIAPYSGNTNYAQKLQGRVTMTTDTSTSTAYMELRSLRSDDTA
VYYCARDAGTYSPFGYGMDVWGQGTTVTVSSASTKGPSVFPLAPSSK
STSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCP
APELLGGPSVFLFPPKPKDTLMISRTPEVTCVVVDVSHEDPEVKFNWY
VDGVEVHNAKTKPREEQYNSTYRVVSVLTVLHQDWLNGKEYKCKVS
NKALPAPIEKTISKAKGQPREPQVYTLPPSREEMTKNQVSLTCLVKGFY
PSDIAVEWESNGQPENNYKTTPPVLDSDGSFFLYSKLTVDKSRWQQG
NVFSCSVMHEALHNHYTQKSLSLSPG
and a light chain sequence that is
DIQMTQSPSTLSASVGDRVTITCQASQSINNWLAWYQQKPGKAPKLLI
YKAYNLESGVPSRFSGSGSGTEFTLTISSLQPDDFATYYCQLFQSLPPFT
FGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYA
CEVTHQGLSSPVTKSFNRGEC,
c. a heavy chain sequence that is
QVQLVQSGAEVKKPGASVKVSCKASGYTFDVYGISWVRQAPGQGLE
WMGWIAPYSGNTNYAQKLQGRVTMTTDTSTSTAYMELRSLRSDDTA
VYYCARDAGTYSPFGYGMDVWGQGTTVTVSSASTKGPSVFPLAPSSK
STSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCP
APELLGGPSVFLFPPKPKDTLMISRTPEVTCVVVDVSHEDPEVKFNWY
VDGVEVHNAKTKPREEQYNSTYRVVSVLTVLHQDWLNGKEYKCKVS
NKALPAPIEKTISKAKGQPREPQVYTLPPSREEMTKNQVSLTCLVKGFY
PSDIAVEWESNGOPENNYKTTPPVLDSDGSFFLYSKLTVDKSRWOOG
NVFSCSVMHEALHNHYTQKSLSLSPG
and a light chain sequence that is
DIQMTQSPSTLSASVGDRVTITCRASQSISSWLAWYQQKPGKAPKLLIY
KASSLESGVPSRFSGSGSGTEFTLTISSLQPDDFATYYCQQFQSLPPFTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYAC
EVTHQGLSSPVTKSFNRGEC,
d. a heavy chain sequence that is
QVQLVQSGAEVKKPGASVKVSCKASGYTFDAYGISWVRQAPGQGLE
WMGWIAPYSGNTNYAQKLQGRVTMTTDTSTSTAYMELRSLRSDDTA
VYYCARDAGTYSPFGYGMDVWGQGTTVTVSSASTKGPSVFPLAPSSK
STSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCP
APELLGGPSVFLFPPKPKDTLMISRTPEVTCVVVDVSHEDPEVKFNWY
VDGVEVHNAKTKPREEQYNSTYRVVSVLTVLHQDWLNGKEYKCKVS
NKALPAPIEKTISKAKGQPREPQVYTLPPSREEMTKNQVSLTCLVKGFY
PSDIAVEWESNGQPENNYKTTPPVLDSDGSFFLYSKLTVDKSRWQQG
NVFSCSVMHEALHNHYTQKSLSLSPG
and a light chain sequence that is
DIQMTQSPSTLSASVGDRVTITCRASESISNWLAWYQQKPGKAPKLLIY
KAYSLEYGVPSRFSGSGSGTEFTLTISSLQPDDFATYYCQQFQKLPPFTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYAC
EVTHQGLSSPVTKSFNRGEC,
e. a heavy chain sequence that is
QVQLVQSGAEVKKPGASVKVSCKASGYTFRSYGISWVRQAPGQGLE
WMGWVAPYSGNTNYAQKLQGRVTMTTDTSTSTAYMELRSLRSDDT
AVYYCARDAGTYSPYGYGMDVWGQGTTVTVSSASTKGPSVFPLAPSS
KSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPC
PAPELLGGPSVFLFPPKPKDTLMISRTPEVTCVVVDVSHEDPEVKFNW
YVDGVEVHNAKTKPREEQYNSTYRVVSVLTVLHQDWLNGKEYKCK
VSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQ
GNVFSCSVMHEALHNHYTQKSLSLSPG
and a light chain sequence that is
DIQMTQSPSTLSASVGDRVTITCRASHSIDSWLAWYQQKPGKAPKLLI
YKASYLESGVPSRFSGSGSGTEFTLTISSLQPDDFATYYCQLFQSLPPFT
FGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYA
CEVTHQGLSSPVTKSFNRGEC,
or
f. a heavy chain sequence that is
QVQLVQSGAEVKKPGASVKVSCKASGYTFDVYGISWVRQAPGQGLE
WMGWIAPYSGNTNYAQKLQGRVTMTTDTSTSTAYMELRSLRSDDTA
VYYCARDAGTYSPFGYGMDVWGQGTTVTVSSASTKGPSVFPLAPSSK
STSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCP
APELLGGPSVFLFPPKPKDTLMISRTPEVTCVVVDVSHEDPEVKFNWY
VDGVEVHNAKTKPREEQYNSTYRVVSVLTVLHQDWLNGKEYKCKVS
NKALPAPIEKTISKAKGQPREPQVYTLPPSREEMTKNQVSLTCLVKGFY
PSDIAVEWESNGQPENNYKTTPPVLDSDGSFFLYSKLTVDKSRWQQG
NVFSCSVMHEALHNHYTQKSLSLSPG
and a light chain sequence that is
DIQMTQSPSTLSASVGDRVTITCRASESISNWLAWYQQKPGKAPKLLIY
KAYSLEYGVPSRFSGSGSGTEFTLTISSLQPDDFATYYCQQFQKLPPFTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYAC
EVTHQGLSSPVTKSFNRGEC.

52. An antibody-drug conjugate of Formula IX: 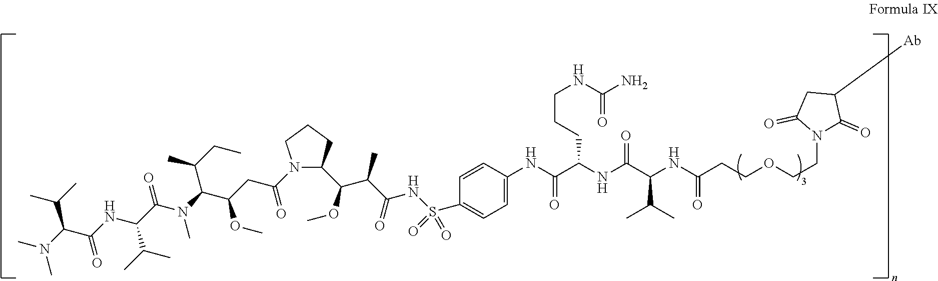
wherein:
Ab is a tissue factor (TF) antibody, wherein the Ab comprises a VH-CDR1, a VH-CDR2, a VH-CDR3, a VL-CDR1, a VL-CDR2, and a VL-CDR3 from the antibody designated 25A3, and
n is an integer greater than or equal to 1.

53. The antibody-drug conjugate of claim 52, wherein n is selected from the group consisting of 1, 2, 3, 4, and 5.

54. The antibody-drug conjugate of claim 52, wherein n is selected from the group consisting of 2, 3, and 4.

55. The antibody-drug conjugate of any one of claims 52-54, wherein the Ab comprises a VH sequence that is SEQ ID NO: 151 and a VL sequence that is SEQ ID NO: 152.

56. The antibody-drug conjugate of any one of claims 52-55, wherein the Ab comprises a full heavy chain sequence that is
QVQLVQSGAEVKKPGASVKVSCKASGYTFDVYGISWVRQAPGQGLEWMGW
IAPYSGNTNYAQKLQGRVTMTTDTSTSTAYMELRSLRSDDTAVYYCARDA
GTYSPFGYGMDVWGQGTTVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PG
and a light chain sequence that is
DIQMTQSPSTLSASVGDRVTITCQASQSINNWLAWYQQKPGKAPKLLIYK
AYNLESGVPSRFSGSGSGTEFTLTISSLQPDDFATYYCQLFQSLPPFTFG
GGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC.

57. An antibody-drug conjugate of Formula IX: 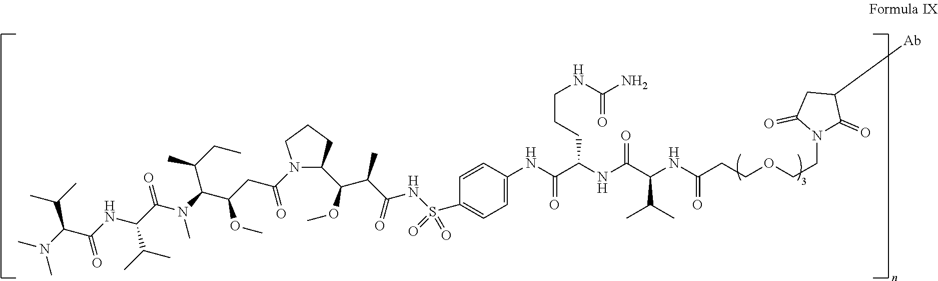
wherein:
Ab is a tissue factor (TF) antibody, wherein the Ab comprises a heavy chain sequence that is
QVQLVQSGAEVKKPGASVKVSCKASGYTFDVYGISWVRQAPGQGLEWMGWI
APYSGNTNYAQKLQGRVTMTTDTSTSTAYMELRSLRSDDTAVYYCARDAGT
YSPFGYGMDVWGQGTTVTVSSASTKGPSVFPLAPSSKSTSGGTAALGCLVK
DYFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPKD
TLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDG
SFFLYSKLTVDKSRWQQGNVFSCSVMHEALHNHYTQKSLSLSPG
and a light chain sequence that is
DIQMTQSPSTLSASVGDRVTITCQASQSINNWLAWYQQKPGKAPKLLIYK
AYNLESGVPSRFSGSGSGTEFTLTISSLQPDDFATYYCQLFQSLPPFTFG
GGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC,
and
n is an integer greater than or equal to 1.

58. The antibody-drug conjugate of claim 57, wherein n is selected from the group consisting of 1, 2, 3, 4, and 5.

59. The antibody-drug conjugate of claim 57, wherein n is selected from the group consisting of 2, 3, and 4.

60. An antibody-drug conjugate comprising an antibody (Ab) and one or more linker-toxins of the following structure: 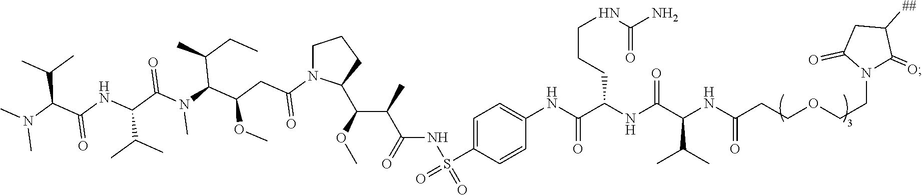
wherein:
Ab is a tissue factor (TF) antibody, wherein the Ab comprises a VH-CDR1, a VH-CDR2, a VH-CDR3, a VL-CDR1, a VL-CDR2, and a VL-CDR3 from the antibody designated 25A3;
the one or more linker-toxins are attached to the Ab through a covalent bond; and
## represents a point of attachment of the linker-toxin to the Ab.

61. An antibody-drug conjugate composition comprising the antibody-drug conjugate of claim 60, wherein the composition comprises a multiplicity of drug-antibody ratio (DAR) species, wherein the average DAR of the composition is 2-4.

62. An antibody-drug conjugate comprising an antibody (Ab) and one or more linker-toxins of the following structure: 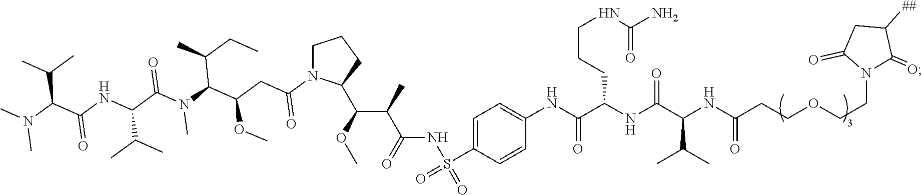
wherein:
Ab is a tissue factor (TF) antibody, wherein the Ab comprises a heavy chain sequence that is
QVQLVQSGAEVKKPGASVKVSCKASGYTFDVYGISWVRQAPGQGLEWMGW
IAPYSGNTNYAQKLQGRVTMTTDTSTSTAYMELRSLRSDDTAVYYCARDA
GTYSPFGYGMDVWGQGTTVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PG
and a light chain sequence that is
DIQMTQSPSTLSASVGDRVTITCQASQSINNWLAWYQQKPGKAPKLLIYK
AYNLESGVPSRFSGSGSGTEFTLTISSLQPDDFATYYCQLFQSLPPFTFG
GGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC,
and
the one or more linker-toxins are attached to the Ab through a covalent bond; and
## represents a point of attachment of the linker-toxin to the Ab.

63. An antibody-drug conjugate composition comprising the antibody-drug conjugate of claim 62, wherein the composition comprises a multiplicity of drug-antibody ratio (DAR) species, wherein the average DAR of the composition is 2-4.

64. The antibody-drug conjugate of any one of the preceding claims, wherein the Ab is multispecific.

65. The antibody-drug conjugate of any one of the preceding claims, wherein the Ab is a Fab, Fab′, F(ab′)₂, Fv, scFv, (scFv)₂, single chain antibody molecule, dual variable domain antibody, single variable domain antibody, linear antibody, or V domain antibody.

66. The antibody-drug conjugate of any one of the preceding claims, wherein the antibody comprises a scaffold, optionally wherein the scaffold is Fc, optionally human Fc.

67. The antibody-drug conjugate of any one of the preceding claims, wherein the antibody comprises a heavy chain constant region of a class selected from IgG, IgA, IgD, IgE, and IgM.

68. The antibody-drug conjugate of claim 67, wherein the antibody comprises a heavy chain constant region of the class IgG, wherein the heavy chain constant region is from a subclass selected from IgG1, IgG2, IgG3, and IgG4.

69. The antibody-drug conjugate of claim 68, wherein the antibody comprises a heavy chain constant region of IgG1.

70. The antibody-drug conjugate of claim 66, wherein the Fc comprises one or more modifications, wherein the one or more modifications result in increased half-life, increased antibody-dependent cellular cytotoxicity (ADCC), increased antibody-dependent cellular phagocytosis (ADCP), increased complement-dependent cytotoxicity (CDC), or decreased effector function, compared with the Fc without the one or more modifications.

71. The antibody-drug conjugate of any one of claims 1-70, wherein, upon administration to a tumor-bearing subject, the antibody-drug conjugate reduces tumor volume or inhibits tumor growth.

72. The antibody-drug conjugate of any one of claims 1-71, wherein, upon administration to a tumor-bearing subject, the antibody-drug conjugate reduces tumor volume by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99%.

73. The antibody-drug conjugate of any one of claims 1-72, wherein, upon administration to a tumor-bearing subject, the antibody-drug conjugate inhibits tumor growth by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99%.

74. The antibody-drug conjugate of any one of claims 1-73, wherein, upon administration of the antibody-drug conjugate to a subject, the antibody-drug conjugate does not result in measurable skin toxicity.

75. The antibody-drug conjugate of any one of claims 1-73, wherein, upon administration of the antibody-drug conjugate to a subject, the antibody-drug conjugate results in reduced skin toxicity relative to a different anti-TF ADC.

76. The antibody-drug conjugate of any one of claims 1-75, wherein administration of the antibody-drug conjugate to a subject does not require administration of one or more anti-inflammatory agents.

77. The antibody-drug conjugate of any one of claims 1-75, wherein administration of the antibody-drug conjugate to a subject results in a reduced need for one or more anti-inflammatory agents relative to a different anti-TF ADC.

78. The antibody-drug conjugate of claim 76 or 77, wherein the one or more anti-inflammatory agents comprises at least one of topical steroids and systemic steroids.

79. The antibody-drug conjugate of any one of claims 1-78, wherein, upon administration of the antibody-drug conjugate to a subject, the antibody-drug conjugate results in reduced or absent neutropenia in the subject relative to baseline levels.

80. The antibody-drug conjugate of any one of claims 1-78, wherein, upon administration of the antibody-drug conjugate to a subject, the antibody-drug conjugate does not alter, increase or decrease the number of monocytes in the subject relative to baseline levels.

81. The antibody-drug conjugate of any one of claims 75-80, wherein the different anti-TF ADC is identical to the antibody-drug conjugate except for being conjugated to MMAE.

82. A pharmaceutical composition comprising the antibody-drug conjugate of any one of the preceding claims and a pharmaceutically acceptable carrier.

83. A method of treating or preventing a disease or condition in a subject in need thereof, comprising administering to the subject an effective amount of the antibody-drug conjugate of any one of claims 1-81 or the pharmaceutical composition of claim 82.

84. A method of treating or delaying the onset of cancer in a subject in need thereof, comprising administering to the subject an effective amount of the antibody-drug conjugate of any one of claims 1-81.

85. The method of claim 83, wherein the disease or condition is cancer.

86. The method of claim 84 or 85, wherein the cancer is selected from the group consisting of: head and neck cancer, ovarian cancer, gastric cancer, esophageal cancer, cervical cancer, prostate cancer, pancreatic cancer, estrogen receptors negative (ER−) breast cancer, progesterone receptors negative (PR−) breast cancer, HER2 negative (HER2−) triple negative breast cancer, glioblastoma, lung cancer, bladder cancer, melanoma, and kidney cancer.

87. The method of claim 83, wherein the disease or condition involves neovascularization.

88. The method of claim 87, wherein the disease or condition involving neovascularization is cancer.

89. The method of claim 83, wherein the disease or condition involves vascular inflammation.

90. The method of any one of claims 83-89, further comprising administering one or more additional therapeutic agents to the subject.

91. The method of claim 90, wherein the composition further comprises the one or more additional therapeutic agents.

92. The method of claim 90, wherein the additional therapeutic agent is formulated in a different pharmaceutical composition.

93. The method of claim 90, wherein the additional therapeutic agent is administered prior to administering the composition.

94. The method of claim 90, wherein the additional therapeutic agent is administered after administering the composition.

95. The method of claim 90, wherein the additional therapeutic agent is administered contemporaneously with the composition.

96. The method of any one of the preceding claims, wherein the subject is a human subject.

97. A method of killing a cancer cell, comprising contacting the cancer cell with an effective amount of the antibody-drug conjugate of any one of claims 1-81.

98. A process for preparing an antibody-drug conjugate, the process comprising:
(A) reacting a nucleophilic or an electrophilic group on an antigen binding protein (Ab) which binds to the extracellular domain of human Tissue Factor (TF) (SEQ ID NO:810) with a bifunctional linker to form an Ab-linker intermediate, and reacting the Ab-linker intermediate with the −NH₂ group of a compound of general Formula I: 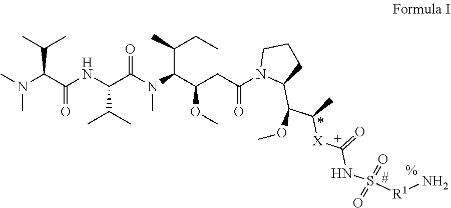
wherein:
X is *—C(O)NHCH(CH₂(R²))—⁺, wherein * and + represent the respective points of attachment as indicated in Formula I, or X is absent;
R¹ is selected from the group consisting of: 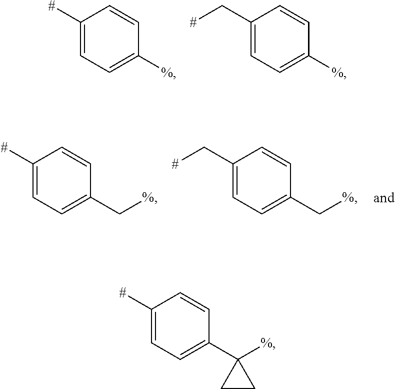
wherein # and % represent the respective points of attachment as indicated in Formula I; and R² is phenyl,
to provide the antibody drug conjugate; or
(B) reacting the —NH₂ group on the compound of general Formula I with a bifunctional linker to form a linker-toxin intermediate, and reacting the linker-toxin intermediate with a nucleophilic or an electrophilic group on an antigen binding protein (Ab) which binds to the extracellular domain of human Tissue Factor (TF) (SEQ ID NO: 810) to provide the antibody-drug conjugate, wherein, in (A) or (B),
(a) the Ab comprises a VH-CDR1, a VH-CDR2, a VH-CDR3, a VL-CDR1, a VL-CDR2, and a VL-CDR3, wherein
i. the VH-CDR1 comprises SEQ ID NO: 872, the VH-CDR2 comprises SEQ ID NO: 873, the VH-CDR3 comprises SEQ ID NO: 874, the VL-CDR1 comprises SEQ ID NO: 875, the VL-CDR2 comprises SEQ ID NO: 876, and the VL-CDR3 comprises SEQ ID NO: 877,
ii. the VH-CDR1, VH-CDR2, VH-CDR3, VL-CDR1, VL-CDR2, and VL-CDR3 are from the antibody designated 25A3,
iii. the VH-CDR1, VH-CDR2, VH-CDR3, VL-CDR1, VL-CDR2, and VL-CDR3 are from the antibody designated 25A,
iv. the VH-CDR1, VH-CDR2, VH-CDR3, VL-CDR1, VL-CDR2, and VL-CDR3 are from the antibody designated 25A5,
v. the VH-CDR1, VH-CDR2, VH-CDR3, VL-CDR1, VL-CDR2, and VL-CDR3 are from the antibody designated 25A5-T, or
vi. the VH-CDR1, VH-CDR2, VH-CDR3, VL-CDR1, VL-CDR2, and VL-CDR3 are from the antibody designated 25G1; and
(b) the antibody-drug conjugate comprises one or more moieties represented by Formula IV: 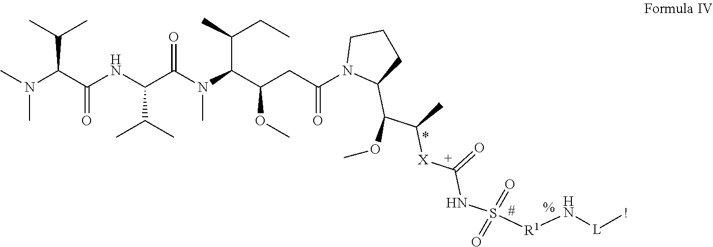
wherein:
X is *—C(O)NHCH(CH₂(R²))—⁺, wherein * and + represent the respective points of attachment as indicated in Formula IV, or X is absent;
L is a linker;
! represents the point of attachment of L to the Ab, where L is attached to the Ab through a covalent bond;
R¹ is selected from the group consisting of: 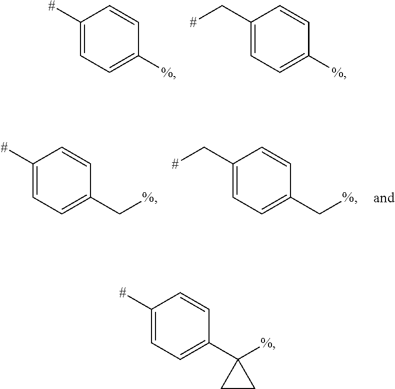
wherein # and % represent the respective points of attachment as indicated in Formula VI; and
R² is phenyl.

99. A process for preparing an antibody-drug conjugate, the process comprising:
(A) reacting a nucleophilic or an electrophilic group on an antigen binding protein (Ab) which binds to the extracellular domain of human Tissue Factor (TF) (SEQ ID NO:810) with a first linker component of a bifunctional linker that comprises two or more linker components followed by sequential addition of the remaining linker component(s) to form an Ab-linker intermediate, and reacting the Ab-linker intermediate with the —NH₂ group of a compound of general Formula I: 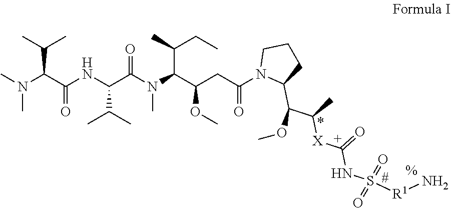
wherein:
X is *—C(O)NHCH(CH₂(R²))—⁺, wherein * and + represent the respective points of attachment as indicated in Formula I, or X is absent;
R¹ is selected from the group consisting of: 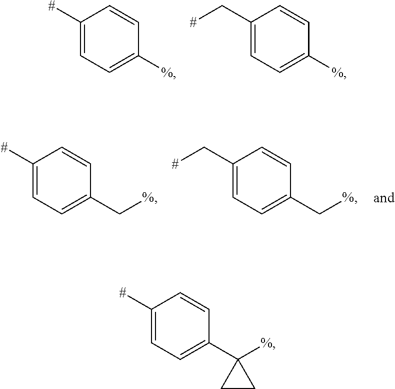
wherein # and % represent the respective points of attachment as indicated in Formula I; and R² is phenyl,
to provide the antibody drug conjugate; or
(B) reacting the —NH₂ group on the compound of general Formula I with a first linker component of a bifunctional linker that comprises two or more linker components followed by sequential addition of the remaining linker component(s) to form a linker-toxin intermediate, and reacting the linker-toxin intermediate with a nucleophilic or an electrophilic group on an antigen binding protein (Ab) which binds to the extracellular domain of human Tissue Factor (TF) (SEQ ID NO: 810) to provide the antibody-drug conjugate, wherein, in (A) or (B),
(a) the Ab comprises a VH-CDR1, a VH-CDR2, a VH-CDR3, a VL-CDR1, a VL-CDR2, and a VL-CDR3, wherein
i. the VH-CDR1 comprises SEQ ID NO: 872, the VH-CDR2 comprises SEQ ID NO: 873, the VH-CDR3 comprises SEQ ID NO: 874, the VL-CDR1 comprises SEQ ID NO: 875, the VL-CDR2 comprises SEQ ID NO: 876, and the VL-CDR3 comprises SEQ ID NO: 877,
ii. the VH-CDR1, VH-CDR2, VH-CDR3, VL-CDR1, VL-CDR2, and VL-CDR3 are from the antibody designated 25A3,
iii. the VH-CDR1, VH-CDR2, VH-CDR3, VL-CDR1, VL-CDR2, and VL-CDR3 are from the antibody designated 25A,
iv. the VH-CDR1, VH-CDR2, VH-CDR3, VL-CDR1, VL-CDR2, and VL-CDR3 are from the antibody designated 25A5,
v. the VH-CDR1, VH-CDR2, VH-CDR3, VL-CDR1, VL-CDR2, and VL-CDR3 are from the antibody designated 25A5-T, or
vi. the VH-CDR1, VH-CDR2, VH-CDR3, VL-CDR1, VL-CDR2, and VL-CDR3 are from the antibody designated 25G1; and
(b) the antibody-drug conjugate comprises one or more moieties represented by Formula IV: 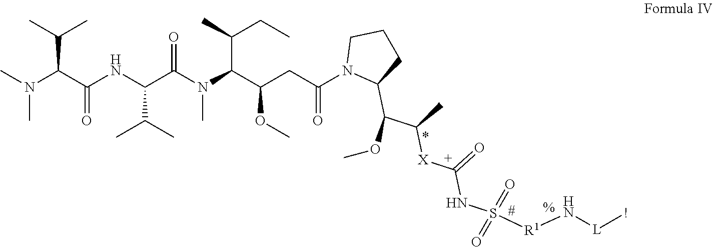
wherein:
X is *—C(O)NHCH(CH₂(R²))—⁺, wherein * and + represent the respective points of attachment as indicated in Formula IV, or X is absent;
L is a linker;
! represents the point of attachment of L to the Ab, where L is attached to the Ab through a covalent bond;
R¹ is selected from the group consisting of: 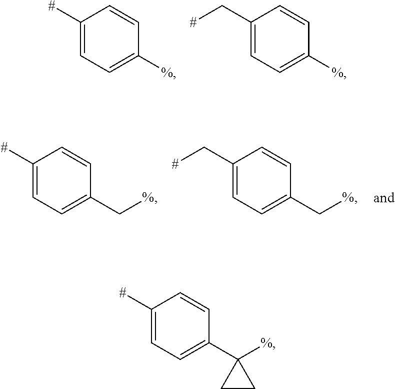
wherein # and % represent the respective points of attachment as indicated in Formula VI; and
R² is phenyl.

100. The process according to claim 98 or claim 99, wherein the nucleophilic or electrophilic group on the Ab is a thiol or an amine.

101. The process according to claim 100, further comprising treating the Ab with a reducing agent to reduce one or more disulfide linkages in the Ab to provide the nucleophilic thiol group.

102. The process according to any one of claims 98-101, wherein L is represented by: 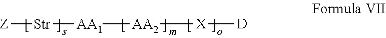
wherein:
Z represents a functional group that binds to a target group of the Ab;
D represents the point of attachment to the amino group as indicated in Formula I;
Str is a stretcher;
AA₁ and AA₂ are each independently an amino acid, wherein AA₁-[AA₂]ₘ forms a protease cleavage site;
X is a self-immolative group;
s is an integer selected from 0 and 1;
m is an integer selected from the group consisting of 1, 2, 3, and 4; and
o is an integer selected from 0, 1, and 2.

103. A kit comprising the antibody-drug conjugate of any one of claims 1-81 or the pharmaceutical composition of claim 82, and instructions for use.


SEQUENCE LISTING (944 sequences)

SEQ ID NO: 1-76 - skipped (76 entries)

SEQ ID NO: 77 - protein, 10 aa, Artificial Sequence
  Description of Artificial Sequence: Synthetic peptide
GYTFDVYGIS

SEQ ID NO: 78 - protein, 17 aa, Artificial Sequence
  Description of Artificial Sequence: Synthetic peptide
WIAPYNGNTNYAQKLQG

SEQ ID NO: 79 - protein, 14 aa, Artificial Sequence
  Description of Artificial Sequence: Synthetic peptide
DAGTYSPFGYGMDV

SEQ ID NO: 80 - protein, 11 aa, Artificial Sequence
  Description of Artificial Sequence: Synthetic peptide
RASQSISSWLA

SEQ ID NO: 81 - protein, 7 aa, Artificial Sequence
  Description of Artificial Sequence: Synthetic peptide
KASSLES

SEQ ID NO: 82 - protein, 10 aa, Artificial Sequence
  Description of Artificial Sequence: Synthetic peptide
QQFQSLPPFT

SEQ ID NO: 83 - protein, 5 aa, Artificial Sequence
  Description of Artificial Sequence: Synthetic peptide
VYGIS

SEQ ID NO: 84 - protein, 17 aa, Artificial Sequence
  Description of Artificial Sequence: Synthetic peptide
WIAPYNGNTNYAQKLQG

SEQ ID NO: 85 - protein, 14 aa, Artificial Sequence
  Description of Artificial Sequence: Synthetic peptide
DAGTYSPFGYGMDV

SEQ ID NO: 86 - protein, 11 aa, Artificial Sequence
  Description of Artificial Sequence: Synthetic peptide
RASQSISSWLA

SEQ ID NO: 87 - protein, 7 aa, Artificial Sequence
  Description of Artificial Sequence: Synthetic peptide
KASSLES

SEQ ID NO: 88 - protein, 10 aa, Artificial Sequence
  Description of Artificial Sequence: Synthetic peptide
QQFQSLPPFT

SEQ ID NO: 89 - protein, 7 aa, Artificial Sequence
  Description of Artificial Sequence: Synthetic peptide
GYTFDVY

SEQ ID NO: 90 - protein, 4 aa, Artificial Sequence
  Description of Artificial Sequence: Synthetic peptide
PYNG

SEQ ID NO: 91 - protein, 12 aa, Artificial Sequence
  Description of Artificial Sequence: Synthetic peptide
AGTYSPFGYGMD

SEQ ID NO: 92 - protein, 7 aa, Artificial Sequence
  Description of Artificial Sequence: Synthetic peptide
SQSISSW

SEQ ID NO: 93 - protein, 3 aa, Artificial Sequence
  Description of Artificial Sequence: Synthetic peptide
KAS

SEQ ID NO: 94 - protein, 7 aa, Artificial Sequence
  Description of Artificial Sequence: Synthetic peptide
FQSLPPF

SEQ ID NO: 95 - protein, 10 aa, Artificial Sequence
  Description of Artificial Sequence: Synthetic peptide
GYTFDVYGIS

SEQ ID NO: 96 - protein, 10 aa, Artificial Sequence
  Description of Artificial Sequence: Synthetic peptide
WIAPYNGNTN

SEQ ID NO: 97 - protein, 14 aa, Artificial Sequence
  Description of Artificial Sequence: Synthetic peptide
DAGTYSPFGYGMDV

SEQ ID NO: 98 - protein, 11 aa, Artificial Sequence
  Description of Artificial Sequence: Synthetic peptide
RASQSISSWLA

SEQ ID NO: 99 - protein, 7 aa, Artificial Sequence
  Description of Artificial Sequence: Synthetic peptide
KASSLES

SEQ ID NO: 100 - protein, 10 aa, Artificial Sequence
  Description of Artificial Sequence: Synthetic peptide
QQFQSLPPFT

SEQ ID NO: 101 - protein, 6 aa, Artificial Sequence
  Description of Artificial Sequence: Synthetic peptide
DVYGIS

SEQ ID NO: 102 - protein, 13 aa, Artificial Sequence
  Description of Artificial Sequence: Synthetic peptide
WMGWIAPYNGNTN

SEQ ID NO: 103 - protein, 15 aa, Artificial Sequence
  Description of Artificial Sequence: Synthetic peptide
ARDAGTYSPFGYGMD

SEQ ID NO: 104 - protein, 7 aa, Artificial Sequence
  Description of Artificial Sequence: Synthetic peptide
SSWLAWY

SEQ ID NO: 105 - protein, 10 aa, Artificial Sequence
  Description of Artificial Sequence: Synthetic peptide
LLIYKASSLE

SEQ ID NO: 106 - protein, 9 aa, Artificial Sequence
  Description of Artificial Sequence: Synthetic peptide
QQFQSLPPF

SEQ ID NO: 107 - protein, 8 aa, Artificial Sequence
  Description of Artificial Sequence: Synthetic peptide
GYTFDVYG

SEQ ID NO: 108 - protein, 8 aa, Artificial Sequence
  Description of Artificial Sequence: Synthetic peptide
IAPYNGNT

SEQ ID NO: 109 - protein, 16 aa, Artificial Sequence
  Description of Artificial Sequence: Synthetic peptide
ARDAGTYSPFGTGMDV

SEQ ID NO: 110 - protein, 6 aa, Artificial Sequence
  Description of Artificial Sequence: Synthetic peptide
QSISSW

SEQ ID NO: 111 - protein, 3 aa, Artificial Sequence
  Description of Artificial Sequence: Synthetic peptide
KAS

SEQ ID NO: 112 - protein, 10 aa, Artificial Sequence
  Description of Artificial Sequence: Synthetic peptide
QQFQSLPPFT

SEQ ID NO: 113 - protein, 123 aa, Artificial Sequence
  Description of Artificial Sequence: Synthetic polypeptide
QVQLVQSGAEVKKPGASVKVSCKASGYTFDVYGISWVRQAPGQGLEWMGWIAPYNGNTNYAQKLQGRVTMTTDTSTSTAYMELRSLRSDDTAVYYCARDAGTYSPFGYGMDVWGQGTTVTVSS

SEQ ID NO: 114 - protein, 108 aa, Artificial Sequence
  Description of Artificial Sequence: Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQFQSLPPFTFGGGTKVEIK

SEQ ID NO: 115 - protein, 10 aa, Artificial Sequence
  Description of Artificial Sequence: Synthetic peptide
GYTFDVYGIS

SEQ ID NO: 116 - protein, 17 aa, Artificial Sequence
  Description of Artificial Sequence: Synthetic peptide
WIAPYSGNTNYAQKLQG

SEQ ID NO: 117 - protein, 14 aa, Artificial Sequence
  Description of Artificial Sequence: Synthetic peptide
DAGTYSPFGYGMDV

SEQ ID NO: 118 - protein, 11 aa, Artificial Sequence
  Description of Artificial Sequence: Synthetic peptide
QASQSINNWLA

SEQ ID NO: 119 - protein, 7 aa, Artificial Sequence
  Description of Artificial Sequence: Synthetic peptide
KAYNLES

SEQ ID NO: 120 - protein, 10 aa, Artificial Sequence
  Description of Artificial Sequence: Synthetic peptide
QLFQSLPPFT

SEQ ID NO: 121 - protein, 5 aa, Artificial Sequence
  Description of Artificial Sequence: Synthetic peptide
VYGIS

SEQ ID NO: 122 - protein, 17 aa, Artificial Sequence
  Description of Artificial Sequence: Synthetic peptide
WIAPYSGNTNYAQKLQG

SEQ ID NO: 123 - protein, 14 aa, Artificial Sequence
  Description of Artificial Sequence: Synthetic peptide
DAGTYSPFGYGMDV

SEQ ID NO: 124 - protein, 11 aa, Artificial Sequence
  Description of Artificial Sequence: Synthetic peptide
QASQSINNWLA

SEQ ID NO: 125 - protein, 7 aa, Artificial Sequence
  Description of Artificial Sequence: Synthetic peptide
KAYNLES

SEQ ID NO: 126 - protein, 10 aa, Artificial Sequence
  Description of Artificial Sequence: Synthetic peptide
QLFQSLPPFT

SEQ ID NO: 127 - protein, 7 aa, Artificial Sequence
  Description of Artificial Sequence: Synthetic peptide
GYTFDVY

SEQ ID NO: 128 - protein, 4 aa, Artificial Sequence
  Description of Artificial Sequence: Synthetic peptide
PYSG

SEQ ID NO: 129 - protein, 12 aa, Artificial Sequence
  Description of Artificial Sequence: Synthetic peptide
AGTYSPFGYGMD

SEQ ID NO: 130 - protein, 7 aa, Artificial Sequence
  Description of Artificial Sequence: Synthetic peptide
SQSINNW

SEQ ID NO: 131 - protein, 3 aa, Artificial Sequence
  Description of Artificial Sequence: Synthetic peptide
KAY

SEQ ID NO: 132 - protein, 7 aa, Artificial Sequence
  Description of Artificial Sequence: Synthetic peptide
FQSLPPF

SEQ ID NO: 133 - protein, 10 aa, Artificial Sequence
  Description of Artificial Sequence: Synthetic peptide
GYTFDVYGIS

SEQ ID NO: 134 - protein, 10 aa, Artificial Sequence
  Description of Artificial Sequence: Synthetic peptide
WIAPYSGNTN

SEQ ID NO: 135 - protein, 14 aa, Artificial Sequence
  Description of Artificial Sequence: Synthetic peptide
DAGTYSPFGYGMDV

SEQ ID NO: 136 - protein, 11 aa, Artificial Sequence
  Description of Artificial Sequence: Synthetic peptide
QASQSINNWLA

SEQ ID NO: 137 - protein, 7 aa, Artificial Sequence
  Description of Artificial Sequence: Synthetic peptide
KAYNLES

SEQ ID NO: 138 - protein, 10 aa, Artificial Sequence
  Description of Artificial Sequence: Synthetic peptide
QLFQSLPPFT

SEQ ID NO: 139 - protein, 6 aa, Artificial Sequence
  Description of Artificial Sequence: Synthetic peptide
DVYGIS

SEQ ID NO: 140 - protein, 13 aa, Artificial Sequence
  Description of Artificial Sequence: Synthetic peptide
WMGWIAPYSGNTN

SEQ ID NO: 141 - protein, 15 aa, Artificial Sequence
  Description of Artificial Sequence: Synthetic peptide
ARDAGTYSPFGYGMD

SEQ ID NO: 142 - protein, 7 aa, Artificial Sequence
  Description of Artificial Sequence: Synthetic peptide
NNWLAWY

SEQ ID NO: 143 - protein, 10 aa, Artificial Sequence
  Description of Artificial Sequence: Synthetic peptide
LLIYKAYNLE

SEQ ID NO: 144 - protein, 9 aa, Artificial Sequence
  Description of Artificial Sequence: Synthetic peptide
QLFQSLPPF

SEQ ID NO: 145 - protein, 8 aa, Artificial Sequence
  Description of Artificial Sequence: Synthetic peptide
GYTFDVYG

SEQ ID NO: 146 - protein, 8 aa, Artificial Sequence
  Description of Artificial Sequence: Synthetic peptide
IAPYSGNT

SEQ ID NO: 147 - protein, 16 aa, Artificial Sequence
  Description of Artificial Sequence: Synthetic peptide
ARDAGTYSPFGTGMDV

SEQ ID NO: 148 - protein, 6 aa, Artificial Sequence
  Description of Artificial Sequence: Synthetic peptide
QSINNW

SEQ ID NO: 149 - protein, 3 aa, Artificial Sequence
  Description of Artificial Sequence: Synthetic peptide
KAY

SEQ ID NO: 150 - protein, 10 aa, Artificial Sequence
  Description of Artificial Sequence: Synthetic peptide
QLFQSLPPFT

SEQ ID NO: 151 - protein, 123 aa, Artificial Sequence
  Description of Artificial Sequence: Synthetic polypeptide
QVQLVQSGAEVKKPGASVKVSCKASGYTFDVYGISWVRQAPGQGLEWMGWIAPYSGNTNYAQKLQGRVTMTTDTSTSTAYMELRSLRSDDTAVYYCARDAGTYSPFGYGMDVWGQGTTVTVSS

SEQ ID NO: 152 - protein, 108 aa, Artificial Sequence
  Description of Artificial Sequence: Synthetic polypeptide
DIQMTQSPSTLSASVGDRVTITCQASQSINNWLAWYQQKPGKAPKLLIYKAYNLESGVPSRFSGSGSGTEFTLTISSLQPDDFATYYCQLFQSLPPFTFGGGTKVEIK

SEQ ID NO: 153 - protein, 10 aa, Artificial Sequence
  Description of Artificial Sequence: Synthetic peptide
GYTFDVYGIS

SEQ ID NO: 154 - protein, 17 aa, Artificial Sequence
  Description of Artificial Sequence: Synthetic peptide
WIAPYSGNTNYAQKLQG

SEQ ID NO: 155 - protein, 14 aa, Artificial Sequence
  Description of Artificial Sequence: Synthetic peptide
DAGTYSPFGYGMDV

SEQ ID NO: 156 - protein, 11 aa, Artificial Sequence
  Description of Artificial Sequence: Synthetic peptide
RASESISNWLA

SEQ ID NO: 157 - protein, 7 aa, Artificial Sequence
  Description of Artificial Sequence: Synthetic peptide
KAYSLEY

SEQ ID NO: 158 - protein, 10 aa, Artificial Sequence
  Description of Artificial Sequence: Synthetic peptide
QQFQKLPPFT

SEQ ID NO: 159 - protein, 5 aa, Artificial Sequence
  Description of Artificial Sequence: Synthetic peptide
VYGIS

SEQ ID NO: 160 - protein, 17 aa, Artificial Sequence
  Description of Artificial Sequence: Synthetic peptide
WIAPYSGNTNYAQKLQG

SEQ ID NO: 161 - protein, 14 aa, Artificial Sequence
  Description of Artificial Sequence: Synthetic peptide
DAGTYSPFGYGMDV

SEQ ID NO: 162 - protein, 11 aa, Artificial Sequence
  Description of Artificial Sequence: Synthetic peptide
RASESISNWLA

SEQ ID NO: 163 - protein, 7 aa, Artificial Sequence
  Description of Artificial Sequence: Synthetic peptide
KAYSLEY

SEQ ID NO: 164 - protein, 10 aa, Artificial Sequence
  Description of Artificial Sequence: Synthetic peptide
QQFQKLPPFT

SEQ ID NO: 165 - protein, 7 aa, Artificial Sequence
  Description of Artificial Sequence: Synthetic peptide
GYTFDVY

SEQ ID NO: 166 - protein, 4 aa, Artificial Sequence
  Description of Artificial Sequence: Synthetic peptide
PYSG

SEQ ID NO: 167 - protein, 12 aa, Artificial Sequence
  Description of Artificial Sequence: Synthetic peptide
AGTYSPFGYGMD

SEQ ID NO: 168 - protein, 7 aa, Artificial Sequence
  Description of Artificial Sequence: Synthetic peptide
SESISNW

SEQ ID NO: 169 - protein, 3 aa, Artificial Sequence
  Description of Artificial Sequence: Synthetic peptide
KAY

SEQ ID NO: 170 - protein, 7 aa, Artificial Sequence
  Description of Artificial Sequence: Synthetic peptide
FQKLPPF

SEQ ID NO: 171 - protein, 10 aa, Artificial Sequence
  Description of Artificial Sequence: Synthetic peptide
GYTFDVYGIS

SEQ ID NO: 172 - protein, 10 aa, Artificial Sequence
  Description of Artificial Sequence: Synthetic peptide
WIAPYSGNTN

SEQ ID NO: 173 - protein, 14 aa, Artificial Sequence
  Description of Artificial Sequence: Synthetic peptide
DAGTYSPFGYGMDV

SEQ ID NO: 174 - protein, 11 aa, Artificial Sequence
  Description of Artificial Sequence: Synthetic peptide
RASESISNWLA

SEQ ID NO: 175 - protein, 7 aa, Artificial Sequence
  Description of Artificial Sequence: Synthetic peptide
KAYSLEY

SEQ ID NO: 176 - protein, 10 aa, Artificial Sequence
  Description of Artificial Sequence: Synthetic peptide
QQFQKLPPFT

SEQ ID NO: 177 - protein, 6 aa, Artificial Sequence
  Description of Artificial Sequence: Synthetic peptide
DVYGIS

SEQ ID NO: 178 - protein, 13 aa, Artificial Sequence
  Description of Artificial Sequence: Synthetic peptide
WMGWIAPYSGNTN

SEQ ID NO: 179 - protein, 15 aa, Artificial Sequence
  Description of Artificial Sequence: Synthetic peptide
ARDAGTYSPFGYGMD

SEQ ID NO: 180 - protein, 7 aa, Artificial Sequence
  Description of Artificial Sequence: Synthetic peptide
SNWLAWY

SEQ ID NO: 181 - protein, 10 aa, Artificial Sequence
  Description of Artificial Sequence: Synthetic peptide
LLIYKAYSLE

SEQ ID NO: 182 - protein, 9 aa, Artificial Sequence
  Description of Artificial Sequence: Synthetic peptide
QQFQKLPPF

SEQ ID NO: 183 - protein, 8 aa, Artificial Sequence
  Description of Artificial Sequence: Synthetic peptide
GYTFDVYG

SEQ ID NO: 184 - protein, 8 aa, Artificial Sequence
  Description of Artificial Sequence: Synthetic peptide
IAPYSGNT

SEQ ID NO: 185 - protein, 16 aa, Artificial Sequence
  Description of Artificial Sequence: Synthetic peptide
ARDAGTYSPFGTGMDV

SEQ ID NO: 186 - protein, 6 aa, Artificial Sequence
  Description of Artificial Sequence: Synthetic peptide
ESISNW

SEQ ID NO: 187 - protein, 3 aa, Artificial Sequence
  Description of Artificial Sequence: Synthetic peptide
KAY

SEQ ID NO: 188 - protein, 10 aa, Artificial Sequence
  Description of Artificial Sequence: Synthetic peptide
QQFQKLPPFT

SEQ ID NO: 189 - protein, 123 aa, Artificial Sequence; identical to SEQ ID NO: 151
  Description of Artificial Sequence: Synthetic polypeptide

SEQ ID NO: 190 - protein, 108 aa, Artificial Sequence
  Description of Artificial Sequence: Synthetic polypeptide
DIQMTQSPSTLSASVGDRVTITCRASESISNWLAWYQQKPGKAPKLLIYKAYSLEYGVPSRFSGSGSGTEFTLTISSLQPDDFATYYCQQFQKLPPFTFGGGTKVEIK

SEQ ID NO: 191 - protein, 10 aa, Artificial Sequence
  Description of Artificial Sequence: Synthetic peptide
GYTFRSYGIS

SEQ ID NO: 192 - protein, 17 aa, Artificial Sequence
  Description of Artificial Sequence: Synthetic peptide
WVAPYNGNTNYAQKLQG

SEQ ID NO: 193 - protein, 14 aa, Artificial Sequence
  Description of Artificial Sequence: Synthetic peptide
DAGTYSPYGYGMDV

SEQ ID NO: 194 - protein, 11 aa, Artificial Sequence
  Description of Artificial Sequence: Synthetic peptide
RASQSISSWLA

SEQ ID NO: 195 - protein, 7 aa, Artificial Sequence
  Description of Artificial Sequence: Synthetic peptide
KASSLES

SEQ ID NO: 196 - protein, 10 aa, Artificial Sequence
  Description of Artificial Sequence: Synthetic peptide
QQFQSLPPFT

SEQ ID NO: 197 - protein, 5 aa, Artificial Sequence
  Description of Artificial Sequence: Synthetic peptide
SYGIS

SEQ ID NO: 198 - protein, 17 aa, Artificial Sequence
  Description of Artificial Sequence: Synthetic peptide
WVAPYNGNTNYAQKLQG

SEQ ID NO: 199 - protein, 14 aa, Artificial Sequence
  Description of Artificial Sequence: Synthetic peptide
DAGTYSPYGYGMDV

SEQ ID NO: 200 - protein, 11 aa, Artificial Sequence
  Description of Artificial Sequence: Synthetic peptide
RASQSISSWLA

SEQ ID NO: 201 - protein, 7 aa, Artificial Sequence
  Description of Artificial Sequence: Synthetic peptide
KASSLES

SEQ ID NO: 202 - protein, 10 aa, Artificial Sequence
  Description of Artificial Sequence: Synthetic peptide
QQFQSLPPFT

SEQ ID NO: 203 - protein, 7 aa, Artificial Sequence
  Description of Artificial Sequence: Synthetic peptide
GYTFRSY

SEQ ID NO: 204 - protein, 4 aa, Artificial Sequence
  Description of Artificial Sequence: Synthetic peptide
PYNG

SEQ ID NO: 205 - protein, 12 aa, Artificial Sequence
  Description of Artificial Sequence: Synthetic peptide
AGTYSPYGYGMD

SEQ ID NO: 206 - protein, 7 aa, Artificial Sequence
  Description of Artificial Sequence: Synthetic peptide
SQSISSW

SEQ ID NO: 207 - protein, 3 aa, Artificial Sequence
  Description of Artificial Sequence: Synthetic peptide
KAS

SEQ ID NO: 208 - protein, 7 aa, Artificial Sequence
  Description of Artificial Sequence: Synthetic peptide
FQSLPPF

SEQ ID NO: 209 - protein, 10 aa, Artificial Sequence
  Description of Artificial Sequence: Synthetic peptide
GYTFRSYGIS

SEQ ID NO: 210 - protein, 10 aa, Artificial Sequence
  Description of Artificial Sequence: Synthetic peptide
WVAPYNGNTN

SEQ ID NO: 211 - protein, 14 aa, Artificial Sequence
  Description of Artificial Sequence: Synthetic peptide
DAGTYSPYGYGMDV

SEQ ID NO: 212 - protein, 11 aa, Artificial Sequence
  Description of Artificial Sequence: Synthetic peptide
RASQSISSWLA

SEQ ID NO: 213 - protein, 7 aa, Artificial Sequence
  Description of Artificial Sequence: Synthetic peptide
KASSLES

SEQ ID NO: 214 - protein, 10 aa, Artificial Sequence
  Description of Artificial Sequence: Synthetic peptide
QQFQSLPPFT

SEQ ID NO: 215 - protein, 6 aa, Artificial Sequence
  Description of Artificial Sequence: Synthetic peptide
RSYGIS

SEQ ID NO: 216 - protein, 13 aa, Artificial Sequence
  Description of Artificial Sequence: Synthetic peptide
WMGWVAPYNGNTN

SEQ ID NO: 217 - protein, 15 aa, Artificial Sequence
  Description of Artificial Sequence: Synthetic peptide
ARDAGTYSPYGYGMD

SEQ ID NO: 218 - protein, 7 aa, Artificial Sequence
  Description of Artificial Sequence: Synthetic peptide
SSWLAWY

SEQ ID NO: 219 - protein, 10 aa, Artificial Sequence
  Description of Artificial Sequence: Synthetic peptide
LLIYKASSLE

SEQ ID NO: 220 - protein, 9 aa, Artificial Sequence
  Description of Artificial Sequence: Synthetic peptide
QQFQSLPPF

SEQ ID NO: 221 - protein, 8 aa, Artificial Sequence
  Description of Artificial Sequence: Synthetic peptide
GYTFRSYG

SEQ ID NO: 222 - protein, 8 aa, Artificial Sequence
  Description of Artificial Sequence: Synthetic peptide
VAPYNGNT

SEQ ID NO: 223 - protein, 16 aa, Artificial Sequence
  Description of Artificial Sequence: Synthetic peptide
ARDAGTYSPYGYGMDV

SEQ ID NO: 224 - protein, 6 aa, Artificial Sequence
  Description of Artificial Sequence: Synthetic peptide
QSISSW

SEQ ID NO: 225 - protein, 3 aa, Artificial Sequence
  Description of Artificial Sequence: Synthetic peptide
KAS

SEQ ID NO: 226 - protein, 10 aa, Artificial Sequence
  Description of Artificial Sequence: Synthetic peptide
QQFQSLPPFT

SEQ ID NO: 227 - protein, 123 aa, Artificial Sequence
  Description of Artificial Sequence: Synthetic polypeptide
QVQLVQSGAEVKKPGASVKVSCKASGYTFRSYGISWVRQAPGQGLEWMGWVAPYNGNTNYAQKLQGRVTMTTDTSTSTAYMELRSLRSDDTAVYYCARDAGTYSPYGYGMDVWGQGTTVTVSS

SEQ ID NO: 228 - protein, 108 aa, Artificial Sequence; identical to SEQ ID NO: 114
  Description of Artificial Sequence: Synthetic polypeptide

SEQ ID NO: 229 - protein, 10 aa, Artificial Sequence
  Description of Artificial Sequence: Synthetic peptide
GYTFRSYGIS

SEQ ID NO: 230 - protein, 17 aa, Artificial Sequence
  Description of Artificial Sequence: Synthetic peptide
WVAPYSGNTNYAQKLQG

SEQ ID NO: 231 - protein, 14 aa, Artificial Sequence
  Description of Artificial Sequence: Synthetic peptide
DAGTYSPYGYGMDV

SEQ ID NO: 232 - protein, 11 aa, Artificial Sequence
  Description of Artificial Sequence: Synthetic peptide
RASHSIDSWLA

SEQ ID NO: 233 - protein, 7 aa, Artificial Sequence
  Description of Artificial Sequence: Synthetic peptide
KASYLES

SEQ ID NO: 234 - protein, 10 aa, Artificial Sequence
  Description of Artificial Sequence: Synthetic peptide
QLFQSLPPFT

SEQ ID NO: 235 - protein, 5 aa, Artificial Sequence
  Description of Artificial Sequence: Synthetic peptide
SYGIS

SEQ ID NO: 236 - protein, 17 aa, Artificial Sequence
  Description of Artificial Sequence: Synthetic peptide
WVAPYSGNTNYAQKLQG

SEQ ID NO: 237 - protein, 14 aa, Artificial Sequence
  Description of Artificial Sequence: Synthetic peptide
DAGTYSPYGYGMDV

SEQ ID NO: 238 - protein, 11 aa, Artificial Sequence
  Description of Artificial Sequence: Synthetic peptide
RASHSIDSWLA

SEQ ID NO: 239 - protein, 7 aa, Artificial Sequence
  Description of Artificial Sequence: Synthetic peptide
KASYLES

SEQ ID NO: 240 - protein, 10 aa, Artificial Sequence
  Description of Artificial Sequence: Synthetic peptide
QLFQSLPPFT

SEQ ID NO: 241 - protein, 7 aa, Artificial Sequence
  Description of Artificial Sequence: Synthetic peptide
GYTFRSY

SEQ ID NO: 242 - protein, 4 aa, Artificial Sequence
  Description of Artificial Sequence: Synthetic peptide
PYSG

SEQ ID NO: 243 - protein, 12 aa, Artificial Sequence
  Description of Artificial Sequence: Synthetic peptide
AGTYSPYGYGMD

SEQ ID NO: 244 - protein, 7 aa, Artificial Sequence
  Description of Artificial Sequence: Synthetic peptide
SHSIDSW

SEQ ID NO: 245 - protein, 3 aa, Artificial Sequence
  Description of Artificial Sequence: Synthetic peptide
KAS

SEQ ID NO: 246 - protein, 7 aa, Artificial Sequence
  Description of Artificial Sequence: Synthetic peptide
FQSLPPF

SEQ ID NO: 247 - protein, 10 aa, Artificial Sequence
  Description of Artificial Sequence: Synthetic peptide
GYTFRSYGIS

SEQ ID NO: 248 - protein, 10 aa, Artificial Sequence
  Description of Artificial Sequence: Synthetic peptide
WVAPYSGNTN

SEQ ID NO: 249 - protein, 14 aa, Artificial Sequence
  Description of Artificial Sequence: Synthetic peptide
DAGTYSPYGYGMDV

SEQ ID NO: 250 - protein, 11 aa, Artificial Sequence
  Description of Artificial Sequence: Synthetic peptide
RASHSIDSWLA

SEQ ID NO: 251 - protein, 7 aa, Artificial Sequence
  Description of Artificial Sequence: Synthetic peptide
KASYLES

SEQ ID NO: 252 - protein, 10 aa, Artificial Sequence
  Description of Artificial Sequence: Synthetic peptide
QLFQSLPPFT

SEQ ID NO: 253 - protein, 6 aa, Artificial Sequence
  Description of Artificial Sequence: Synthetic peptide
RSYGIS

SEQ ID NO: 254 - protein, 13 aa, Artificial Sequence
  Description of Artificial Sequence: Synthetic peptide
WMGWVAPYSGNTN

SEQ ID NO: 255 - protein, 15 aa, Artificial Sequence
  Description of Artificial Sequence: Synthetic peptide
ARDAGTYSPYGYGMD

SEQ ID NO: 256 - protein, 7 aa, Artificial Sequence
  Description of Artificial Sequence: Synthetic peptide
DSWLAWY

SEQ ID NO: 257 - protein, 10 aa, Artificial Sequence
  Description of Artificial Sequence: Synthetic peptide
LLIYKASYLE

SEQ ID NO: 258 - protein, 9 aa, Artificial Sequence
  Description of Artificial Sequence: Synthetic peptide
QLFQSLPPF

SEQ ID NO: 259 - protein, 8 aa, Artificial Sequence
  Description of Artificial Sequence: Synthetic peptide
GYTFRSYG

SEQ ID NO: 260 - protein, 8 aa, Artificial Sequence
  Description of Artificial Sequence: Synthetic peptide
VAPYSGNT

SEQ ID NO: 261 - protein, 16 aa, Artificial Sequence
  Description of Artificial Sequence: Synthetic peptide
ARDAGTYSPYGYGMDV

SEQ ID NO: 262 - protein, 6 aa, Artificial Sequence
  Description of Artificial Sequence: Synthetic peptide
HSIDSW

SEQ ID NO: 263 - protein, 3 aa, Artificial Sequence
  Description of Artificial Sequence: Synthetic peptide
KAS

SEQ ID NO: 264 - protein, 10 aa, Artificial Sequence
  Description of Artificial Sequence: Synthetic peptide
QLFQSLPPFT

SEQ ID NO: 265 - protein, 123 aa, Artificial Sequence
  Description of Artificial Sequence: Synthetic polypeptide
QVQLVQSGAEVKKPGASVKVSCKASGYTFRSYGISWVRQAPGQGLEWMGWVAPYSGNTNYAQKLQGRVTMTTDTSTSTAYMELRSLRSDDTAVYYCARDAGTYSPYGYGMDVWGQGTTVTVSS

SEQ ID NO: 266 - protein, 108 aa, Artificial Sequence
  Description of Artificial Sequence: Synthetic polypeptide
DIQMTQSPSTLSASVGDRVTITCRASHSIDSWLAWYQQKPGKAPKLLIYKASYLESGVPSRFSGSGSGTEFTLTISSLQPDDFATYYCQLFQSLPPFTFGGGTKVEIK

SEQ ID NO: 267 - protein, 10 aa, Artificial Sequence
  Description of Artificial Sequence: Synthetic peptide
GYTFRSYGIS

SEQ ID NO: 268 - protein, 17 aa, Artificial Sequence
  Description of Artificial Sequence: Synthetic peptide
WVAPYSGNTNYAQKLQG

SEQ ID NO: 269 - protein, 14 aa, Artificial Sequence
  Description of Artificial Sequence: Synthetic peptide
DAGTYSPYGYGMDV

SEQ ID NO: 270 - protein, 11 aa, Artificial Sequence
  Description of Artificial Sequence: Synthetic peptide
QASQSIDSWLA

SEQ ID NO: 271 - protein, 7 aa, Artificial Sequence
  Description of Artificial Sequence: Synthetic peptide
SASYLES

SEQ ID NO: 272 - protein, 10 aa, Artificial Sequence
  Description of Artificial Sequence: Synthetic peptide
QRFQSLPPFT

SEQ ID NO: 273 - protein, 5 aa, Artificial Sequence
  Description of Artificial Sequence: Synthetic peptide
SYGIS

SEQ ID NO: 274 - protein, 17 aa, Artificial Sequence
  Description of Artificial Sequence: Synthetic peptide
WVAPYSGNTNYAQKLQG

SEQ ID NO: 275 - protein, 14 aa, Artificial Sequence
  Description of Artificial Sequence: Synthetic peptide
DAGTYSPYGYGMDV

SEQ ID NO: 276 - protein, 11 aa, Artificial Sequence
  Description of Artificial Sequence: Synthetic peptide
QASQSIDSWLA

SEQ ID NO: 277 - protein, 7 aa, Artificial Sequence
  Description of Artificial Sequence: Synthetic peptide
SASYLES

SEQ ID NO: 278 - protein, 10 aa, Artificial Sequence
  Description of Artificial Sequence: Synthetic peptide
QRFQSLPPFT

SEQ ID NO: 279 - protein, 7 aa, Artificial Sequence
  Description of Artificial Sequence: Synthetic peptide
GYTFRSY

SEQ ID NO: 280 - protein, 4 aa, Artificial Sequence
  Description of Artificial Sequence: Synthetic peptide
PYSG

SEQ ID NO: 281 - protein, 12 aa, Artificial Sequence
  Description of Artificial Sequence: Synthetic peptide
AGTYSPYGYGMD

SEQ ID NO: 282 - protein, 7 aa, Artificial Sequence
  Description of Artificial Sequence: Synthetic peptide
SQSIDSW

SEQ ID NO: 283 - protein, 3 aa, Artificial Sequence
  Description of Artificial Sequence: Synthetic peptide
SAS

SEQ ID NO: 284 - protein, 7 aa, Artificial Sequence
  Description of Artificial Sequence: Synthetic peptide
FQSLPPF

SEQ ID NO: 285 - protein, 10 aa, Artificial Sequence
  Description of Artificial Sequence: Synthetic peptide
GYTFRSYGIS

SEQ ID NO: 286 - protein, 10 aa, Artificial Sequence
  Description of Artificial Sequence: Synthetic peptide
WVAPYSGNTN

SEQ ID NO: 287 - protein, 14 aa, Artificial Sequence
  Description of Artificial Sequence: Synthetic peptide
DAGTYSPYGYGMDV

SEQ ID NO: 288 - protein, 11 aa, Artificial Sequence
  Description of Artificial Sequence: Synthetic peptide
QASQSIDSWLA

SEQ ID NO: 289 - protein, 7 aa, Artificial Sequence
  Description of Artificial Sequence: Synthetic peptide
SASYLES

SEQ ID NO: 290 - protein, 10 aa, Artificial Sequence
  Description of Artificial Sequence: Synthetic peptide
QRFQSLPPFT

SEQ ID NO: 291 - protein, 6 aa, Artificial Sequence
  Description of Artificial Sequence: Synthetic peptide
RSYGIS

SEQ ID NO: 292 - protein, 13 aa, Artificial Sequence
  Description of Artificial Sequence: Synthetic peptide
WMGWVAPYSGNTN

SEQ ID NO: 293 - protein, 15 aa, Artificial Sequence
  Description of Artificial Sequence: Synthetic peptide
ARDAGTYSPYGYGMD

SEQ ID NO: 294 - protein, 7 aa, Artificial Sequence
  Description of Artificial Sequence: Synthetic peptide
DSWLAWY

SEQ ID NO: 295 - protein, 10 aa, Artificial Sequence
  Description of Artificial Sequence: Synthetic peptide
LLIYSASYLE

SEQ ID NO: 296 - protein, 9 aa, Artificial Sequence
  Description of Artificial Sequence: Synthetic peptide
QRFQSLPPF

SEQ ID NO: 297 - protein, 8 aa, Artificial Sequence
  Description of Artificial Sequence: Synthetic peptide
GYTFRSYG

SEQ ID NO: 298 - protein, 8 aa, Artificial Sequence
  Description of Artificial Sequence: Synthetic peptide
VAPYSGNT

SEQ ID NO: 299 - protein, 16 aa, Artificial Sequence
  Description of Artificial Sequence: Synthetic peptide
ARDAGTYSPYGYGMDV

SEQ ID NO: 300 - protein, 6 aa, Artificial Sequence
  Description of Artificial Sequence: Synthetic peptide
QSIDSW

SEQ ID NO: 301 - protein, 3 aa, Artificial Sequence
  Description of Artificial Sequence: Synthetic peptide
SAS

SEQ ID NO: 302 - protein, 10 aa, Artificial Sequence
  Description of Artificial Sequence: Synthetic peptide
QRFQSLPPFT

SEQ ID NO: 303 - protein, 123 aa, Artificial Sequence; identical to SEQ ID NO: 265
  Description of Artificial Sequence: Synthetic polypeptide

SEQ ID NO: 304 - protein, 108 aa, Artificial Sequence
  Description of Artificial Sequence: Synthetic polypeptide
DIQMTQSPSTLSASVGDRVTITCQASQSIDSWLAWYQQKPGKAPKLLIYSASYLESGVPSRFSGSGSGTEFTLTISSLQPDDFATYYCQRFQSLPPFTFGGGTKVEIK

SEQ ID NO: 305-762 - skipped (458 entries)

SEQ ID NO: 763 - protein, 123 aa, Artificial Sequence
  Description of Artificial Sequence: Synthetic polypeptide
  MOD_RES (30)..(30): Asp or Arg
  MOD_RES (31)..(31): Ser, Val, or Ala
  MOD_RES (51)..(51): Ile or Val
  MOD_RES (55)..(55): Ser or Asn
  MOD_RES (106)..(106): Phe or Tyr
QVQLVQSGAEVKKPGASVKVSCKASGYTFXXYGISWVRQAPGQGLEWMGWXAPYXGNTNYAQKLQGRVTMTTDTSTSTAYMELRSLRSDDTAVYYCARDAGTYSPXGYGMDVWGQGTTVTVSS

SEQ ID NO: 764 - protein, 108 aa, Artificial Sequence
  Description of Artificial Sequence: Synthetic polypeptide
  MOD_RES (24)..(24): Arg or Gln
  MOD_RES (27)..(27): Gln, Glu, or His
  MOD_RES (30)..(30): Ser, Asp, or Asn
  MOD_RES (31)..(31): Ser or Asn
  MOD_RES (50)..(50): Lys or Ser
  MOD_RES (52)..(52): Ser or Tyr
  MOD_RES (53)..(53): Ser, Tyr, or Asn
  MOD_RES (56)..(56): Ser or Tyr
  MOD_RES (90)..(90): Gln, Leu, or Arg
  MOD_RES (93)..(93): Ser or Lys
DIQMTQSPSTLSASVGDRVTITCXASXSIXXWLAWYQQKPGKAPKLLIYXAXXLEXGVPSRFSGSGSGTEFTLTISSLQPDDFATYYCQXFQXLPPFTFGGGTKVEIK

SEQ ID NO: 765-772 - skipped (8 entries)

SEQ ID NO: 773 - protein, 10 aa, Artificial Sequence
  Description of Artificial Sequence: Synthetic peptide
  MOD_RES (6)..(6): Asp or Ser
  MOD_RES (10)..(10): Ala or Gly
GFTFSXYAMX

SEQ ID NO: 774 - protein, 17 aa, Artificial Sequence
  Description of Artificial Sequence: Synthetic peptide
  MOD_RES (1)..(1): Ala or Thr
XISGSGGLTYYADSVKG

SEQ ID NO: 775 - protein, 9 aa, Artificial Sequence
  Description of Artificial Sequence: Synthetic peptide
APYGYYMDV

SEQ ID NO: 776 - protein, 11 aa, Artificial Sequence
  Description of Artificial Sequence: Synthetic peptide
RASQSISSWLA

SEQ ID NO: 777 - protein, 7 aa, Artificial Sequence
  Description of Artificial Sequence: Synthetic peptide
KASSLES

SEQ ID NO: 778 - protein, 8 aa, Artificial Sequence
  Description of Artificial Sequence: Synthetic peptide
QQYKSYIT

SEQ ID NO: 779 - protein, 10 aa, Artificial Sequence
  Description of Artificial Sequence: Synthetic peptide
  MOD_RES (5)..(5): Asp or Arg
  MOD_RES (6)..(6): Ser or Val
GYTFXXYGIS

SEQ ID NO: 780 - protein, 17 aa, Artificial Sequence
  Description of Artificial Sequence: Synthetic peptide
  MOD_RES (2)..(2): Ile or Val
  MOD_RES (6)..(6): Ser or Asn
WXAPYXGNTNYAQKLQG

SEQ ID NO: 781 - protein, 14 aa, Artificial Sequence
  Description of Artificial Sequence: Synthetic peptide
  MOD_RES (8)..(8): Phe or Tyr
DAGTYSPXGYGMDV

SEQ ID NO: 782 - protein, 11 aa, Artificial Sequence
  Description of Artificial Sequence: Synthetic peptide
  MOD_RES (1)..(1): Arg or Gln
  MOD_RES (4)..(4): Gln, Glu, or His
  MOD_RES (7)..(7): Ser, Asp, or Asn
  MOD_RES (8)..(8): Ser or Asn
XASXSIXXWLA

SEQ ID NO: 783 - protein, 7 aa, Artificial Sequence
  Description of Artificial Sequence: Synthetic peptide
  MOD_RES (1)..(1): Lys or Ser
  MOD_RES (3)..(3): Ser or Tyr
  MOD_RES (4)..(4): Ser, Tyr, or Asn
  MOD_RES (7)..(7): Ser or Tyr
XAXXLEX

SEQ ID NO: 784 - protein, 10 aa, Artificial Sequence
  Description of Artificial Sequence: Synthetic peptide
  MOD_RES (2)..(2): Gln, Leu, or Arg
  MOD_RES (5)..(5): Ser or Lys
QXFQXLPPFT

SEQ ID NO: 785 - protein, 10 aa, Artificial Sequence
  Description of Artificial Sequence: Synthetic peptide
  MOD_RES (5)..(5): His or Arg
  MOD_RES (7)..(7): Arg or Tyr
GFTFXSXGMH

SEQ ID NO: 786 - protein, 17 aa, Artificial Sequence
  Description of Artificial Sequence: Synthetic peptide
VITYDGINKYYADSVEG

SEQ ID NO: 787 - protein, 10 aa, Artificial Sequence
  Description of Artificial Sequence: Synthetic peptide
DGVYYGVYDY

SEQ ID NO: 788 - protein, 17 aa, Artificial Sequence
  Description of Artificial Sequence: Synthetic peptide
KSSQSVLFSSNNKNYLA

SEQ ID NO: 789 - protein, 7 aa, Artificial Sequence
  Description of Artificial Sequence: Synthetic peptide
WASTRES

SEQ ID NO: 790 - protein, 9 aa, Artificial Sequence
  Description of Artificial Sequence: Synthetic peptide
QQFHSYPLT

SEQ ID NO: 791 - protein, 10 aa, Artificial Sequence
  Description of Artificial Sequence: Synthetic peptide
GGTFSSNAIG

SEQ ID NO: 792 - protein, 17 aa, Artificial Sequence
  Description of Artificial Sequence: Synthetic peptide
SIIPIIGFANYAQKFQG

SEQ ID NO: 793 - protein, 15 aa, Artificial Sequence
  Description of Artificial Sequence: Synthetic peptide
DSGYYYGASSFGMDV

SEQ ID NO: 794 - protein, 11 aa, Artificial Sequence
  Description of Artificial Sequence: Synthetic peptide
RASQSVSSNLA

SEQ ID NO: 795 - protein, 7 aa, Artificial Sequence
  Description of Artificial Sequence: Synthetic peptide
GASTRAT

SEQ ID NO: 796 - protein, 9 aa, Artificial Sequence
  Description of Artificial Sequence: Synthetic peptide
EQYNNLPLT

SEQ ID NO: 797 - protein, 11 aa, Artificial Sequence
  Description of Artificial Sequence: Synthetic peptide
  MOD_RES (4)..(4): Ile or Leu
  MOD_RES (8)..(8): Gln or Tyr
GGSXSSGXYWS

SEQ ID NO: 798 - protein, 16 aa, Artificial Sequence
  Description of Artificial Sequence: Synthetic peptide
  MOD_RES (3)..(3): Tyr or Gly
  MOD_RES (4)..(4): Tyr or Ala
EIXXSGSTRYNPSLKS

SEQ ID NO: 799 - protein, 16 aa, Artificial Sequence
  Description of Artificial Sequence: Synthetic peptide
  MOD_RES (2)..(2): Thr or Ala
  MOD_RES (7)..(7): Glu, Gly, or Asp
DXPYYYXGGYYYYMDV

SEQ ID NO: 800 - protein, 12 aa, Artificial Sequence
  Description of Artificial Sequence: Synthetic peptide
  MOD_RES (4)..(4): Gln, Glu, or Asp
  MOD_RES (7)..(7): Ser or Asp
  MOD_RES (10)..(10): Tyr or Phe
RASXSVXSSXLA

SEQ ID NO: 801 - protein, 7 aa, Artificial Sequence
  Description of Artificial Sequence: Synthetic peptide
  MOD_RES (3)..(3): Ser, Asp, Phe, or Tyr
  MOD_RES (4)..(4): Ser or Thr
  MOD_RES (6)..(6): Ala or Gln
  MOD_RES (7)..(7): Thr or Asn
GAXXRXX

SEQ ID NO: 802 - protein, 9 aa, Artificial Sequence
  Description of Artificial Sequence: Synthetic peptide
  MOD_RES (3)..(3): Val, Ala, or Asp
QQXGVVPYT

SEQ ID NO: 803 - protein, 10 aa, Artificial Sequence
  Description of Artificial Sequence: Synthetic peptide
GYTFANYYMH

SEQ ID NO: 804 - protein, 17 aa, Artificial Sequence
  Description of Artificial Sequence: Synthetic peptide
IINPSGGITVYAQKFQG

SEQ ID NO: 805 - protein, 12 aa, Artificial Sequence
  Description of Artificial Sequence: Synthetic peptide
GGSKVAALAFDI

SEQ ID NO: 806 - protein, 11 aa, Artificial Sequence
  Description of Artificial Sequence: Synthetic peptide
QASQDISNSLN

SEQ ID NO: 807 - protein, 7 aa, Artificial Sequence
  Description of Artificial Sequence: Synthetic peptide
DASNLET

SEQ ID NO: 808 - protein, 9 aa, Artificial Sequence
  Description of Artificial Sequence: Synthetic peptide
QQYNFHPLT

SEQ ID NO: 809 - protein, 295 aa, Homo sapiens
METPAWPRVPRPETAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREIFYIIGAVVFVVIILVIILAISLHKCRKAGVGQSWKENSPLNVS

SEQ ID NO: 810 - protein, 219 aa, Homo sapiens
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 811 - protein, 227 aa, Artificial Sequence
  Description of Artificial Sequence: Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRETGHHHHHH

SEQ ID NO: 812 - protein, 455 aa, Artificial Sequence
  Description of Artificial Sequence: Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRETGENLYFQGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 813 - protein, 296 aa, Macaca fascicularis
METPAWPRVPRPETAVARTLLLGWVFAQVAGASGTTNTVAAYNLTWKSTNFKTILEWEPKPINQVYTVQISTKSGDWKSKCFYTADTECDLTDEIVKDVKQTYLARVFSYPAGHVESTGSTEEPPYENSPEFTPYLETNLGQPTIQSFEQVGTKVNVTVQDEWTLVRRNDTFLSLRDVFGKDLIYTLYYWKSSSSGKKTAKTNTNEFLIDVDKGENYCFSVQAVIPSRRTANRKSTDSPVECMGHEKGESREIFYIIGAVVFVVIILVIILAISLHKCKKARVGRSWKENSPLNVA

SEQ ID NO: 814 - protein, 220 aa, Macaca fascicularis
SGTTNTVAAYNLTWKSTNFKTILEWEPKPINQVYTVQISTKSGDWKSKCFYTADTECDLTDEIVKDVKQTYLARVFSYPAGHVESTGSTEEPPYENSPEFTPYLETNLGQPTIQSFEQVGTKVNVTVQDEWTLVRRNDTFLSLRDVFGKDLIYTLYYWKSSSSGKKTAKTNTNEFLIDVDKGENYCFSVQAVIPSRRTANRKSTDSPVECMGHEKGESRE

SEQ ID NO: 815 - protein, 228 aa, Artificial Sequence
  Description of Artificial Sequence: Synthetic polypeptide
SGTTNTVAAYNLTWKSTNFKTILEWEPKPINQVYTVQISTKSGDWKSKCFYTADTECDLTDEIVKDVKQTYLARVFSYPAGHVESTGSTEEPPYENSPEFTPYLETNLGQPTIQSFEQVGTKVNVTVQDEWTLVRRNDTFLSLRDVFGKDLIYTLYYWKSSSSGKKTAKTNTNEFLIDVDKGENYCFSVQAVIPSRRTANRKSTDSPVECMGHEKGESRETGHHHHHH

SEQ ID NO: 816 - protein, 456 aa, Artificial Sequence
  Description of Artificial Sequence: Synthetic polypeptide
SGTTNTVAAYNLTWKSTNFKTILEWEPKPINQVYTVQISTKSGDWKSKCFYTADTECDLTDEIVKDVKQTYLARVFSYPAGHVESTGSTEEPPYENSPEFTPYLETNLGQPTIQSFEQVGTKVNVTVQDEWTLVRRNDTFLSLRDVFGKDLIYTLYYWKSSSSGKKTAKTNTNEFLIDVDKGENYCFSVQAVIPSRRTANRKSTDSPVECMGHEKGESRETGENLYFQGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 817 - protein, 294 aa, Mus musculus
MAILVRPRLLAALAPTFLGCLLLQVTAGAGIPEKAFNLTWISTDFKTILEWQPKPTNYTYTVQISDRSRNWKNKCFSTTDTECDLTDEIVKDVTWAYEAKVLSVPRRNSVHGDGDQLVIHGEEPPFTNAPKFLPYRDTNLGQPVIQQFEQDGRKLNVVVKDSLTLVRKNGTFLTLRQVFGKDLGYIITYRKGSSTGKKTNITNTNEFSIDVEEGVSYCFFVQAMIFSRKTNQNSPGSSTVCTEQWKSFLGETLIIVGAVVLLATIFIILLSISLCKRRKNRAGQKGKNTPSRLA

SEQ ID NO: 818 - protein, 223 aa, Mus musculus
AGIPEKAFNLTWISTDFKTILEWQPKPTNYTYTVQISDRSRNWKNKCFSTTDTECDLTDEIVKDVTWAYEAKVLSVPRRNSVHGDGDQLVIHGEEPPFTNAPKFLPYRDTNLGQPVIQQFEQDGRKLNVVVKDSLTLVRKNGTFLTLRQVFGKDLGYIITYRKGSSTGKKTNITNTNEFSIDVEEGVSYCFFVQAMIFSRKTNQNSPGSSTVCTEQWKSFLGE

SEQ ID NO: 819 - protein, 231 aa, Artificial Sequence
  Description of Artificial Sequence: Synthetic polypeptide
AGIPEKAFNLTWISTDFKTILEWQPKPTNYTYTVQISDRSRNWKNKCFSTTDTECDLTDEIVKDVTWAYEAKVLSVPRRNSVHGDGDQLVIHGEEPPFTNAPKFLPYRDTNLGQPVIQQFEQDGRKLNVVVKDSLTLVRKNGTFLTLRQVFGKDLGYIITYRKGSSTGKKTNITNTNEFSIDVEEGVSYCFFVQAMIFSRKTNQNSPGSSTVCTEQWKSFLGETGHHHHHH

SEQ ID NO: 820 - protein, 459 aa, Artificial Sequence
  Description of Artificial Sequence: Synthetic polypeptide
AGIPEKAFNLTWISTDFKTILEWQPKPTNYTYTVQISDRSRNWKNKCFSTTDTECDLTDEIVKDVTWAYEAKVLSVPRRNSVHGDGDQLVIHGEEPPFTNAPKFLPYRDTNLGQPVIQQFEQDGRKLNVVVKDSLTLVRKNGTFLTLRQVFGKDLGYIITYRKGSSTGKKTNITNTNEFSIDVEEGVSYCFFVQAMIFSRKTNQNSPGSSTVCTEQWKSFLGETGENLYFQGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 821 - protein, 120 aa, Artificial Sequence
  Description of Artificial Sequence: Synthetic polypeptide
EVQLVQSGAEVKKPGESLRISCKGSGYTFAPYWIEWVRQMPGKGLEWMGDILPGTGFTTYSPSFQGHVTISADKSISTAYLQWSSLKASDTAMYYCARSGYYGNSGFAYWGQGTLVTVSS

SEQ ID NO: 822 - protein, 113 aa, Artificial Sequence
  Description of Artificial Sequence: Synthetic polypeptide
DIVMTQTPLSLPVTPGEPASISCKSSQSLLSSGNQKNYLTWYLQKPGQSPQLLIYWASTRESGVPDRFSGSGSGTDFTLKISRVEAEDVGVYYCQNDYTYPLTFGQGTKLEIK

SEQ ID NO: 823 - protein, 5 aa, Artificial Sequence
  Description of Artificial Sequence: Synthetic peptide
GGGGS

SEQ ID NO: 824 - protein, 292 aa, Sus scrofa
MATPTGPPVSCPKAAVARALLLGWVLVQVAGATGTTDVIVAYNLTWKSTNFKTILEWEPKPINYVYTVQISPRLGDWKNKCFHTTDTECDVTDEIMRNVKETYVARVLSYPADTVLTAQEPPFTNSPPFTPYLDTNLGQPVIQSFEQVGTKLNVTVEAARTLVRVNGTFLRLRDVFGKDLNYTLYYWRASSTGKKKATTNTNEFLIDVDKGENYCFSVQAVIPSRRVNQKSPESRIECTSQEKAVSRELFLIVGAVVFAVIVFVLVLSVSLYKCRKERAGPSGKENAPLNVA

SEQ ID NO: 825 - protein, 216 aa, Sus scrofa
TGTTDVIVAYNLTWKSTNFKTILEWEPKPINYVYTVQISPRLGDWKNKCFHTTDTECDVTDEIMRNVKETYVARVLSYPADTVLTAQEPPFTNSPPFTPYLDTNLGQPVIQSFEQVGTKLNVTVEAARTLVRVNGTFLRLRDVFGKDLNYTLYYWRASSTGKKKATTNTNEFLIDVDKGENYCFSVQAVIPSRRVNQKSPESRIECTSQEKAVSRE

SEQ ID NO: 826 - protein, 224 aa, Artificial Sequence
  Description of Artificial Sequence: Synthetic polypeptide
TGTTDVIVAYNLTWKSTNFKTILEWEPKPINYVYTVQISPRLGDWKNKCFHTTDTECDVTDEIMRNVKETYVARVLSYPADTVLTAQEPPFTNSPPFTPYLDTNLGQPVIQSFEQVGTKLNVTVEAARTLVRVNGTFLRLRDVFGKDLNYTLYYWRASSTGKKKATTNTNEFLIDVDKGENYCFSVQAVIPSRRVNQKSPESRIECTSQEKAVSRETGHHHHHH

SEQ ID NO: 827 - protein, 452 aa, Artificial Sequence
  Description of Artificial Sequence: Synthetic polypeptide
TGTTDVIVAYNLTWKSTNFKTILEWEPKPINYVYTVQISPRLGDWKNKCFHTTDTECDVTDEIMRNVKETYVARVLSYPADTVLTAQEPPFTNSPPFTPYLDTNLGQPVIQSFEQVGTKLNVTVEAARTLVRVNGTFLRLRDVFGKDLNYTLYYWRASSTGKKKATTNTNEFLIDVDKGENYCFSVQAVIPSRRVNQKSPESRIECTSQEKAVSRETGENLYFQGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 828 - protein, 118 aa, Artificial Sequence
  Description of Artificial Sequence: Synthetic polypeptide
EVQLLESGGGLVQPGGSLRLSCAASGFTFSNYAMSWVRQAPGKGLEWVSSISGSGDYTYYTDSVKGRFTISRDNSKNTLYLQMNSLRAEDTAVYYCARSPWGYYLDSWGQGTLVTVSS

SEQ ID NO: 829 - protein, 107 aa, Artificial Sequence
  Description of Artificial Sequence: Synthetic polypeptide
DIQMTQSPPSLSASAGDRVTITCRASQGISSRLAWYQQKPEKAPKSLIYAASSLQSGVPSRFSGSGSGTDFTLTISSLQPEDFATYYCQQYNSYPYTFGQGTKLEIK

SEQ ID NO: 830 - protein, 117 aa, Artificial Sequence
  Description of Artificial Sequence: Synthetic polypeptide
QVQLVESGGGVVQPGRSLRLSCKASGFNIKDYYMHWVRQAPGKGLEWIGLIDPENGNTIYDPKFQGRFTISADNSKNTLFLQMDSLRPEDTAVYYCARDNSYYFDYWGQGTPVTVSS

SEQ ID NO: 831 - protein, 107 aa, Artificial Sequence
  Description of Artificial Sequence: Synthetic polypeptide
DIQMTQSPSSLSASVGDRVTITCKASQDIRKYLNWYQQKPGKAPKLLIYYATSLADGVPSRFSGSGSGTDYTFTISSLQPEDIATYYCLQHGESPYTFGQGTKLEIT

SEQ ID NO: 832 - protein, 292 aa, Oryctolagus cuniculus
MAPPTRLQVPRPGTAVPYTVLLGWLLAQVARAADTTGRAYNLTWKSTNFKTILEWEPKSIDHVYTVQISTRLENWKSKCFLTAETECDLTDEVVKDVGQTYMARVLSYPARNGNTTGFPEEPPFRNSPEFTPYLDTNLGQPTIQSFEQVGTKLNVTVQDARTLVRRNGTFLSLRAVFGKDLNYTLYYWRASSTGKKTATTNTNEFLIDVDKGENYCFSVQAVIPSRKRKQRSPESLTECTSREQGRAREMFFIIGAVVVVALLIIVLSVTVYKCRKARAGPSGKESSPLNIA

SEQ ID NO: 833 - protein, 218 aa, Oryctolagus cuniculus
ADTTGRAYNLTWKSTNFKTILEWEPKSIDHVYTVQISTRLENWKSKCFLTAETECDLTDEVVKDVGQTYMARVLSYPARNGNTTGFPEEPPFRNSPEFTPYLDTNLGQPTIQSFEQVGTKLNVTVQDARTLVRRNGTFLSLRAVFGKDLNYTLYYWRASSTGKKTATTNTNEFLIDVDKGENYCFSVQAVIPSRKRKQRSPESLTECTSREQGRAREM

SEQ ID NO: 834 - protein, 226 aa, Artificial Sequence
  Description of Artificial Sequence: Synthetic polypeptide
ADTTGRAYNLTWKSTNFKTILEWEPKSIDHVYTVQISTRLENWKSKCFLTAETECDLTDEVVKDVGQTYMARVLSYPARNGNTTGFPEEPPFRNSPEFTPYLDTNLGQPTIQSFEQVGTKLNVTVQDARTLVRRNGTFLSLRAVFGKDLNYTLYYWRASSTGKKTATTNTNEFLIDVDKGENYCFSVQAVIPSRKRKQRSPESLTECTSREQGRAREMTGHHHHHH

SEQ ID NO: 835 - protein, 452 aa, Artificial Sequence
  Description of Artificial Sequence: Synthetic polypeptide
ADTTGRAYNLTWKSTNFKTILEWEPKSIDHVYTVQISTRLENWKSKCFLTAETECDLTDEVVKDVGQTYMARVLSYPARNGNTTGFPEEPPFRNSPEFTPYLDTNLGQPTIQSFEQVGTKLNVTVQDARTLVRRNGTFLSLRAVFGKDLNYTLYYWRASSTGKKTATTNTNEFLIDVDKGENYCFSVQAVIPSRKRKQRSPESLTECTSREQGRAREMENLYFQGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 836 - protein, 123 aa, Artificial Sequence
  Description of Artificial Sequence: Synthetic polypeptide
QVQLVQSGAEVKKPGASVKVSCKASGYTFDAYGISWVRQAPGQGLEWMGWIAPYSGNTNYAQKLQGRVTMTTDTSTSTAYMELRSLRSDDTAVYYCARDAGTYSPFGYGMDVWGQGTTVTVSS

SEQ ID NO: 837 - protein, 108 aa, Artificial Sequence; identical to SEQ ID NO: 190
  Description of Artificial Sequence: Synthetic polypeptide

SEQ ID NO: 838 - protein, 224 aa, Rattus sp.
AGTPPGKAFNLTWISTDFKTILEWQPKPTNYTYTVQISDRSRNWKYKCTGTTDTECDLTDEIVKDVNWTYEARVLSVPWRNSTHGKETLFGTHGEEPPFTNARKFLPYRDTKIGQPVIQKYEQGGTKLKVTVKDSFTLVRKNGTFLTLRQVFGNDLGYILTYRKDSSTGRKTNTTHTNEFLIDVEKGVSYCFFAQAVIFSRKTNHKSPESITKCTEQWKSVLGE

SEQ ID NO: 839 - protein, 224 aa, Artificial Sequence
  Description of Artificial Sequence: Synthetic polypeptide
AGTPPGKAFNLTWISTDFKTILEWQPKPTNYTYTVQISDRSRNWKYKCTGTTDTECDLTDEIVKDVNWTYEARVLSVPWRNSTHGKETLFGTHGEEPPFTNARKFLPYRDTKLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 840 - protein, 218 aa, Artificial Sequence
  Description of Artificial Sequence: Synthetic polypeptide
AGTPPGKAFNLTWISTDFKTILEWQPKPTNYTYTVQISDRSRNWKYKCTGTTDTECDLTDEIVKDVNWTYEARVLSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 841 - protein, 218 aa, Artificial Sequence
  Description of Artificial Sequence: Synthetic polypeptide
AGTPPGKAFNLTWISTDFKTILEWQPKPTNYTYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 842 - protein, 219 aa, Artificial Sequence
  Description of Artificial Sequence: Synthetic polypeptide
SGTTNTVAAYNLTWKSTNFKTILEWEPKPVNQVYTVQISDRSRNWKYKCTGTTDTECDLTDEIVKDVNWTYEARVLSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 843 - protein, 219 aa, Artificial Sequence
  Description of Artificial Sequence: Synthetic polypeptide
SGTTNTVAAYNLTWKSTNFKTILEWEPKPVNQVYTVQISTKSGDWKSKCFYTTDTECDLTDEIVKDVKQTYLARVFSVPWRNSTHGTHGEEPPFTNARKFLPYRDTKLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 844 - protein, 225 aa, Artificial Sequence
  Description of Artificial Sequence: Synthetic polypeptide
SGTTNTVAAYNLTWKSTNFKTILEWEPKPVNQVYTVQISTKSGDWKSKCFYTTDTECDLTDEIVKDVKQTYLARVFSVPWRNSTHGKETLFGTHGEEPPFTNARKFLPYRDTKLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 845 - protein, 225 aa, Artificial Sequence
  Description of Artificial Sequence: Synthetic polypeptide
SGTTNTVAAYNLTWKSTNFKTILEWEPKPVNQVYTVQISTKSGDWKSKCFYTTDTECDLTDEIVKDVKQTYLARVFSVPWRNSTHGKETLFGTHGEEPPYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 846 - protein, 219 aa, Artificial Sequence
  Description of Artificial Sequence: Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLFTNARKFLPYRDTKLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 847 - protein, 219 aa, Artificial Sequence
  Description of Artificial Sequence: Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNIGQPVIQKYEQGGTKLKVTVKDSFTLVRKNGTFLTLRQVFGNDLGYILTYRKDSSTGRKTNTTHTNEFLIDVEKGVSYCFFAQAVIFSRKTNHKSPESITKCTEQWKSVLGE

SEQ ID NO: 848 - protein, 219 aa, Artificial Sequence
  Description of Artificial Sequence: Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNIGQPVIQKYEQGGTKLKVTVKDSFTLVRKNGTFLTLRQVFGNDLGYILTYRKSSSSGKKTAKTNTNEFLIDVDKGENYCFSVQAVIPSRTVNRKSTDSPVECMGQEKGEFRE

SEQ ID NO: 849 - protein, 219 aa, Artificial Sequence
  Description of Artificial Sequence: Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNIGQPVIQKYEQGGTKLKVTVKDSFTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 850 - protein, 219 aa, Artificial Sequence
  Description of Artificial Sequence: Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRKNGTFLTLRQVFGNDLGYILTYRKSSSSGKKTAKTNTNEFLIDVDKGENYCFSVQAVIPSRTVNRKSTDSPVECMGQEKGEFRE

SEQ ID NO: 851 - protein, 219 aa, Artificial Sequence
  Description of Artificial Sequence: Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRKNGTFLTLRQVFGKDLIYTLYYWKSSSSGKKTAKTNTNEFLIDVDKGENYCFSVQAVIPSRTVNRKSTDSPVECMGQEKGEFRE

SEQ ID NO: 852 - protein, 219 aa, Artificial Sequence
  Description of Artificial Sequence: Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRKNGTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 853 - protein, 219 aa, Artificial Sequence
  Description of Artificial Sequence: Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLTLRQVFGKDLIYTLYYWKSSSSGKKTAKTNTNEFLIDVDKGENYCFSVQAVIPSRTVNRKSTDSPVECMGQEKGEFRE

SEQ ID NO: 854 - protein, 219 aa, Artificial Sequence
  Description of Artificial Sequence: Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGNDLGYILTYRKSSSSGKKTAKTNTNEFLIDVDKGENYCFSVQAVIPSRTVNRKSTDSPVECMGQEKGEFRE

SEQ ID NO: 855 - protein, 219 aa, Artificial Sequence
  Description of Artificial Sequence: Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGNDLIYTLYYWKSSSSGKKTAKTNTNEFLIDVDKGENYCFSVQAVIPSRTVNRKSTDSPVECMGQEKGEFRE

SEQ ID NO: 856 - protein, 219 aa, Artificial Sequence
  Description of Artificial Sequence: Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLGYILTYRKSSSSGKKTAKTNTNEFLIDVDKGENYCFSVQAVIPSRTVNRKSTDSPVECMGQEKGEFRE

SEQ ID NO: 857 - protein, 219 aa, Artificial Sequence
  Description of Artificial Sequence: Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKDSSTGRKTNTTHTNEFLIDVEKGVSYCFFAQAVIFSRKTNHKSPESITKCTEQWKSVLGE

SEQ ID NO: 858 - protein, 219 aa, Artificial Sequence
  Description of Artificial Sequence: Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKDSSTGRKTNTTHTNEFLIDVEKGVSYCFFAQAVIPSRTVNRKSTDSPVECMGQEKGEFRE

SEQ ID NO: 859 - protein, 219 aa, Artificial Sequence
  Description of Artificial Sequence: Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKDSSTGRKTNTTHTNEFLIDVDKGENYCFSVQAVIPSRTVNRKSTDSPVECMGQEKGEFRE

SEQ ID NO: 860 - protein, 219 aa, Artificial Sequence
  Description of Artificial Sequence: Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKDSSTGRKTAKTNTNEFLIDVDKGENYCFSVQAVIPSRTVNRKSTDSPVECMGQEKGEFRE

SEQ ID NO: 861 - protein, 219 aa, Artificial Sequence
  Description of Artificial Sequence: Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTNTTHTNEFLIDVDKGENYCFSVQAVIPSRTVNRKSTDSPVECMGQEKGEFRE

SEQ ID NO: 862 - protein, 219 aa, Artificial Sequence
  Description of Artificial Sequence: Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVEKGVSYCFFAQAVIPSRTVNRKSTDSPVECMGQEKGEFRE

SEQ ID NO: 863 - protein, 219 aa, Artificial Sequence
  Description of Artificial Sequence: Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIFSRKTNHKSPESITKCTEQWKSVLGE

SEQ ID NO: 864 - protein, 224 aa, Artificial Sequence
  Description of Artificial Sequence: Synthetic polypeptide
AGTPPGKAFNLTWISTDFKTILEWQPKPTNYTYTVQISDRSRNWKYKCTGTTDTECDLTDEIVKDVNWTYEARVLSVPWRNSTHGKETLFGTHGEEPPFTNARKFLPYRDTKIGQPVIQKYEQGGTKLKVTVKDSFTLVRRNNTFLSLRDVFGKDLIYTLYYWKSSSSGKKTAKTNTNEFLIDVDKGENYCFSVQAVIFSRKTNHKSPESITKCTEQWKSVLGE

SEQ ID NO: 865 - protein, 219 aa, Artificial Sequence
  Description of Artificial Sequence: Synthetic polypeptide
SGTTNTVAAYNLTWKSTNFKTILEWEPKPVNQVYTVQISTKSGDWKSKCFYTTDTECDLTDEIVKDVNQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 866 - protein, 219 aa, Artificial Sequence; identical to SEQ ID NO: 855
  Description of Artificial Sequence: Synthetic polypeptide

SEQ ID NO: 867 - protein, 219 aa, Artificial Sequence
  Description of Artificial Sequence: Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTHTNEFLIDVDKGENYCFSVQAVIPSRKVNRKSTDSPVECMGQEKGEFRE

SEQ ID NO: 868 - protein, 123 aa, Artificial Sequence
  Description of Artificial Sequence: Synthetic polypeptide
  MOD_RES (31)..(31): Val or Ala
  MOD_RES (55)..(55): Asn or Ser
QVQLVQSGAEVKKPGASVKVSCKASGYTFDXYGISWVRQAPGQGLEWMGWIAPYXGNTNYAQKLQGRVTMTTDTSTSTAYMELRSLRSDDTAVYYCARDAGTYSPFGYGMDVWGQGTTVTVSS

SEQ ID NO: 869 - protein, 108 aa, Artificial Sequence
  Description of Artificial Sequence: Synthetic polypeptide
  MOD_RES (24)..(24): Arg or Gln
  MOD_RES (27)..(27): Gln or Glu
  MOD_RES (30)..(30): Ser or Asn
  MOD_RES (31)..(31): Ser or Asn
  MOD_RES (52)..(52): Ser or Tyr
  MOD_RES (53)..(53): Ser or Asn
  MOD_RES (56)..(56): Ser or Tyr
  MOD_RES (90)..(90): Gln or Leu
  MOD_RES (93)..(93): Ser or Lys
DIQMTQSPSTLSASVGDRVTITCXASXSIXXWLAWYQQKPGKAPKLLIYKAXXLEXGVPSRFSGSGSGTEFTLTISSLQPDDFATYYCQXFQXLPPFTFGGGTKVEIK

SEQ ID NO: 870 - protein, 123 aa, Artificial Sequence
  Description of Artificial Sequence: Synthetic polypeptide
  MOD_RES (55)..(55): Asn or Ser
QVQLVQSGAEVKKPGASVKVSCKASGYTFRSYGISWVRQAPGQGLEWMGWVAPYXGNTNYAQKLQGRVTMTTDTSTSTAYMELRSLRSDDTAVYYCARDAGTYSPYGYGMDVWGQGTTVTVSS

SEQ ID NO: 871 - protein, 108 aa, Artificial Sequence
  Description of Artificial Sequence: Synthetic polypeptide
  MOD_RES (24)..(24): Arg or Gln
  MOD_RES (27)..(27): Gln or His
  MOD_RES (30)..(30): Ser or Asp
  MOD_RES (50)..(50): Lys or Ser
  MOD_RES (53)..(53): Ser or Tyr
  MOD_RES (90)..(90): Gln, Leu, or Arg
DIQMTQSPSTLSASVGDRVTITCXASXSIXSWLAWYQQKPGKAPKLLIYXASXLESGVPSRFSGSGSGTEFTLTISSLQPDDFATYYCQXFQSLPPFTFGGGTKVEIK

SEQ ID NO: 872 - protein, 10 aa, Artificial Sequence
  Description of Artificial Sequence: Synthetic peptide
  MOD_RES (6)..(6): Val or Ala
GYTFDXYGIS

SEQ ID NO: 873 - protein, 17 aa, Artificial Sequence
  Description of Artificial Sequence: Synthetic peptide
  MOD_RES (6)..(6): Asn or Ser
WIAPYXGNTNYAQKLQG

SEQ ID NO: 874 - protein, 14 aa, Artificial Sequence
  Description of Artificial Sequence: Synthetic peptide
DAGTYSPFGYGMDV

SEQ ID NO: 875 - protein, 11 aa, Artificial Sequence
  Description of Artificial Sequence: Synthetic peptide
  MOD_RES (1)..(1): Arg or Gln
  MOD_RES (4)..(4): Gln or Glu
  MOD_RES (7)..(7): Ser or Asn
  MOD_RES (8)..(8): Ser or Asn
XASXSIXXWLA

SEQ ID NO: 876 - protein, 7 aa, Artificial Sequence
  Description of Artificial Sequence: Synthetic peptide
  MOD_RES (3)..(3): Ser or Tyr
  MOD_RES (4)..(4): Ser or Asn
  MOD_RES (7)..(7): Ser or Tyr
KAXXLEX

SEQ ID NO: 877 - protein, 10 aa, Artificial Sequence
  Description of Artificial Sequence: Synthetic peptide
  MOD_RES (2)..(2): Gln or Leu
  MOD_RES (5)..(5): Ser or Lys
QXFQXLPPFT

SEQ ID NO: 878 - protein, 10 aa, Artificial Sequence
  Description of Artificial Sequence: Synthetic peptide
GYTFRSYGIS

SEQ ID NO: 879 - protein, 17 aa, Artificial Sequence
  Description of Artificial Sequence: Synthetic peptide
  MOD_RES (6)..(6): Asn or Ser
WVAPYXGNTNYAQKLQG

SEQ ID NO: 880 - protein, 14 aa, Artificial Sequence
  Description of Artificial Sequence: Synthetic peptide
DAGTYSPYGYGMDV

SEQ ID NO: 881 - protein, 11 aa, Artificial Sequence
  Description of Artificial Sequence: Synthetic peptide
  MOD_RES (1)..(1): Arg or Gln
  MOD_RES (4)..(4): Gln or His
  MOD_RES (7)..(7): Ser or Asp
XASXSIXSWLA

SEQ ID NO: 882 - protein, 7 aa, Artificial Sequence
  Description of Artificial Sequence: Synthetic peptide
  MOD_RES (1)..(1): Lys or Ser
  MOD_RES (4)..(4): Ser or Tyr
XASXLES

SEQ ID NO: 883 - protein, 10 aa, Artificial Sequence
  Description of Artificial Sequence: Synthetic peptide
  MOD_RES (2)..(2): Gln, Leu, or Arg
QXFQSLPPFT

SEQ ID NO: 884 - protein, 10 aa, Artificial Sequence
  Description of Artificial Sequence: Synthetic peptide
GYTFDAYGIS

SEQ ID NO: 885 - protein, 17 aa, Artificial Sequence
  Description of Artificial Sequence: Synthetic peptide
WIAPYSGNTNYAQKLQG

SEQ ID NO: 886 - protein, 14 aa, Artificial Sequence
  Description of Artificial Sequence: Synthetic peptide
DAGTYSPFGYGMDV

SEQ ID NO: 887 - protein, 11 aa, Artificial Sequence
  Description of Artificial Sequence: Synthetic peptide
RASESISNWLA

SEQ ID NO: 888 - protein, 7 aa, Artificial Sequence
  Description of Artificial Sequence: Synthetic peptide
KAYSLEY

SEQ ID NO: 889 - protein, 10 aa, Artificial Sequence
  Description of Artificial Sequence: Synthetic peptide
QQFQKLPPFT

SEQ ID NO: 890 - protein, 5 aa, Artificial Sequence
  Description of Artificial Sequence: Synthetic peptide
AYGIS

SEQ ID NO: 891 - protein, 17 aa, Artificial Sequence
  Description of Artificial Sequence: Synthetic peptide
WIAPYSGNTNYAQKLQG

SEQ ID NO: 892 - protein, 14 aa, Artificial Sequence
  Description of Artificial Sequence: Synthetic peptide
DAGTYSPFGYGMDV

SEQ ID NO: 893 - protein, 11 aa, Artificial Sequence
  Description of Artificial Sequence: Synthetic peptide
RASESISNWLA

SEQ ID NO: 894 - protein, 7 aa, Artificial Sequence
  Description of Artificial Sequence: Synthetic peptide
KAYSLEY

SEQ ID NO: 895 - protein, 10 aa, Artificial Sequence
  Description of Artificial Sequence: Synthetic peptide
QQFQKLPPFT

SEQ ID NO: 896 - protein, 7 aa, Artificial Sequence
  Description of Artificial Sequence: Synthetic peptide
GYTFDAY

SEQ ID NO: 897 - protein, 4 aa, Artificial Sequence
  Description of Artificial Sequence: Synthetic peptide
PYSG

SEQ ID NO: 898 - protein, 12 aa, Artificial Sequence
  Description of Artificial Sequence: Synthetic peptide
AGTYSPFGYGMD

SEQ ID NO: 899 - protein, 7 aa, Artificial Sequence
  Description of Artificial Sequence: Synthetic peptide
SESISNW

SEQ ID NO: 900 - protein, 3 aa, Artificial Sequence
  Description of Artificial Sequence: Synthetic peptide
KAY

SEQ ID NO: 901 - protein, 7 aa, Artificial Sequence
  Description of Artificial Sequence: Synthetic peptide
FQKLPPF

SEQ ID NO: 902 - protein, 10 aa, Artificial Sequence
  Description of Artificial Sequence: Synthetic peptide
GYTFDAYGIS

SEQ ID NO: 903 - protein, 10 aa, Artificial Sequence
  Description of Artificial Sequence: Synthetic peptide
WIAPYSGNTN

SEQ ID NO: 904 - protein, 14 aa, Artificial Sequence
  Description of Artificial Sequence: Synthetic peptide
DAGTYSPFGYGMDV

SEQ ID NO: 905 - protein, 11 aa, Artificial Sequence
  Description of Artificial Sequence: Synthetic peptide
RASESISNWLA

SEQ ID NO: 906 - protein, 7 aa, Artificial Sequence
  Description of Artificial Sequence: Synthetic peptide
KAYSLEY

SEQ ID NO: 907 - protein, 10 aa, Artificial Sequence
  Description of Artificial Sequence: Synthetic peptide
QQFQKLPPFT

SEQ ID NO: 908 - protein, 6 aa, Artificial Sequence
  Description of Artificial Sequence: Synthetic peptide
DAYGIS

SEQ ID NO: 909 - protein, 13 aa, Artificial Sequence
  Description of Artificial Sequence: Synthetic peptide
WMGWIAPYSGNTN

SEQ ID NO: 910 - protein, 15 aa, Artificial Sequence
  Description of Artificial Sequence: Synthetic peptide
ARDAGTYSPFGYGMD

SEQ ID NO: 911 - protein, 7 aa, Artificial Sequence
  Description of Artificial Sequence: Synthetic peptide
SNWLAWY

SEQ ID NO: 912 - protein, 10 aa, Artificial Sequence
  Description of Artificial Sequence: Synthetic peptide
LLIYKAYSLE

SEQ ID NO: 913 - protein, 9 aa, Artificial Sequence
  Description of Artificial Sequence: Synthetic peptide
QQFQKLPPF

SEQ ID NO: 914 - protein, 8 aa, Artificial Sequence
  Description of Artificial Sequence: Synthetic peptide
GYTFDAYG

SEQ ID NO: 915 - protein, 8 aa, Artificial Sequence
  Description of Artificial Sequence: Synthetic peptide
IAPYSGNT

SEQ ID NO: 916 - protein, 16 aa, Artificial Sequence
  Description of Artificial Sequence: Synthetic peptide
ARDAGTYSPFGTGMDV

SEQ ID NO: 917 - protein, 6 aa, Artificial Sequence
  Description of Artificial Sequence: Synthetic peptide
ESISNW

SEQ ID NO: 918 - protein, 3 aa, Artificial Sequence
  Description of Artificial Sequence: Synthetic peptide
KAY

SEQ ID NO: 919 - protein, 10 aa, Artificial Sequence
  Description of Artificial Sequence: Synthetic peptide
QQFQKLPPFT

SEQ ID NO: 920 - protein, 4 aa, Homo sapiens
ELLG

SEQ ID NO: 921 - protein, 4 aa, Homo sapiens
EFLG

SEQ ID NO: 922 - protein, 4 aa, Artificial Sequence
  Description of Artificial Sequence: Synthetic peptide
GFLG

SEQ ID NO: 923 - protein, 4 aa, Artificial Sequence
  Description of Artificial Sequence: Synthetic peptide
ALAL

SEQ ID NO: 924 - protein, 452 aa, Artificial Sequence
  Description of Artificial Sequence: Synthetic polypeptide
  MOD_RES (31)..(31): Val or Ala
  MOD_RES (55)..(55): Asn or Ser
QVQLVQSGAEVKKPGASVKVSCKASGYTFDXYGISWVRQAPGQGLEWMGWIAPYXGNTNYAQKLQGRVTMTTDTSTSTAYMELRSLRSDDTAVYYCARDAGTYSPFGYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 925 - protein, 215 aa, Artificial Sequence
  Description of Artificial Sequence: Synthetic polypeptide
  MOD_RES (24)..(24): Arg or Gln
  MOD_RES (27)..(27): Gln or Glu
  MOD_RES (30)..(30): Ser or Asn
  MOD_RES (31)..(31): Ser or Asn
  MOD_RES (52)..(52): Ser or Tyr
  MOD_RES (53)..(53): Ser or Asn
  MOD_RES (56)..(56): Ser or Tyr
  MOD_RES (90)..(90): Gln or Leu
  MOD_RES (93)..(93): Ser or Lys
DIQMTQSPSTLSASVGDRVTITCXASXSIXXWLAWYQQKPGKAPKLLIYKAXXLEXGVPSRFSGSGSGTEFTLTISSLQPDDFATYYCQXFQXLPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 926 - protein, 452 aa, Artificial Sequence
  Description of Artificial Sequence: Synthetic polypeptide
QVQLVQSGAEVKKPGASVKVSCKASGYTFDVYGISWVRQAPGQGLEWMGWIAPYSGNTNYAQKLQGRVTMTTDTSTSTAYMELRSLRSDDTAVYYCARDAGTYSPFGYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 927 - protein, 215 aa, Artificial Sequence
  Description of Artificial Sequence: Synthetic polypeptide
DIQMTQSPSTLSASVGDRVTITCQASQSINNWLAWYQQKPGKAPKLLIYKAYNLESGVPSRFSGSGSGTEFTLTISSLQPDDFATYYCQLFQSLPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 928 - protein, 215 aa, Artificial Sequence
  Description of Artificial Sequence: Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQFQSLPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 929 - protein, 452 aa, Artificial Sequence
  Description of Artificial Sequence: Synthetic polypeptide
QVQLVQSGAEVKKPGASVKVSCKASGYTFDAYGISWVRQAPGQGLEWMGWIAPYSGNTNYAQKLQGRVTMTTDTSTSTAYMELRSLRSDDTAVYYCARDAGTYSPFGYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 930 - protein, 215 aa, Artificial Sequence
  Description of Artificial Sequence: Synthetic polypeptide
DIQMTQSPSTLSASVGDRVTITCRASESISNWLAWYQQKPGKAPKLLIYKAYSLEYGVPSRFSGSGSGTEFTLTISSLQPDDFATYYCQQFQKLPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 931 - protein, 452 aa, Artificial Sequence
  Description of Artificial Sequence: Synthetic polypeptide
QVQLVQSGAEVKKPGASVKVSCKASGYTFRSYGISWVRQAPGQGLEWMGWVAPYSGNTNYAQKLQGRVTMTTDTSTSTAYMELRSLRSDDTAVYYCARDAGTYSPYGYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 932 - protein, 215 aa, Artificial Sequence
  Description of Artificial Sequence: Synthetic polypeptide
DIQMTQSPSTLSASVGDRVTITCRASHSIDSWLAWYQQKPGKAPKLLIYKASYLESGVPSRFSGSGSGTEFTLTISSLQPDDFATYYCQLFQSLPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 933 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 934 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 935 - protein, 113 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 936 - protein, 106 aa, Homo sapiens [sequence omitted]

SEQ ID NO: 937 - protein, 219 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 938 - protein, 231 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 939 - protein, 106 aa, Homo sapiens
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 940 - protein, 219 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 941 - protein, 231 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 942 - protein, 215 aa, Artificial Sequence; identical to SEQ ID NO: 925
  Description of Artificial Sequence: Synthetic polypeptide
  MOD_RES (24)..(24): Arg or Gln
  MOD_RES (27)..(27): Gln or Glu
  MOD_RES (30)..(30): Ser or Asn
  MOD_RES (31)..(31): Ser or Asn
  MOD_RES (52)..(52): Ser or Tyr
  MOD_RES (53)..(53): Ser or Asn
  MOD_RES (56)..(56): Ser or Tyr
  MOD_RES (90)..(90): Gln or Leu
  MOD_RES (93)..(93): Ser or Lys

SEQ ID NO: 943 - protein, 452 aa, Artificial Sequence
  Description of Artificial Sequence: Synthetic polypeptide
QVQLVQSGAEVKKPGASVKVSCKASGYTFRSYGISWVRQAPGQGLEWMGWVAPYNGNTNYAQKLQGRVTMTTDTSTSTAYMELRSLRSDDTAVYYCARDAGTYSPYGYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 944 - protein, 215 aa, Artificial Sequence
  Description of Artificial Sequence: Synthetic polypeptide
DIQMTQSPSTLSASVGDRVTITCQASQSIDSWLAWYQQKPGKAPKLLIYSASYLESGVPSRFSGSGSGTEFTLTISSLQPDDFATYYCQRFQSLPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC